(19) United States
(12) Patent Application Publication
(10) Pub. No.: US 2009/0162400 A1
(43) Pub. Date: Jun. 25, 2009

(54) Compositions of influenza viral proteins and methods of use thereof

(76) Inventors: Thomas J. Powell, Madison, CT (US); James W. Huleatt, Nazareth, PA (US); Valerian Nakaar, Hamden, CT (US); Langzhou Song, Freehold, NJ (US); William F. McDonald, Madison, CT (US); Albert E. Price, New Haven, CT (US); Duane D. Hewitt, Hamilton (CA)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 11/820,148

(22) Filed: Jun. 18, 2007

Related U.S. Application Data
(63) Continuation-in-part of application No. PCT/US2005/046662, filed on Dec. 21, 2005.
Application No. PCT/US2005/046662 is a continuation-in-part of application No. 11/714,873, filed on Mar. 6, 2007.
(60) Provisional application No. 60/638,254, filed on Dec. 21, 2004.
(60) Provisional application No. 60/638,350, filed on Dec. 21, 2004.
(60) Provisional application No. 60/645,067, filed on Jan. 19, 2005.
(60) Provisional application No. 60/653,207, filed on Feb. 15, 2005.
(60) Provisional application No. 60/666,878, filed on Mar. 31, 2005.
(60) Provisional application No. 60/682,077, filed on May 18, 2005.
(60) Provisional application No. 60/741,202, filed on Nov. 30, 2005.
(60) Provisional application No. 60/779,854, filed on Mar. 7, 2006.
(60) Provisional application No. 60/784,497, filed on Mar. 20, 2006.
(60) Provisional application No. 60/790,457, filed on Apr. 7, 2006.
(60) Provisional application No. 60/814,292, filed on Jun. 16, 2006.
(60) Provisional application No. 60/830,881, filed on Jul. 14, 2006.
(60) Provisional application No. 60/838,007, filed on Aug. 16, 2006.
(60) Provisional application No. 60/856,451, filed on Nov. 3, 2006.
(60) Provisional application No. 60/933,554, filed on Jun. 6, 2007.

(51) Int. Cl. A61K 39/145; A61P 37/04

(52) U.S. Cl. 424/210.1

(57) ABSTRACT

Compositions, fusion proteins and polypeptides comprise at least one pathogen-associated molecular pattern and at least a portion of at least one integral membrane protein of an influenza viral antigen. The compositions, fusion proteins and polypeptides are used to stimulate an immune response in a subject.

### RELATED APPLICATIONS

This application is a continuation-in-part of International Application No. PCT/US2005/046662, which designated the United States and was filed on Dec. 21, 2005, published in English, which claims the benefit of U.S. Provisional Application Nos. 60/638,254, filed on Dec. 21, 2004; 60/638,350, filed on Dec. 21, 2004; 60/645,067, filed on Jan. 19, 2005; 60/653,207, filed on Feb. 15, 2005; 60/666,878, filed on Mar. 31, 2005; 60/682,077, filed on May 18, 2005; and 60/741,202, filed Nov. 30, 2005; this application is also a continuation-in-part of U.S. application Ser. No. 11/714,873, filed on Mar. 6, 2007, which claims the benefit of U.S. Provisional Application Nos. 60/779,854, filed on Mar. 7, 2006; 60/784,497, filed on Mar. 20, 2006; 60/790,457, filed on Apr. 7, 2006; 60/814,292, filed on Jun. 16, 2006; 60/830,881, filed on Jul. 14, 2006; 60/838,007, filed on Aug. 16, 2006; and 60/856,451, filed on Nov. 3, 2006; and this application also claims the benefit of U.S. Provisional Application, Attorney Docket No.: 3710.1036-000, filed Jun. 6, 2007 entitled “Methods of Making Immunogens.” The entire teachings of all of the above applications are incorporated herein by reference.

### BACKGROUND OF THE INVENTION

Influenza is a contagious disease that usually results from an RNA virus. Three types of influenza viruses are known—influenza type A, B and C. The natural host for influenza type A is the aquatic bird. Influenza type A viruses can infect humans, birds, farm animals (e.g., pigs, horses) and aquatic animals (e.g., seals). Influenza type B viruses are usually found only in humans. Infection with influenza is generally characterized by fever, myalgia, headache, cough and muscle aches. In the elderly and infirm, influenza type B infection can result in disability and death. Influenza type B viruses can cause epidemics in humans. Influenza type C viruses can cause mild illness in humans and do not cause epidemics. Strategies to prevent and manage influenza infection include vaccines with inactivated viruses, nasal sprays and drugs, such as amantadine (1-aminoadamantine hydrochloride), rimantadine, zanamivir and oseltamivir. However, such strategies can be costly to maintain supply with demand and, thus, be limited in supply; may result in variable protection and less than satisfactory alleviation of symptoms, thereby ineffectively preventing or treating illness and, in some instances death, consequent to influenza infection. Thus, there is a need to develop new, improved and effective methods of treatment for preventing and managing influenza infection.

### SUMMARY OF THE INVENTION

The present invention relates to compositions, fusion proteins and polypeptides comprising pathogen-associated molecular patterns (PAMPs) and influenza viral proteins. The compositions, fusion proteins and polypeptides of the invention can be employed in methods to stimulate an immune response in a subject.

In one embodiment, the invention is a composition comprising at least one Pam3Cys and at least a portion of at least one integral membrane protein of an influenza viral protein.

In another embodiment, the invention is a fusion protein comprising at least one pathogen-associated molecular pattern (PAMP) and at least one influenza M2 protein, wherein the pathogen-associated molecular pattern is not a Pam2Cys.

In a further embodiment, the invention is a composition comprising a pathogen-associated molecular pattern and an M2 protein, wherein the pathogen-associated molecular pattern is not a Pam2Cys.

In still another embodiment, the invention is a composition comprising at least a portion of at least one pathogen-associated molecular pattern and at least a portion of at least one influenza M2 protein, wherein, if the pathogen-associated molecular pattern includes a Pam2Cys, at least a portion of the Pam2Cys is not fused to the influenza M2 protein and at least a portion of the influenza M2 protein is not fused to the Pam2Cys.

In yet another embodiment, the invention is a fusion protein comprising at least a portion of at least one pathogen-associated molecular pattern and at least a portion of at least one influenza M2 protein, wherein, if the pathogen-associated molecular pattern includes a Pam2Cys, at least a portion of the Pam2Cys is not fused to the influenza M2 protein and at least a portion of the influenza M2 protein is not fused to the Pam2Cys.

In yet another embodiment, the invention is a method of stimulating an immune response in a subject, comprising the step of administering to the subject a composition that includes at least one Pam3Cys and at least a portion of at least one integral membrane protein of an influenza viral protein.

In still another embodiment, the invention is a method of stimulating an immune response in a subject, comprising the step of administering to the subject a composition that includes a fusion protein comprising at least one pathogen-associated molecular pattern and at least one influenza M2 protein, wherein the pathogen-associated molecular pattern is not a Pam2Cys.

In an additional embodiment, the invention is a method of stimulating an immune response in a subject, comprising the step of administering to the subject a composition that includes at least one pathogen-associated molecular pattern and at least one influenza M2 protein, wherein the pathogen-associated molecular pattern is not a Pam2Cys and the M2 protein is not an M2e protein.

In still another embodiment, the invention is a method of stimulating an immune response in a subject, comprising the step of administering to the subject a composition that includes a composition comprising at least a portion of at least one pathogen -associated molecular pattern and at least a portion of at least one influenza M2 protein, wherein, if the pathogen-associated molecular pattern includes a Pam2Cys, at least a portion of the Pam2Cys is not fused to the influenza M2 protein and at least a portion of the influenza M2 protein is not fused to the Pam2Cys.

In a further embodiment, the invention is a method of stimulating an immune response in a subject, comprising the step of administering to the subject a composition that includes a fusion protein comprising at least a portion of at least one pathogen-associated molecular pattern and at least a portion of at least one influenza M2 protein, wherein, if the pathogen-associated molecular pattern includes a Pam2Cys, at least a portion of the Pam2Cys is not fused to the influenza M2 protein and at least a portion of the influenza M2 protein is not fused to the Pam2Cys.

In yet another embodiment, the invention is a method of decreasing an antibody response to at least a portion of a flagellin that is a component of a fusion protein, wherein the fusion protein activates a Toll-like Receptor 5 and includes at least one antigen, comprising the step of deleting at least a portion of a hinge region of the flagellin.

In still another embodiment, the invention is a method of increasing the in vitro yield of a fusion protein, wherein the fusion protein activates a Toll-like Receptor 5 and includes at least a portion of at least one flagellin and at least a portion of at least one antigen, comprising the step of forming a fusion protein lacking at least a portion of a naturally occurring hinge region.

The compositions, fusion proteins and polypeptides of the invention can be employed to stimulate an immune response in a subject. Advantages of the claimed invention include, for example, cost effective compositions, fusion proteins and polypeptides that can be produced in relatively large quantities for use in the prevention and treatment of influenza infection. The claimed compositions, fusion proteins, polypeptides and methods can be employed to prevent or treat influenza infection and, therefore, avoid serious illness and death consequent to influenza infection.

### BRIEF DESCRIPTION OF THE FIGURES

FIG. 1 depicts the amino acid sequence of Salmonella typhimurium flagellin type 2 (fljB/STF2) with the hinge region underlined (SEQ ID NO: 1).

FIG. 2 depicts the nucleic acid sequence (SEQ ID NO: 2) encoding SEQ ID NO: 1. The nucleic acid sequence encoding the hinge region is underlined.

FIG. 3 depicts the amino acid sequence of fljB/STF2 without the hinge region (also referred to herein as “fljB/STF2Δ” or “STF2Δ”) (SEQ ID NO: 3).

FIG. 4 depicts the nucleic acid sequence (SEQ ID NO: 4) encoding SEQ ID NO: 3.

FIG. 5 depicts the amino acid sequence of E. coli flagellin fliC (also referred to herein as “E. coli fliC”) with the hinge region underlined (SEQ ID NO: 5).

FIG. 6 depicts the nucleic acid sequence (SEQ ID NO: 6) encoding SEQ ID NO: 5. The nucleic acid sequence encoding the hinge region is underlined.

FIG. 7 depicts the amino acid sequence of S. muenchen flagellin fliC (also referred to herein as “S. muenchen fliC”) with the hinge region underlined (SEQ ID NO: 7).

FIG. 8 depicts the nucleic acid sequence (SEQ ID NO: 8) encoding SEQ ID NO: 7. The nucleic acid sequence encoding the hinge region is underlined.

FIG. 9 depicts the amino acid sequence of pMT/STF2. The linker is underlined and the sequence of the BiP secretion signal is bolded (SEQ ID NO: 9).

FIG. 10 depicts the nucleic acid sequence (SEQ ID NO: 10) of SEQ ID NO: 9. The nucleic acid sequence encoding the linker is underlined and the nucleic acid sequence encoding the BiP sequence is bolded.

FIG. 11 depicts the nucleic acid sequence (SEQ ID NO: 17) encoding a multimer (4 units) of the amino-terminus of an M2 protein (also referred to herein as “4×M2e”).

FIG. 12 depicts an amino acid sequence (SEQ ID NO: 18) encoded by SEQ ID NO: 17.

FIG. 13 depicts the amino acid sequence (SEQ ID NO: 31) of a fusion protein (referred to herein as “fljB/STF2-4×M2e” or “fljB/STF2.4×M2e”) comprising fljB/STF2 and four, 24-amino acid sequences of an amino-terminus of an M2 protein.

FIG. 14 depicts the nucleic acid sequence (SEQ ID NO: 32) encoding SEQ ID NO: 31.

FIG. 15 depicts a Pam3Cys.M2e fusion protein. The amino acid sequence (SEQ ID NO: 13) of M2e is shown in bold type.

FIG. 16 depicts the activation of an antigen-presenting cell (APC) by Toll-like receptor (TLR) signaling.

FIGS. 17A and 17B depict plasmid constructs to express an amino-terminus of an M2 (e.g., SEQ ID NOS: 13, 47) of H1 and H5 (SEQ ID NO: 39) influenza A viral isolates. pMT: metallothionein promoter-based expression vector. BiP: secretion signal sequence of immunoglobulin-binding protein. STF2: full-length flagellin of S. typhimurium. STF2Δ: hinge region-deleted STF2. MCS: multiple cloning site.

FIG. 18 depicts plasmid constructs designed to express HA of H1 and H5 influenza A virus isolates. AOX1: AOX1 promoter of pPICZα expression vector (Invitrogen Corporation, Carlsbad, Calif.). αf: secretion signal sequence of yeast. STF2: full-length flagellin of S. typhimurium. STF2Δ: hinge region-deleted STF2. MCS: multiple cloning site.

FIG. 19 depicts the amino acid sequence (SEQ ID NO: 60) of the STF2Δ.HA fusion protein with the linker between STF2Δ(STF2 minus its hinge region) and HA underlined.

FIG. 20 depicts the nucleic acid sequence (SEQ ID NO: 61) encoding SEQ ID NO: 60. The linker is underlined.

FIG. 21 depicts the amino acid sequence (SEQ ID NO: 62) of the STF2Δ.HA (Puerto Rico 8 (PR8) strain of influenza A virus) fusion protein with the linker between STF2Δ and HA underlined.

FIG. 22 depicts the nucleic acid sequence (SEQ ID NO: 63) encoding SEQ ID NO: 62. The linker is underlined.

FIG. 23 depicts the amino acid sequence (SEQ ID NO: 64) of HA (PR8).

FIG. 24 depicts the nucleic acid sequence (SEQ ID NO: 65) encoding SEQ ID NO: 64.

FIG. 25 depicts the amino acid sequence (SEQ ID NO: 66) of E. coli fliC without the hinge region.

FIG. 26 depicts the amino acid sequence of influenza A H5N1 HA (SEQ ID NO: 67).

FIG. 27 depicts the nucleic acid sequence (SEQ ID NO: 68) encoding SEQ ID NO: 67.

FIG. 28 depicts the amino acid sequence of pMT/STF2.4×M2e (H1) (SEQ ID NO: 82). The linker sequence between STF2 and 4×M2e is underlined and the Drosophila BiP secretion signal is bolded.

FIG. 29 depicts the nucleic acid sequence (SEQ ID NO: 83) encoding SEQ ID NO: 82. The nucleic acid sequence encoding the linker is underlined and the nucleic acid sequence encoding the BiP secretion signal is bolded.

FIG. 30 depicts the amino acid sequence pMT/STF2.4×M2e (H5) (SEQ ID NO: 84). The linker sequence between STF2 and 4×M2e is underlined and the BiP secretion signal is bolded.

FIG. 31 depicts the nucleic acid sequence (SEQ ID NO: 85) encoding SEQ ID NO: 84. The nucleic acid sequence encoding the linker is underlined and the nucleic acid sequence encoding the BiP secretion signal is bolded.

FIG. 32 depicts the amino acid sequence of pMT/STF2.4×M2e (H1H5) (SEQ ID NO: 86). The linker sequence between the STF2 and 4×M2e sequence is underlined and the BiP secretion signal is bolded.

FIG. 33 depicts the nucleic acid sequence (SEQ ID NO: 87) encoding SEQ ID NO: 86. The nucleic acid sequence encoding the linker is underlined and the nucleic acid sequence encoding the BiP secretion signal is bolded.

FIG. 34 depicts the amino acid sequence of pMT/STF2Δ (SEQ ID NO: 88). The linker sequence is underlined and the BiP secretion signal is bolded.

FIG. 35 depicts the nucleic acid sequence (SEQ ID NO: 89) encoding SEQ ID NO: 88. The nucleic acid sequence encoding the linker is underlined and the nucleic acid sequence encoding the BiP secretion signal is bolded.

FIG. 36 depicts the amino acid sequence of pMT/STF2Δ.4×M2e (H1) (SEQ ID NO: 90). The linker sequence is underlined and the BiP secretion signal sequence is bolded.

FIG. 37 depicts the nucleic acid sequence (SEQ ID NO: 91) encoding SEQ ID NO: 90. The nucleic acid sequence encoding the linker is underlined and the nucleic acid sequence encoding the BiP secretion signal is bolded.

FIG. 38 depicts the amino acid sequence of pMT/STF2Δ.4×M2e (H5) (SEQ ID NO: 92). The linker sequence is underlined and the BiP secretion signal is bolded.

FIG. 39 depicts the nucleic acid sequence (SEQ ID NO: 93) encoding SEQ ID NO: 92. The nucleic acid sequence encoding the linker is underlined and the nucleic acid sequence encoding the BiP secretion signal is bolded.

FIG. 40 depicts the amino acid sequence pMT/STF2Δ.4×M2e (H1H5) (SEQ ID NO: 94). The linker sequence is underlined and the BiP secretion signal is bolded.

FIG. 41 depicts the nucleic acid sequence (SEQ ID NO: 95) encoding SEQ ID NO: 94. The nucleic acid sequence encoding the linker is underlined and the nucleic acid sequence encoding the BiP secretion signal is bolded.

FIG. 42 depicts the amino acid sequence (SEQ ID NO: 98) of the Salmonella muenchen fliC without the hinge region, which is also referred to herein as “S. muenchen fliCΔ.”

FIG. 43 depicts the nucleic acid sequence of Salmonella muenchen fliC (SEQ ID NO: 99) encoding SEQ ID NO: 98.

FIG. 44 depicts IL-8 secretion following stimulation of TLR5+ cells.

FIG. 45 depicts TNF secretion following stimulation of TLR2+ cells.

FIG. 46 depicts M2e-specific IgG.

FIG. 47 depicts the OVA-specific IgG.

FIG. 48 depicts the M2e-specific IgG serum titers.

FIG. 49 depicts the M2e-specific serum IgG titer post-boost.

FIG. 50 depicts the Pam3Cys.M2e dose response.

FIG. 51 depicts the M2e-specific serum IgG titer.

FIG. 52 depicts the rabbit IgG response to M2e.

FIG. 53 depicts the immunogenicity of STF2.4×M2e in a rabbit 14 days post-prime.

FIG. 54 depicts survival following viral challenge.

FIG. 55 depicts the D1 domain, D2 domain, TLR5 activation domain and hypervariable (D3 domain) of flagellin.

FIG. 56 depicts the D1 domain, D2 domain, TLR5 activation domain and hypervariable (D3 domain) of flagellin (Yonekura, et al. Nature 424, 643-650 (2003)).

FIG. 57 depicts the amino acid sequence of Pseudomonas aeruginosa flagellin (SEQ ID NO: 241).

FIG. 58 depicts the amino acid sequence (SEQ ID NO: 242) of Listeria monocytogenes flagellin (GenBank Accession No: Q92DW3).

FIGS. 59A and 59B depict the amino acid sequences of PR8 (SEQ ID NO: 107), New Caledonia (SEQ ID NO: 108), Solomon Island (SEQ ID NO: 109) hemagglutinins and conserved amino acids (SEQ ID NO: 110).

FIG. 60 depicts the amino acids sequences (SEQ ID NOs: 111, 112 and 113) of fusion proteins.

FIGS. 61A, 61B, 61C and 61D depict the antigenicity of STF2.HA1-2(SI) and STF2Δ.HA1-2(SI) proteins. Results reflect the OD450 of samples performed in replicate.

FIGS. 62A, 62B, 62C and 62D depict cross-reactivity of rabbit polyclonal anti-HA with STF2.HA1-2(SI) and STF2Δ.HA1-2(SI) proteins.

FIGS. 63A and 63B depict serum antibody response in mice immunized with recombinant HA(SI) fusion proteins. Results show mean±SD of 10 individual sera per treatment group.

FIG. 64 depicts virus neutralization titers of sera from mice immunized with STF2.HA1-2(SI) and STF2Δ.HA1-2(SI) fusion proteins. Naïve ferret serum yielded a titer of about 86, and immune ferret serum yielded a titer of about 1215.

FIG. 65 depicts hemagglutination inhibition titers of sera from mice immunized with STF2.HA1-2(SI) and STF2Δ.HA1-2(SI) fusion proteins. Results show the geometric mean±SD of 10 individual sera per group, with the exception of the single ferret immune serum. Naïve mouse sera failed to inhibit hemagglutination in contrast to the high background of naïve mouse serum in the neutral red assay (FIG. 64).

FIG. 66 depicts HI titers of the individual mice shown in FIG. 65.

FIGS. 67A and 67B depict antigenicity of STF2Δ.HA1-2(PR8) fusion proteins. Results reflect the OD450 of samples performed in replicate.

FIGS. 68A and 68B depict immunogenicity of STF2Δ.HA1-2 (PR8) fusion proteins in BALB/c mice. The data depict the mean±SD of 10 individual sera per group.

FIGS. 69A and 69B depict antigenicity of STF2Δ.4×M2e and STF2.4×M2e fusion proteins. Proteins were coated on ELISA plates at the concentrations indicated on the x-axes, then probed with antibody to M2e (FIG. 69A) or flagellin (FIG. 69B).

FIGS. 70A and 70B depict immunogenicity of STF2Δ.4×M2e and STF2.4×M2e fusion proteins. Sera were analyzed for M2e-specific antibodies (FIG. 70A) and flagellin-specific antibodies (FIG. 70B) by ELISA. Results represent the mean±SD of 10 samples per group.

FIG. 71 depicts efficacy of STF2Δ.4×M2e and STF2.4×M2e fusion proteins. BALB/c mice from FIGS. 70A and 70B were challenged 7 days post-boost by intranasal administration of an LD₉₀ of influenza A/Puerto Rico/8/34 virus and monitored for survival for 21 days post-challenged.

FIGS. 72A, 72B and 72C depict TLR5 bioactivity and antigenicity of STF2.4×M2e.

FIGS. 73A, 73B and 73C depicts immunogenicity of STF2.4×M2e

FIGS. 74A and 74B depict immunogenicity of STF2.4×M2e in rabbits. Values are ΔOD means (individual pre-bleeds subtracted) of 6 rabbits per group.

FIG. 75 depicts epitope specificity of antibody response induced by STF2.4×M2e of the anti-M2e IgG induced by STF2.4×M2e as described in FIGS. 74A and 74B by probing an overlapping peptide array spanning the entire 24 residue sequence (SEQ ID NOS: 245-257).

FIG. 76 depicts recognition of influenza-infected cells by antisera of STF2.4×M2e-immunized mice. Data represent the ΔOD₄₅₀ (infected minus mock infected).

FIGS. 77A, 77B and 77C depict efficacy of STF2.4×M2e immunization. Data reflect results of 10 mice/group. Clinical Score 4=healthy, 3=ruffled coat, 2=reduced motility and 1=moribund).

FIGS. 78A, 78B and 78C depict immunogenicity and efficacy of STF2 fused to 4×M2e.

### DETAILED DESCRIPTION OF THE INVENTION

The features and other details of the invention, either as steps of the invention or as combinations of parts of the invention, will now be more particularly described and pointed out in the claims. It will be understood that the particular embodiments of the invention are shown by way of illustration and not as limitations of the invention. The principle features of this invention can be employed in various embodiments without departing from the scope of the invention.

In one embodiment, the invention is a composition comprising at least one Pam3Cys ([Palmitoyl]-Cys((RS)-2,3-di(palmitoyloxy)-propyl cysteine) and at least a portion of at least one integral membrane protein of an influenza viral protein. Pam3Cys (also referred to herein as “P2”) is a Toll-like receptor 2 (TLR2) agonist.

The compositions can include, for example, two, three, four, five, six or more pathogen-associated molecular patterns (e.g., Pam2Cys, Pam3Cys) and two, three, four (e.g., SEQ ID NOS: 17 and 18), five, six or more integral membrane proteins of an influenza viral protein. When two or more PAMPs and/or two or more influenza viral proteins comprise the compositions, fusion proteins and polypeptides of the invention, they are also referred to as “multimers.” For example, a multimer of the amino-terminus of an M2 protein can be four, 24-amino acid sequences (total of 96 amino acids), which is referred to herein as 4×M2 or 4×M2e (“M2e” refers to the 24 amino acid amino-terminus of the M2 protein or its ectodomain).

Pathogen-associated molecular pattern (PAMP) refers to a class of molecules (e.g., proteins, peptide, carbohydrates, lipids) found in microorganisms that when bound to a pattern recognition receptor (PRR) can trigger an innate immune response. The PRR can be a Toll-like receptor (TLR). Toll-like receptors refer to a family of receptor proteins that are homologous to the Drosophila melangogaster Toll protein. Toll-like receptors are type I transmembrane signaling receptor proteins characterized by an extracellular leucine-rich repeat domain and an intracellular domain homologous of that of the interleukin 1 receptor. Toll-like receptors include TLR1, TLR2, TLR3, TLR4, TLR5, TLR6, TLR7, TLR8, TLR9, TLR10, TLR11 and TLR12.

The pathogen-associated molecular pattern can be an agonist of a toll-like receptor, for example, a TLR2 agonist, such as Pam3Cys. “Agonist,” as used herein in referring to a TLR, means a molecule that activates a TLR signaling pathway. A TLR signaling pathway is an intracellular signal transduction pathway employed by a particular TLR that can be activated by a TLR ligand or a TLR agonist. Common intracellular pathways are employed by TLRs and include, for example, NF-κB, Jun N-terminal kinase and mitogen-activated protein kinase. The pathogen-associated molecular pattern can include at least one member selected from the group consisting of a TLR1 agonist, a TLR2 agonist (e.g., Pam3Cys, Pam2Cys), a TLR 3 agonist (e.g., dsRNA), a TLR 4 agonist (e.g., bacterial lipopolysaccharide), a TLR 5 agonist (e.g., flagellin), a TLR 6 agonist, a TLR 7 agonist, a TLR 8 agonist, a TLR 9 agonist (e.g., unmethylated DNA motifs), TLR10 agonist, a TLR11 agonist and a TLR12 agonist.

TLR4 ligands (e.g., TLR4 agonists) for use in the compositions and methods of the invention can include at least one member selected from the group consisting of SEQ ID NOS: 359-406 (see, PCT/US 2006/002906/WO 2006/083706; PCT/JUS 2006/003285/WO 2006/083792; PCT/US 2006/041865; PCT/US 2006/042051; U.S. application Ser. No. 11/714,873).

| | |
|---|---|
| GGKSGRTG | (SEQ ID NO: 141) |
| KGYDWLVVG | (SEQ ID NO: 142) |
| EDMVYRIGVP | (SEQ ID NO: 143) |
| VKLSGS | (SEQ ID NO: 144) |
| GMLSLALF | (SEQ ID NO: 145) |
| CVVGSVR | (SEQ ID NO: 146) |
| IVRGCLGW | (SEQ ID NO: 147) |
| AAEERTLG | (SEQ ID NO: 148) |
| WARVVGWLR | (SEQ ID NO: 149) |
| SEGYRLFGG | (SEQ ID NO: 150) |
| LVGGVVRRGS | (SEQ ID NO: 151) |
| GRVNDLWLAA | (SEQ ID NO: 152) |
| SGWMLWREGS | (SEQ ID NO: 153) |
| ERMEDRGGDL | (SEQ ID NO: 154) |
| KLCCFTECM | (SEQ ID NO: 155) |
| AVGSMERGRG | (SEQ ID NO: 156) |
| RDWVGGDLV | (SEQ ID NO: 157) |
| FFEVAKISQQ | (SEQ ID NO: 158) |
| WWYWC | (SEQ ID NO: 159) |
| MHLCSHA | (SEQ ID NO: 160) |
| WLFRRIG | (SEQ ID NO: 161) |
| YWFWRIG | (SEQ ID NO: 162) |
| MHLYCIA | (SEQ ID NO: 163) |
| WPLFPWIV | (SEQ ID NO: 164) |
| DMRSHAR | (SEQ ID NO: 165) |
| MHLCTHA | (SEQ ID NO: 166) |
| NLFPFY | (SEQ ID NO: 167) |
| MHLCTRA | (SEQ ID NO: 168) |
| RHLWYHA | (SEQ ID NO: 169) |
| WPFSAYW | (SEQ ID NO: 170) |
| WYLRGS | (SEQ ID NO: 171) |
| GKGTDLG | (SEQ ID NO: 172) |
| IFVRMR | (SEQ ID NO: 173) |
| WLFRPVF | (SEQ ID NO: 174) |
| FLGWLMG | (SEQ ID NO: 175) |
| MHLWHHA | (SEQ ID NO: 176) |
| WWFPWKA | (SEQ ID NO: 177) |
| WYLPWLG | (SEQ ID NO: 178) |
| WPFPRTF | (SEQ ID NO: 179) |
| WPFPAYW | (SEQ ID NO: 180) |
| FLGLRWL | (SEQ ID NO: 181) |
| SRTDVGVLEV | (SEQ ID NO: 182) |
| REKVSRGDKG | (SEQ ID NO: 183) |
| DWDAVESEYM | (SEQ ID NO: 184) |
| VSSAQEVRVP | (SEQ ID NO: 185) |
| LTYGGLEALG | (SEQ ID NO: 186) |
| VEEYSSSGVS | (SEQ ID NO: 187) |
| VCEVSDSVMA | (SEQ ID NO: 188) |

TLR2 ligands (e.g., TLR2 agonists) for use in the compositions and methods of the invention can also include at least one member selected from the group consisting of SEQ ID NOS: 455-494 (see, PCT/US 2006/002906/WO 2006/083706; PCT/US 2006/003285/WO 2006/083792; PCT/US 2006/041865; PCT/US 2006/042051; U.S. application Ser. No. 11/714,873).

| | |
|---|---|
| NPPTT | (SEQ ID NO: 189) |
| MRRIL | (SEQ ID NO: 190) |
| MISS | (SEQ ID NO: 191) |
| RGGSK | (SEQ ID NO: 192) |
| RGGF | (SEQ ID NO: 193) |
| NRTVF | (SEQ ID NO: 194) |
| NRFGL | (SEQ ID NO: 195) |
| SRHGR | (SEQ ID NO: 196) |
| IMRHP | (SEQ ID NO: 197) |
| EVCAP | (SEQ ID NO: 198) |
| ACGVY | (SEQ ID NO: 199) |
| CGPKL | (SEQ ID NO: 200) |
| AGCFS | (SEQ ID NO: 201) |
| SGGLF | (SEQ ID NO: 202) |
| AVRLS | (SEQ ID NO: 203) |
| GGKLS | (SEQ ID NO: 204) |
| VSEGV | (SEQ ID NO: 205) |
| KCQSF | (SEQ ID NO: 206) |
| FCGLG | (SEQ ID NO: 207) |
| PESGV | (SEQ ID NO: 208) |
| DPDSG | (SEQ ID NO: 209) |
| IGRFR | (SEQ ID NO: 210) |
| MGTLP | (SEQ ID NO: 211) |
| ADTHQ | (SEQ ID NO: 212) |
| HLLPG | (SEQ ID NO: 213) |
| GPLLH | (SEQ ID NO: 214) |
| NYRRW | (SEQ ID NO: 215) |
| LRQGR | (SEQ ID NO: 216) |
| IMWFP | (SEQ ID NO: 217) |
| RVVAP | (SEQ ID NO: 218) |
| IHVVP | (SEQ ID NO: 219) |
| MFGVP | (SEQ ID NO: 220) |
| CVWLQ | (SEQ ID NO: 221) |
| IYKLA | (SEQ ID NO: 222) |
| KGWF | (SEQ ID NO: 223) |
| KYMPH | (SEQ ID NO: 224) |
| VGKND | (SEQ ID NO: 225) |
| THKPK | (SEQ ID NO: 226) |
| SHIAL | (SEQ ID NO: 227) |
| AWAGT | (SEQ ID NO: 228) |

The TLR2 ligand (e.g., TLR2 agonist) can also include at least a portion of at least one member selected from the group consisting of flagellin modification protein FlmB of Caulobacter crescentus; Bacterial Type III secretion system protein; invasin protein of Salmonella; Type 4 fimbrial biogenesis protein (PilX) of Pseudomonas; Salmonella SciJ protein; putative integral membrane protein of Streptomyces; membrane protein of Pseudomonas; adhesin of Bordetella pertusis; peptidase B of Vibrio cholerae; virulence sensor protein of Bordetella; putative integral membrane protein of Neisseria meningitidis; fusion of flagellar biosynthesis proteins FliR and FlhB of Clostridium; outer membrane protein (porin) of Acinetobacter; flagellar biosynthesis protein FlhF of Helicobacter; ompA related protein of Xanthomonas; omp2a porin of Brucella; putative porin/fimbrial assembly protein (LHrE) of Salmonella; wbdk of Salmonella; Glycosyltransferase involved in LPS biosynthesis; Salmonella putative permease.

The TLR2 ligand (e.g., TLR agonist) can include at least a portion of at least one member selected from the group consisting of lipoprotein/lipopeptides (a variety of pathogens); peptidoglycan (Gram-positive bacteria); lipoteichoic acid (Gram-positive bacteria); lipoarabinomannan (mycobacteria); a phenol-soluble modulin (Staphylococcus epidermidis); glycoinositolphospholipids (Trypanosoma Cruzi); glycolipids (Treponema maltophilum); porins (Neisseria); zymosan (fungi) and atypical LPS (Leptospira interrogans and Porphyromonas gingivalis).

The TLR2 ligand (e.g., TLR2 agonist) can also include at least one member selected from the group consisting of SEQ ID NOS: 495-497 (see, PCT/US 2006/002906/WO 2006/083706; PCT/US 2006/003285/WO 2006/083792; PCT/US 2006/041865; PCT/US 2006/042051; U.S. application Ser. No. 11/714,873).

| | |
|---|---|
| KGGVGPVRRSSRLRRTTQPG | (SEQ ID NO: 229) |
| GRRGLCRGCRTRGRIKQLQSAHK | (SEQ ID NO: 230) |
| RWGYHLRDRKYKGVRSHKGVPR | (SEQ ID NO: 231) |

Influenza viruses are divided into three types (i.e., A, B, C) determined by the antigenic differences in ribonucleoprotein (RNP) and matrix (M) antigens of the viruses. Influenza A virus can cause epidemics and pandemics and has an avian intermediate host. Influenza B virus appears to naturally infect only humans and can cause epidemics in humans. It naturally infects humans and several other mammalian species, including swine and horses, and a wide variety of avian species. Influenza C virus has been isolated from humans and swine, but generally does not occur in epidemics and usually results in mild disease in humans.

Influenza A virus, influenza B virus and influenza C virus belong to the viral family Orthomyxoviridae. Virions of the genera influenza A virus, influenza B virus and influenza C virus contain a single stranded, negative sense, segmented RNA genome and are enveloped with a pleomorphic structure ranging in diameter from 80-120 nm. The single-stranded RNA genome is closely associated with a helical nucleoprotein and is present in seven (influenza C) or eight (influenza A and B) separate segments of ribonucleoprotein (RNP), each of which has to be present for successful replication of the virus. The segmented genome is enclosed within an outer lipoprotein envelope. Matrix protein 1 (MP1 or also referred to herein as “M1”) lines the inside of the outer lipoprotein envelope and is bound to the RNP.

The outer lipoprotein envelope of the influenza virus has two types of protruding spikes. One of the protruding spikes is the integral membrane protein neuraminidase (NA), which has enzymatic properties. The other envelope spike is the trimeric integral membrane protein haemagglutinin (HA), which participates in attachment of the virus particle to a cell membrane and can combine with specific receptors on a variety of cells, including red blood cells. The outer lipoprotein envelope makes the virion labile and susceptible to heat, drying, detergents and solvents.

Matrix protein 2 (M2 or M2 protein) is a proton-selective integral membrane ion channel protein of the influenza A virus. M2 is abundantly expressed at the plasma membrane of virus-infected cells, but is generally underexpressed by virions. For example, a portion of an M2 sequence of influenza A is MSLLTEVETPIRNEWGCRCNDSSDPLVVAASIIGILHLILWILDRLFFKClYRLFK HGLKRGPSTEGVPESMREEYRKEQQNAVDADDSHFVSIELE (SEQ ID NO: 11), which is encoded by ATGAGCCTTCTAACCGAGGTCGAAACACCTATCAGAAACGAATGGGGGTGC AGATGCAACGATTCAAGTGACCCGCTTGTTGTTGCCGCGAGTATCATTGGGA TCTTGCACTTGATATTGTGGATTCTTGATCGTCTTTTTTTCAAATGCATCTAT CGACTCTTCAAACACGGCCTTAAAAGAGGGCCTTCTACGGAAGGAGTACCT GAGTCTATGAGGGAAGAATATCGAAAGGAACAGCAGAATGCTGTGGATGCT GACGACAGTCATTTTGTCAGCATAGAGTTGGAGTAA (SEQ ID NO: 12). The native form of the M2 protein is a homotetramer (i.e., four identical disulfide-linked M2 protein molecules). Each of the units are helices stabilized by two disulfide bonds. M2 is activated by low pH. Each of the M2 protein molecules in the homotetramer consists of three domains: a 24 amino acid outer or N (amino)-terminal domain (e.g., SLLTEVETPIRNEWGCRCNDSSDP (SEQ ID NO: 13; also referred to herein as a “human consensus sequence”), which is encoded by ATGAGCCTGCTGACCGAGGTCGAAACACCGATCCGCAACGAATGGGGGTGC CGCTGCAACGATTCAAGTGACCCG (SEQ ID NO: 14); a 19 hydrophobic amino acid transmembrane region, and a 54 amino acid inner or C (carboxy)-terminal domain. The M2 protein can vary depending upon the influenza viral subtype (e.g., H1 and H5 subtypes of influenza A) and influenza viral source (e.g., Puerto Rico, Thailand, New York, Hong Kong), as shown, for example, in exemplary amino-terminal sequences of M2 proteins in Table 1 (infra).

The M2 protein has an important role in the life cycle of the influenza A virus. It is important in the uncoating stage where it permits the entry of protons into the viral particle, which lowers the pH inside the virus, resulting in dissociation of the viral matrix protein M1 from the ribonucleoprotein RNP. As a consequence, the virus coat is removed and the contents of the virus are released from the endosome into the cytoplasm of the host cell for infection.

The function of the M2 channel can be inhibited by antiviral drugs, such as amantadine and rimantadine, which prevent the virus from infecting the host cell. Such antiviral drugs usually bind the transmembrane region of the M2 protein and sterically block the ion channel created by the M2 protein, which prevents protons from entering and uncoating the virion.

As discussed above, M2, HA and NA are integral membrane proteins (e.g., proteins that extend from the outer surface of the virus to the inner surface of the virus) of influenza viruses (influenza A, B, C). “At least a portion,” as used herein in reference to an integral membrane protein of an influenza virus, means any part of an entire integral membrane protein. For example, the 24 amino acid N-terminus of the M2 protein (e.g., SEQ ID NO: 13), EVETPIRNEWG (SEQ ID NO: 15), EVETPIRNE (SEQ ID NO: 19), EVETPIRNEW (SEQ ID NO: 34) or EVETPIRN (SEQ ID NO: 20) is at least a portion of an M2 protein; and PAKLLKERGRRGAIAGFLE (SEQ ID NO: 33) is at least a portion of an HA protein. SEQ ID NO: 15 encoded by GAGGTTGAGACCCCGATTCGCAACGAATGGGGT (SEQ ID NO: 96). The protein encoded by GAGGTCGAAACACCTATCAGAAACGAATGG (SEQ ID NO: 16) is also at least a portion of M2.

At least a portion of a hemagglutinin (e.g., an influenza A, an influenza B and an influenza C viral hemagglutinin) can include at least one member selected from the group consisting of protein portions referred to herein as “HA1-1,” “HA1-2” and “HA1-3.”

“HA1-1,” as used herein, refers to a portion of a viral hemagglutinin that includes at least about one β-sandwich that includes the substrate binding site, which includes at least about two β-sheets, at least about two to about three short α-helixes, at least one small β-sheet and at least one additional small β-sandwich at the bottom of the molecule and at least about four disulfide bonds. The β-sandwich that includes the substrate binding site of the HA 1-1 includes at least about four β-strands as the top sheet and at least about three to about four β-strands as the bottom sheet. At least about one α-helix of the HA1-1 portion is located by the side of β-sandwich that includes the substrate binding site and at least about one to about two are located at the bottom of the β-sandwich that includes the substrate binding site. The small β-sandwich of the HA 1-1 can include at least about two to about three β-strands in each β-sheet; or about three to about four β-strands. Exemplary HA1-1 protein portions include SEQ ID NOS: 114-122.

“HA1-2,” as used herein, refers to a portion of a viral hemagglutinin that includes at least about one β-sandwich that includes the substrate binding site, at least about two to about three short α-helixes, at least about one small β-sheet at the bottom of the molecule and at least about two disulfide bonds. A β-strand in a viral hemagglutinin can include between about two to about 15 amino acids. A small β-strand can include about two amino acids; or between about two to about three amino acids; or between about two to four amino acids or between about two to about five amino acids. A small β-sheet can include between about two to about three β-strands; or between about three to about four β-strands. The β-sandwich that includes the substrate binding site of HA1-2 can further include at least about four β-strands as the top sheet and at least about three to about four β-strands as the bottom sheet. At least about one α-helix of the HA1-2 portion is located by the side of the β-sandwich that includes the substrate binding site and at least about one to about two are located at the bottom of the β-sandwich that includes the substrate binding site. Exemplary HA1-2 protein portions include SEQ ID NOS: 123-132.

“HA1-3,” as used herein, refers to a portion of a viral hemagglutinin that includes at least one β-sandwich that includes the substrate binding site, at least about two short α-helixes and at least one disulfide bond. “β-sandwich,” as used herein, refers to at least about two sets of beta-sheets that form at least about one interactive layer. “Substrate binding site,” as used herein in reference to the β-sandwich, means any part of the portion of the naturally occurring viral hemagglutinin that has the capacity to interact or bind to a molecule. For example, the β-sandwich that includes the substrate binding site of the portion can include a portion that binds sialic acid. The β-sandwich that includes the substrate binding site of HA1-3 can further include at least about four β-strands as the top sheet and at least about three β-strands as the bottom sheet. At least about one α-helix of the HA1-1 portion is located by the side of the β-sandwich that includes the substrate binding site and at least one other α-helix is located at the bottom of the β-sandwich that includes the substrate binding site. A short α-helix can include less than about 5 turns (2, 3, 4, 5 turns) in an α-helix. An α-helix in a viral hemagglutinin can be between one to about 15 turns; or between about two to 15 turns. Exemplary HA1-3 portions include SEQ ID NOS: 133-140.

The compositions, fusion proteins and polypeptides of the invention can include at least one member selected from the group consisting of an influenza A viral protein, influenza B viral protein and an influenza C viral protein. The influenza viral protein can include an integral membrane protein that includes at least one member selected from the group consisting of a haemagglutinin integral membrane protein, a neuraminidase integral membrane protein and an M2 integral membrane protein.

The integral membrane protein can include an M2 protein that includes at least a portion of SLLTEVETPIRNEWGCRCNDSSDP (SEQ ID NO: 13) encoded by SEQ ID NO: 14 or at least a portion of SEQ ID NO: 47, encoded by AGCTTGCTGACTGAGGTTGAGACCCCGATTCGCAACGAATGGGGTTCCCGTT CCAACGATTCTTCCGACCCG (SEQ ID NO: 106). The M2 protein can further include at least one member selected from the group consisting of EVETPIRNEWG (SEQ ID NO: 15), EVETPIRNE (SEQ ID NO: 19), EVETPIRNEW (SEQ ID NO: 34); SLLTEVETPTRNEWESRSSDSSDP (SEQ ID NO: 39) (Flu A H5N1 M2e, 2004 Viet Nam Isolate with serine replacing cysteine); SLLTEVETPTRNEWECRCSDSSDP (SEQ ID NO: 40) (Flu A H5N1 M2e, 2004 Viet Nam Isolate); SLLTEVETLTRNGWGSRSSDSSDP (SEQ ID NO: 41) (Flu A H5N1 M2e, Hong Kong 97 Isolate with serine replacing cysteine); SLLTEVETLTRNGWGCRCSDSSDP (SEQ ID NO: 42) (Flu A H5N1 M2e, Hong Kong 97 Isolate); SLLTEVETPTRNGWESKSSDSSDP (SEQ ID NO: 43) (Flu A H7N2 M2e Chicken/New York 95 Isolate with serine replacing cysteine); SLLTEVETPTRNGWECKCSDSSDP (SEQ ID NO: 44) (Flu A H7N2 M2e, Chicken/New York 95 Isolate); SLLTEVETLTRNGWESKSRDSSDP (SEQ ID NO: 45) (Flu A H9N2 M2e, Hong Kong 99 Isolate with serine replacing cysteine); and SLLTEVETLTRNGWECKCRDSSDP (SEQ ID NO: 46) (Flu A, Hong Kong 99 Isolate). Certain cysteine residues, for example, amino acids 16 and 18 of SEQ ID NO: 40; amino acids 17 and 19 of SEQ ID NOS: 42, 44 and 46 in the naturally occurring sequence of at least a portion of M2 protein are replaced with a serine (see, SEQ ID NOS: 41, 43, 45 and 47, respectively).

The integral membrane protein can include a haemagglutinin protein that includes, for example, at least a portion of SEQ ID NOS: 64 and 67, encoded by SEQ ID NOS: 65 and 68, respectively. The haemagglutinin protein can include at least a portion of at least one member selected from the group consisting of PAKLLKERGRRGAIAGFLE (SEQ ID NO: 33) (Influenza B); SLWSEEPAKLLKERGFFGAIAGFLEE (SEQ ID NO: 35) (Flu B); SLWSEENIPSIQSRGLFGAIAGFIEE (SEQ ID NO: 36) (FluA H1/H0); SLWSEENVPEKQTRGIFGAIAGFIEE (SEQ ID NO: 37) (Flu A H3/H0); SLWSEEEWEERERRRKKRGLFGAIAGFIEE (SEQ ID NO: 38) (Flu A H5/H0); PAKLLKERGFFGAIAGFLEE (SEQ ID NO: 102) (Flu B); NIPSIQSRGLFGAIAGFIEE (SEQ ID NO: 103) (Flu A H1/H0); NVPEKQTRGIFGAIAGFIEE (SEQ ID NO: 104) (Flu A H3/H0); and RERRRKKRGLFGAIAGFIEE (SEQ ID NO: 105) (Flu A H5/H0).

The composition comprising at least one Pam3Cys and at least a portion of at least one integral membrane protein of an influenza viral protein can further include at least one Pam2Cys (S—[2,3-bis(palmitoyloxy)propyl] cysteine). The composition of at least one Pam3Cys, at least one Pam2Cys and at least a portion of at least one integral membrane protein can be components of a fusion protein. The composition comprising at least one Pam3Cys and at least a portion of at least one integral membrane protein of an influenza viral protein can also be components of a fusion protein.

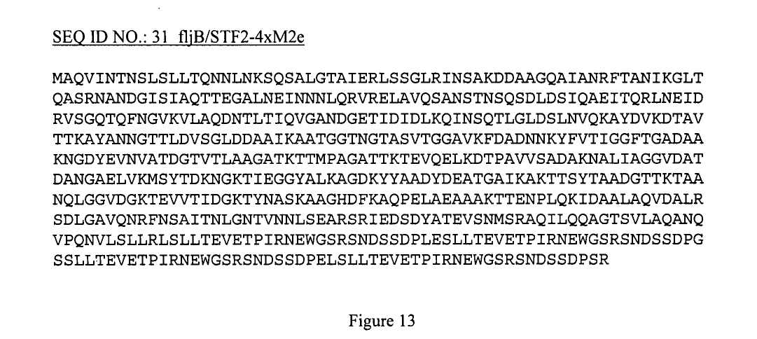

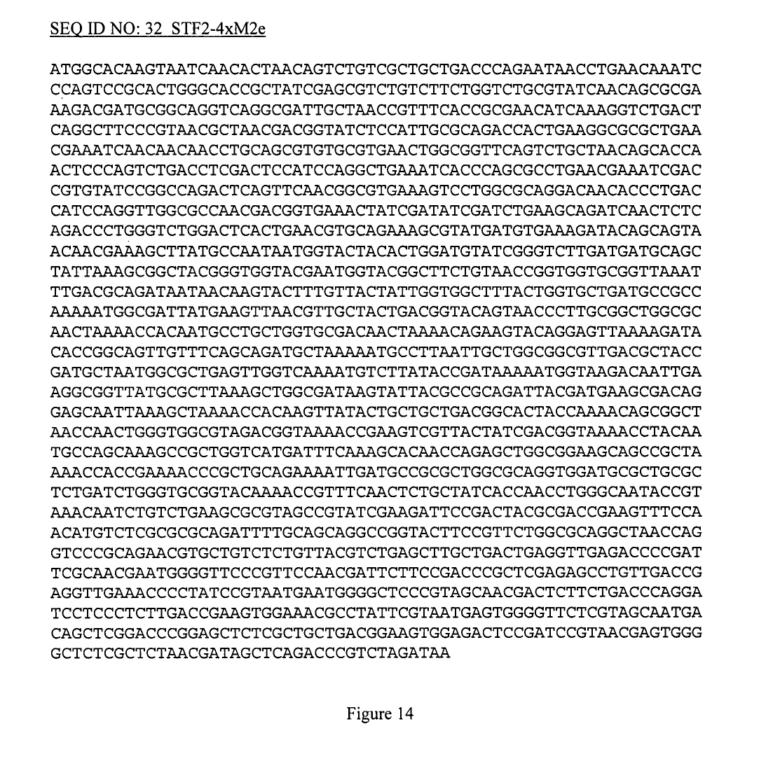

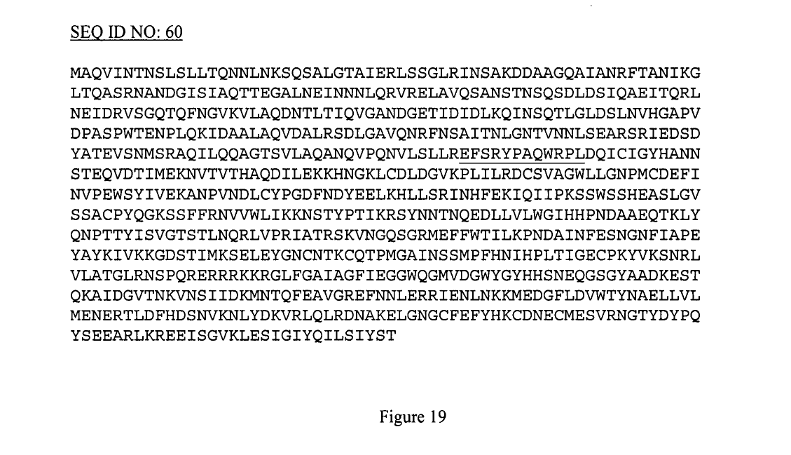

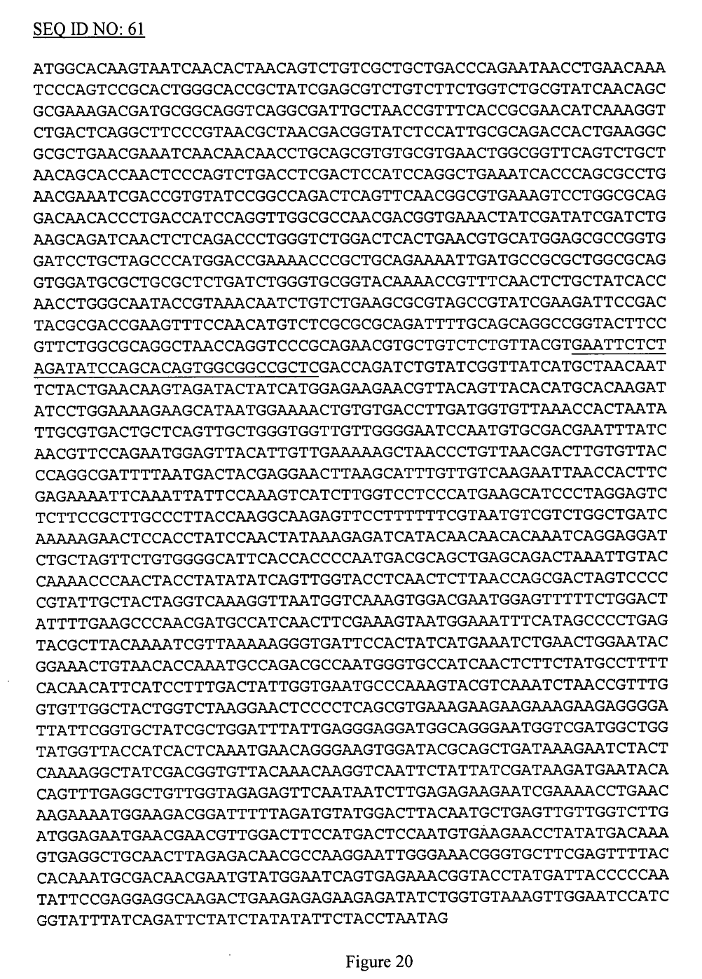

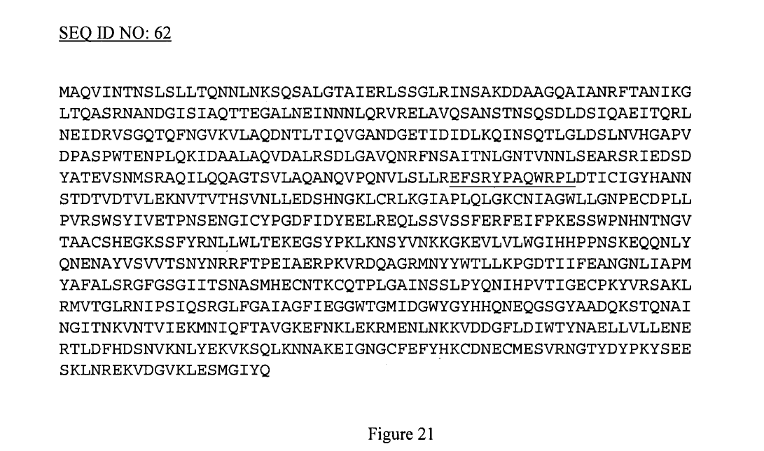

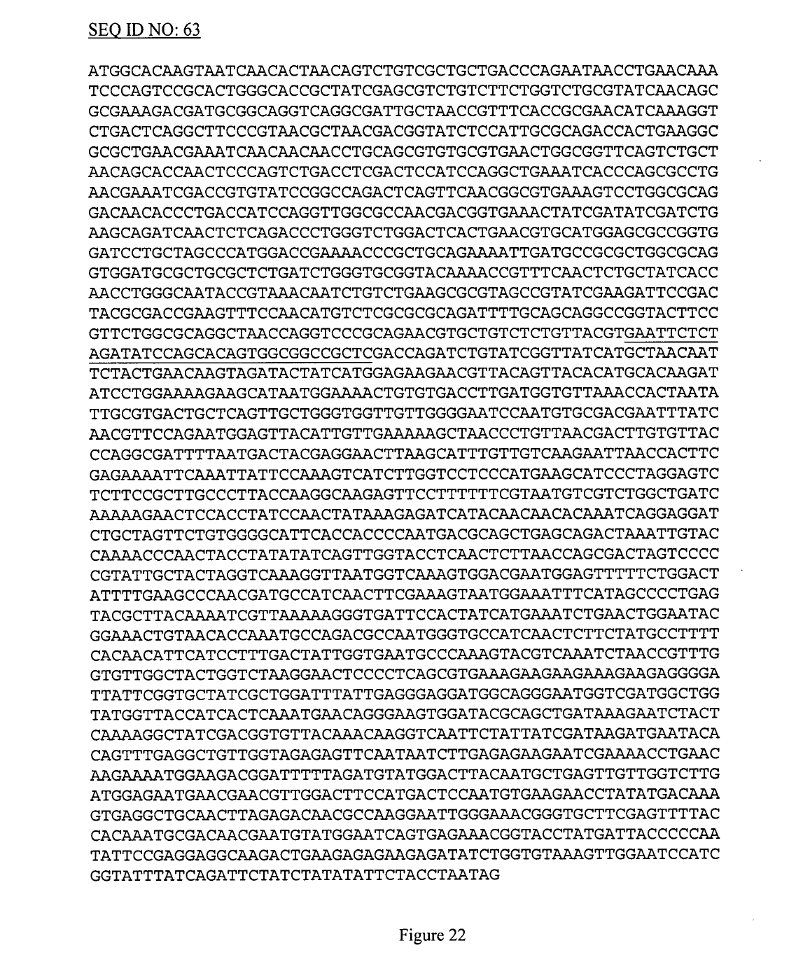

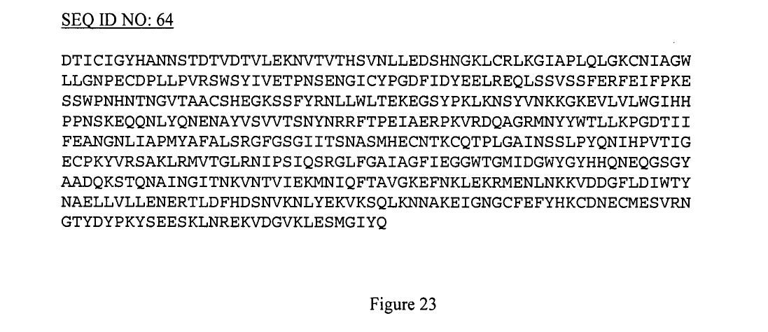

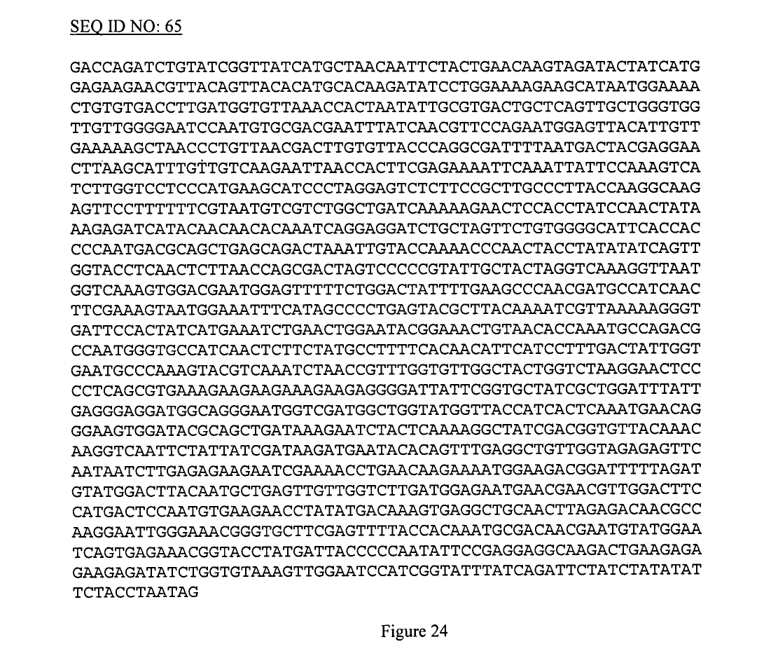

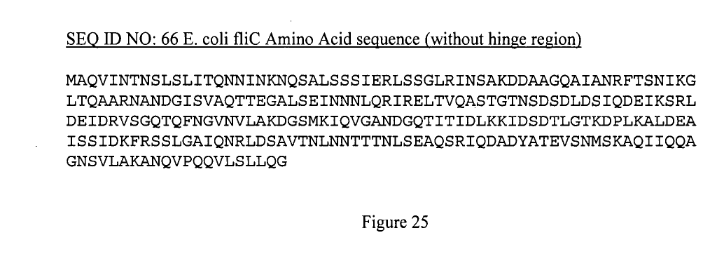

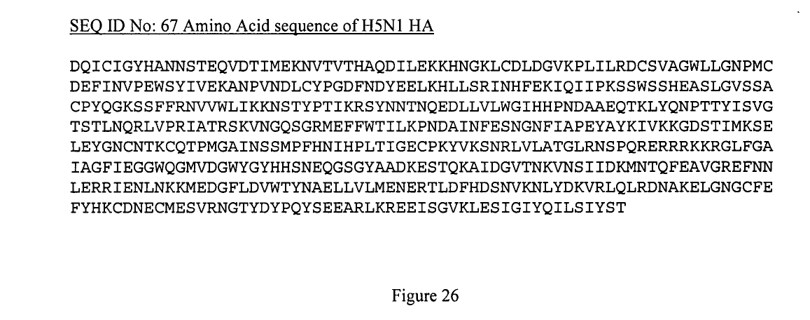

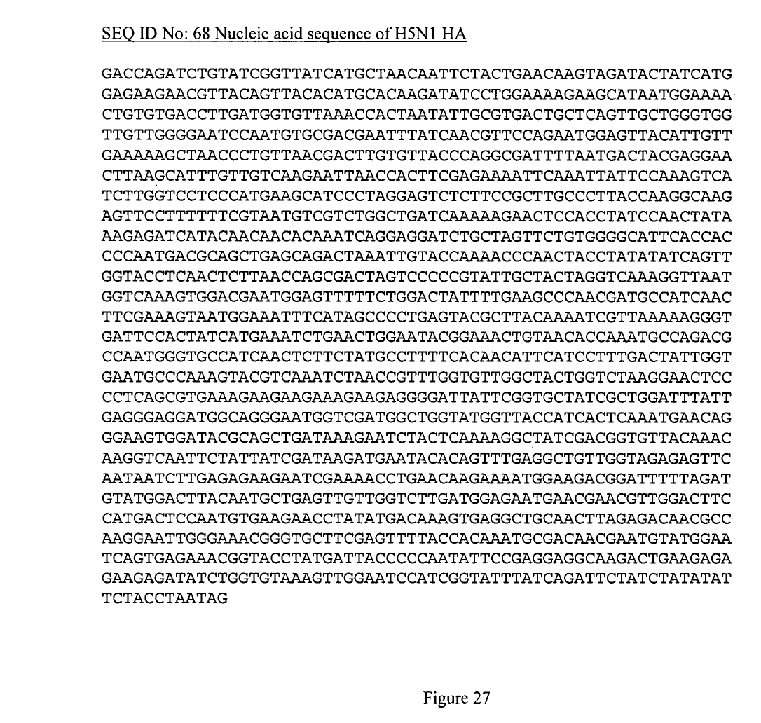

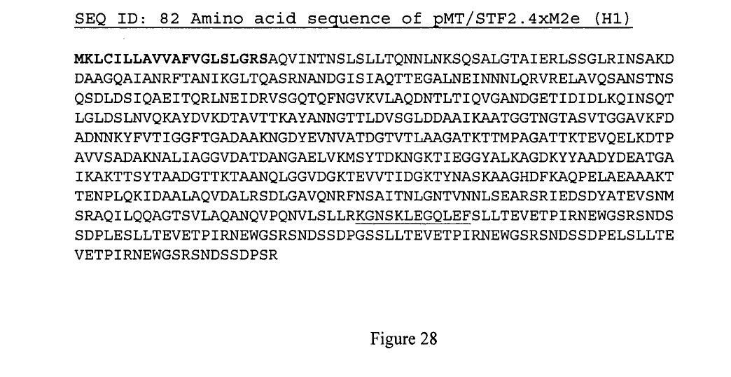

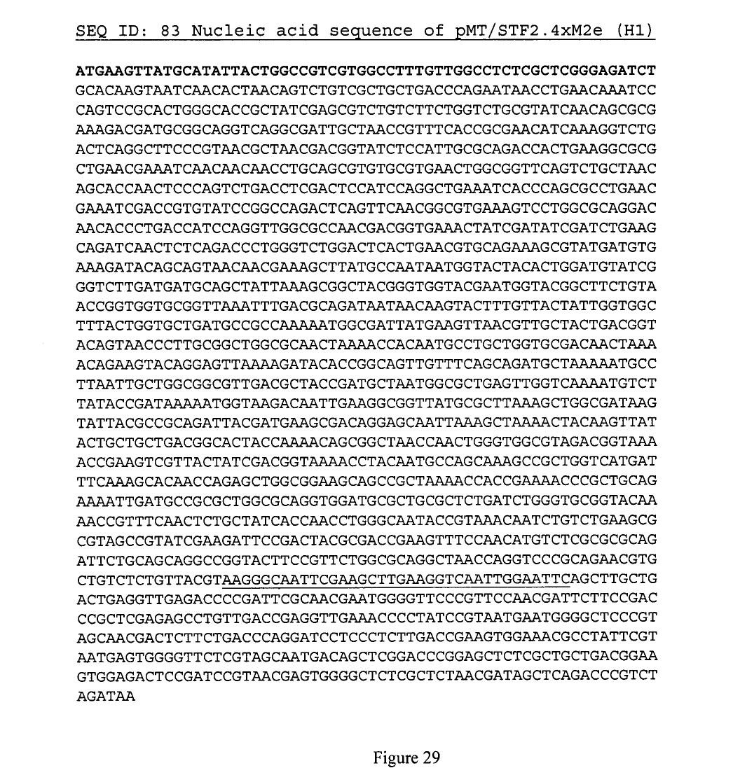

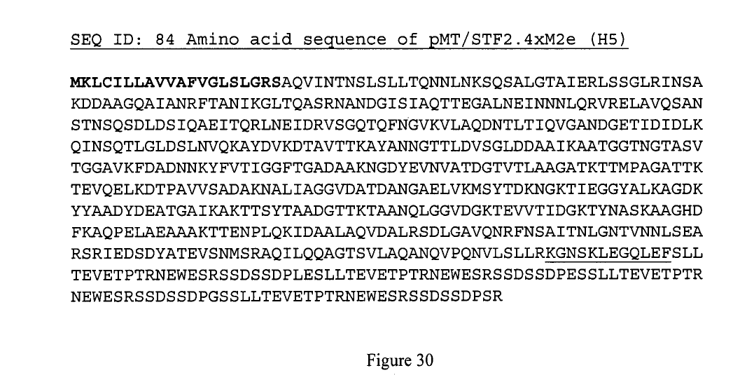

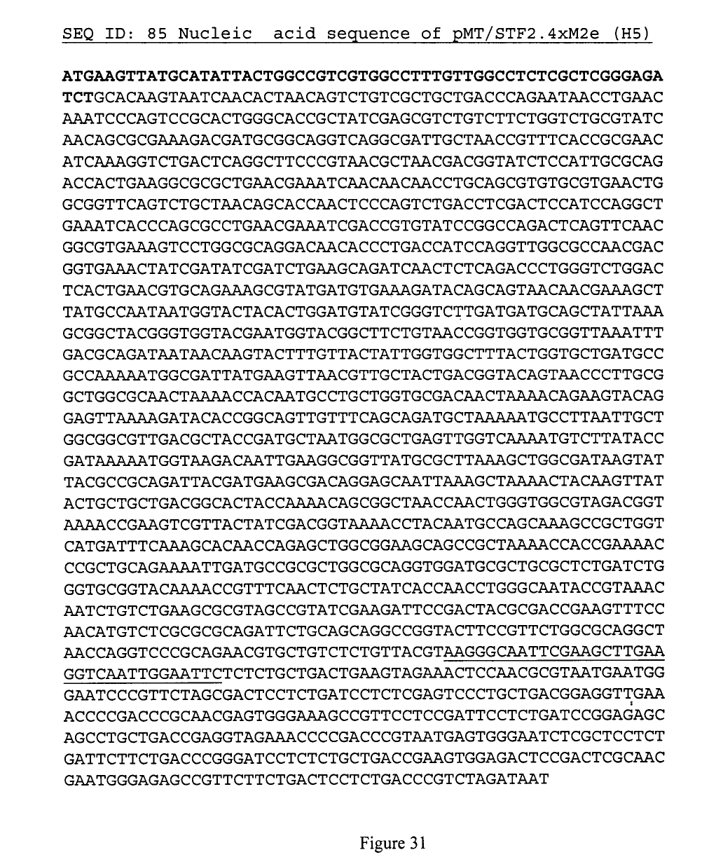

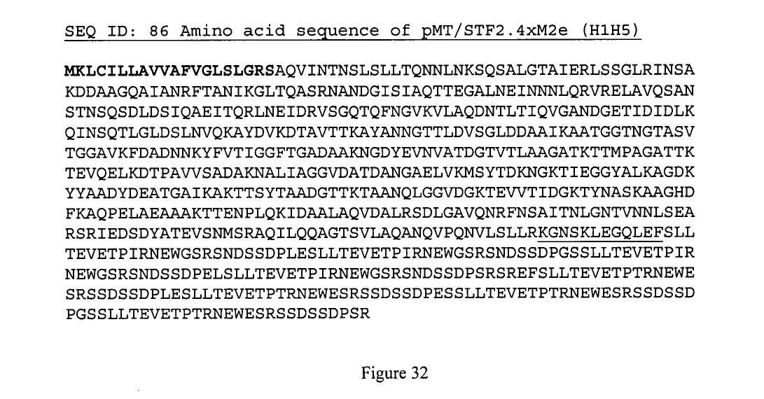

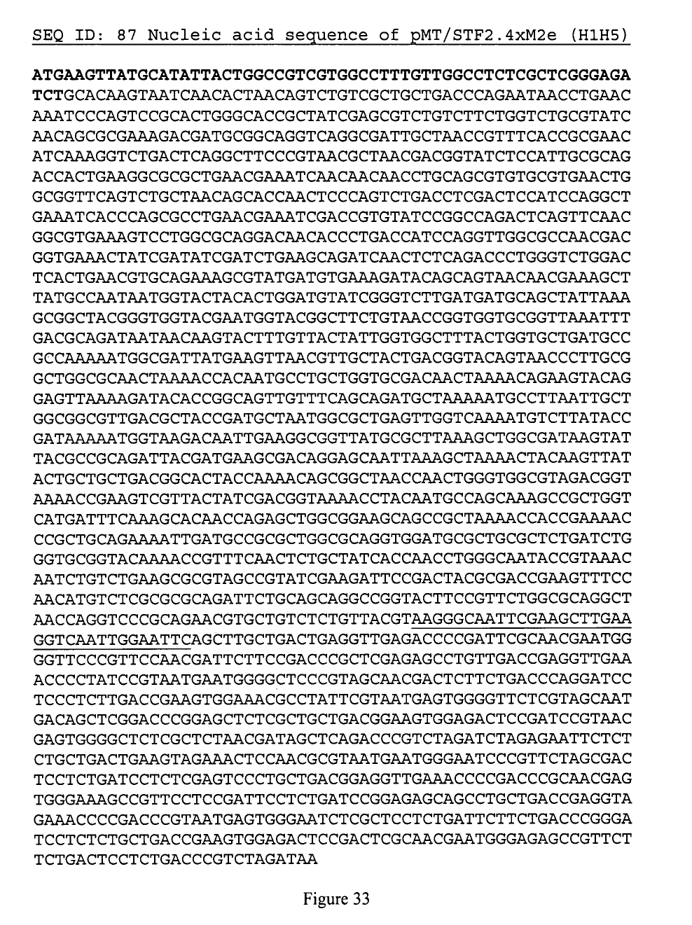

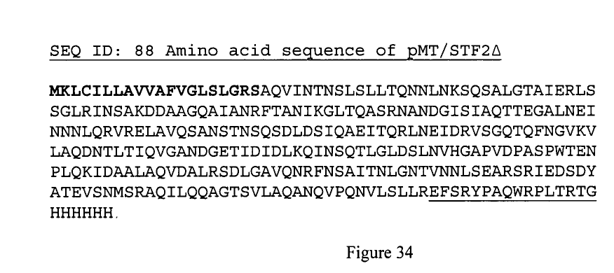

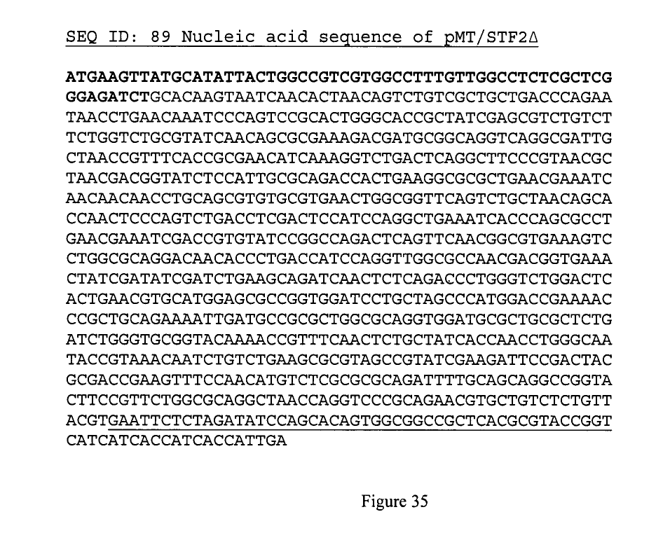

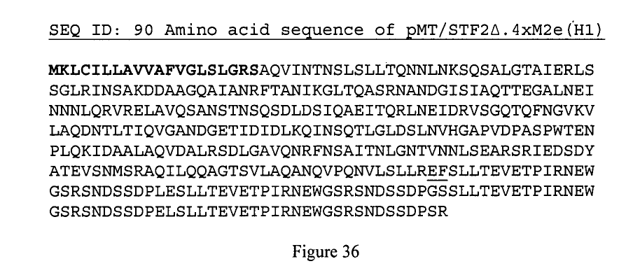

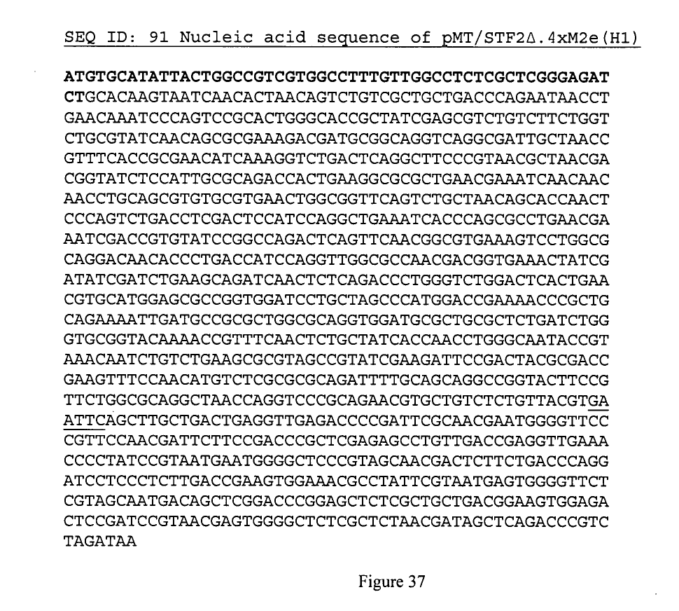

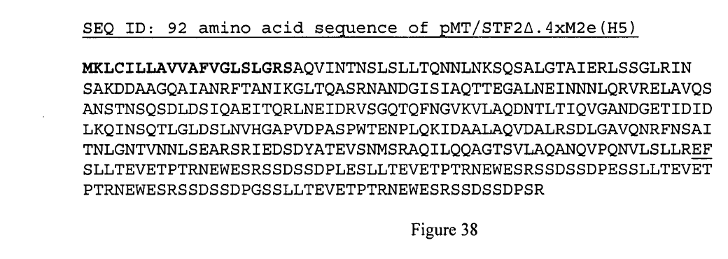

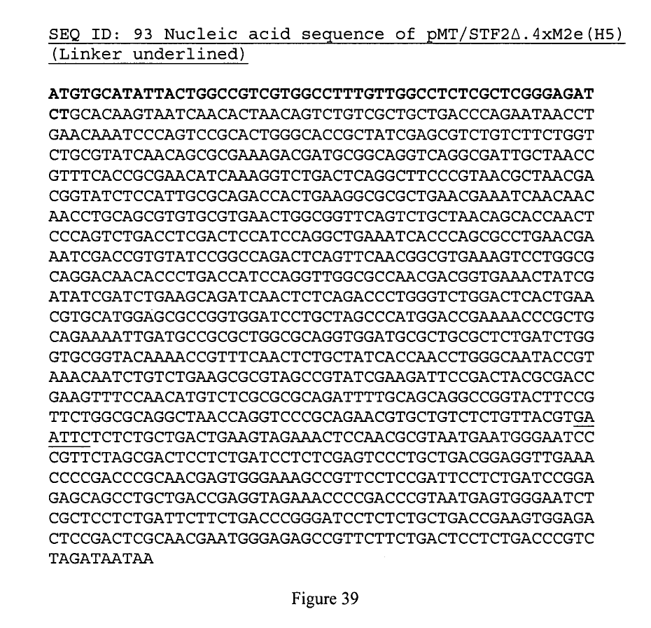

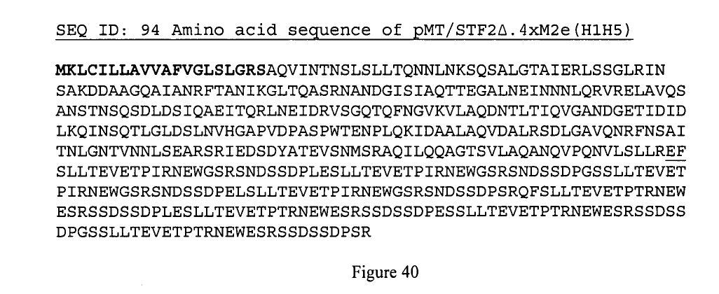

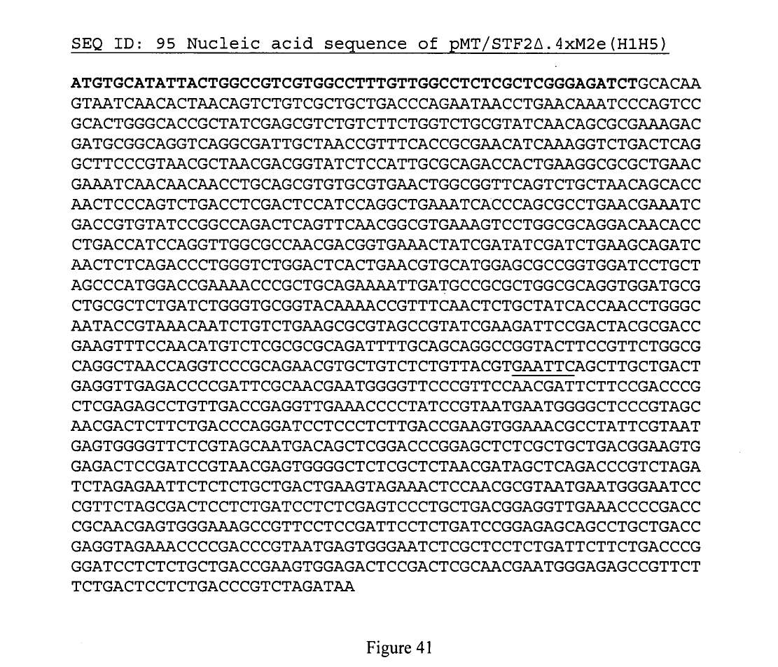

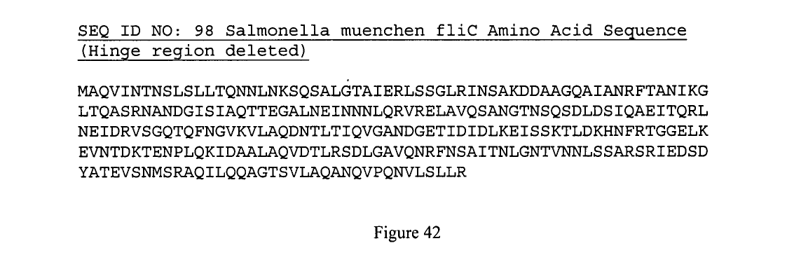

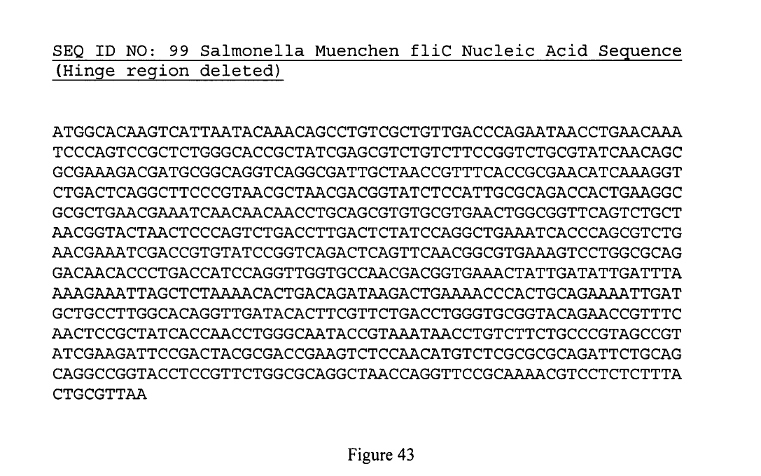

“Fusion protein,” as used herein, refers to a protein generated from at least two similar or distinct components (e.g., Pam2Cys, Pam3Cys, PAMP, at least a portion of an integral membrane protein of an influenza viral protein) that are linked covalently or noncovalently. The components of the fusion protein can be made, for example, synthetically (e.g., Pam3Cys, Pam2Cys) or by recombinant nucleic acid techniques (e.g., transfection of a host cell with a nucleic acid sequence encoding a component of the fusion protein, such as at least a portion of a PAMP, or at least a portion of an integral membrane protein of an influenza viral protein). One component of the fusion protein (e.g., Pam2Cys, Pam3Cys, PAMP, at least a portion of an integral membrane protein of an influenza viral protein) can be linked to another component of the fusion protein (e.g., Pam2Cys, Pam3Cys, PAMP, at least a portion of an integral membrane protein of an influenza viral protein) using chemical conjugation techniques, including peptide conjugation, or using molecular biological techniques, including recombinant technology, such as the generation of a fusion protein construct. Exemplary fusion proteins of the invention include SEQ ID NO: 31 (FIG. 13), encoded by SEQ ID NO: 32 (FIG. 14); SEQ ID NO: 62 (FIG. 21), encoded by SEQ ID NO: 63 (FIG. 22); SEQ ID NO: 60 (FIG. 19), encoded by SEQ ID NO: 61 (FIG. 20); SEQ ID NO: 82 ((FIG. 28), encoded by SEQ ID NO: 83 (FIG. 29); SEQ ID NO: 84 (FIG. 30), encoded by SEQ ID NO: 85 (FIG. 31); SEQ ID NO: 86 (FIG. 32), encoded by SEQ ID NO: 87 (FIG. 33); SEQ ID NO: 90 (FIG. 36), encoded by SEQ ID NO: 91 (FIG. 37); SEQ ID NO: 92 (FIG. 38), encoded by SEQ ID NO: 93 (FIG. 39); SEQ ID NO: 94 (FIG. 40), encoded by SEQ ID NO: 95 (FIG. 41); SEQ ID NO: 111; SEQ ID NO: 112; and SEQ ID NO: 113.

Fusion proteins of the invention can be designated by components of the fusion proteins separated by a “.” or “-.” For example, “STF2.M2e” refers to a fusion protein comprising one fljB/STF2 protein and one M2e protein; and “STF2Δ.4×M2e” refers to a fusion protein comprising one fljB/STF2 protein without the hinge region and (4) 24-amino acid sequences of the N-terminus of the M2 protein (SEQ ID NO: 47).

A component of the fusion protein can include MKATKLVLGAVILGSTLLAGCSSN (SEQ ID NO: 21) encoded by ATGAAAGCTACTAAACTGGTACTGGGCGCGGTAATCCTGGGTTCTACTCTGC TGCTGGCAGGTTGCTCCAGCAAC (SEQ ID NO: 22).

The fusion proteins of the invention can further include a linker between at least one component of the fusion protein (e.g., Pam3Cys, Pam2Cys, PAMP) and at least one other component of the fusion protein (e.g., at least a portion of an integral membrane protein of an influenza viral protein) of the composition, a linker between at least two of similar components of the fusion protein (e.g., Pam3Cys, Pam2Cys, PAMP, at least a portion of an integral membrane protein of an influenza viral protein) or any combination thereof. “Linker,” as used herein in reference to a fusion protein of the invention, refers to a connector between components of the fusion protein in a manner that the components of the fusion protein are not directly joined. For example, one component of the fusion protein (e.g., Pam3Cys, Pam2Cys, PAMP) can be linked to a distinct component (e.g., at least a portion of an integral membrane protein of an influenza viral protein) of the fusion protein. Likewise, at least two or more similar or like components of the fusion protein can be linked (e.g., two PAMPs can further include a linker between each PAMP, or two integral membrane proteins can further include a linker between each integral membrane protein).

Additionally or alternatively, the fusion proteins of the invention can include a combination of a linker between distinct components of the fusion protein and similar or like components of the fusion protein. For example, a fusion protein can comprise at least two PAMPs, Pam3Cys and/or Pam2Cys components that further includes a linker between, for example, two or more PAMPs; at least two integral membrane proteins of an influenza viral antigen that further include a linker between them; a linker between one component of the fusion protein (e.g., PAMP) and another distinct component of the fusion protein (e.g., at least a portion of at least one integral membrane protein of an influenza viral protein), or any combination thereof.

The linker can be an amino acid linker. The amino acid linker can include synthetic or naturally occurring amino acid residues. The amino acid linker employed in the fusion proteins of the invention can include at least one member selected from the group consisting of a lysine residue, a glutamic acid residue, a serine residue and an arginine residue. The amino acid linker can include, for example, SEQ ID NOS: 24 (KGNSKLEGQLEFPRTS), 26 (EFCRYPAQWRPL), 27 (EFSRYPAQWRPL) and 29 (KGNSKLEGQLEFPRTSPVWWNSADIQHSGGRQCDGYLQNSPLRPL), encoded by the nucleic acid sequences of SEQ ID NOS: 23 (AAGGGCAATTCGAAGCTTGAAGGTCAATTGGAATTCCCTAGGACTAGT), 25 (GAATTCTGCAGATATCCAGCACAGTGGCGGCCGCTC), 28 (GAATTCTCTAGATATCCAGCACAGTGGCGGCCGCTC) and 30 (AAGGGCAATTCGAAGCTTGAAGGTCAATTGGAATTCCCTAGGACTAGTCCA GTGTGGTGGAATTCTGCAGATATCCAGCACAGTGGCGGCCGCCAGTGTGAT GGATATCTGCAGAATTCGCCCTTGCGGCCGCTC), respectively.

The compositions of the invention can further include a linker between at least two integral membrane proteins of the composition.

The compositions, fusion proteins and polypeptides of the invention can further include a PAMP that is a TLR5 agonist. The TLR5 agonist can be a flagellin. The flagellin can be at least one member selected from the group consisting of fljB/STF2 (S. typhimurium flagellin B, Genbank Accession Number AF045151), at least a portion of fljB/STF2, E. coli flagellin fliC (also referred to herein as “E. coli fliC”) (Genbank Accession Number AB028476), at least a portion of E. coli flagellin fliC, S. muenchen flagellin fliC (also referred to herein as “S. muenchen fliC”) and at least a portion of S. muenchen flagellin fliC.

In one embodiment, the flagellin includes the polypeptides of SEQ ID NO: 1, SEQ ID NO: 3, SEQ ID NO: 5, and SEQ ID NO: 7; at least a portion of SEQ ID NO: 1, at least a portion of SEQ ID NO: 3, at least a portion of SEQ ID NO: 5, at least a portion of SEQ ID NO: 7; and a polypeptide encoded by SEQ ID NO: 2, SEQ ID NO: 4, SEQ ID NO: 6 and SEQ ID NO: 8; or at least a portion of a polypeptide encoded by SEQ ID NO: 2, SEQ ID NO: 4, SEQ ID NO: 6 and SEQ ID NO: 8. “At least a portion,” as used herein in reference to a flagellin (e.g., fljB/STF2, E. coli fliC, S. muenchen fliC), refers to any part or the entirety of the flagellin that can initiate an intracellular signal transduction pathway for a TLR. “At least a portion,” is also referred to herein as a “fragment.”

The pathogen-associated molecular pattern can be a TLR2 agonist. The TLR2 agonist can include at least a portion of a bacterial lipoprotein (BLP), such as SEQ ID NO: 21 or a polypeptide encoded by SEQ ID NO: 22.

In another embodiment, the invention is a fusion protein comprising at least one pathogen-associated molecular pattern and at least one influenza M2 protein, wherein the pathogen-associated molecular pattern is not Pam2Cys or is not a Pam3Cys. The fusion proteins of the invention can further include at least a portion of at least one member selected from the group consisting of an M2 protein, an HA protein and an NA protein. The M2 protein can include at least a portion of SEQ ID NO: 13, EVETPIRNEWG (SEQ ID NO: 15), EVETPTRNE (SEQ ID NO: 19) or EVETPIRNEW (SEQ ID NO: 34). The HA protein can include at least a portion of PAKLLKERGRRGAIAGFLE (SEQ ID NO: 33).

The fusion proteins of the invention can further include a linker between at least one pathogen-associated molecular pattern and at least one M2 protein; a linker between at least two M2 proteins; a linker between at least two PAMPs or any combination thereof.

In still another embodiment, the invention is a fusion protein comprising at least two Pam2Cys and at least one influenza M2 protein.

The pathogen-associated molecular pattern of the compositions, fusion proteins and polypeptides of the invention can include a TLR5 agonist, such as a flagellin. The flagellin can include at least one member selected from the group consisting of fljB/STF2, E. coli fliC, and S. muenchen fliC.

In one embodiment, the compositions, fusion proteins and polypeptides of the invention can include a flagellin that includes fljB/STF2 that includes at least a portion of SEQ ID NO: 1, such as the fljB/STF2 that includes SEQ ID NO: 3 or a nucleic acid sequence that encodes at least of portion of SEQ ID NO: 2, such as SEQ ID NO: 4.

In another embodiment, the compositions, fusion proteins and polypeptides of the invention can include a flagellin that includes E. coli fliC that includes at least a portion of SEQ ID NOS: 5, 9, such as E. coli fliC that includes SEQ ID NO: 66 or a nucleic acid sequence that encodes at least of portion of SEQ ID NOS: 6, 10.

In yet another embodiment, the compositions, fusion proteins and polypeptides of the invention can include a flagellin that includes S. muenchen fliC that includes at least a portion of SEQ ID NO: 7, such as S. muenchen fliC that includes SEQ ID NO: 98 or a nucleic acid sequence that encodes at least of portion of SEQ ID NO: 8, such as SEQ ID NO: 99.

The flagellin employed in the compositions, fusion proteins and polypeptides of the invention can lack a hinge region or at least a portion of a hinge region. Hinge regions are the hypervariable regions of a flagellin that link the amino-terminus and carboxy-terminus of the flagellin. Hinge regions of flagellin are also referred to as “D3 domain or region,” “propellor domain or region,” “hypervariable domain or region,” and “variable domain or region.” “Lack” of a hinge region of a flagellin, means that at least one amino acid or at least one nucleic acid codon encoding at least one amino acid that comprises the hinge region of a flagellin is absent in the flagellin. Example of hinge regions include amino acids 177-416 of SEQ ID NO: 1 that are encoded by nucleic acids 531-1248 of SEQ ID NO: 2; amino acids 174-422 of SEQ ID NO: 5 that are encoded by nucleic acids 522-1266 of SEQ ID NO: 6; or amino acids 173-464 of SEQ ID NO: 60 that are encoded by nucleic acids 519-1392 of SEQ ID NO: 61.

“At least a portion of a hinge region,” as used herein, refers to any part of the hinge region of the PAMP that is less than the entire hinge region. “At least a portion of a hinge region” is also referred to herein as a “fragment of a hinge region.” For example, the hinge region of S. typhimurium flagellin B (fljB, also referred to herein as fljB/STF2 or STF2) is amino acids 175-415 of SEQ ID NO: 1, which are encoded by nucleic acids at position 541-1246 of SEQ ID NO: 2. A fragment of the hinge region of fljB/STF2 can be, for example, amino acids 200-300 of SEQ ID NO: 1.

In another embodiment, at least a portion of a naturally occurring flagellin can be replaced with at least a portion of an artificial hinge region. “Naturally occurring,” in reference to a flagellin amino acid sequence, means the amino acid sequence present in the native flagellin (e.g., S. typhimurium flagellin, S. muenchin flagellin, E. coli flagellin). The naturally occurring hinge region is the hinge region that is present in the native flagellin. “Artificial,” as used herein in reference to a hinge region of a flagellin, means a hinge region that is inserted in the native flagellin in any region of the flagellin that contains or contained the native hinge region.

An artificial hinge region may be employed in a flagellin that lacks at least a portion of a hinge region, which may facilitate interaction of the carboxy- and amino-terminus of the flagellin for binding to TLR5 and, thus, activation of the TLR5 innate signal transduction pathway. A flagellin lacking at least a portion of a hinge region is designated by the name of the flagellin followed by a “Δ.” For example, an STF2 that lacks at least a portion of a hinge region is referenced to as “STF2Δ” or “fljB/STF2Δ.

The compositions, fusion proteins and polypeptides of the invention can also include at least a portion of an influenza viral protein placed in or fused to a portion of the pathogen-associated molecular pattern, such as a region of the pathogen-associated molecular pattern that contains or contained a hinge region. For example, the hinge region of the pathogen-associated molecular pattern or at least a portion of the hinge region of the pathogen-associated molecular pattern can be removed from the pathogen-associated molecular pattern and replaced with at least a portion of an influenza viral antigen (e.g., M2, such as SEQ ID NOS: 13, 19 and 39-59). A linker can further be included between the influenza viral antigen and the pathogen-associated molecular pattern in such a replacement.

The pathogen-associated molecular pattern of the fusion proteins of the invention can be fused to a carboxy-terminus, the amino-terminus or both the carboxy- and amino-terminus of an influenza protein, such as an integral membrane protein of an influenza viral protein (e.g., M2, HA, NA). The fusion proteins of the invention can include at least one pathogen-associated molecular pattern between at least two influenza M2 proteins, which can, optionally, include a linker between the pathogen-associate molecular pattern and the M2 protein.

The pathogen-associated molecular pattern of the fusion proteins of the invention can include a TLR2 agonist, such as at least one Pam2Cys, at least one Pam3Cys or any combination thereof. Thus, the fusion proteins of the invention can include at least one member selected from the group consisting of Pam2Cys and a Pam3Cys.

The fusion proteins comprising at least one pathogen-associated molecular pattern and at least a portion of at least one M2 protein can further include at least a portion of a haemagglutinin membrane protein; at least a portion of a neuraminidase membrane protein; at least one member selected from the group consisting of an influenza B viral protein and an influenza C viral protein; or any combination thereof. The influenza B viral protein and/or influenza C viral protein can be an integral membrane protein.

In yet another embodiment, the invention is a composition comprising a pathogen-associated molecular pattern and an M2 protein.

In an additional embodiment, the invention is a composition comprising at least a portion of at least one pathogen-associated molecular pattern and at least a portion of at least one influenza M2 protein, wherein, if the pathogen-associated molecular pattern includes a Pam2Cys, at least a portion of the Pam2Cys is not fused to the influenza M2 protein and at least a portion of the influenza M2 protein is not fused to the Pam2Cys or is not fused to a Pam3Cys.

“Fused to,” as used herein means covalently or noncovalently linked or recombinantly produced together.

In another embodiment, the invention is a fusion protein comprising at least a portion of at least one pathogen-associated molecular pattern and at least a portion of at least one influenza M2 protein, wherein, if the pathogen-associated molecular pattern includes a Pam2Cys, at least a portion of the Pam2Cys is not fused to the influenza M2 protein and at least a portion of the influenza M2 protein is not fused to the Pam2Cys or is not fused to a Pam3Cys.

In still another embodiment, the invention includes a polypeptide that includes SEQ ID NOS: 9, 31, 64, 60, 82, 84, 86, 88, 90, 92 and 94 and a polypeptide encoded by SEQ ID NOS: 10, 32, 63, 61, 83, 85, 87, 89, 91, 93 and 95.

In an additional embodiment, the invention includes a polypeptide having at least about 70%, at least about 75%, at least about 80%, at least about 85%, at least about 90%, at least about 95%, at least about 98% and at least about 99% sequence identity to the polypeptides described herein, such as SEQ ID NOS: 9, 31, 64, 60, 82, 84, 86, 88, 90, 92, 94, 111, 112 and 113 and the nucleic acids of SEQ ID NOS: 10, 32, 63, 61, 83, 85, 87, 89, 91, 93 and 95.

The percent identity of two amino acid sequences (or two nucleic acid sequences) can be determined by aligning the sequences for optimal comparison purposes (e.g., gaps can be introduced in the sequence of a first sequence). The amino acid sequence or nucleic acid sequences at corresponding positions are then compared, and the percent identity between the two sequences is a function of the number of identical positions shared by the sequences (i.e., % identity=# of identical positions/total # of positions ×100). The length of the protein or nucleic acid encoding a PAMP, at least a portion of an influenza viral protein, a fusion protein of the invention or a polypeptide of the invention aligned for comparison purposes is at least 30%, preferably, at least 40%, more preferably, at least 60%, and even more preferably, at least 70%, 75%, 80%, 85%, 90%, 95%, 98%, 99% or 100%, of the length of the reference sequence, for example, the nucleic acid sequence of a PAMP, at least a portion of an integral membrane protein of an influenza viral protein, or a polypeptide or fusion protein, for example, as depicted in SEQ ID NOS: 9, 31, 64, 60, 82, 84, 86, 88, 90, 92 and 94 and SEQ ID NOS: 10, 32, 63, 61, 83, 85, 87, 89, 91, 93, 95, 111, 112 and 113.

The actual comparison of the two sequences can be accomplished by well-known methods, for example, using a mathematical algorithm. A preferred, non-limiting example of such a mathematical algorithm is described in Karlin et al. (Proc. Natl. Acad. Sci. USA, 90:5873-5877 (1993), the teachings of which are hereby incorporated by reference in its entirety). Such an algorithm is incorporated into the BLASTN and BLASTX programs (version 2.2) as described in Schaffer et al. (Nucleic Acids Res., 29:2994-3005 (2001), the teachings of which are hereby incorporated by reference in its entirety). When utilizing BLAST and Gapped BLAST programs, the default parameters of the respective programs (e.g., BLASTN; available at the Internet site for the National Center for Biotechnology Information) can be used. In one embodiment, the database searched is a non-redundant (NR) database, and parameters for sequence comparison can be set at: no filters; Expect value of 10; Word Size of 3; the Matrix is BLOSUM62; and Gap Costs have an Existence of 11 and an Extension of 1.

Another mathematical algorithm employed for the comparison of sequences is the algorithm of Myers and Miller, CABIOS (1989), the teachings of which are hereby incorporated by reference in its entirety. Such an algorithm is incorporated into the ALIGN program (version 2.0), which is part of the GCG (Accelrys, San Diego, Calif.) sequence alignment software package. When utilizing the ALIGN program for comparing amino acid sequences, a PAM120 weight residue table, a gap length penalty of 12, and a gap penalty of 4 is used. Additional algorithms for sequence analysis are known in the art and include ADVANCE and ADAM as described in Torellis and Robotti (Comput. Appl. Biosci., 10: 3-5 (1994), the teachings of which are hereby incorporated by reference in its entirety); and FASTA described in Pearson and Lipman (Proc. Natl. Acad. Sci. USA, 85: 2444-2448 (1988), the teachings of which are hereby incorporated by reference in its entirety).

In a further embodiment, the invention is host cells and vectors that include the nucleic acid sequences of the invention. The host cells can be prokaryotic or eukaryotic host cells.

The eukaryotic host cells employed in the methods of the invention can include a Saccharomyces eukaryotic host cell, an insect eukaryotic host cell (e.g., at least one member selected from the group consisting of a Baculovirus infected insect cell, such as Spodoptera frugiperda (Sf9) or Trichhoplusia ni (High5) cells; and a Drosophila insect cell, such as Dme12 cells), a fungal eukaryotic host cell, a parasite eukaryotic host cell (e.g., a Leishmania tarentolae eukaryotic host cell), CHO cells, yeast cells (e.g., Pichia) and a Kluyveromyces lactis host cell.

Suitable eukaryotic host cells and vectors can also include plant cells (e.g., tomato; chloroplast; mono- and dicotyledonous plant cells; Arabidopsis thaliana; Hordeum vulgare; Zea mays; potato, such as Solanum tuberosum; carrot, such as Daucus carota L.; and tobacco, such as Nicotiana tabacum, Nicotiana benthamiana (Gils, M., et al., Plant Biotechnol J. 3:613-20 (2005); He, D. M., et al., Colloids Surf B Biointerfaces, (2006); Huang, Z., et al., Vaccine 19:2163-71 (2001); Khandelwal, A., et al., Virology. 308:207-15 (2003); Marquet-Blouin, E., et al., Plant Mol Biol 51:459-69 (2003); Sudarshana, M. R., et al. Plant Biotechnol J 4:551-9 (2006); Varsani, A., et al., Virus Res, 120:91-6 (2006); Kamarajugadda S., et al., Expert Rev Vaccines 5:839-49 (2006); Koya V, et al., Infect Immun. 73:8266-74 (2005); Zhang, X., et al., Plant Biotechnol J. 4:419-32 (2006)).

The prokaryotic host cell can be at least one member selected from the group consisting of an E. coli prokaryotic host cell, a Pseudomonas prokaryotic host cell, a Bacillus prokaryotic host cell, a Salmonella prokaryotic host cell and a P. fluorescens prokaryotic host cell.

The percent identity between two amino acid sequences can also be accomplished using the GAP program in the GCG software package (Accelrys, San Diego, Calif.) using either a Blossom 63 matrix or a PAM250 matrix, and a gap weight of 12, 10, 8, 6, or 4 and a length weight of 2, 3, or 4. In yet another embodiment, the percent identity between two nucleic acid sequences can be accomplished using the GAP program in the GCG software package (Accelrys, San Diego, Calif.), using a gap weight of 50 and a length weight of 3.

The nucleic acid sequence encoding a PAMP, at least a portion of an integral membrane protein of an influenza viral protein, fusion proteins of the invention and polypeptides of the invention can include nucleic acid sequences that hybridize to, for example, a fljB/STF2 (e.g., SEQ ID NOS: 2, 4), a fliC (e.g., SEQ ID NOs: 6, 8, 99), at least a portion of an integral membrane protein of an influenza viral protein (e.g., SEQ ID NOS: 11, 13, 15, 18, 19, 21, 33, 35-59, 64 and 67) and fusion proteins of the invention (e.g., SEQ ID NOS: 31, 64 and 60) under selective hybridization conditions (e.g., highly stringent hybridization conditions). As used herein, the terms “hybridizes under low stringency,” “hybridizes under medium stringency,” “hybridizes under high stringency,” or “hybridizes under very high stringency conditions,” describe conditions for hybridization and washing of the nucleic acid sequences. Guidance for performing hybridization reactions, which can include aqueous and nonaqueous methods, can be found in Aubusel, F. M., et al., Current Protocols in Molecular Biology, John Wiley & Sons, N.Y. (2001), the teachings of which are hereby incorporated herein in its entirety.

For applications that require high selectivity, relatively high stringency conditions to form hybrids can be employed. In solutions used for some membrane based hybridizations, addition of an organic solvent, such as formamide, allows the reaction to occur at a lower temperature. High stringency conditions are, for example, relatively low salt and/or high temperature conditions. High stringency are provided by about 0.02 M to about 0.10 M NaCl at temperatures of about 50° C. to about 70° C. High stringency conditions allow for limited numbers of mismatches between the two sequences. In order to achieve less stringent conditions, the salt concentration may be increased and/or the temperature may be decreased. Medium stringency conditions are achieved at a salt concentration of about 0.1 to 0.25 M NaCl and a temperature of about 37° C. to about 55° C., while low stringency conditions are achieved at a salt concentration of about 0.15 M to about 0.9 M NaCl, and a temperature ranging from about 20° C. to about 55° C. Selection of components and conditions for hybridization are well known to those skilled in the art and are reviewed in Ausubel et al. (1997, Short Protocols in Molecular Biology, John Wiley & Sons, New York N.Y., Units 2.8-2.11, 3.18-3.19 and 4-64.9).

In a further embodiment, the compositions, fusion proteins and polypeptides of the invention can be employed in methods of stimulating an immune response in a subject. In one embodiment, the method of the invention can include a method of stimulating an immune response in a subject, comprising the step of administering to the subject a composition that includes at least one Pam3Cys and at least a portion of at least one integral membrane protein of an influenza viral protein. In another embodiment, the invention can include a method of stimulating an immune response in a subject, comprising the step of administering to the subject a composition that includes a fusion protein comprising at least one pathogen-associated molecular pattern and at least one influenza M2 protein. In a further embodiment, the invention can include a method of stimulating an immune response in a subject, comprising the step of administering to the subject a composition that includes at least one pathogen-associated molecular pattern and at least one influenza M2 protein, wherein the pathogen-associated molecular pattern is not a Pam2Cys or is not a Pam3Cys and the M2 protein is not an M2e.

In yet another embodiment, the invention is a method of stimulating an immune response in a subject, comprising the step of administering to the subject a composition that includes a composition comprising at least a portion of at least one pathogen-associated molecular pattern and at least a portion of at least one influenza M2 protein, wherein, if the pathogen-associated molecular pattern includes a Pam2Cys, at least a portion of the Pam2Cys is not fused to the influenza M2 protein and at least a portion of the influenza M2 protein is not fused to the Pam2Cys or is not fused to a Pam3Cys.

In a further embodiment, the invention is a method of stimulating an immune response in a subject, comprising the step of administering to the subject a composition that includes a fusion protein comprising at least a portion of at least one pathogen-associated molecular pattern and at least a portion of at least one influenza M2 protein, wherein, if the pathogen-associated molecular pattern includes a Pam2Cys, at least a portion of the Pam2Cys is not fused to the influenza M2 protein and at least a portion of the influenza M2 protein is not fused to the Pam2Cys or is not fused to a Pam3Cys.

A subject treated by the methods of the invention can be a mammal, such as a primate or a rodent (e.g., mouse, rat). In a particular embodiment, the subject is a human. A subject is also referred to herein as “an individual.”

“Stimulating an immune response,” as used herein, refers to the generation of antibodies and/or T-cells to at least a portion of an influenza viral protein (e.g., an integral membrane, such as M2, HA, NA of influenza A, B and/or C). Stimulating an immune response in a subject can include the production of humoral and/or cellular immune responses that are reactive against the influenza viral protein. In stimulating an immune response in the subject, the subject may be protected from infection by the influenza virus or conditions associated with infection by the influenza virus that may diminish or be halted as a consequence of stimulating an immune response in the subject.

The compositions of the invention for use in methods to stimulate immune responses in subjects, can be evaluated for the ability to stimulate an immune response in a subject using well-established methods. Exemplary methods to determine whether the compositions of the invention stimulate an immune response in a subject, include measuring the production of antibodies specific to the antigen (e.g., IgG antibodies) by a suitable technique such as, ELISA assays; the potential to induce antibody-dependent enhancement (ADE) of a secondary infection; macrophage-like assays; neutralization assessed by using the Plaque Reduction Neutralization Test (PRNT₈₀); and the ability to generate serum antibodies in non-human models (e.g., mice, rabbits, monkeys) (Putnak, et al., Vaccine 23:4442-4452 (2005)).

“Stimulates a protective immune response,” as used herein, means administration of the compositions of the invention, such as hemagglutinin (HA) proteins (e.g., HA1-1, HA1-2 proteins described herein), results in production of antibodies to the protein to thereby cause a subject to survive challenge by an otherwise lethal dose of a viral protein, such as viral HA. Techniques to determine a lethal dose of a virus (e.g., an influenza virus) are known to one of skill in the art (see, for example, WHO/CDS/CSR/NCS2002.5 “WHO Manual on Animal Influenza Diagnosis and Surveillance” World Health Organization, Dept of Communicable Disease Surveillance and Response, WHO Global Influenza Programme; Harmon, M. W., et al., J. Clin. Microbiol. 26:333-337 (1988); Reed, L. J., et al., Am. J. Hyg. 27:493-497 (1938); Rose, T., et al., J. Clin. Microbiol. 37:937-943 (1999); Walls, H. H. et al., J. Clin. Microbiol. 23:240-245 (1986); Current Protocols in Immunology, 19.11.1-19.11.32, Cottey, R., et al., John Wiley & Sons, Inc (2001)). Exemplary techniques for determining a lethal dose can include administration of varying doses of virus and a determination of the percent of subjects that survive following administration of the dose of virus (e.g., LD₁₀ LD₂₀, LD₄₀, LD₅₀, LD₆₀, LD₇₀, LD₈₀, LD₉₀). For example, a lethal dose of a virus that results in the death of 50% of a population of subjects is referred to as an “LD₅₀”; a lethal dose of a virus that results in the death of 80% of a population of subjects is referred to herein as “LD₈₀”; a lethal dose of a virus that results in death of 90% of a population of subjects is referred to herein as “LD₉₀.”

For example, determination of the LD₉₀ can be conducted in subjects (e.g., mice) by administering intranasally varying doses (e.g., dilutions, such as log and half-log dilutions of 8×10³ egg-infectious doses (EID)) followed by an assessment of the survival of the subjects about 14 days to about 21 days after infection with the virus. Protective immunity can be assessed by physical appearance of the subject, general demeanor (active), weight (initial loss of weight followed by return to a weight about the weight of the subject prior to infection with the virus) and survival after about 14 to about 21 days following infection with the virus.

Assessment of stimulation of protective immunity can also be made by employing assays that assess the ability of the antibodies produced in response to the compositions of the invention (e.g., a portion of the protein of the naturally occurring virus, such as a protein portion of hemagglutinin) to neutralize binding of the viral protein (e.g., hemagglutinin protein) to a host cell (see, for example, Current Protocols in Immunonology, 19.11.1-19.11.32, Cottey, R., et al., John Wiley & Sons, Inc (2001)). Assessment of stimulation of protective immunity can also be made by employing assays that measure the ability of antibodies to inhibit hemagglutinin binding (see, for example, Burnett, F. M., et al., J. exp. Biol. Med. Sci. 25:227-233 (1947); Salk, J. E. J. Immunol. 49:87-98 (1944); Current Protocols in Immunology, 19.11.1-19.11.32, Cottey, R., et al., John Wiley & Sons, Inc (2001)).

It is believed that inhibition of hemagglutinin binding is indicative of the ability of antibodies, formed from the compositions and by the methods of the invention, to neutralize the sialic acid binding sites of the naturally occurring viral hemagglutinin (“neutralization of HA binding”) and, thereby, prevent infection of the host cell as a consequence of stimulating a protective immune response. Inhibition or neutralization of hemagglutinin binding is believed to correlate with an ability of an immune response to protect against a lethal dose of virus.

Neutralization of HA binding can be assessed by in vitro assays (See, for example, Current Protocols in Immunology 19.11.1-19.11.32, Cottey, R., et al., Suppl. 42, John Wiley & Sons, Inc. (2001) and WHO Manual on Animal Influenza Diagnosis and Surveillance, Webster, R., et al., pages 28-36, 48-54, 82-92 (2002)). Exemplary viral neutralization assays rely on the ability of serum to specifically bind and prevent replication of influenza virus in culture, such as in the Madin-Darby Canine Kidney (MDCK) cell line. Briefly, cells are cultured in 96 well plates in the presence of a previously titered virus and the cytopathic effect of the replicating virus is observed under a microscope. To test serum, serial dilutions of the serum are prepared and preincubated with the viral stock for 2 hours at 37° C. prior to infecting the MDCK cells. The mixture is incubated for an additional 2 hours after which the virus/serum mixture is removed and replaced with fresh media. The cells are grown for 4 days. Wells are scored as positive for viral growth if at least about 50% of the cells are dead in at least about half of the wells for a given serum dilution. The reciprocal of the highest dilution of serum which protects at least about half of the cells from death, in at least about half of the wells, is considered the neutralization titer.

Alternatively, a micro-neutralization in vitro assay can be performed to assess neutralization of HA binding. For example, serum is diluted and preincubated with a known titer of virus and mixed with MDCK cells, as described above. After 2 days of incubation, cells are washed and fixed with acetone. The plates are developed as an ELISA using a monoclonal antibody to the influenza nuclear antigen NP. A microneutralization titer is determined as the reciprocal of the highest dilution which yields less than about 50% of the anti-NP reading of the virus-only control wells.

The Hemagglutination Inhibition (HAI) assay is based on the HA antigen on the surface of the influenza virus agglutinating red blood cells (RBC) and preventing red blood cells from precipitating. Antibodies that specifically bind the sialic acid-binding regions of HA prevent agglutination allowing precipitation. The assay is performed in 96 well V bottom plates with fresh chicken RBC. A stock of viral antigen is titered so that about a 4-fold excess of antigen is present relative to the minimum amount needed to prevent precipitation. The test serum, which can be from several species including mouse, ferret, poultry or human, is heated to about 56° C. to inactivate complement. Serial 2-fold dilutions of the inactivated serum are performed and mixed with the stock HA. After about 30 minutes at room temperature, the RBCs are added and the plate is incubated for about 30 to about 45 minutes. Results are scored by observations: agglutination results in cloudy wells while inhibition results in a “button” of red cells precipitated at the bottom of the well. Controls include RBC with no HA, which forms a button, and HA and RBC with no serum, which remains cloudy. The HAI titer of a particular serum sample is the reciprocal of the last dilution which prevents agglutination (i.e., forms a button). For example, if about a 1:128 dilution reads as a button but the 1:256 dilution does not, the HAI titer is about 128.

The compositions, fusion proteins and polypeptides of the invention can be administered to a subject with or without an adjuvant to coordinate the innate and adaptive immune mechanisms and induce a potent antibody response accompanied by minimal non-specific inflammation. The induced immune response may provide protection against homologous and heterologous strains of influenza viruses and thereby may provide protection against circulating influenza viruses and against potential pandemic influenza caused by introduction of the H5 avian strain into the human population.

In yet another embodiment, the invention is a method of decreasing an antibody response (immune response) to at least a portion of a flagellin that is a component of a fusion protein, wherein the fusion protein activates a Toll-like Receptor 5 and includes at least one antigen, comprising the step of deleting at least a portion of a hinge region of the flagellin.

“Component of a fusion protein,” as used herein in reference to a fusion protein, means that the flagellin or antigen comprises a part of the fusion protein, for example, STF2 is a flagellin component of the fusion protein of ST2.4×M2e. Likewise, M2e is an antigen component of the fusion protein.

In one embodiment, the hinge region of the flagellin is deleted prior to fusion of the flagellin component of the fusion protein to the antigen component of the fusion protein. In another embodiment, the hinge region of the flagellin is deleted after fusion of the flagellin component of the fusion protein to the antigen component of the fusion protein.

In a further embodiment, the invention is a method of increasing in vitro yield of a fusion protein, wherein the fusion protein activates a Toll-like Receptor 5 and includes at least a portion of at least one flagellin and at least a portion of at least one antigen, comprising the step of forming a fusion protein lacking at least a portion of a naturally occurring hinge region.

“In vitro yield,” as used herein in reference to a fusion protein, means production of the fusion protein under in vitro condition (e.g., in a cultured host cell, such as, a prokaryotic or eukaryotic host cell).

“Naturally occurring,” as used herein in reference to a hinge region of a flagellin, refers to the hinge region of a flagellin that occurs in nature.

As described herein, fusion proteins that include a flagellin component that lacks a hinge region have reduced immunogenicity to the flagellin component of the fusion protein, yet, maintain TLR5 activity and immunogenicity to the antigen component of the fusion protein. In addition, fusion proteins that include a flagellin component of the fusion protein that lacks the hinge region can increase the yield (e.g., in vitro yield) of fusion proteins that comprise a flagellin component and an antigen component, thereby resulting in high yield of fusion proteins for use in the methods described herein. In addition, fusion proteins that include a flagellin component that lacks a hinge region can form solid inclusion bodies (IBs) when expressed in prokaryotic cells (e.g., E. coli cells), which facilitates large scale production of the fusion proteins for use in the methods described herein, in part, because the fusion proteins can be obtained by washing the inclusion bodies prior to chromaticgraphic processing or large scale production.

Strategies to manage infection and illness consequent to influenza viral infection have not changed significantly in the past four decades. Due to the seasonal nature of the disease, the distinct types of influenza virus (A and B) that threaten the human population, and the genetic instability of each type, it is necessary to reformulate a multivalent compositions (e.g., compositions containing more than one type of influenza viral protein) for immunizing and vaccinating subjects each year, based on epidemiological prediction of strains likely to be circulating in a population in the an upcoming flu season. Certain compositions, such as vaccines are produced from stocks of selected prototype viral strains grown in embryonated chicken eggs. Limitations of the currently available techniques include, for example, uncertain prediction of circulating strains; the ability to grow the appropriate strains in chicken eggs; the egg-based production system carries risks of product contamination; the product produced in eggs cannot be used in subjects with egg allergies; and risk that the multivalent composition will not confer protection against a pandemic strain of virus to which the a subject has no pre-existing immunity.

Generally, the dominant protective component of an influenza composition, such as a vaccine, is the viral haemagglutinin, the major virulence factor associated with the influenza A virus. Neutralizing antibodies to HA arise in response to natural infection or administration with influenza A virus and provide sterilizing immunity to subsequent exposure to a virus expressing that particular HA.

There are several antigenically distinct phenotypes of HA. Most human influenza isolates express the H1 or H3 phenotype, while avian viral strains may express H5, H7, or H9. Even within a particular phenotype such as H1, the virus may change by “antigenic drift” (point mutation) and “antigenic shift” (genetic re-assortment) of the HA antigen that may render the virus resistant to immune responses directed against earlier virus strains, whether that immunity arose in response to infection or to vaccination. Thus, the efficacy of traditional compositions employed to prevent influenza infection is limited against a pandemic strain such as one of the avian strains to which the human population has not developed immunity. The long manufacturing process prevents the efficient production of traditional compositions to prevent influenza infection against an emerging pandemic strain. The compositions, fusion proteins and polypeptides of the invention may prevent influenza infection in a manner that is cost-effective to produce and that can be stockpiled in preparation for an influenza pandemic.

Subtypes of the influenza A virus are generally named according to the particular antigenic determinants of hemagglutinin (H, about 13 major types) and neuraminidase (N, about 9 major types). For example, subtypes include influenza A (H2N1), A(H3N2), A(H5N1), A(H7N2), A(H9N2), A(H1/H1), A(H3/H0) and A(H5/H0). In the last century, three subtypes of influenza A resulted in pandemics: H1 in 1918 and 1977; H2 in 1957 and H3 in 1968. In 1997, an H5 avian virus and in 1999, an H9 virus resulted in outbreaks of respiratory disease in Hong Kong.

New strains of the influenza virus emerge due to antigenic drift, a process whereby mutations within the virus antibody-binding sites accumulate over time. As a consequence of antigenic drift, the influenza virus can circumvent the infected subject's immune system, which may not be able to recognize and confirm immunity to a new influenza strain despite the immunity to different strains of the virus. Influenza A and B undergo antigenic drift.

Influenza A can also undergo antigenic shift resulting in a new virus subtype. Antigenic shift is a sudden change in viral antigenicity usually associated with recombination of the influenza genome that can occur when a cell is simultaneously infected by two different strains of influenza A virus.

In the 20th century, three influenza pandemics occurred in 1918, 1957, and 1968. The 1918 “Spanish flu” pandemic was clearly the most lethal, causing more than 500,000 deaths in the U.S. and as many as 50,000,000 deaths worldwide. Recent sequence and phylogenetic analysis suggest that the causative agent of the 1918 pandemic was an avian strain that adapted to humans (Taubenberger, J. K., et al., Nature 437:889). A similar threat may be occurring today.

Since 1996, there have been nearly 200 confirmed cases of avian influenza infection in humans with an apparent increase in incidence in southeast Asia in 2004 (Zeitlin, G. A., et al., Curr Infect Dis Rep 7:193). More recently, migratory wild birds have carried the disease as far as the Middle East and Eastern Europe (Fereidouni, S. R. et al., Vet Rec 157:526; Al-Natour, M. Q., et al., Prev Vet Med 70:45; Liu, J., et al. Science 309:1206; Chen, H., et al. Nature 436.191). With the growing incidence of human cases, close proximity of humans and domesticated bird flocks that are potential carriers of the disease, spread through migratory fowl, and the ease of human-to-human spread on a global scale (as experienced with severe acute respiratory syndrome (Poutanen, S. M., et al. N Engl J Med 348: 1995; MMWR Morb Mortal Wkly Rep 52: 1157)), there is a need to develop new, improved compositions, fusion proteins and polypeptides to protect subjects, in particular humans, from the potentially disastrous effects of another influenza pandemic.

The compositions, fusion proteins and polypeptides of the invention may be refractory to the genetic instability of the prototypical influenza targets, HA and neuraminidase (NA), which requires annual selection of multiple strains for use in preventing influenza infection. A composition, fusion protein and polypeptide based on a genetically stable antigen may provide long-lasting immunity to influenza infection, be useful year after year, and be particularly valuable in case of an influenza A pandemic.

M2 has genetic stability. The amino terminal 24 amino acid sequence (SEQ ID NO: 13, also referred to herein as “M2e”) has changed little in human pathogenic influenza virus strains isolated since 1933 (Neirynck, S., et al. Nature Medicine 5:1157). In mammals, M2 is poorly immunogenic in its native form; however, when administered with adjuvants or conjugated to an appropriate carrier backbone, M2e induces the production of specific antibodies that correlate with protection from subsequent live virus challenge (Neirynck, S., et al. Nature Medicine 5:1157; Frace, A. M., et al. Vaccine 17:2237; Mozdzanowska, K. et al. Vaccine 21: 2616; Fran, J., et al. Vaccine 22:2993). Antibodies to M2e also confer passive protection in animal models of influenza A infection (Treanor, J. J., et al. J. Virol 64:1375; Liu, W., et al. Immunol Lett 93:131), not by neutralizing the virus and preventing infectivity, but rather by killing infected cells and disrupting the viral life cycle (Zebedee, S. L., et al. J. Virol 62:I2762; Jegerlehner, A., et al. J. Immunol 172.5598). It has been proposed that one mechanism of protection is antibody-dependent NK cell activity (Jegerlehner, A., et al. J. Immunol 172.5598).

Immunization of pigs with an M2-nucleoprotein fusion protein exacerbated disease rather than protecting (Heinen, P. P., et al. J. Gen Virol 83.1851). However, these data were confounded by the multiple variables examined (fusion protein linking M2 to hepatitis B core antigen versus DNA immunization linking M2 to nucleoprotein), the dose of viral challenge, and the virus strain. More recently, immunization of ferrets with M2e peptide in the context of a complex carrier resulted in reduced lung viral titers upon subsequent challenge without exacerbation of clinical symptoms (Fran, J., et al. Vaccine 22:2993). Compositions, fusion proteins and polypeptides of the invention that include M2, in particular M2e, may limit the severity of influenza illness while allowing the host immune response to develop adaptive immunity to the dominant neutralizing influenza antigen, HA.

The compositions, fusion proteins and polypeptides of the invention can be employed in methods of stimulating an immune response in a subject. The compositions, fusion proteins and polypeptides of the invention can be administered alone or with currently available influenza vaccines and drugs. However, because the sequence of M2e is highly conserved across strains, HA/NA subtypes, and geographically and temporally-distinct isolates, the compositions, fusion proteins and polypeptides of the invention that include M2e may stimulate an immune response in a subject to M2e that may provide protection against a possible pandemic arising from the introduction of a totally new HA/NA subtype into a population nature to that subtype. The same genetic conservation lends itself to providing broad protection against a potential bioterrorism use of any influenza strain, such as influenza A.

The M2e sequence of certain avian influenza A isolates differs slightly from that of human isolates, but is highly-conserved among the avian isolates, as shown in Table 1 (infra). The compositions, fusion proteins and polypeptides of the invention that include M2e may target circulating human pathogenic strains of influenza A (H1 and H3 subtypes) as well as avian strains that present a pandemic threat (H5 subtypes).

Exemplary M2e amino acid sequences of the compositions, fusion proteins and polypeptides of the invention are shown in Table 1. The M2e amino acid sequences were based on Fan, et al. Vaccine 22:2993 (2004) or the NCBI Protein Database (http://www.ncbi.nlm.nih.gov/genomes/FLU/FLU.html). Variants in reference to A/New Caledonia/20/99 sequence are denoted by bolded and underlined letters. A cysteine (C) residue in the naturally occurring M2 sequence (e.g., SEQ ID NOS: 40, 42, 44 and 46, supra; and SEQ ID NOS: 48, 49 and 50, in Table 1, infra) can be substituted with serine (S) residue (e.g., SEQ ID NOS: 39, 41, 43 and 45, supra; and SEQ ID NOS: 54, 73 and 74 in Table 1, infra). Such substitution may improve solubility and structural integrity of the compositions, fusion proteins and polypeptides of the invention.

TABLE 1

| Representative | | | Amino acid |
|---|---|---|---|
| source | Subtype | Host | sequences |
| Human with serine | | | SLLTEVETPIRNEWGSR |
| replacing cysteine | | | SNDSSDP |
| | | | (SEQ ID NO: 47) |
| A/Puerto Rico/8/34 | H1N1 | Human | SLLTEVETPIRNEWGCR |
| | | | CNGSSDP |
| | | | (SEQ ID NO: 48) |
| | | | SLLTEVETPIRNEWGSR |
| | | | SNGSSDP |
| | | | (SEQ ID NO: 54) |
| A/Wisconsin/3523/ | H1N1 | Human | SLLTEVETPIRNEWGCK |
| 88 | | | CNDSSDP |
| | | | (SEQ ID NO: 49) |
| | | | SLLTEVETPIRNEWGSK |
| | | | SNDSSDP |
| | | | (SEQ ID NO: 73) |
| A/New Caledonia/ | H1N1 | Human | SLLTEVETPIRNEWGCR |
| 20/99 | | | CNDSSDP |
| | | | (SEQ ID NO: 50) |
| | | | SLLTEVETPIRNEWGSR |
| | | | SNDSSDP |
| | | | (SEQ ID NO: 74) |
| A/Aichi/470/68 | H3N1 | human | SLLTEVETPIRNEWGCR |
| | | | CNDSSDP |
| | | | (SEQ ID NO: 51) |
| A/Hebei/19/95 | H3N2 | human | SLLTEVETPIRNEWECR |
| | | | CNGSSDP |
| | | | (SEQ ID NO: 52) |
| | | | SLLTEVETPIRNEWESR |
| | | | SNGSSDP |
| | | | (SEQ ID NO: 75) |
| A/Chicken/Nakorn- | H5N1 | avian | SLLTEVETPTRNEWECR |
| Patom/Thailand | | | CSDSSDP |
| | | | (SEQ ID NO: 53) |
| A/Thailand/1 | H5N1 | avian | SLLTEVETPTRNEWECR |
| (KAN-1)/04 | | | CSDSSDP |
| | | | (SEQ ID NO: 53) |
| | | | SLLTEVETPTRNEWESR |
| | | | SSDSSDP |
| | | | (SEQ ID NO: 76) |
| A/Hong Kong/156/97 | H5N1 | human | SLLTEVETLTRNGWGCR |
| | | | CSDSSDP |
| | | | (SEQ ID NO: 55) |
| | | | SLLTEVETLTRNGWGSR |
| | | | SSDSSDP |
| | | | (SEQ ID NO: 77) |
| A/Viet Nam/1203/ | H5N1 | human | SLLTEVETPTRNEWECR |
| 2004 | | | CSDSSDP |
| | | | (SEQ ID NO: 56) |
| | | | SLLTEVETPTRNEWESR |
| | | | SSDSSDP |
| | | | (SEQ ID NO: 78) |
| A/Chicken/New | H7N2 | avian | SLLTEVETPTRNGWECK |
| York/95 | | | CSDSSDP |
| | | | (SEQ ID NO: 57) |
| | | | SLLTEVETPTRNGWESK |
| | | | SSDSSDP |
| | | | (SEQ ID NO: 79) |
| A/Chicken/Hong | H9N2 | avian | SLLTEVETPTRNGWGCR |
| Kong/G9/97 | | | CSGSSDP |
| | | | (SEQ ID NO: 58) |
| | | | SLLTEVETPTRNGWGSR |
| | | | SSGSSDP |
| | | | (SEQ ID NO: 80) |
| A/Hong Kong/1073/ | H9N2 | human | SLLTEVETLTRNGWECK |
| 99 | | | CRDSSDP |
| | | | (SEQ ID NO: 59) |
| | | | SLLTEVETLTRNGWESK |
| | | | SRDSSDP |
| | | | (SEQ ID NO: 81) |

In a particular embodiment, the compositions, fusion proteins and polypeptides of the invention include a pathogen-associated molecular pattern. Certain PAMPs (e.g., TLR ligands, TLR agonists) bind TLR, which act as initiators of the innate immune response and gatekeepers of the adaptive immune response (Medzhitov, R., et al. Nature: 388:394; Medzhitov, R., et al., Cold Spring Harb Symp Quant Biol 64:429; Pasare, C., et al. Semin Immunol 16.23; Barton, G. M., et al. Curr Opin Immunol 14:380; Bendelac, A., et al. J Exp Med 195:F19). TLRs are the best characterized type of Pattern Recognition Receptor (PRR) expressed on antigen-presenting cells (APC). APC utilize TLRs to survey the microenvironment and detect signals of pathogenic infection by engaging the cognate ligands of TLRs, Pathogen-Associated Molecular Patterns (PAMPs). PAMP and TLR interaction triggers the innate immune response, the first line of defense against pathogenic insult, manifested as release of cytokines, chemokines and other inflammatory mediators; recruitment of phagocytic cells; and important cellular mechanisms which lead to the expression of costimulatory molecules and efficient processing and presentation of antigens to T-cells. TLRs control both innate and the adaptive immune responses.

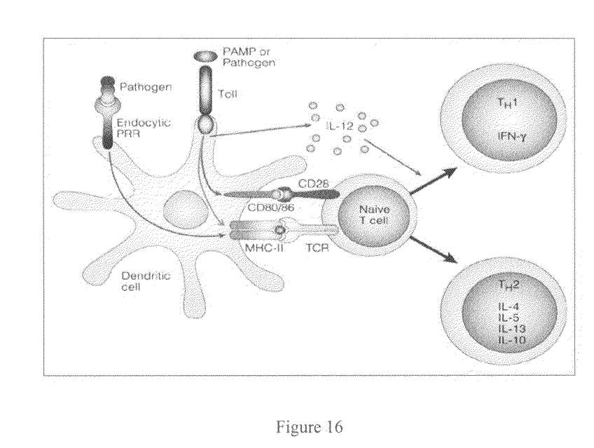

TLRs recognize PAMPs including bacterial cell wall components such as lipoproteins (TLR2) and lipopolysaccharides (TLR4), bacterial DNA sequences that contain unmethylated CpG residues (TLR9), and bacterial flagellin (TLR5). The binding of PAMPs to TLRs activates well-characterized immune pathways that can be mobilized for the development of more potent compositions, fusion proteins and polypeptides of the invention. The compositions, fusion proteins and polypeptides can be generated in a manner that ensure that those cells that are exposed to protective antigen(s) of the pathogenic agent also receive an innate immune signal (TLR activation) and vice versa. This can be effectively achieved by designing the compositions, fusion proteins and polypeptides to include at least a portion of at least one PAMP and at least a portion of at least one influenza viral protein (e.g., an integral membrane protein). The compositions, fusion proteins and polypeptides of the invention can trigger signal transduction pathways in their target cells that result in the display of co-stimulatory molecules on the cell surface, as well as antigenic peptide in the context of major histocompatibility complex molecules (see FIG. 16).

FIG. 16 depicts the activation of an APC by TLR signaling. The composition, fusion protein or polypeptide of the invention includes a PAMP that binds to a TLR, promoting differentiation and maturation of the APC, including production and display of co-stimulatory signals. The composition, fusion protein or polypeptide can be internalized by its interaction with the TLR and processed through the lysosomal pathway to generate antigenic peptides, which are displayed on the surface in the context of the major histocompatibility complex.

An “effective amount,” when referring to the amount of a composition, fusion protein or a polypeptide of the invention, refers to that amount or dose of the composition, fusion protein, or a polypeptide, that, when administered to the subject is an amount sufficient for therapeutic efficacy (e.g., an amount sufficient to stimulate an immune response in the subject). The compositions, fusion proteins, or polypeptides of the invention can be administered in a single dose or in multiple doses.

The methods of the present invention can be accomplished by the administration of the compositions, fusion proteins or polypeptides of the invention by enteral or parenteral means. Specifically, the route of administration is by oral ingestion (e.g., drink, tablet, capsule form) or intramuscular injection of the composition, fusion protein or polypeptide. Other routes of administration as also encompassed by the present invention including intravenous, intradermal, intraarterial, intraperitoneal, or subcutaneous routes, and nasal administration. Suppositories or transdermal patches can also be employed.

The compositions, fusion proteins or polypeptides of the invention can be administered ex vivo to a subject's autologous dendritic cells. Following exposure of the dendritic cells to the composition, fusion protein or polypeptide of the invention, the dendritic cells can be administered to the subject.

The compositions, fusion proteins or polypeptides of the invention can be administered alone or can be coadministered to the patient. Coadministration is meant to include simultaneous or sequential administration of the composition, fusion protein or polypeptide of the invention individually or in combination. Where the composition, fusion protein or polypeptide are administered individually, the mode of administration can be conducted sufficiently close in time to each other (for example, administration of the composition close in time to administration of the fusion protein) so that the effects on stimulating an immune response in a subject are maximal. It is also envisioned that multiple routes of administration (e.g., intramuscular, oral, transdermal) can be used to administer the compositions and fusion proteins of the invention.

The compositions, fusion proteins or polypeptide of the invention can be administered alone or as admixtures with conventional excipients, for example, pharmaceutically, or physiologically, acceptable organic, or inorganic carrier substances suitable for enteral or parenteral application which do not deleteriously react with the extract. Suitable pharmaceutically acceptable carriers include water, salt solutions (such as Ringer's solution), alcohols, oils, gelatins and carbohydrates such as lactose, amylose or starch, fatty acid esters, hydroxymethycellulose, and polyvinyl pyrrolidine. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like which do not deleteriously react with the compositions, fusion proteins or polypeptides of the invention. The preparations can also be combined, when desired, with other active substances to reduce metabolic degradation. The compositions, fusion proteins or polypeptides of the invention can be administered by is oral administration, such as a drink, intramuscular or intraperitoneal injection. The compositions, fusion proteins, or polypeptides alone, or when combined with an admixture, can be administered in a single or in more than one dose over a period of time to confer the desired effect (e.g., alleviate prevent viral infection, to alleviate symptoms of viral infection).

When parenteral application is needed or desired, particularly suitable admixtures for the compositions, fusion proteins or polypeptides are injectable, sterile solutions, preferably oily or aqueous solutions, as well as suspensions, emulsions, or implants, including suppositories. In particular, carriers for parenteral administration include aqueous solutions of dextrose, saline, pure water, ethanol, glycerol, propylene glycol, peanut oil, sesame oil, polyoxyethylene-block polymers, and the like. Ampules are convenient unit dosages. The compositions, fusion proteins or polypeptides can also be incorporated into liposomes or administered via transdermal pumps or patches. Pharmaceutical admixtures suitable for use in the present invention are well-known to those of skill in the art and are described, for example, in Pharmaceutical Sciences (17th Ed., Mack Pub. Co., Easton, Pa.) and WO 96/05309 the teachings of which are hereby incorporated by reference.

The compositions, fusion proteins and polypeptides of the invention can be administered to a subject on a presenting carrier. “Presenting carrier,” as used herein, means any composition that presents the compositions, fusion proteins and polypeptides of the invention to the immune system of the subject to generate an immune response in the subject. The presentation of the compositions, fusion proteins and polypeptides of the invention would preferably include exposure of antigenic portions of the influenza viral protein to generate antibodies. The components (PAMP and an integral membrane protein of an influenza virus) of the compositions, fusion proteins and polypeptides of the invention are in close physical proximity to one another on the presentation carrier. The compositions, fusion proteins and polypeptides of the invention can be attached to the presentation carrier by covalent or noncovalent attachment. Preferably, the presentation carrier is biocompatible. “Biocompatible,” as used herein, means that the presentation carrier does not generate an immune response in the subject (e.g., the production of antibodies). The presentation carrier can be a biodegradable substrate carrier, such as a polymer bead or a liposome. The presentation carrier can further include alum or other suitable adjuvants.

The compositions and methods of the invention can further include a carrier. “Carrier,” as used herein, refers to a molecule (e.g., protein, peptide) that can enhance stimulation of a protective immune response. Carriers can be physically attached (e.g., linked by recombinant technology, peptide synthesis, chemical conjugation or chemical reaction) to a composition (e.g., a protein portion of a naturally occurring viral hemagglutinin) or admixed with the composition.

Carriers for use in the methods and compositions described herein can include, for example, at least one member selected from the group consisting of Tetanus toxoid (TT), Vibrio cholerae toxoi d, Diphtheria toxoid (DT), a cross-reactive mutant (CRM) of diphtheria toxoid, E. coli enterotoxin, E. coli B subunit of heat labile enterotoxin (LTB), Tobacco mosaic virus (TMV) coat protein, protein Rabies virus (RV) envelope protein (glycoprotein), thyroglobulin (Thy), heat shock protein HSP 60 Kda, Keyhole limpet hemocyamin (KLH), an early secreted antigen tuberculosis-6 (ESAT-6), exotoxin A, choleragenoid, hepatitis B core antigen, and the outer membrane protein complex of N. meningiditis (OMPC) (see, for example, Schneerson, R., et al., Prog Clin Biol Res 47:77-94 (1980); Schneerson, R., et al., J Exp Med 152:361-76 (1980); Chu, C., et al., Infect Immun 40: 245-56 (1983); Anderson, P., Infect Immun 39:233-238 (1983); Anderson, P., et al., J Clin Invest 76:52-59 (1985); Fenwick, B. W., et al., 54: 583-586 (1986); Que, J. U., et al. Infect Immun 56:2645-9 (1988); Que, J. U., et al. Infect Immun 56:2645-9 (1988); (Que, J. U., et al. Infect Immun 56:2645-9 (1988); Murray, K., et al., Biol Chem 380:277-283 (1999); Fingerut, E., et al., Vet Immunol Immunopathol 112:253-263 (2006); and Granoff, D. M., et al., Vaccine 11:Suppl 1:S46-51 (1993)).

Exemplary carrier proteins for use in the methods and compositions described herein can include at least one member selected from the group consisting of SEQ ID NOS: 233-240:

Cross-reactive mutant (CRM) of diphtheria toxin
including, CRM197

(SEQ ID NO: 233)

GADDVVDSSKSFVMENFSSYHGTKPGYVDSIQKGIQKPKSGTQGNYDDDW
KGFYSTDNKYDAAGYSVDNENPLSGKAGGVVKVTYPGLTKVLALKVDNAE
TIKKELGLSLTEPLMEQVGTEEFIKRFGDGASRVVLSLPFAEGSSSVEYI
NNWEQAKALSVELEINFETRGKRGQDAMYEYMAQACAGNRVRRSVGSSLS
CINLDWDVIRDKTKTKIESLKEHGPIKNKMSESPNKTVSEEKAKQYLEEF
HQTALEHPELSELKTVTGTNPVFAGANYAAWAVNVAQVIDSETADNLEKT
TAALSILPGIGSVMGIADGAVHHNTEEIVAQSIALSSLMVAQAIPLVGEL
VDIGFAAYNFVESIINLFQVVHNSYNRPAYSPGHKTQPFLHDGYAVSWNT
VEDSIIRTGFQGESGHDIKITAENTPLPIAGVLLPTIPGKLDVNKSKTHI
SVNGRKIRMRCRAIDGDVTFCRPKSPVYVGNGVHANLHVAFHRSSSEKIH
SNEISSDSIGVLGYQKTVDHTKVNSKLSLFFEIKS
Coat protein of Tobacco mosaic virus (TMV) coat
protein

(SEQ ID NO: 234)

MMAYSIPTPSQLVYFTENYADYIPFVNRLINARSNSFQTQSGRDELREIL
IKSQVSVVSPISRFPAEPAYYIYLRDPSISTVYTALLQSTDTRNRVIEVE
NSTNVTTAEQLNAVRRTDDASTAIHNNLEQLLSLLTNGTGVFNRTSFESA
SGLTWLVTTTPRTA
Coat protein of alfalfa mosaic virus (AMV)

(SEQ ID NO: 235)

MSSSQKKAGGKAGKPTKRSQNYAALRKAQLPKPPALKVPVAKPTNTILPQ
TGCVWQSLGTPLSLSSSNGLGARFLYSFLKDFAAPRILEEDLIFRMVFSI
TPSHAGSFCLTDDVTTEDGRAVAHGNPMQEFPHGAFHANEKFGFELVFTA
PTHAGMQNQNFKHSYAVALCLDFDALPEGSRNPSYRFNEVWVERKAFPRA
GPLRSLITVGLFDDADDLDRQ
Coat protein of Potato virus X

(SEQ ID NO: 236)

MTTPANTTQATGSTTSTTTKTAGATPATTSGLFTIPDGEFFSTARAIVAS
NAVATNEDLSKIEAIWKDMKVPTDTMAQAAWDLVRHCADVGSSAQTEMID
TGPYSNGISRARLAAAIKEVCTLRQFCMKYAPVVWNWMLTNNSPPANWQA
QGFKPEHKFAAFDFFNGVTNPAAIMPKEGLIRPPSEAEMNAAQTAAFVKI
TKARAQSNDFASLDAAVTRGRITGTTTAEAVVTLPPP
Porins from Neisseria sp, e.g.,
class I outer membrane protein of Neisseria
meningitides

(SEQ ID NO: 237)

MRKKLTALVLSALPLAAVADVSLYGEIKAGVEGRNYQLQLTEAQAANGGA
SGQVKVTKVTKAKSRIRTKISDFGSFIGFKGSEDLGEGLKAVWQLEQDVS
VAGGGATQWGNRESFIGLAGEFGTLRAGRVANQFDDASQAIDPWDSNNDV
ASQLGIFKRHDDMPVSVRYDSPEFSGFSGSVQFVPAQNSKSAYKPAYWTT
VNTGSATTTTFVPAVVGKPGSDVYYAGLNYKNGGFAGNYAFKYARHANVG
RDAFELFLLGSGSDQAKGTDPLKNHQVHRLTGGYEEGGLNLALAAQLDLS
ENGDKTKNSTTEIAATASYRFGNAVPRISYAHGFDFIERGKKGENTSYDQ
IIAGVDYDFSKRTSAIVSGAWLKRNTGIGNYTQINAASVGLRHKF
Major fimbrial subunit protein type I (Fimbrillin)

(SEQ ID NO: 238)

MVLKTSNSNRAFGVGDDESKVAKLTVMVYNGEQQEAIKSAENATKVEDIK
CSAGQRTLVVMANTGAMELVGKTLAEVKALTTELTAENQEAAGLIMTAEP
KTIVLKAGKNYIGYSGTGEGNHIENDPLKIKRVHARMAFTEIKVQMSAAY
DNIYTFVPEKIYGLIAKKQSNLFGATLVNADANYLTGSLTTFNGAYTPAN
YANVPWLSRNYVAPAADAPQGFYVLENDYSANGGTIHPTILCVYGKLQKN
GADLAGADLAAAQAANWVDAEGKTYYPVLVNFNSNNYTYDSNYTPKNKIE
RNHKYDIKLTITGPGTNNPENPITESAHLNVQCTVAEWVLVGQNATW
Mycoplasma fermentans macrophage activating
lipopeptide (MALP-2)

(SEQ ID NO: 239)

MKKSKKILLGLSPIAAVLPAVAVSCGNNDESNISFKEKDISKYTTTNANG
KQVVKNAELLKLKPVLITDEGKIDDKSFNQSAFEALKAINKQTGIEINSV
EPSSNFESAYNSALSAGHKIWVLNGFKHQQSIKQYIDAHREELERNQIKI
IGIDFDIETEYKWFYSLQFNIKESAFTTGYAIASWLSEQDESKRVVASFG
VGAFPGVTTFNEGFAKGILYYNQKHKSSKIYHTSPVKLDSGFTAGEKMNT
VINNVLSSTPADVKYNPHVILSVAGPATFETVRLANKGQYVIGVDSDQGM
IQDKDRILTSVLKHIKQAVYETLLDLILEKEEGYKPYVVKDKKADKKWSH
FGTQKEKWIGVAENHFSNTEEQAKINNKIKEAIKMFKELPEDFVKYINSD
KALKDGNKIDNVSERLEAIISAINKAAK
p19 protein of Mycobacterium tuberculosis

(SEQ ID NO: 240)

ATTLPVQRHPRSLFPEFSELFAAFPSFAGLRPTFDTRLMRLEDEMKEGRY
EVRAELPGVDPDKDVDIMVRDGQLTIKAERTEQKDFDGRSEFAYGSFVRT
VSLPVGADEDDIKATYDKGILTVSVAVSEGKPTEKHIQIRSTN

The compositions of the invention can further include at least one adjuvant. Adjuvants contain agents that can enhance the immune response against substances that are poorly immunogenic on their own (see, for example, Immunology Methods Manual, vol. 2, I. Lefkovits, ed., Academic Press, San Diego, Calif., 1997, ch. 13). Immunology Methods Manual is available as a four volume set, (Product Code Z37, 435-0); on CD-ROM, (Product Code Z37, 436-9); or both, (Product Code Z37, 437-7). Adjuvants can be, for example, mixtures of natural or synthetic compounds that, when administered with compositions of the invention, such as proteins that stimulate a protective immune response made by the methods described herein, further enhance the immune response to the protein. Compositions that further include adjuvants may further increase the protective immune response stimulated by compositions of the invention by, for example, stimulating a cellular and/or a humoral response (i.e., protection from disease versus antibody production). Adjuvants can act by enhancing protein uptake and localization, extend or prolong protein release, macrophage activation, and T and B cell stimulation. Adjuvants for use in the methods and compositions described herein can be mineral salts, oil emulsions, mycobacterial products, saponins, synthetic products and cytokines. Adjuvants can be physically attached (e.g., linked by recombinant technology, by peptide synthesis or chemical reaction) to a composition described herein or admixed with the compositions described herein.

The dosage and frequency (single or multiple doses) administered to a subject can vary depending upon a variety of factors, including prior exposure to a viral antigen, the duration of viral infection, prior treatment of the viral infection, the route of administration of the composition, fusion protein or polypeptide; size, age, sex, health, body weight, body mass index, and diet of the subject; nature and extent of symptoms of influenza exposure, influenza infection and the particular influenza virus responsible for the infection (e.g., influenza A, B, C), the source of the influenza virus (e.g., Hong Kong, Puerto Rico, Wisconsin, Thailand) kind of concurrent treatment (e.g., nasal sprays and drugs, such as amantadine, rimantadine, zanamivir and oseltamivir), complications from the influenza exposure, influenza infection or other health-related problems. Other therapeutic regimens or agents can be used in conjunction with the methods and compositions, fusion proteins or polypeptides of the present invention. For example, the administration of the compositions, fusion proteins or polypeptides can be accompanied by other viral therapeutics or use of agents to treat the symptoms of the influenza infection (e.g., nasal sprays and drugs, such as amantadine, rimantadine, zanamivir and oseltamivir). Adjustment and manipulation of established dosages (e.g., frequency and duration) are well within the ability of those skilled in the art.

The present invention is further illustrated by the following examples, which are not intended to be limiting in any way.

### EXEMPLIFICATION

### Example 1

### Flagellin-M2e Fusion Proteins

M2e is conserved across multiple influenza A subtypes (also referred to herein as “strain”). M2e is at least a portion of the M2 protein, in particular, a 24 amino-terminus (also referred to herein as an “ectodomain”) of the M2 protein. The M2 ectodomain is relatively small amino acid sequence (24 amino acids) compared to HA (about 566 amino acids) and NA (about 469 amino acids). The M2e sequence of exemplary avian influenza A isolates differs from that of human isolates, but is highly-conserved among the avian isolates (see Table 1, supra). Four tandem copies of M2e fused to the carboxy terminus of a flagellin STF2 (full-length or STF2 hinge region-deleted) were generated. The STF2 without the hinge region is also referred to herein as “STF2Δ.”

### Construction of Fusion Protein

The carboxy-terminal fusion of the synthetic 4×M2e sequence (4 consecutive 24 amino acid sequences) with STF2 was constructed as follows. The pET24A vector was purchased from Novagen, San Diego, Calif. The strategy employed the Seamless Cloning Kit (Catalog number 214400) from Stratagene (La Jolla, Calif. www.stratagene.com) performed by DNA 2.0 Inc. (Menlo Park, Calif.). The gene encoding the fusion protein was in pDrive 4×M2E G00448 and was used as a PCR template for insert preparation for construction of the C-terminal fusion expression construct with STF2. The synthetic 4×M2E construct pDrive 4×M2E G00448 was used as a template for PCR as outlined in the Seamless Cloning Kit (Catalog number 214400) from Stratagene (La Jolla, Calif.). The expected product from this amplification includes the 318 bp and the restriction enzyme sites incorporated into the oligonucleotides used to amplify this insert. The procedure was as follows:

### PCR Conditions

1 μL −20 ng of pDrive 4×M2E G00448
5 μL of 10× cloned Pfu polymerase buffer
1 μL of 40 mM dNTP mix
1 μL −10 pmol of forward primer 4×M2Eforbs1
1 μL −10 pmol of reverse primer 4×M2Erevwsto
40 μL ddH₂O

Immediately before starting the thermal cycling 1 μL of PfuTurbo DNA Polymerase the following were added.

4xM2Eforbs1 primer sequence:

(SEQ ID NO: 69)

5′-CGCTCTTCAMTGAGCTTGCTGACTGAGGTTGAGACCCCGATTC
4xM2Erevwsto primer sequence:

(SEQ ID NO: 70)

5′-CGCTCTTCACGCTTATTATCTAGACGGGTCTGAGCTATCGTTAGAG
CGAG

This reaction was cycled as follows on a Thermo Hybaid P×E thermal cycler (Waltham, Mass.).

Initial Cycle

| Temperature | Duration |
|---|---|
| 95° | 3 minutes |
| 65° | 1 minute |
| 72° | 1 minute |

Subsequent Nine Cycles

| Temperature | Duration |
|---|---|
| 95° | 45 seconds |
| 65° | 35 seconds |
| 72° | 1 minute |

At this point the following was added to each reaction.

5 μL of 10× cloned Pfu polymerase buffer
1 μL of 5-methyl dNTP mix
44 μL ddH₂O

Subsequently the following thermal cycling was repeated five times.

| Temperature | Duration |
|---|---|
| 95° | 45 seconds |
| 65° | 35 seconds |
| 72° | 1 minute |

The 100 μL product was brought to a volume of 300 μL by the addition of TE buffer. The resulting product was phenol chloroform (Invitrogen Carlsbad, Calif.-Catalog number 15593-031) extracted once and chloroform extracted once. The amplification product was then ethanol precipitated by addition of 30 μL of Sodium acetate buffer pH 5.2 and 750 μL of 100% Ethanol. The DNA pellet was washed twice with 300 μL 70% Ethanol allowed to air dry for ten minutes and then resuspended in 50 μL TE buffer.

Amplification of Vector STF2 in pET24.

The previously constructed pET24a/STF2.M2e construct was used as a template for PCR as outlined in the Seamless Cloning Kit (Catalog number 214400) from Stratagene (La Jolla, Calif.). The expected product from this amplification includes the whole of the pET24 plasmid plus the STF2 sequences but does not include the single copy of M2E that exists in this construct. The procedure was as follow:

### Pcr Conditions

1 μL −40 ng of STF2.M2E pET22-2
5 μL of 10× cloned Pfu polymerase buffer
1 μL of 40 mM dNTP mix
1 μL −10 pmol of primer 4×MECpET24
1 μL −10 pmol of primer 4×M2EC-STF2
40 μL ddH₂O

Immediately before starting the thermal cycling the following were added:

### 1 μL of PfuTurbo DNA Polymerase

4× MECpET24 primer sequence:

4xMECpET24 primer sequence:

(SEQ ID NO: 71)

5′-GCTCTTCAGCGGCTGAGCAATAACTAGCATAACCCCTTGGG
4xM2EC-STF2 primer sequence:

(SEQ ID NO: 72)

5′-CGCTCTTCACAGACGTAACAGAGACAGCACGTTCTGCGG

This reaction was cycled as follows on a Thermo Hybaid P×E thermal cycler (Waltham, Mass.).

### Initial Cycle

| Temperature | Duration |
|---|---|
| 95° | 3 minutes |
| 65° | 1 minute |
| 72° | 18 minutes |

### Subsequent Nine Cycles

| Temperature | Duration |
|---|---|
| 95° | 45 seconds |
| 65° | 35 seconds |
| 72° | 18 minutes |

At this point the following was added to each reaction.

5 μL of 10× cloned Pfu polymerase buffer
1 μL of 5-methyl dNTP mix
44 μL ddH₂O

Subsequently the following thermal cycling was repeated five times.

| Temperature | Duration |
|---|---|
| 95° | 45 seconds |
| 65° | 35 seconds |
| 72° | 18 minutes |

The 100 μL product was brought to a volume of 300 μL by the addition of TE buffer. The resulting product was phenol chloroform (Invitrogen Carlsbad, Calif.-Catalog number 15593-031) extracted once and chloroform extracted once. The amplification product was then ethanol precipitated by addition of 30 μL of Sodium acetate buffer pH 5.2 and 750 μL of 100% Ethanol. The DNA pellet was washed twice with 300 μL 70% Ethanol allowed to air dry for ten minutes and then resuspended in 50 μL TE buffer.

### Digestion and Ligation of Vector and Insert Amplifications

Eam 1104 I digests were set up separately for vector and insert as follows:

30 μL of amplified product after ethanol precipitation
5 μL of 10× Universal buffer (Supplied with Seamless Cloning Kit)
4 μL Eam 1104 I restriction enzyme (Supplied with Seamless Cloning Kit)
11 μL ddH₂O

Digests were mixed gently and incubated at 37° C. for one hour and ligation reactions of vector and insert products were prepared as above performed as follows (Reagents supplied with Seamless Cloning Kit):

Ingredients added in order listed:

9 μL ddH₂O
5 μL of Eam 1104 I digested 4×M2E amplified insert
5 μL of Eam 1104 I digested STF2.M2E pET22-2 amplified vector
2 μL 10× Ligase buffer
2 μL 10 mM rATP
1 μL T4 DNA Ligase (diluted from stock 1:16)
1 μL Eam 1104 I restriction enzyme

The ligation reactions were mixed gently and incubated for 30 minutes at 37° C. The ligations were then stored on ice until transformed into XL-10 competent cells (Stratagene Catalog number 200314) later than same day.

Transformation of Ligation into XL-10 Competent Cells

Eppendorf tubes were chilled for ten minutes while the XL-10 (Stratagene Catalog number 200314) competent cells thawed on ice.

50 μL of competent cells were aliquoted from the stock tube per ligation.
2 μL of β-mercaptoethanol stock which is provided with the XL-10 cells.

This mixture was incubated for ten minutes on ice gently mixing every 2 minutes. Seamless cloning ligation reaction (4 μl) was added, swirled gently and then incubated on ice for 30 minutes. The tubes were heat shocked for 35 seconds at 42° C. in a water bath. The tubes were incubated on ice for at least two minutes. SOC medium (400 μL) were added to the cells and incubated for one hour at 37° C. with agitation. Two LB agar kanamycin (50 μg/mL) plates are used to plate 200 μL and 10 μL of the transformed cells and allowed to grow overnight.

### Screening of Kanamycin Resistant Clones

Recombinant candidates were grown up for minipreps in Luria Broth containing Kanamycin (25 ug/mL) and extracted using the QIAprep Spin Miniprep Kit (Qiagen Valencia, Calif. Catalog Number 27106). Candidate clones were screened by restriction enzymes (New England Biolabs Beverly, Mass.) and positive clones were grown up in 100 mL of Luria Broth containing kanamycin (25 ug/mL) and extracted using the Qiagen HiSpeed Plasmid Midi Kit (Catalog number 12643). These clones were submitted to GENEWIZ (North Brunswick, N.J.) for sequencing.

### Production and Purification of STF2.4×M2E Fusion Protein

STF2.4×M2e in E. coli BLR(DE3)pLysS host (Novagen, San Diego, Calif., Catalog #69053) was retrieved from glycerol stock and scaled up to 5 L. Cells were grown in LB medium containing 15 μg/ml Kanamycin and 12.5 μg/ml Teteracycline to OD₆₀₀=0.4 and induced with 1 mM IPTG for 3 h at 37° C. The cells were harvested by centrifugation (7000 rpm×7 minutes in a Sorvall RC5C centrifuge) and resuspended in 2×PBS, 1% glycerol, DNAse, 1 mM PMSF, protease inhibitor cocktail and 1 mg/ml lysozyme. The suspension was passed through a microfluidizer to lyse the cells. The lysate was centrifuged (45,000 g for one hour in a Beckman Optima L ultracentrifuge) to separate the soluble fraction from inclusion bodies. Protein was detected by SDS-PAGE in the soluble and insoluble fractions.

The soluble fraction was applied to Sepharose Q resin in the presence of high salt via batch method to reduce DNA, endotoxin, and other contaminants. The flow through containing the protein of interest was loaded onto 30 ml Q Sepharose column (Amersham Biosciences). Bound protein was eluted using a linear gradient from Buffer A to B. (Buffer A: 100 mM Tris-Cl, pH 8.0. Buffer B: 100 mM Tris-Cl, 1 M NaCl, pH 8.0). Eluted protein was further purified using a 45 ml Source Q column that provided greater resolution needed to resolve contaminating proteins. Bound protein was eluted with a linear gradient from Buffer A to B (Buffer A: 100 mM Tris-Cl, pH 8.0 Buffer B: 100 mM Tris-Cl, 1 M NaCl, pH 8.0).

Final purification of protein was completed using Superdex-200 gel filtration chromatography. The column was developed with 100 mM Tris, 150 mM NaCl and 1% glycerol plus 1% Na-deoxycholate to remove the LPS. Buffer exchange was carried out using overnight dialysis against buffer containing 50 mM Tris, 100 mM NaCl and 1% glycerol was done to remove Na-deoxycholate. Protein concentration was determined by the MicroBCA Protein Assay Reagent Kit (Pierce Biotechnology). Purified preparations of STF2.4×M2e yielded a single band visible with Coomassie stain that migrated with an apparent molecular weight of about 64 kDa on 12% SDS polyacrylamide gels.

### Example 2

### Expression and Purification of Flagellin (STF2 and STF2Δ) Fusion Protein Constructs Encoding Influenza a M2 Ectodomain Sequences

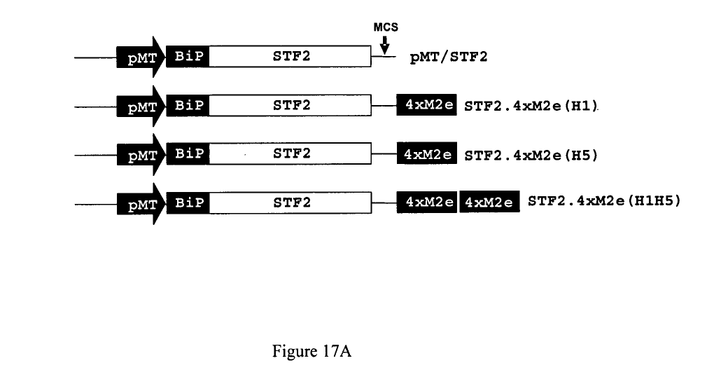

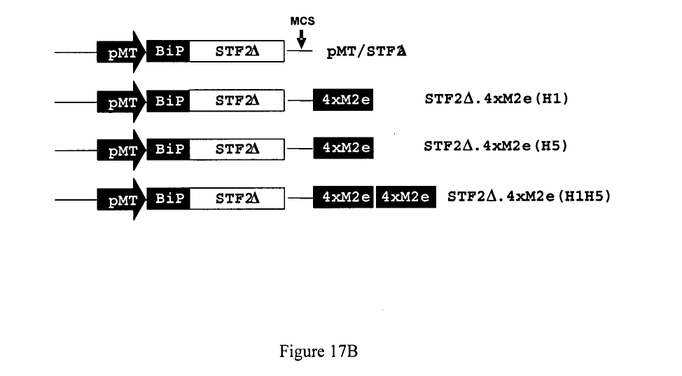

The consensus M2e sequences from several influenza A strains of human and avian origin are depicted in Table 1. To facilitate the cloning of the M2e sequence, two vector cassettes, pMT/STF2 and pMT/STF2Δ, each containing a multiple cloning site (MCS) were generated (See FIGS. 17A and 17B). To generate pMT/STF2, the 1.5 kb gene encoding full length flagellin of Salmonella typhimurium fljb type 2, or STF2, was fused to the Ig binding protein (BIP) secretion signal of pMTBIP/V5-His vector (Invitrogen Corporation, Carlsbad, Calif.) for expression in Drosophila. The BiP sequence is included at the 5′ end of the construct as a secretion signal for expression in Drosophila. A chemically-synthesized 4×M2e gene representing the H1, H2 and H3 consensus sequence, SLLTEVETPIRNEWGSRSNDSSDP (SEQ ID NO: 47, Table 1), was cloned into the MCS of pMT/STF2 to create pMT/STF2.4×M2e(H1).

A similar strategy prophetically is employed to clone two H5-associated M2e sequences, SLLTEVETPTRNEWECRCSDSSDP (SEQ ID NO: 56) (A/Viet Nam/1203/2004) and SLLTEVETLTRNGWGCRCSDSSDP (SEQ ID NO: 55) (A/Hong Kong/156/97). Codon-optimized chemically synthesized genes containing four tandemly repeated copies of the indicated H5-associated M2e sequence prophetically are cloned into pMT/STF2 to generate STF2.4×M2e(H5VN) and STF2.4×M2e(H5HK), respectively. To generate a construct that contains multiple M2e forms, the heterologous 4×M2e sequence(s) prophetically are inserted into either of the primary constructs.

“Heterologous sequences,” as used herein, means sequences from different species. For example, the H1 sequence is a human sequence and the H5 sequence is an avian sequence. Thus, the H1 and H5 sequences are heterologous sequences (e.g., SLLTEVETPTRNEWESRSSDSSDPLESLLTEVETPTRNEWESRSSDSSDPESSLLT EVETPTRNEWESRSSDSSDPGSSLLTEVETPTRNEWESRSSDSSDP (SEQ ID NO: 100), encoded by tctctgctgactgaagtagaaactccaacgcgtaatgaatgggaatcccgttctagcgactcctctgatcctctcgagtccctgct gacggaggttgaaaccccgacccgcaacgagtgggaaagccgttcctccgattcctctgatccggagagcagcctgctgac cgaggtagaaaccccgacccgtaatgagtgggaatctcgctcctctgattcttctgacccgggatcctctctgctgaccgaagt ggagactccgactcgcaacgaatgggagagccgttcftctgactcctctgacccg (SEQ ID NO: 101).

Primary constructs comprise at least one pathogen-associated molecular pattern (e.g., STF2, STF2Δ) and at least a portion of at least one integral membrane protein (e.g., M2e, such as SEQ ID NOS: 13 and 47). If there is more than one integral membrane in a primary construct, the integral membrane proteins are from the same species.

A heterologous construct includes at least two integral membrane proteins such as Hi (human) and H5 (avian), for example, in SEQ ID NOS: 86 and 87.

To generate pMT/STF2Δ, the hyper-variable region that spans amino acids 170 to 415 of the full-length flagellin gene of SEQ ID NO: 2 was deleted and replaced with a short (10 amino acid) flexible linker (GAPVDPASPW, SEQ ID NO: 97) designed to facilitate interactions of the amino and carboxy terminal sequences necessary for TLR5 signaling. The protein expressed from this construct retains potent TLR5 activity whether expressed alone or in fusion with test antigen. Thus, a second series of M2e constructs prophetically is generated based on pMT/STF2Δ. Drosophila Dmel-2 cells (Invitrogen Corporation, Carlsbad, Calif.) grown at room temperature in Schneider's medium supplemented with 10% FBS and antibiotics prophetically is transfected with the constructs described above using Cellfectin reagent (Invitrogen) according to the manufacturer's instructions. Twenty-four hours post transfection, cells prophetically is induced with 0.5 mM CuSO₄ in medium lacking FBS and incubated for an additional 48 hours. Conditioned media (CM) prophetically is harvested from induced cultures and screened for protein expression by SDS-PAGE and Western blot analyses using anti-flagellin and anti-M2e specific antibodies. The identity, TLR bioactivity of the fusion protein, antigenicity assessed by ELISA and in vivo mouse studies for immunogenicity prophetically is performed.

### Example 3

### Construction and Expression of Flagellin-Hemaglutinin (Ha) Constructs

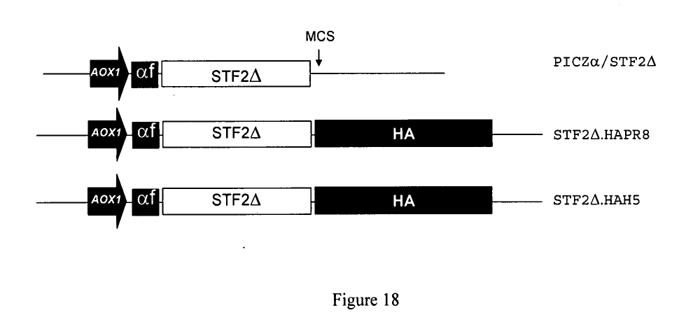

The gene encoding HA from genomic DNA from the in-house laboratory strain PR8, an attenuated derivative of A/Puerto Rico/8/34 was isolated (SEQ ID NO: 68, encoding SEQ ID NO: 67). The gene was fused to the STF2Δ cassette that has been previously constructed in pPICZΔ generating STF2Δ.HAPR8 (SEQ ID NO: 63, encoding SEQ ID NO: 62) (See FIG. 18). Purified recombinant protein was tested for immunogenicity and efficacy in BALB/c mice. The gene encoding H5N1 of the A/Vietnam/1203/04 strain was custom synthesized and fused to STF2Δ cassette generating STF2Δ.HAH5 (SEQ ID NO: 61, encoding SEQ ID NO: 60). Both human and avian HA constructs were transformed into Pichia pastoris strains GS115 and X-33 (Invitrogen Corporation, Carlsbad, Calif.). Selected clones were screened for expression by fractionation on SDS-PAGE gel and staining by Coommassie Blue and Western blot analysis using anti-HA and anti-flagellin antibodies.

### Example 4

### Generation of a Pam3Cys Fusion Protein

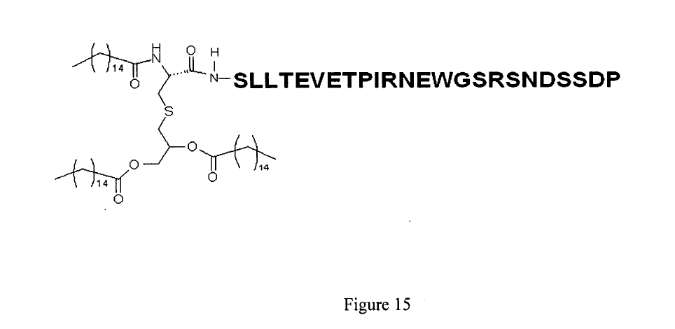

M2e (SEQ ID NO: 47) was chemically coupled to a tri-palmitoylcysteine (Pam3Cys) moiety through the amino terminal serine residue of the peptide. The structure of the fusion protein (Pam3Cys.M2e) is shown in FIG. 15. The chemical name for Pam3Cys.M2e is [Palmitoyl-Cys((RS)-2,3-di(palmitoyloxy)-propyl)-Ser-Leu-Leu-Thr-Glu-Val-Glu-Thr-Pro-IIe-Arg-Asn-Glu-Trp-Gly-Ser-Arg-Ser-Asn-Asp-Ser-Ser-Asp-Pro-OH acetate salt]. The molecular mass of Pam3Cys.M2e is 3582.3 daltons.

Pam3Cys.M2e was synthesized using a solid phase peptide synthesis methodology based on a well established Fmoc-strategy (Houben-Weyl, 2004. Synthesis of peptides and peptidomimetics, Vol. 22, Georg Thieme Verlag Stuttgart, N.Y.). The synthetic scheme and manufacturing process for Pam3Cys.M2e is diagrammed in the flow chart below. The Pam3Cys.M2e is a fusion protein (chemically linked) and is also referred to herein as a “lipidated peptide.”

The first step in the synthesis included solid phase peptide synthesis. The amino acid sequence of Pam3Cys.M2e was assembled on an H-Pro-2-chlorotrityl chloride resin by solid phase peptide synthesis. This resin is highly suitable for the formation of peptides with the Fmoc-strategy. The peptide chain was elongated by successive coupling of the amino acid derivatives. Each coupling step was preceded by an Fmoc-deprotection step and both steps were accompanied by repeated washing of the resin. After coupling of the last amino acid derivative, the final Fmoc-deprotection step was performed. Finally, the peptide resin was washed and dried under reduced pressure. During solid phase peptide synthesis color indicator tests were performed for each step to monitor the completion of the Fmoc-cleavage and the subsequent coupling of the amino acid derivatives.

Stage 2 of the synthesis included coupling of Pam3Cys-OH. Pam3Cys-OH was pre-activated with N,N′-dicyclohexyl-carbodiimide (DCCI) in the presence of 1-hydroxybenzotriazole (HOBt). The resulting solution was filtered and added to the peptide resin. At the end of the reaction time the peptide resin was washed and dried under reduced pressure. Color indicator tests were performed to control the coupling of Pam3Cys-OH.

Stage 3 of the synthesis included cleavage from the resin including cleavage of the side chain protecting groups. The peptide resin was treated with trifluoroacetic acid (TFA). The product was precipitated from the reaction mixture and lyophilized.

Stage 4 of the synthesis included purification by preparative reverse phase HPLC. The crude material obtained from Stage 3 was purified by preparative HPLC on a reverse phase column using a TFA system. The fractions were collected, checked by analytical HPLC and pooled accordingly. Pooled fractions from the TFA runs were lyophilized.

Stage 5 of the synthesis included precipitation in the presence of EDTA. The purified material from Stage 4 was precipitated from an aqueous solution of EDTA. The product was filtered off and dried under reduced pressure.

Stage 6 of the synthesis included ion exchange chromatography. The last stage of manufacturing Pam3Cys.M2e was the exchange from the trifluoroacetate salt into the acetate salt by ion exchange. The material from Stage 5 was loaded onto an ion exchange column and eluted with acetic acid. Fractions were checked by thin layer chromatography and the combined product-containing fractions were filtered and lyophilized to yield the final product.

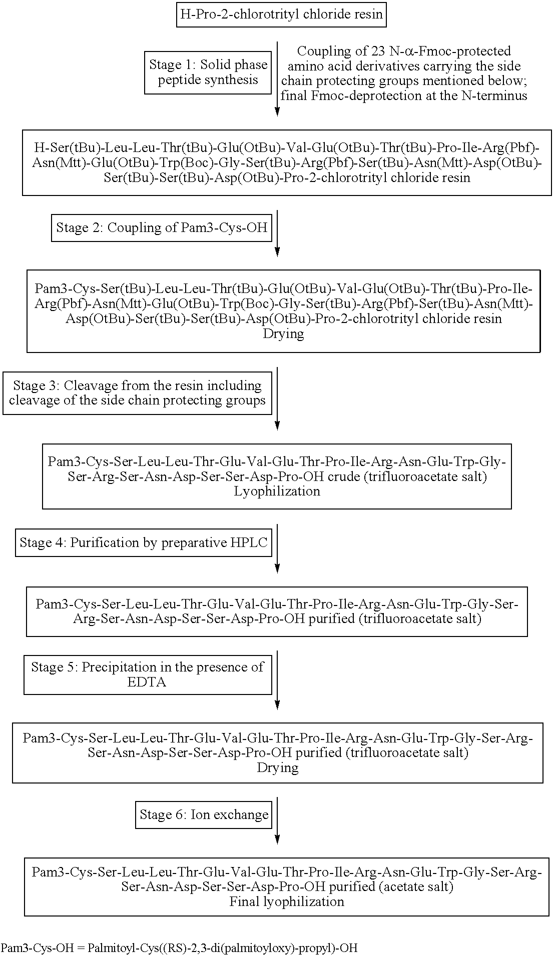

The purity specification for the Pam3Cys.M2e drug substance was ≧80% by RP-HPLC. The specification was based on the purity achieved with three non-GMP lots of Pam3Cys.M2e made from the same GMP batch of M2e-peptide intermediate resin. The purity of the three non-GMP lots of Pam3Cys.M2e was 80.2%, 80.3% and 80.8%, for lots D.001.Pam3Cys.M2e, D.002.Pam3Cys.M2e and D.003.Pam3Cys.M2e, respectively.

### Example 5

### Immunogenicity

### Materials and Methods

### Synthesis and Purification of Pam3Cys.M2e

Pam3Cys.M2e was prepared by Genemed Synthesis and Bachem using solid phase synthesis methodologies and FMOC chemistry as described above. Mass spectroscopy analysis was used to verify the molecular weight of the final product.

### Endotoxin Assay

Endotoxin levels of the STF2.4×M2e and the Pam3Cys.M2e were measured using the QCL-1000 Quantitative Chromogenic LAL test kit (BioWhittaker #50-648U), following the manufacturer's instructions for the microplate method.

### TLR5Bioactivity Assay

HEK293 cells constitutively express TLR5 and secrete several soluble factors, including IL-8, in response to TLR5 signaling. HEK293 cells were seeded in 96-well microplates (50,000 cells/well) and test proteins were added and incubated overnight. The next day, the conditioned medium was harvested, transferred to a clean 96-well microplate and frozen at −20° C. After thawing, the conditioned medium was assayed for the presence of IL-8 in a sandwich ELISA using an anti-human IL-8 matched antibody pair (Pierce, #M801E and #M802B) following the manufacturer's instructions. Optical density was measured using a microplate spectrophotometer (FARCyte, Amersham). Results are reported as pg of IL8 per ml as determined by inclusion of a standard curve for IL8 in the assay.

### TLR2Bioactivity Assay

RAW264.7 cells (ATCC) express TLR2 and secrete several soluble factors, including TNFα, in response to TLR2 signaling. RAW264.7 cells were seeded in 96-well microplates (50,000 cells/well), test compounds were added and incubated overnight. The next day, the conditioned medium was harvested, transferred to a clean 96-well microplate and frozen at −20° C. After thawing, the conditioned medium was assayed for the presence of TNFα in a sandwich ELISA using an anti-mouse TNFα matched antibody pair (Pierce) following the manufacturer's instructions. Optical density was measured using a microplate spectrophotometer (FARCyte, Amersham). Results are reported as ng of TNF per ml as determined by reference to a standard curve for TNF included in the assay.

### Mouse Immunogenicity

Female BALB/c mice (National Cancer Institute) were used at the age of about 6-8 weeks. Mice were divided into groups of 5 to 10 mice per group, and immunized subcutaneously on each side of the base of the tail on days 0 and 21 with the indicated concentrations of STF2.4×M2e or Pam3Cys.M2e fusion protein. On days 10 (primary) and 28 (boost), individual mice were bled by retro-orbital puncture. Sera were harvested by clotting and centrifugation of the heparin-free blood samples.

### Mouse Serum Antibody Determination

M2e-specific IgG levels were determined by ELISA. 96-well ELISA plates were coated overnight at 4° C. with 100 μl/well of a 5 μg/ml solution of the M2e peptide in PBS. Plates were blocked with 200 μl/well of Assay Diluent Buffer (ADB; BD Pharmingen) for one hour at room temperature. The plates were washed three times in PBS containing 0.05% Tween-20 (PBS-T). Dilutions of the sera in ADB were added (100 μl/well) and the plates were incubated overnight at 4° C. The plates were washed three times with PBS-T. Horse radish peroxidase, or HRP-labeled goat anti-mouse IgG antibodies (Jackson Immunochemical) diluted in ADB were added (100 μl/well) and the plates were incubated at room temperature for 1 hour. The plates were washed three times with PBS-T. After adding TMB Ultra substrate (3,3′,5,5′-tetramentylbenzidine; Pierce) and monitoring color development, the O.D. 450 was measured on a Tecan Farcyte microspectrophotometer.

### Rabbit Immunogenicity

Female and male NZW rabbits (Covance Research Products) were used at the age of about 13-17 weeks. Rabbits were divided into groups of 3 male and 3 female per group, and immunized i.m. on alternating thighs on days 0 and 21 and 42 with the indicated concentrations of Pam3Cys.M2e peptide or STF2.4×M2e fusion protein. Animals were bled on day-1 (prebleed), 14 (primary) and 28 and 42 (boost). Sera were prepared by clotting and centrifugation of samples.

### Rabbit Serum Antibody Determination

M2e-specific IgG levels were determined by ELISA. 96-well ELISA plates were coated overnight at about 4° C. with 100 μl/well M2e peptide in PBS (5 μg/ml). Plates were blocked with 200 μl/well of Assay Diluent Buffer (ADB; BD Pharmingen) for one hour at room temperature. The plates were washed three times in PBS-T. Dilutions of the sera in ADB were added (100 μl/well) and the plates were incubated overnight at about 4° C. The plates were washed 3× with PBS-T. Bound IgG was detected using HRP-conjugated goat anti-rabbit IgG (Jackson Immunochemical). The plates were washed three times with PBS-T. After adding TMB Ultra substrate (Pierce) and monitoring color development, O.D. 450 was measured on a Molecular Devices Spectramax microspectrophotometer. Results are reported as the Delta O.D. which is determined by subtracting the O.D. 450 reading for the prebleed of each animal from the O.D. 450 for each animal post-immunization.

### BALB/c Mouse Efficacy Model

In a typical experiment, about 5-6 week old female BALB/c mice (10-20 per group) were obtained and allowed to acclimate for one week. Fusion proteins formulated in PBS or other suitable formulation were administered by s.c. injection. Mice were immunized on days 0 and 14. On day 21, sera was harvested by retro-orbital puncture and evaluated for M2e specific IgG by ELISA. Mice were challenged by intranasal administration of 1×LD90 of the well characterized mouse adapted Influenza A strain, A/Puerto Rico/8/34 (H1N1). Mice were monitored daily for 14 days for survival and weight loss. Mice that lost about 30% of their initial body weight were humanely sacrificed, and the day of sacrifice recorded as the day of death. Efficacy data were reported as survival times.

### Results

### In Vitro Bioactivity

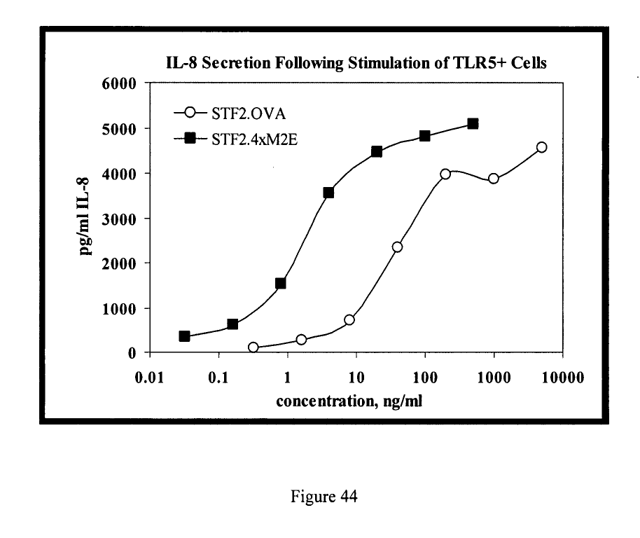

These assays were based on cell lines expressing the relevant TLR and screened for the ability to produce either IL8 or TNF-α in response to TLR triggering. In FIG. 44, the ability of STF2.4×M2e (▪) or STF2.0VA(◯) to stimulate TLR5 dependent IL8 production was evaluated following the stimulation of TLR5 positive, HEK293 cells. The results indicate that both fusion proteins stimulated IL8 production in a dose dependent manner and that the activity of the PAMP was retained in the context of the fusion.

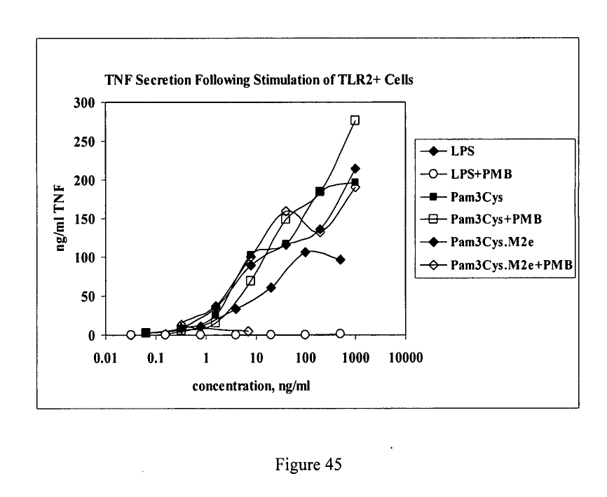

TLR2 activity was similarly evaluated for Pam3Cys.M2e following stimulation of TLR2 positive RAW264.7cells. In FIG. 45, the experimental groups are: the known endotoxin, LPS, as a positive control (♦), LPS plus the inhibitor of endotoxin polymixin B (PMB) as a negative control (◯), free Pam3Cys as a positive control for TLR2 signalling (▪), free Pam3Cys plus PMB (□), Pam3Cys.M2e (♦) and Pam3Cys.M2e plus PMB (⋄). The results showed similar activity profiles for Pam3Cys.M2e and the free TLR2 ligand Pam3Cys. The addition of polymyxin B (PMB) did not reduce its activity, indicating that there is no or low endotoxin contamination.

### Physical Linkage of Pamp and Antigen Enhances Immunogenicity

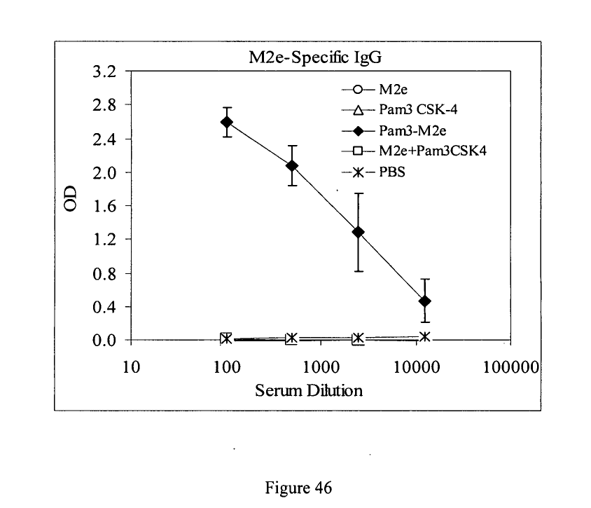

Using mouse models of immunogenicity, chemical coupling of Pam3Cys to M2e enhances the immunogenicity of the M2e antigen as compared to either the M2e peptide delivered alone or the M2e peptide co-delivered with free Pam3Cys. In the experiment shown in FIG. 46, groups of mice were immunized on days 0 and 21 with PBS as a negative control (*), the free TLR2 ligand, Pam3CSK-4 (( ), M2e peptide alone (◯), free Pam3CSK-4 mixed with M2e peptide (□), or the fusion of Pam3Cys and M2e referred to as Pam3.M2e (♦). The relevant the molar ratio of M2e peptide delivered was held constant. On day 28, sera were harvested and analyzed for M2e-specific antibody titers by ELISA. The results show that chemical coupling of Pam3Cys to the M2e (Pam3Cys.M2e) generates a detectable serum antibody response to the M2e antigen.

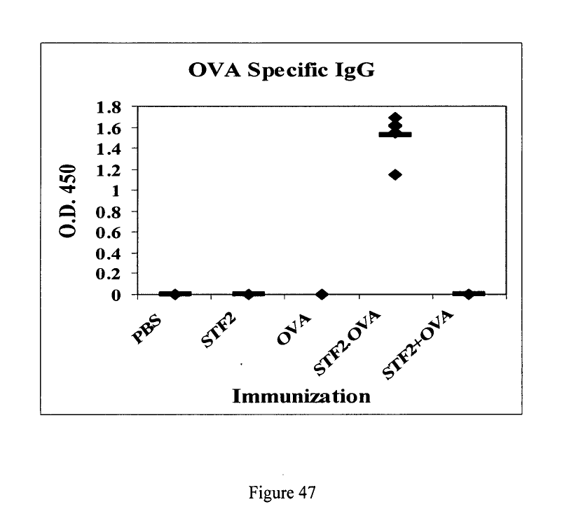

Physical linkage between the TLR5 ligand STF2 and antigen was demonstrated using the model antigen ovalbumin (OVA). Mice received a single s.c. immunization with STF2, OVA, STF2.0VA fusion protein, STF2+OVA mixture or PBS alone. Dosages were calculated to deliver 12 μg equivalents of STF2 and OVA per group. Seven days later, sera were harvested and OVA-specific antibodies were examined by ELISA. Data shown in FIG. 47 depict IgG1 titers at a 1:100 dilution of the sera. These results demonstrate that physical linkage of the TLR5 ligand and antigen results in optimal immunogenicity in vivo.

Pamp Linked Antigens are More Immunogenic than Conventional Adjuvant

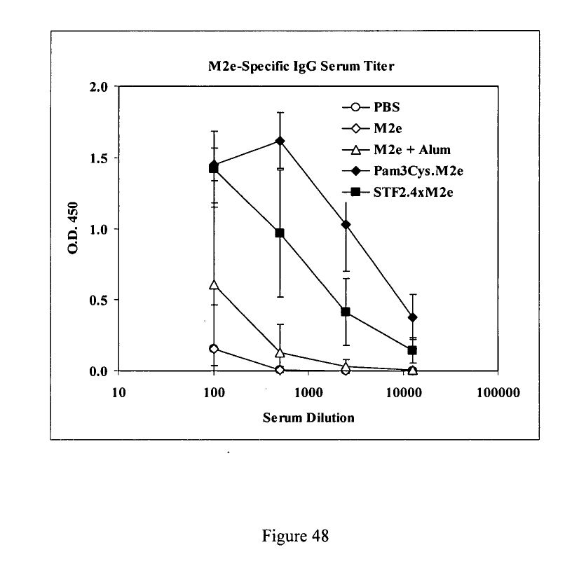

Groups of 5 BALB/c mice were immunized on day 0 and 14 with 30 μg of Pam3Cys.M2e (♦), 22.5 μg of M2e which is the molar equivalent of M2e in 30 μg of Pam3Cys.M2e (⋄), 22.5 mg of M2e adsorbed to the conventional adjuvant Alum (□), or 25 mg of the recombinant protein STF2.4×M2e (▪). A group receiving PBS was included as a negative control (◯). Sera were harvested 7 days post the second dose and M2e specific IgG were evaluated by ELISA. The results shown in FIG. 48 indicate that M2e alone is poorly immunogenic in that it failed to elicit antibody titers above background. The conventional adjuvant Alum provided a modest enhancement in the immune response to M2e. The PAMP linked M2e constructs; however, provided the greatest enhancement in immunogenicity. These results indicate direct linkage of PAMPs with portions of an integral membrane protein of an influenza viral protein can elicit immune responses that are more potent than those elicited by the conventional adjuvant Alum.

### Dose and Immunogenicity

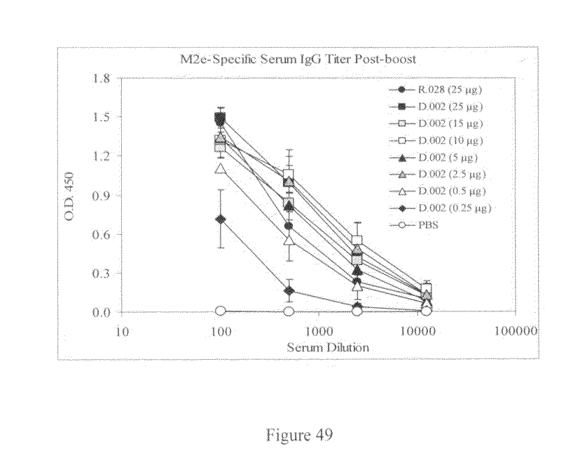

Dose ranging studies were carried out to further assess the potency of Pam3Cys.M2e and STF2.4×M2e. For STF2.4×M2e, BALB/c mice were immunized on day 0 and 14 with dilutions of STF2.4×M2e that ranged from 0.25 to 25 μg of STF2.4×M2e per immunization. The prefix D002 refers to the specific batch of STF2.4×M2e used in this experiment, while R-028 refers to a historical reference batch of STF2.4×M2e used in this experiment. Seven days following the last immunization (Day 21) mice were bled and M2e-specific IgG responses were evaluated by ELISA. The results shown in FIG. 49 demonstrate that immunization with doses as low as 0.25 μg per immunization of STF2.4×M2e induced detectable levels of M2e-specific IgG, with the optimal dose in mice falling in the range of about 2.5 to about 25 μg.

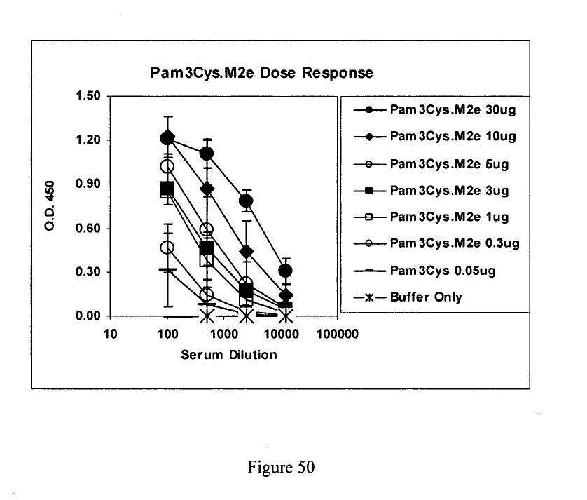

For Pam3Cys.M2e, BALB/c mice were immunized on day 0 and 14 with 0.05 to 30 μg of Pam3Cys.M2e per immunization. Seven days following the last immunization (Day 21) mice were bled and M2e-specific IgG responses were evaluated by ELISA. The results shown in FIG. 50 demonstrate that immunization with concentrations as low as 0.05 μg of Pam3Cys.M2e induced detectable levels of M2e-specific IgG, with the optimal dose for mice in this study of about 30 μg.

### Immunogenicity in Multiple Mouse Strains

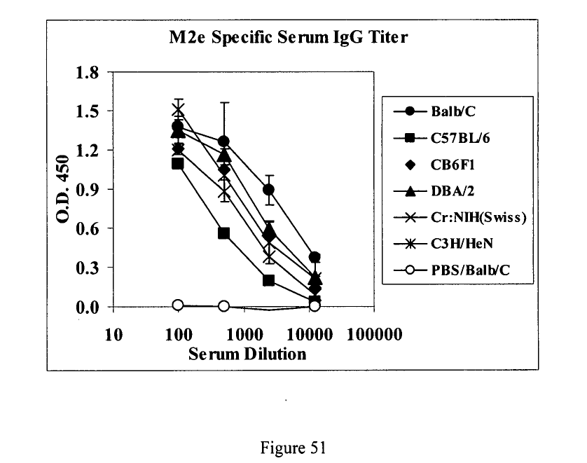

The immunogenicity of Pam3Cys.M2e was evaluated in multiple mouse strains including BALB/c (), C57BL/6 (▪), CB6/F1 (♦), DBA/2 (▴), Cr:NIH (Swiss) (X) and C3H/HeN (*). Groups of five for each strain were immunized on day 0 and 14 with 30 μg of Pam3Cys.M2e per immunization. Sera were harvested on day 21 and levels of M2e-specific IgG evaluated by ELISA. All strains exhibited significant levels of M2e-specific IgG indicating that the immunogenicity of Pam3Cys.M2e is not dependent on a particular MHC (FIG. 51).

### Immunogenicity in Rabbits

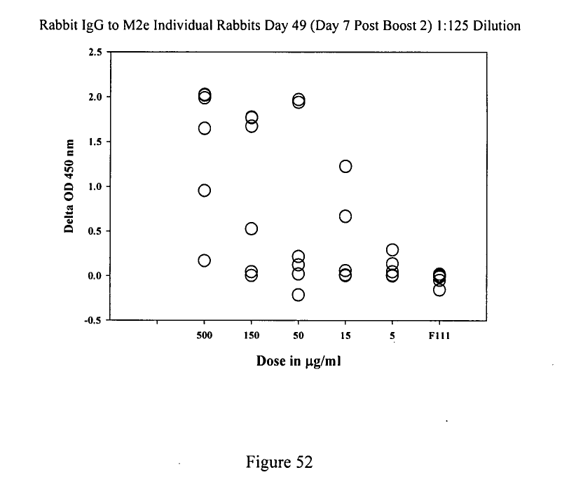

Studies aimed at evaluating the immunogenicity of Pam3Cys.M2e and STF2.4×M2e in a second species, rabbit, were carried out. In the first study, rabbits (3 females and 3 males/group) were immunized with 500, 150, 50, 15 or 5 μg (i.m.) of Pam3Cys.M2e on day 0, 21 and 42. As a control, an additional group received the formulation buffer F111 (10 mM Tris, 10 mM histidine, 75 mM NaCl, 5% sucrose, 0.02% Polysorbate-80, 0.1 mM EDTA, 0.5% ethanol, 20 mg/mL hydroxypropyl-beta-cyclodextrin, pH 7.2). On day 7 post-boost 2, peripheral blood was obtained and the anti-M2e antibody titers were evaluated by ELISA. The results shown in FIG. 52 depict the individual rabbit antibody titers at a 1:125 dilution of the sera. The data suggest a dose-response relationship between the amount of Pam3Cys.M2e used for prime/boost vaccinations and the level of the antibody titer achieved.

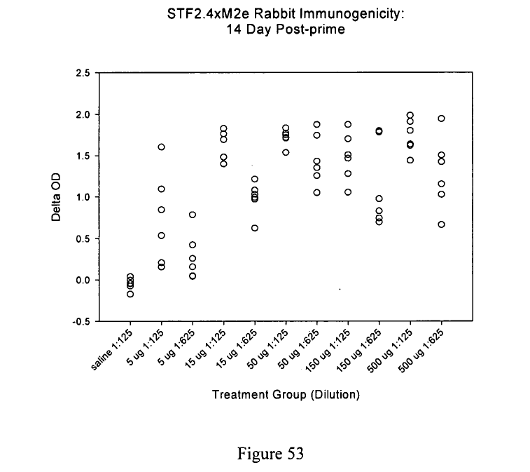

In the second study, rabbits (3 females and 3 males/group) were immunized with 500, 150, 50, 15 or 5 μg (i.m.) of STF2.4×M2e. As a control, an additional group received saline alone. On day 14 post-immunization, peripheral blood was obtained and the anti-M2e antibody titers were evaluated by ELISA. Notably, significant M2e-specific IgG responses were detectable by day 14 post-prime in all animals immunized (FIG. 53). The results indicate that STF2.4×M2e elicits a rapid and consistent immune response in rabbits.

### Efficacy in the Mouse Challenge Model

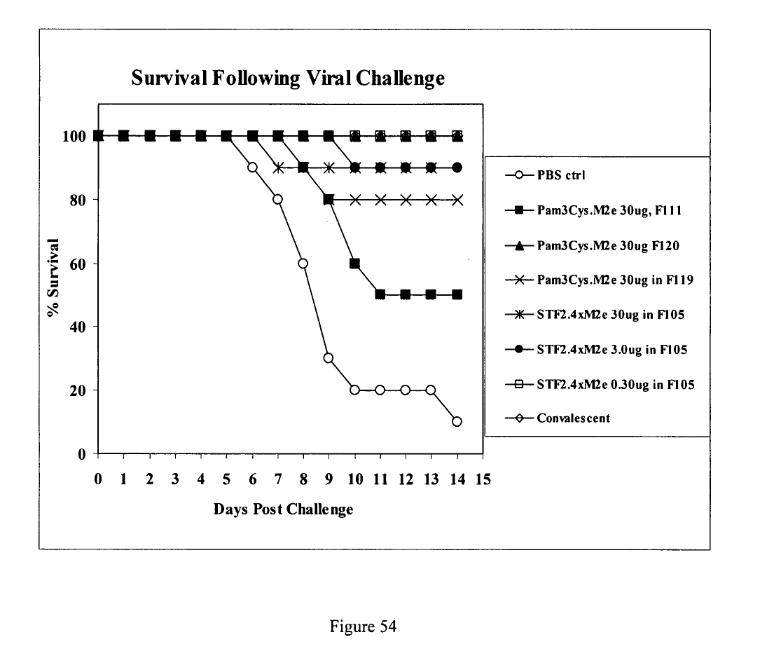

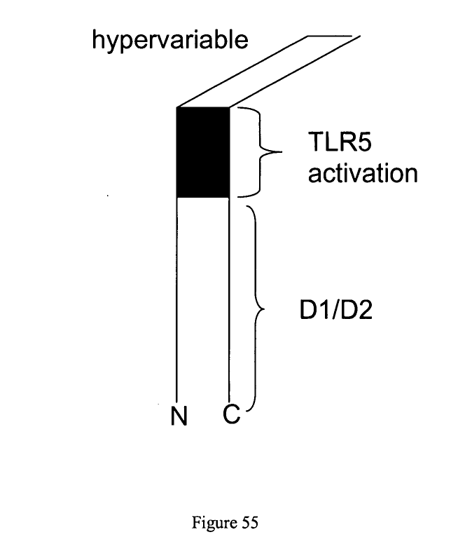

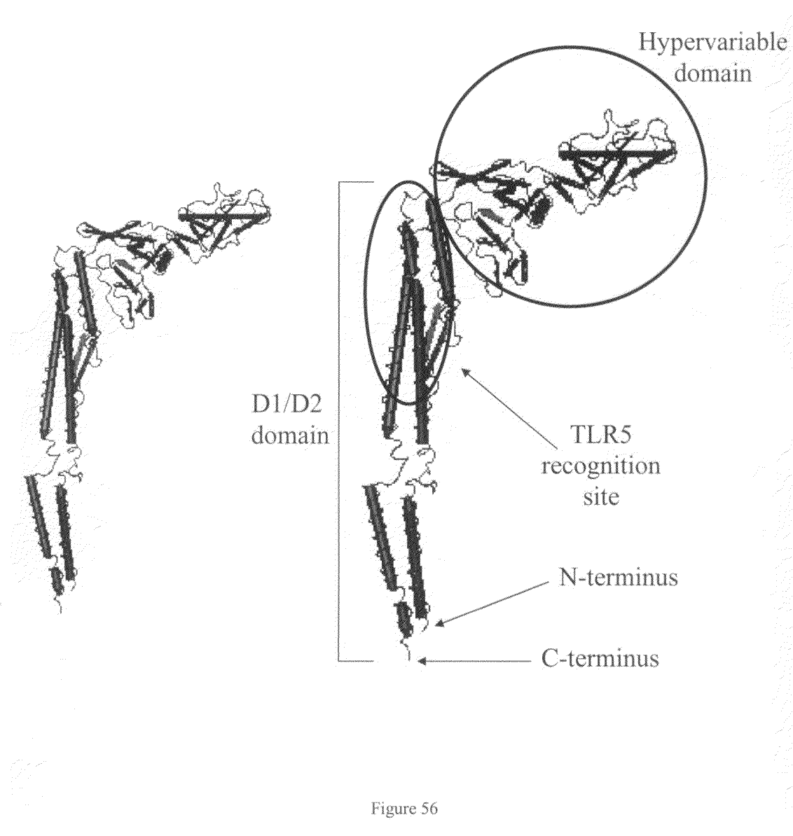

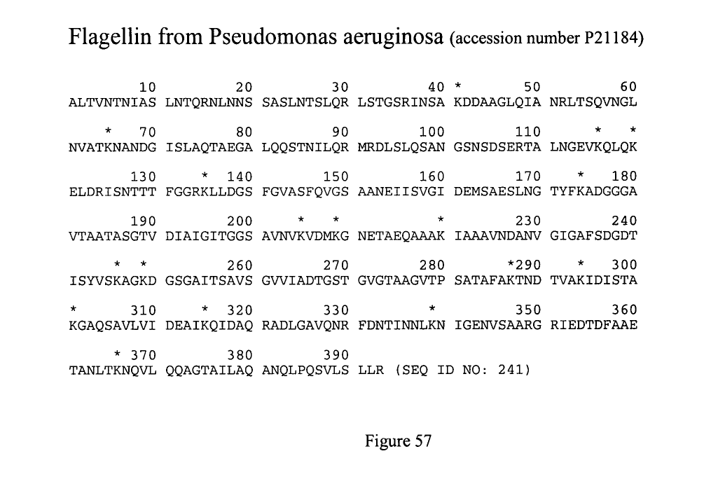

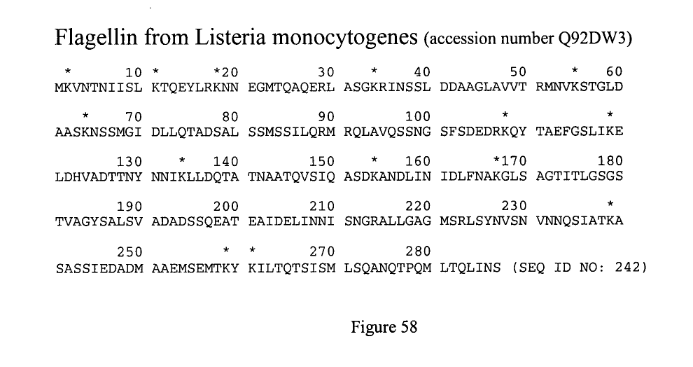

The efficacy of the Pam3Cys.M2e and STF2.4×M2e was evaluated in BALB/c mice using the well characterized mouse adapted strain, Influenza A/Puerto Rico/8/34 (PR/8) as the challenge virus. Groups of ten mice were immunized s.c. on day 0 and 14 with 30 μg of Pam3Cys.M2e in the formulation buffer F111 (▪), 30 μg of Pam3Cys.M2e in the proprietary buffer F120 (10 mM Tris, 10 mM histidine, 10% sucrose, 0.02% Polysorbate-80, 0.1 mM EDTA, 0.5% ethanol, 0.075% docusate sodium, pH 7.2) (▴), 30 μg of Pam3Cys.M2e in the buffer F119 (10 mM Tris, 10 mM histidine, 75 mM NaCl, 5% sucrose, 0.02% Polysorbate-80, 0.1 mM EDTA, 0.5% ethanol, 0.1% docusate sodium, pH 7.2), 30 μg of STF2.4×M2e in the buffer F105 (10 mM Tris, 10 mM histidine, 75 mM NaCl, 5% sucrose, 0.02% Polysorbate-80, 0.1 mM EDTA, 0.5% ethanol, pH 7.2), 3 μg of STF2.4×M2e in buffer F105 (10 mM Tris, 10 mM histidine, 75 mM NaCl, 5% sucrose, 0.02% Polysorbate-80, 0.1 mM EDTA, 0.5% ethanol, pH 7.2) () or 0.3 μg of STF2.4×M2e in buffer F105 (□). A group receiving PBS alone was included as a negative control (◯), and a convalescent group with immunity to PR/8 following a sublethal challenge with the virus was included as a positive control (⋄). On day 28, animals were challenge with an LD90 of the PR/8 challenge stock. Weight loss and survival was followed for 14 days post challenge (FIG. 54).

Animals in the convalescent group which had successfully cleared an earlier non-lethal infection with PR/8 demonstrated 100% protection to a subsequent viral challenge. Animals receiving the PBS buffer alone exhibited morbidity beginning on days 7 and 8, with 80% lethality occurring by day 10, while animals immunized with 30 μg of Pam3Cys.M2e in F111 demonstrated enhanced survival, with 50% of mice surviving the challenge. Animals receiving Pam3Cys.M2e in F119 exhibited morbidity beginning on days 8 and 9 with 80% of the mice surviving. Animals receiving Pam3Cys.M2e in buffer F120 (10 mM Tris, 10 mM histidine, 10% sucrose, 0.02% Polysorbate-80, 0.1 mM EDTA, 0.5% ethanol, 0.075% docusate sodium, pH 7.2) or the STF2.4×M2e protein exhibited the mildest disease course with 90 to 100% of the mice in these groups surviving the lethal challenge. These results demonstrate that both Pam3Cys.M2e and STF2.4×M2e can confer protective immunity to a challenge with influenza A in vivo.

### Discussion

Salmonella typhimurium flagellin (fljB) is a ligand for TLR5. A recombinant protein consisting of full-length flijB (STF2) fused to four tandem repeats of M2e was expressed in E. coli and purified to >95% purity with low endotoxin levels. In reporter cell lines, this protein (STF2.4×M2e) triggered IL8 production in a TLR5-dependent fashion. Mice immunized with dilutions of STF2.4×M2e that ranged from 0.25 μg to 25 μg, formulated in the buffer F105 which is without a conventional adjuvant or carrier, mounted a vigorous antibody response. The potency of the recombinant protein was further demonstrated in rabbit immunogenicity studies where animals receiving as little as 5 μg of protein seroconverted after a single dose. The efficacy of the PAMP fusion protein was demonstrated in the mouse challenge model using Influenza A/Puerto Rico/8/34 as the challenge virus. Mice immunized with as little as about 0.3 μg of the protein per dose exhibited mild morbidity with 100% of the mice surviving the challenge.

Synthetic tripalmitoylated peptides mimic the acylated amino terminus of lipidated bacterial proteins and are potent activators of TLR2. In these studies, a tripalmitoylated peptide consisting of three fatty acid chains linked to a cysteine residue and the amino terminus of the Influenza A M2 ectodomain (M2e) was synthesized using standard solid-phase peptide chemistries. This peptide (Pam3Cys.M2e) triggered TNFα production in a TLR2-dependent fashion in reporter cell lines. When used to immunize mice without adjuvant, Pam3Cys.M2e generated an antibody response that was more potent than M2e when mixed with free Pam3CSK-4. Pam3Cys.M2e was also found to be immunogenic in rabbits where a dose response relationship was observed between the amount of Pam3Cys.M2e used for immunization and the antibody titer achieved. The efficacy of the Pam3Cys.M2e peptide in a number of different formulations was evaluated in the mouse challenge model using Influenza A/Puerto Rico/8/34 as the challenge virus. Pam3Cys.M2e formulated in F119 and F120 exhibited the mildest morbidity with about 80 to about 100% of the mice surviving the challenge.

### Example 6

### Construction and Expression of Fusion Proteins of a Flagellin without a Hinge Region and Hemagglutinin

### Materials and Methods

Virus and ferret antiserum: A stock of influenza A/Solomon Islands (SI)/3/2006 virus was obtained from the Centers for Disease Control (CDC). The titer of the virus was determined to be about 1:1024 by hemagglutination assay. A stock of ferret antiserum raised against the virus was obtained from the CDC, with a reported hemagglutination inhibition (HI) titer of about 1:640. Both reagents were aliquoted and frozen at about −80° C. Individual aliquots were thawed as needed, and in the case of virus, used once and discarded. Antiserum was stored at about 4° C. after thawing until consumption of the aliquot.

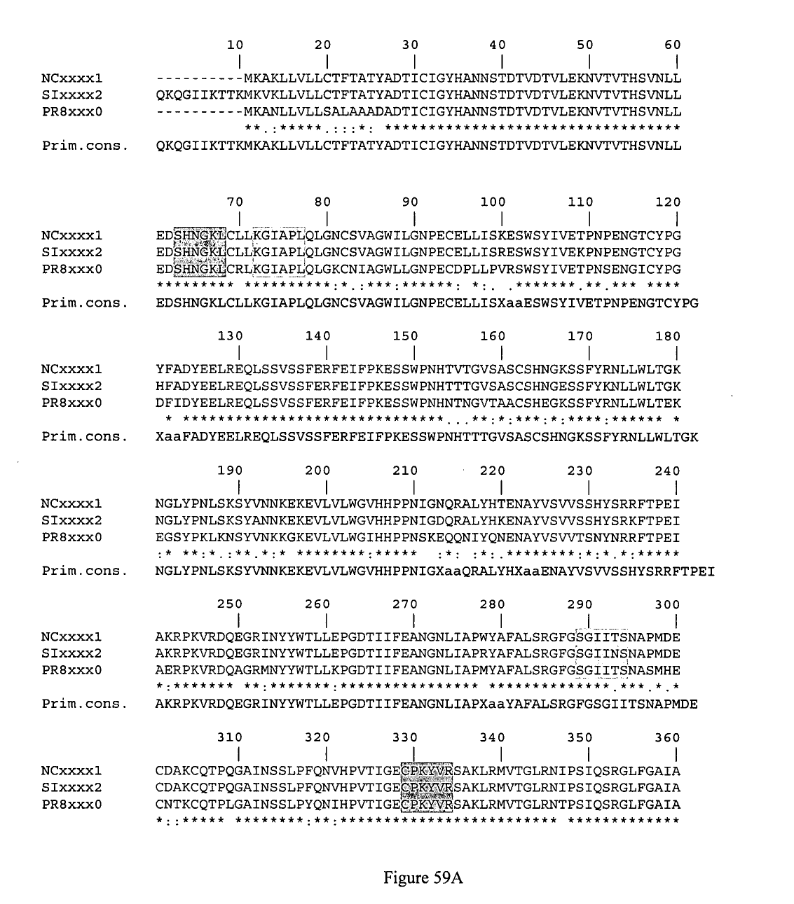

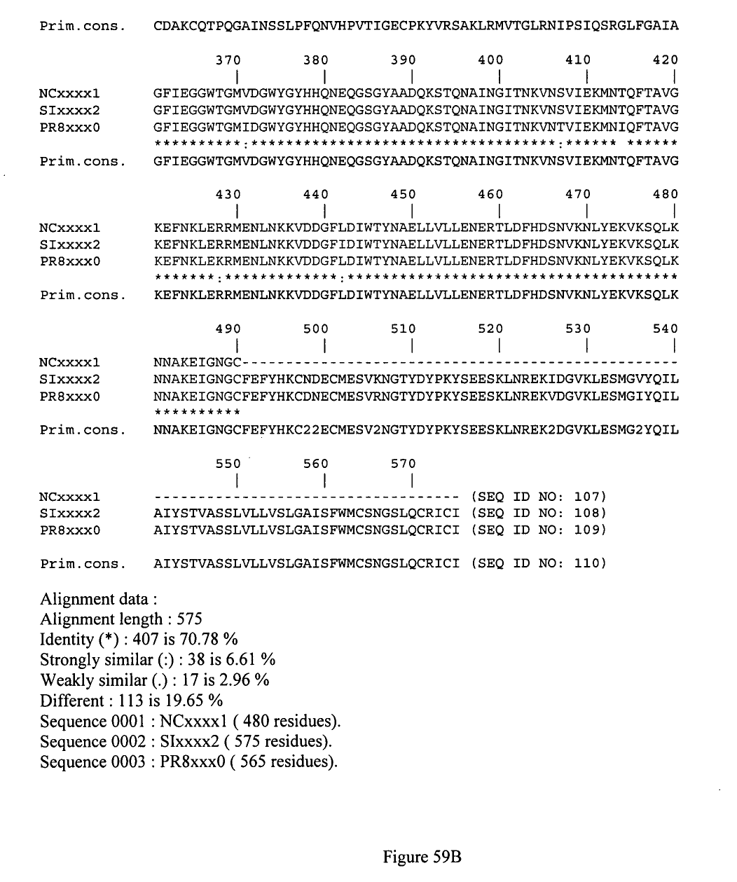

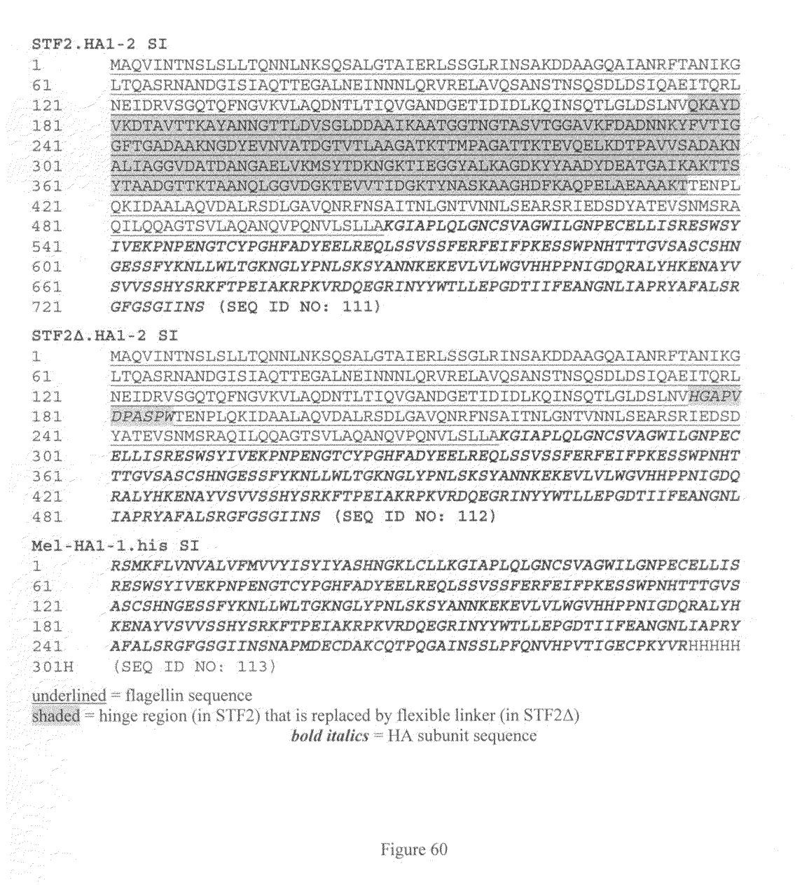

Construct design: The HA subunits for the HA component of the fusion protein was made employing a method as described in International Patent Application No: PCT/US2007/005611, filed Mar. 6, 2007; U.S. patent application Ser. No. 11/714,873, filed Mar. 6, 2007; and U.S. patent application Ser. No. 11/714,684, filed Mar. 6, 2007, the teachings of all of which are hereby incorporated by reference in their entirety. The method aligned the crystal structure of the A/Puerto Rico/8/34 (PR8) strain of influenza A with the SI hemagglutinin protein as shown in FIGS. 59A and 59B. The SI component of the fusion proteins are depicted in FIG. 60 and include a portion of SI HA (e.g., amino acids 53-324 and amino acids 62-284).

Cloning and expression of STF2.HA1-2(SI) and STF2Δ.HA1-2(SI): To facilitate rapid cloning of target genes fused with STF2 or STF2Δ, two cassette vectors, pET/STF2blp and pET/STF2Δblp were generated. In each cassette, a silent mutation was introduced near the 3′ end of the STF2 or STF2Δ gene to create a BlpI restriction site without changing the amino acid sequence of the flagellin gene. Both cassettes contain an StyI site in the vector sequence adjacent to the 3′ end of the flagellin gene, thus creating a region bounded by BlpI and StyI where a target gene can be inserted.

A synthetic gene encoding HA1-2 of influenza virus strain A/Solomon Islands/3/2006 (SI) was designed based on comparison of the SI sequence to PR8 and New Caledonia (NC) The codon-optimized gene, which includes cloning sites compatible with the STF2blp and STF2Δblp vector cassettes, was obtained commercially (DNA2.0, Menlo Park, Calif.) and cloned into both expression vectors to yield the following constructs: pET/STF2.HA1-2(SI) and pET/STF2Δ.HA1-2(SI). The constructs were verified by DNA sequencing and used to transform the expression host, BLR3(DE3). Transformants were selected on plates containing kanamycin (about 50 μg/ml), tetracycline (about 5 μg/ml) and glucose (about 0.5%). Several colonies were picked for overnight cultures which were then used to inoculate fresh LB cultures supplemented with about 25 μg/ml kanamycin, about 12.5 μg/ml tetracycline and about 0.5% glucose. Protein expression was induced with 1 mM IPTG for about 3 hours at about 37° C. The cells were harvested and protein expression was monitored by Coomassie blue staining of the SDS-PAGE gel and by Western blot analysis using the anti-flagellin mab, 6H11 (Inotek Pharmaceuticals, Beverly, Mass., Catalog #1030). Positive clones were selected for large scale production. The amino acid sequences of constructs are presented in FIG. 60.

Cloning and expression of HA1-1(SI): A synthetic gene encoding a portion of HA (HA1-1) from A/Solomon Islands/3/2006 was codon-optimized for Baculovirus expression and cloned into pFastBac™, which has a strong AcMNPV polyhedrin (PH) promoter for high level protein expression. The honey bee mellitin sequence (MKFLVNVALVFMVVYISYIYADPS; SEQ ID NO: 232) was fused to the amino terminus of HA1-1 to provide a secretion signal. To facilitate purification of the secreted protein, a hexahistidine tag was fused to the carboxy terminus of HA 1-1. The synthetic gene was excised from the commercially supplied plasmid with BglII and SphI enzymes and cloned by compatible ends to the pFastbac1 vector that was previously digested with the same enzymes, generating the construct pFastBac/HA1-1(SI). The ligation mix was used to transform TOP10 cells and several transformants were picked and screened by restriction analysis to confirm the presence and correct orientation of the insert. The construct was then used to transform MAX Efficiency® DH10Bac™ competent E. coli to generate a recombinant bacmid. Recombinant bacteria were screened for positive bacmids by blue/white selection Recombinant bacmid DNA was prepared and used to transfect the insect cell line of choice (Sf9) to generate a recombinant Baculovirus. The Baculovirus stock was amplified and titered to generate an expression stock. The amino acid sequence of the protein is presented in FIG. 60.

Purification of STF2.HA1-2(SI): A 12 L culture of E. coli cells expressing STF2.HA1-2(SI) was induced with about 1 mM IPTG at an O.D₆₀₀=0.8 and cells were harvested after about 4 hours of continued incubation at about 37° C. Following induction, cells were collected by low-speed centrifugation, suspended in 50 mM Tris-HCl pH 8.0, (100 ml/10 L of cell paste) and disrupted by passing the suspension twice through a microfluidizer at about 18K psi. The insoluble material (P) was separated from the soluble protein (S) by centrifugation and analyzed by SDS-PAGE. Under these induction conditions, STF2.HA1-2(SI) fractionated into both soluble and insoluble fractions at a ratio of about 70:30, respectively. The insoluble material was washed sequentially as described previously. As observed with other STF2.HA1-2 fusion proteins, the insoluble material was easily liberated from the pellet during the wash steps indicating that this material is weakly associated with the pellet fraction and likely represents highly aggregated material rather than true inclusion bodies.

To minimize poor protein yields from the insoluble fraction, the soluble or lysate fraction was subsequently processed as follows. The lysate was first applied to Q Sepharose under native conditions at low conductivity (about <2 mS/cm) and the unbound protein was collected. A considerable amount of material was present in the flow-through fraction under these conditions. Because nucleic acids and endotoxin typically bind at this pH, this initial chromatography step partially removed nucleic acid and endotoxin from the lysate. The flow-through fraction was adjusted to about 8 M urea and the pH was subsequently adjusted to about pH 4.0 with glacial acetic acid. This material was applied to SP source resin under denaturing conditions (8 M urea, 50 mM NaAcetate, pH 4.0) and bound protein was bumped from the resin with about 0.5 M NaCl.

The eluted material was extensively dialyzed against 8 M urea, 50 mM Tris-HCl, pH 8.0 to adjust the pH and then rapidly diluted (about 1:10) in 50 mM Tris-HCl, pH 8.0. The refolded material was applied to Source Q, and monomeric protein was eluted with a linear salt gradient from 0-0.5 M NaCl. During this gradient elution, a single protein peak was evident. The remaining bound material was eluted step-wise with 1.0 M NaCl. This yielded a significant peak that had a high A260:280 ratio suggesting that this material was predominantly nucleic acid. Based on non-reducing SDS-PAGE analysis of this fraction, this material also contains a significant amount of disulfide aggregated protein. Peak 1 fractions (monomeric STF2.HA1-2(SI)) were pooled and dialyzed against TBS, sterile filtered using 0.22 μm filters and assayed for protein and endotoxin levels. The protein yield for this first batch was 8 mg and the endotoxin levels were acceptable.

Purification of STF2Δ.HA1-2(SI): A 12 L culture of E. coli cells expressing STF2Δ.HA1-2(SI) was induced with IPTG at OD_{600 nm}=0.7 for 3 hours. Cells were harvested and cell paste was stored at about −80° C. The cell paste was resuspended in 100 ml Buffer A (20 mM Tris, pH 8.0)+1 mg/ml lysozyme/0.5 mM PMSF/50 μl SIGMA protease inhibitor cocktail/10 mg (total) DNAse I. The cells were lysed with 2 passes through the microfluidizer at 16,000 psi. The lysate was centrifuged for 1 hour at 38,500×g at 4° C. The pellet fraction was resuspended in 50 ml Buffer B (Buffer A+5 mM EDTA) using a glass-ball homogenizer and centrifuged at 38,500×g for 10 minutes. The pellet was washed 3 times (as described above) in 50 ml Buffer C (Buffer B+1% (w/v) Triton X-100). After a final wash in Buffer B, the pellet was dissolved in Buffer D (50 mM acetic acid, pH 4.0+8 M urea) and centrifuged, as described above. The supernatant fraction from this step was applied to a 50 ml Source S column equilibrated in Buffer D.

After washing with 10 column volumes buffer E (Buffer D+1% (w/v) Triton X-100), the column was washed with Buffer D and eluted with a 5 column-volume gradient Buffer D: Buffer F (Buffer D+1M NaCl). The Source S eluate was supplemented with Tris-HCl to a final concentration of 20 mM and adjusted to about pH 8.0. Protein (about 25 mg) was refolded by rapid dilution to a final concentration of 0.1 mg/ml in Refolding Buffer (0.1M Tris-HCl, pH 8.0/0.1M NaCl/1% (w/v) glycerol) supplemented with 1× redox buffer (5 mM reduced glutathione, 1 mM oxidized glutathione). After incubating overnight at about 4° C., the refolded protein was diluted about 1:4 with distilled water and applied to a 65 mL Source S column equilibrated in Buffer A and eluted in a 5 column-volume gradient Buffer A: Buffer G (Buffer A+1M NaCl). The Source Q eluate was concentrated using Amicon 15 spin concentrators (about 5 k mwco) and applied to a Superdex 200 gel filtration column equilibrated in 1×TBS, pH 8.0. Peak fractions were pooled, aliquoted and stored at about −80° C.

Purification of HA1-1(SI): Bacmid HA1-1(SI)-His BV was transfected into Sf9 cells to generate P1 viral stock. P1 viral stock titer was determined to be about 2.75×10⁷ pfu/mL by traditional plaque assay. Sf9 cells were infected with P1 viral stock at MOI of 0.1 to generate higher titer and larger volume of P2 viral stock. P2 viral stock titer was determined to be about 1.1×10⁸ pfu/mL by traditional plaque assay. Protein was expressed by infecting 2 L of High-5 cells at about 2×10⁶ cells/mL with P2 viral stock at MOI of 2.

The infected culture was harvested about 24-hours post-infection by centrifugation at about 8000 rpm for about 10 minutes at about 6-8° C. Supernatant was sterile-filtered through 0.2 μM filter units and 0.5 mM PMSF final and 200 μL protease inhibitor cocktail was added to each liter for immediate purification. 1 mM NiSO₄ final was added to each liter of clarified sample and then loaded onto 200 ml Ni NTA (Sigma) column, which was equilibrated with 5 column volumes of buffer A (20 mM Tris, pH 8, 0.5 M NaCl buffer). The column with bound protein was washed with equilibration Buffer A and then protein was eluted in linear gradient with Buffer B (Buffer A+0.5 M imidazole). Peak fractions were pooled and dialyzed into Buffer A with 2 changes going overnight at room temperature. Purification was repeated over 10 ml Ni NTA column to increase resolution and further fractionate impurities from protein. Peak fractions were pooled and dialyzed into 1×TBS, pH 8, buffer with 3 changes of buffer going overnight in 4° C.

Protein QC: All proteins were tested for concentration (BCA), purity and identity (SDS-PAGE and western blot with ferret antisera), TLR5 activity (bioassay), and endotoxin (LAL).
Protein concentration: Total protein concentration was determined using the Micro BCA Assay (Pierce) in the microplate format, using bovine serum albumin as a standard, according to the manufacturer's instructions.
SDS-PAGE and Western blot: An aliquot of 3-5 μg of each sample was diluted in SDS-PAGE sample buffer with or without 100 mM DTT as a reductant. The samples were boiled for 5 minutes and loaded onto a 10% SDS polyacrylamide gel and electrophoresed. The gel was stained with Coomassie R-250 to visualize protein bands. For Western blots, about 0.3-0.5 μg/lane total protein was electrophoresed and the proteins were electro-transferred to a PVDF membrane and blocked with 5% (w/v) dry milk before probing with the indicated antibodies. After probing with alkaline phosophatase-conjugated secondary antibodies, protein bands were visualized with an alkaline phosphatase chromogenic substrate (Promega Corporation, Madison, Wis.).
TLR5 bioassay: HEK293 cells constitutively express TLR5, and secrete several soluble factors, including IL-8, in response to TLR5 signaling. Cells were seeded in 96-well microplates (about 50,000 cells/well), and test proteins were added and incubated overnight. The next day, the conditioned media were harvested, transferred to a clean 96-well microplate, and frozen at about −20° C. After thawing, the conditioned media were assayed for the presence of IL-8 in a sandwich ELISA using an anti-human IL-8 matched antibody pair (Pierce Biotechnology, St. Louis, Mo., Catalog #M801E and #M802B) following the manufacturer's instructions. Optical density was measured using a microplate spectrophotometer (FARCyte, Amersham).
Protein antigenicity ELISA: The epitope integrity of the purified STF2.HA1-2(SI) and STF2Δ.HA1-2(SI) proteins was evaluated by ELISA. Plates were coated with serial dilutions of each protein, examined for reactivity with anti-SI ferret immune sera, anti-flagellin mAb (6H11), mouse PR8 convalescent sera, and naïve sera; and examined for cross-reactivity with rabbit PR8, New Caledonia (NC), Viet Nam (VN), and Indonesia (IN) HA1-1 immune sera.
Endotoxin measurement: Endotoxin levels were measured using the QCL-1000 Quantitative Chromogenic LAL test kit (BioWhittaker, Inc. Walkersville, Md. Catalog #50-648U), following the manufacturer's instructions for the microplate method.

Immunization of mice: Female BALB/c mice (National Cancer Institute) were used at the age of 6-8 weeks. Groups of 10 mice were immunized subcutaneoulsy (s.c.) (inguinal) on days 0 and 14 with about 3 or about 0.3 μg of STF2.HA1-2(SI) or STF2Δ.HA1-2(SI), each in PBS. Mice were bled on days 12 (prime) and 21 (boost), and sera were individually collected and stored.

Serum antibody determination (ELISA): ELISA plates (96 well) (Costar catalog #9018, Corning, N.Y.) were coated overnight at 4° C. with about 100 μl/well HA1-1(SI) (produced in Baculovirus) or STF2 (produced in E. coli) in PBS (5 μg/ml). Plates were blocked with 200 μl/well of Assay Diluent Buffer (ADB; BD Pharmingen catalog #555213, San Diego, Calif.) for 1 hour at room temperature. The plates were washed three times in PBS-T. Dilutions of non-immune and immune sera in ADB were added (100 μl/well) and the plates were incubated overnight at about 4° C. The plates were washed three times with PBS-T. HRP-labeled goat anti-mouse IgG antibodies (Jackson Immunochemical catalog #115-035-146, West Grove, Pa.) diluted in ADB were added (100 μl/well) and the plates were incubated at room temperature for 1 hour. The plates were washed three times with PBS-T. After adding TMB Ultra substrate (Pierce catalog #34028, Rockford, Ill.) and monitoring color development, A₄₅₀ was measured on a Tecan Farcyte microplate spectrophotometer.

Virus neutralization assay (neutral red readout): Due to the lack of mouse-adapted virus of the SI strain, an in vivo challenge was not possible. Therefore, in vitro surrogate markers of efficacy were measured, specifically hemagglutination inhibition (HI) and virus neutralization (neutral red assay). The virus neutralization assay was adapted with modifications from WHO Manual on Animal Influenza Diagnosis and Surveillance, p. 86-88 (WHO/CDS/CSR/NCS2002.5). The neutral red assay is adapted from a protocol of the Cell Lab at Gettysburg College (http://www3.gettysburg.edu/˜sorense/Cellab04/neutralred.htm). Test reagents (experimental mouse sera and naïve and SI-immune ferret sera) were heat-inactivated by incubating for 30 minutes in a water bath heated to about 56° C. Sera were serially titrated in 3-fold dilutions in DMEM+0.3% BSA and 1 mg/ml TPCK-treated trypsin (USB Corporation catalog #22725, Cleveland, Ohio). An equal volume of SI virus diluted 1:250 in the same medium was added to each serum dilution to achieve a final viral concentration of 1:500 (the pre-determined TCID₅₀ for our current stock of virus). Wells containing medium only and virus only were included as negative and positive controls, respectively. The plates were incubated for about 30 minutes at 37° C./5% CO₂.

Nearly confluent monolayers of MDCK cells (ATCC catalog # CCL-34, Manassas, Va.) in 96-well tissue culture plates were washed once with 200 μl/well PBS, then 100 μl/well of serum:virus mixtures and control reagents prepared as described above were added and incubated for about 2 hours at about 37° C./5% CO₂. Following incubation, the serum:virus mixtures were removed and replaced with 100 μl/well DMEM+0.3% BSA and 1 mg/ml TPCK-treated trypsin. Following incubation at 37° C./5% CO₂ for 3 days, the medium was aspirated from the wells and replaced with fresh medium containing 40 μg/ml neutral red (Sigma Aldrich catalog #N2889, St. Louis, Mo.). To determine maximum lysis, 2 μl lysis solution (9% Triton X-100 in water, weight/vol) was added to triplicate wells that had been incubated with medium only. Following a 1 hour incubation, the cells were fixed by the addition of 100 μl/well 1% formaldehyde/1% CaCl₂ for 5 minutes at room temperature; this fix step was performed twice in succession. The fix solution was aspirated and the neutral red was released by the addition of 100 μl/well of extraction medium (50% ethanol/1% acetic acid). The plate was incubated at room temperature for about 20 minutes, with shaking for the final 2 minutes. The amount of dye released was determined by measuring absorbance at a wavelength of 540 nm using a microplate spectrophotometer. Cell death (and hence, viral infectivity) was measured as a decrease in the amount of dye released as compared to media control. The percentage lysis of each serum dilution was calculated as:

% reduction=100×((sample-virus)/(med-virus))

where sample, max, and med refer to the absorbance values in wells representing experimental samples, virus only, and medium only, respectively. The neutralizing titer of each sample was defined as the dilution of serum which resulted in at least 50% reduction in viral infectivity.

Hemagglutination inhibition: For the HI assay, naïve and immune sera were diluted 1:3 and treated with receptor destroying enzyme (RDE II) (Denka Seiken, UK) at 37° C. overnight. Samples were then heat-inactivated at 56° C. for 1 hour, and pre-adsorbed with chicken red blood cells (Rockland, Pa.) (20:1) to remove nonspecific agglutinins. Two-fold serial dilutions of the above treated serum samples (25 μl/well) were mixed with 8 HA units of SI virus in 96-well “V” bottom plates and incubated for about 1 hour at about 37° C. Following incubation, 0.5% RBCs (about 50 μl/well) was added to the wells, samples were mixed, and wells were incubated for 2 hours at room temperature to allow RBC agglutination. HI titers were determined for individual samples as the highest serum dilution which prevented agglutination, where a ‘button’ of RBC in the bottom of the well represented prevention of agglutination.

### Results

Expression and purification of HA1-1(SI): HA1-1(SI) protein was expressed in insect cells using a Baculovirus vector and purified from the cell culture medium. Total yield was about 2.4 mg with about 0.006 EU/μg endotoxin. Purity was estimated to be about 90%. Protein was aliquoted and stored at −80° C. This protein was used as coating reagent for ELISAs to detect HA-specific antibodies in sera of mice immunized with vaccine candidates, as described below.

Expression and purification of STF2.HA1-2(SI): STF2.HA1-2(SI) was expressed in E. coli and purified from the soluble fraction of the cell lysate. Final purity was greater than about 95%, the yield was about 8 mg, endotoxin levels were about 0.04 EU/μg protein, and TLR5 bioactivity was positive.

Expression and purification of STF2Δ.HA1-2(SI): STF2Δ.HA1-2(SI) was expressed in E. coli and purified from the insoluble inclusion bodies (IBs). Final purity was about 85%, the yield was about 24 mg, endotoxin levels were about <0.02 EU/μg protein, and TLR5 bioactivity was positive.

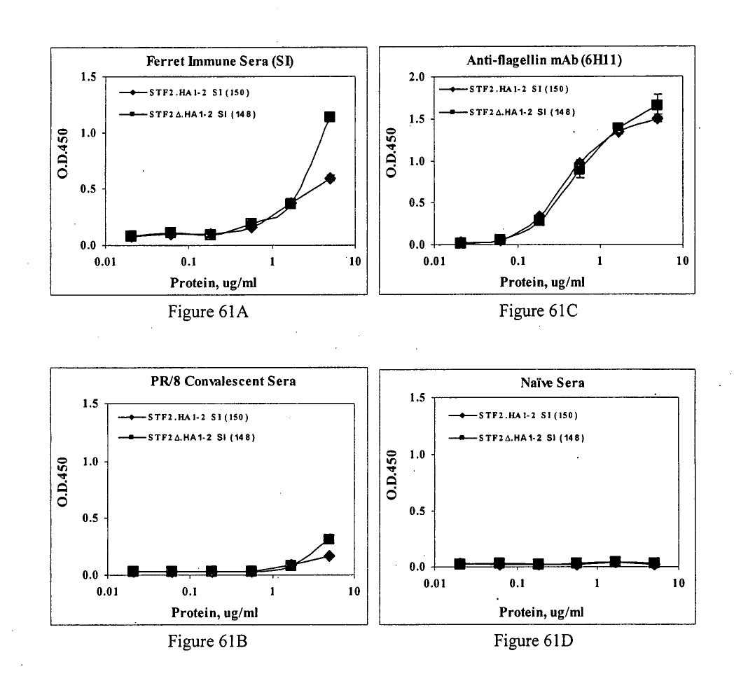

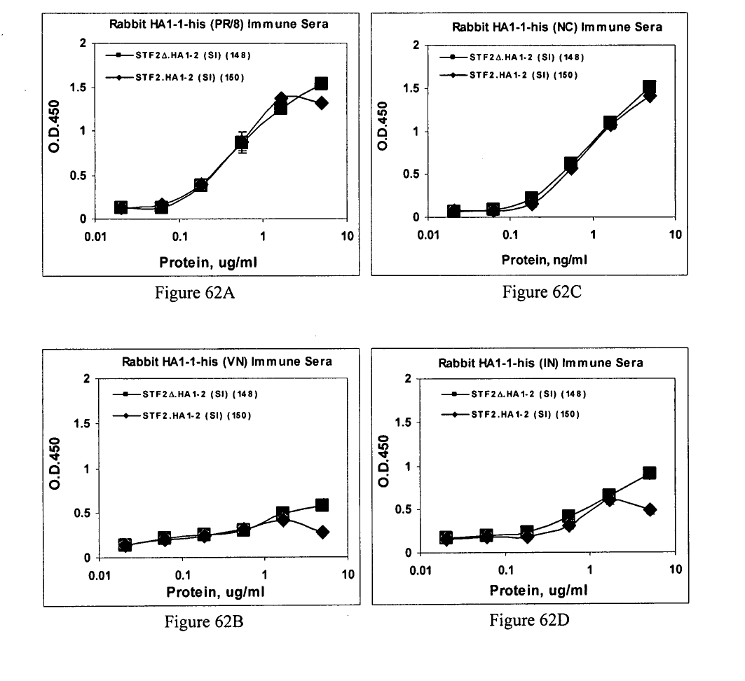

Antigenicity of fusion proteins: Both recombinant fusion proteins were recognized by the ferret SI immune sera and anti-flagellin mAb (6H11). There was little to no cross reactivity with PR8 convalescent serum (FIGS. 61A, 61B, 61C and 61D), but the proteins were recognized by rabbit antisera raised against HA 1-1(PR8) and HA1-1(NC). By contrast, the proteins were more weakly recognized by rabbit antisera raised against the H5 proteins, HA1-1(VN) and HA1-1(IN) (FIGS. 62A, 62B, 62C and 62D). Mouse PR8 convalescent sera reacted appears to react poorly while rabbit HA1-1 (PR8) sera reacted more strongly, perhaps because the rabbit antiserum was obtained after hyper-immunization rather than a single infection as for the convalescent sera.

Immunogenicity in BALB/c mice (ELISA): BALB/c mice were immunized with 3.0 or 0.3 μg of either vaccine candidate on days 0 and 14, and bled on day 21. Sera were analyzed by ELISA to determine the titers of flagellin-specific and HA1-1 (SI)-specific IgG titers. FIGS. 63A and 63B depict that all immunized mice generated antibody titers to both components of the fusion proteins, flagellin (FIG. 63A) and HA (FIG. 63B). The relative intensity of antibody responses to flagellin and HA differ between the two candidates. Specifically, mice immunized with STF2Δ.HA1-2(SI) generated flagellin-specific antibody titers that were at least 10-fold lower than titers in mice immunized with the equivalent dose of STF2.HA1-2(SI) (compare black circles to orange triangles, or open circles to open triangles in FIG. 63A), suggesting that deletion of the hinge region of flagellin (STF2Δ) significantly reduced the immunogenicity of the flagellin component of the fusion protein. By contrast, the HA-specific titers were identical in groups of mice immunized with equivalent doses of each protein (compare black circles to orange triangles, or open circles to open triangles in the FIG. 63B). Thus, the STF2Δ.HA1-2(SI) protein was not less immunogenic overall, but rather it reduced the immunogenicity of the flagellin component while maintaining immunogenicity of the HA component, compared to STF2.HA 1-2(SI).

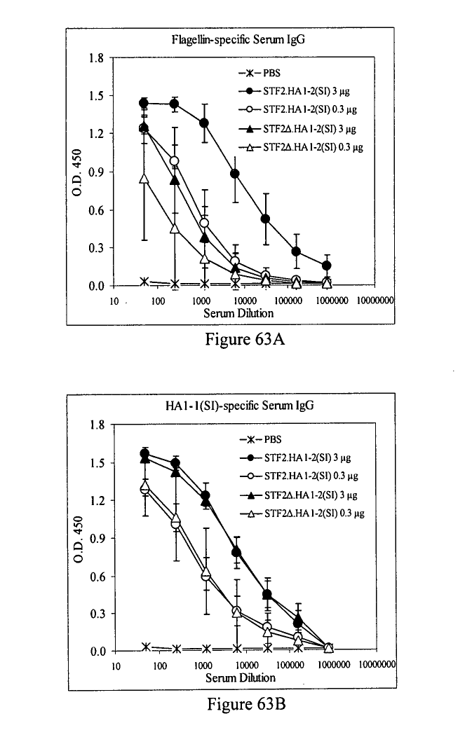

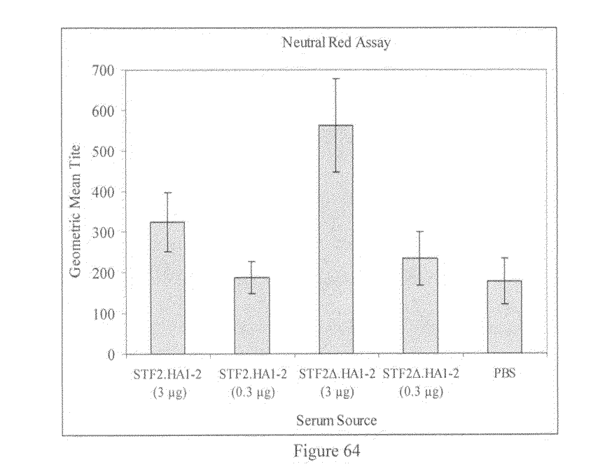

Virus neutralization (neutral red): A neutral red cell viability assay was also used to characterize the virus-neutralizing capacity of the mouse immune sera. In this assay, sera are pre-incubated with virus before the virus is added to monolayers of MDCK cells, and cell death is measured as a sign of viral infectivity. The data presented in FIG. 64 show that mice immunized with 3 μg of either vaccine construct generated antibody responses that inhibit viral infectivity in vitro. In contrast to the HI assay described herein (infra), normal mouse serum shows a high degree of background activity (i.e., inhibition of viral infectivity) in this assay, with geometric mean titers of the PBS-treated control mice approaching about 1:200. Mice immunized with either vaccine construct showed titers measurably higher than that background (about >1:300 for STF2.HA1-2(SI) and about >1:500 for STF2Δ.HA1-2(SI)).

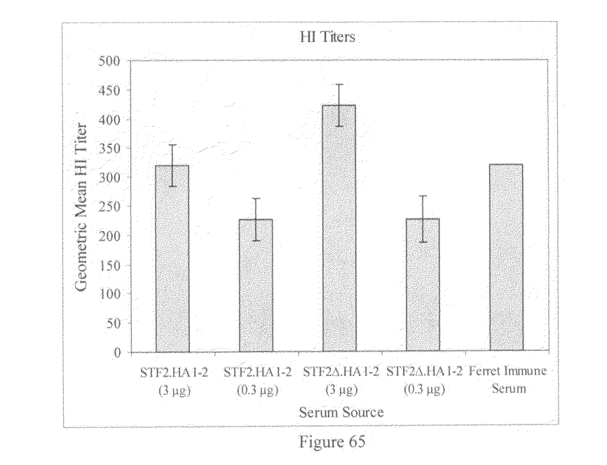

HI titers: The direct ELISA results previously demonstrated that the immune sera from STF2.HA 1-2(SI)- or STF2Δ.HA1-2(SI)-immunized mice recognize recombinant Baculovirus-expressed HA. In order to demonstrate that the anti-HA antibodies recognize native viral HA, the same sera were examined for the ability to specifically inhibit hemagglutination by A/SI virus. The results summarized in FIG. 65 demonstrate that the anti-HA antibodies elicited following immunization with STF2.HA1-2(SI) or STF2Δ.HA1-2(SI) potently inhibited virus-induced hemagglutination at serum titers ranging from about 1:80 to about 1:1280, with geometric mean titers at least about 1:320 in the about 3 μg dose groups. The complete table of individual titers in presented in FIG. 66.

### Discussion

The gene encoding HA1-2(SI) was fused to the 3′ end of the gene encoding full-length flagellin (STF2) and to the 3′ end of the gene encoding hinge region-deleted flagellin (STF2Δ), to yield two fusion protein constructs, STF2.HA1-2(SI) and STF2Δ.HA1-2(SI). These proteins were expressed in E. coli and purified from the soluble and insoluble fractions, respectively. STF2 fusion proteins generally remain largely in the soluble fraction, while STF2Δfusion proteins tend to favor the insoluble fraction (inclusion bodies), and this pattern held true in the current situation. While standard chromatographic methods were applied to both proteins, the purification of STF2Δ.HA1-2(SI) was greatly facilitated by the convenience of washing inclusion bodies as a way of enriching the target protein and reducing levels of contaminants including host cell proteins, DNA, and endotoxin, prior to chromatographic processing. The final yield of protein was also affected by the switch from STF2 to STF2Δ. From 11 L of E. coli culture of STF2.HA1-2(SI), the total cell paste was processed to yield 8 mg of pure protein. By contrast, only a portion of the cell paste of the STF2Δ.HA1-2(SI) culture was purified to yield about 24 mg of protein; this was equivalent to about 320 mg from the entire 12 L culture, thus representing a about 40-fold increase in yield pure unit of cell culture volume. Both proteins appeared to be properly refolded as they were recognized by SI-immune ferret antiserum by western blot, and both retained TLR5 bioactivity in vitro.

The immunogenicity of the proteins was tested in BALB/c mice. ELISA results demonstrate that immunization with either protein yields equivalent and potent antibody responses to HA, while the antibody response to flagellin is significantly less potent in mice immunized with STF2Δ.HA1-2(SI) than in mice immunized with STF2.HA1-2(SI). These results indicate that deletion of the flagellin hinge region in STF2Δdoes not adversely affect immunogenicity of the fused target antigen (in this case, HA1-2) and reduces the immunogenicity of the flagellin component.

While the ELISA data are useful in understanding immunogenicity, that assay does not distinguish between biologically relevant and irrelevant antibody responses. In other words, the ELISA will detect all antibodies that bind to HA1-1 on the plate, without regard to the particular site of HA that is bound or the neutralization of receptor-binding by the HA molecule. To measure biologically-relevant HA antibody titers, two additional assays were performed. In the first (neutral red virus neutralization assay), the ability of antisera to prevent or reduce virus infectivity in MDCK cell culture was measured by a calorimetric readout. This is a useful assay, but limited by the high background inhibition of viral infectivity by naïve mouse serum. Nevertheless, the results demonstrated that immunization of mice with about 3 μg of either vaccine candidate induced antibody responses that inhibited virus infectivity in vitro, above the background level of naïve mouse serum.

A more stringent assay for biologically-relevant HA antibodies is the hemagglutination inhibition (HI) assay. Agglutination of erythrocytes is mediated by HA molecules on the surface of influenza virions binding to sialic acid residues on erythrocytes; since each virion expresses hundreds of copies of HA, a single virus can bind to multiple erythrocytes, which can each be bound, by multiple virions, thus forming a lattice or network of agglutinated erythrocytes. This can be visualized in a microtiter plate quite easily: the absence of hemagglutination results in sedimentation of the erythrocytes into a tight ‘button’ at the bottom of the well, while the presence of hemagglutination maintains the dispersion of erythrocytes and the loss of the ‘button’. Thus, an antibody source that blocks the interaction between virion-associated HA and erythrocyte-associated sialic acid will favor the ‘button’ rather than the dispersion. Since the interaction of HA and sialic acid is dependent on specific conformational epitopes in HA, this assay is used to measure biologically-relevant HA antibodies. Using this assay, the date described herein demonstrate that mice immunized with either vaccine candidate, even at a dose of 0.3 μg, generated potent HI titers that were above about 1:200. In this assay, in contrast to the neutral red assay, naïve mouse serum is inactive at a dilution as low as about 1:40. An HI titer of at least about 1:40 is generally considered positive. Both STF2.HA1-2(SI) and STF2Δ.HA1-2(SI) induced titers much higher than that benchmark.

### Example 7

### Construction and Expression of Fusion Proteins of a Flagellin without a Hinge Region and Hemagglutinin

### Materials and Methods

Cloning of STF2Δ.HA1-2(PR8): The method described in detail in Example 6 was utilized. A synthetic gene encoding HA1-2 of influenza virus strain A/Puerto Rico/8/34 (PR8) was designed with cloning sites compatible with the STF2.blp and STF2Δ.blp vector cassettes. The codon-optimized gene was obtained commercially (DNA2.0, Menlo Park, Calif.) and cloned into pET/STF2Δblp to yield the construct pET/STF2Δ.HA1-2(SI). The construct was verified by DNA sequencing and used to transform the expression host, BLR3(DE3). Transformants were selected on plates containing kanamycin (about 50 μg/ml), tetracycline (about 5 μg/ml) and glucose (about 0.5%). Several colonies were picked for overnight cultures which were then used to inoculate fresh LB cultures supplemented with about 25 μg/ml kanamycin, about 12.5 μg/ml tetracycline and about 0.5% glucose. At an OD₆₀₀=0.6 protein expression was induced with about 1 mM IPTG for about 3 hours at about 37° C. The cells were harvested and protein expression was monitored by Coomassie blue staining of the SDS-PAGE gel and by Western blot analysis using the anti-flagellin mab, 6H11. Positive clones were selected for large scale production.

Purification of STF2Δ.HA1-2(PR8) expressed in E. coli: STF2Δ.HA1-2(PR8) expressed in E. coli was insoluble and formed inclusion bodies (IB). The protein was purified essentially as described in Example 6. After sequential wash steps with and without detergent to remove contaminants, the IB fraction was solubilized in 8 M urea. The protein was then bound to a Source S column and washed with Triton X-100 to remove endotoxin and eluted. The Source S eluate was then refolded by rapid dilution in the presence of glutathione redox buffer. The refolded protein was captured on a Source Q column and eluted. The protein was concentrated and fractionated on an S200 gel filtration column.

Protein QC: Methods for determining protein concentration (BCA), purity and identity (SDS-PAGE and Western blot with ferret antisera), TLR5 activity (bioassay), and endotoxin (LAL) are described in Example 6.

Protein antigenicity ELISA: The antigenicity of the purified STF2Δ.HA1-2(PR8) fusion protein was examined by ELISA with the anti-flagellin mAb (6H11), PR8 convalescent sera, or naïve sera. Plates were coated with serial dilutions of STF2.HA1-2(PR8) and STF2Δ.HA1-2(PR8) recombinant proteins. Following incubation with primary antibody or immune sera, reactivity was detected with HRP-conjugated anti-mouse IgG and developed with TMB substrate.

Assessment of immunogenicity: Mice were immunized on days 0 and 14, and bled on day 21, as described in Example 6. Methods for measuring serum antibody by ELISA are also described in Example 6.

### Results

Expression and purification of STF2Δ.HA1-2(PR8): Previous preparations of flagellin-linked HA1-2 proteins have shown a mixture of monomeric and aggregated protein upon Q chromatography. STF2Δ.HA1-2(PR8) eluted in a single peak from the Source Q column, suggesting that the protein mixture was homogeneous. No disulfide-linked aggregates were seen in the Q eluate fractions by non-reducing SDS-PAGE. Subsequent S200 size exclusion chromatography (SEC) of the Source Q eluate showed a single species eluting in the approximate range of a monomer. These data indicate that refolding of STF2Δ.HA 1-2(PR8) is highly efficient, with little or no aggregate formation. SDS-PAGE and Western blot analysis of the purified protein showed a clear mobility shift in the absence of reductant, and a corresponding increase in immunoreactivity with PR8 convalescent mouse serum, indicating the correct reformation of disulfide bonds.

The final yield for STF2Δ.HA1-2(PR8) was about 18 mg (about 2.6 mg/ml×7 mls). About 7.5% of the solubilized inclusion body fraction was carried all the way through the purification and refolding process. Thus, the equivalent yield from the entire 12 L of E. coli culture for this protein was about 240 mg. This is a dramatic increase in yield compared to the full-length STF2.HA1-2 proteins, which average about 15 mg from about 12 L of E. coli culture. This increase in efficiency is attributable to the removal of nucleic acid and other non-proteinaceous contaminants during the IB wash step with little or no loss of target protein.

Antigenicity of STF2Δ.HA1-2(PR8): The results demonstrate that both proteins exhibit similar levels of reactivity with the flagellin-specific antibody and PR8 convalescent sera (FIGS. 67A and 67B).

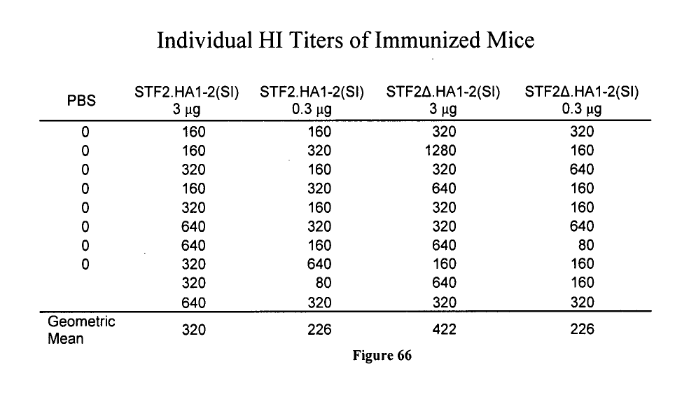

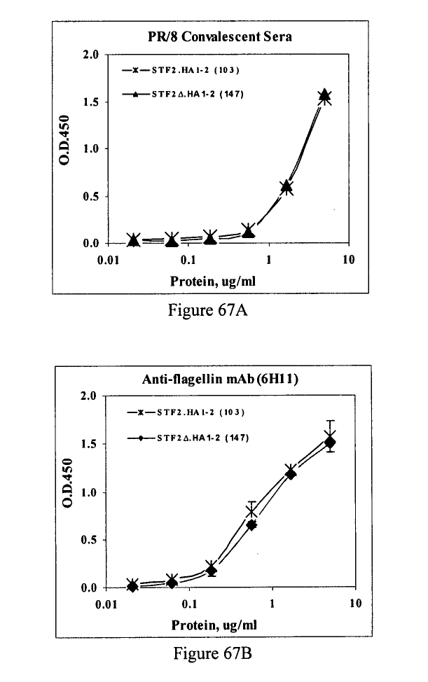

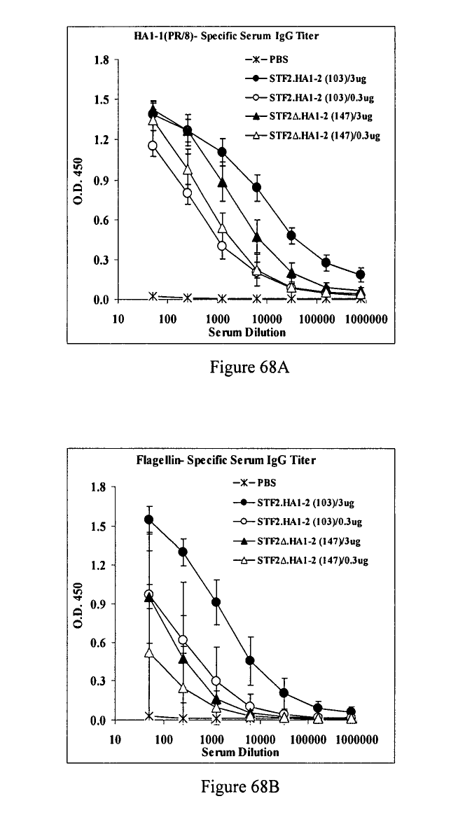

Immunogenicity of STF2Δ.HA1-2(PR8) expressed in E. coli: Mice (10/group) were immunized s.c. on days 0 and 14 with 3 or 0.3 μg of STF2.HA1-2(PR8) or STF2Δ.HA1-2(PR8) or PBS. On day 21, sera were harvested and HA1-1his(PR8)- and flagellin-specific IgG responses were examined by ELISA. The results demonstrate that immunization with about 3 or about 0.3 μg of either protein induced HA antibody titers that were equipotent on a dose basis, while the flagellin antibody titers were significantly lower in the mice immunized with STF2Δ.HA1-2 than in mice immunized with STF2.HA1-2 (FIGS. 68A and 68B). The data suggest that deletion of the hinge region of flagellin (STF2Δ) reduces the immunogenicity of the flagellin component of the vaccine without sacrificing immunogenicity of the target antigen, HA 1-2. These mice were challenged intranasally on day 28 with a LD₉₀ of PR8 virus and are being monitored for survival, clinical scores, and weight.

### Discussion

A synthethic gene encoding the HA1-2 subunit from influenza PR8 virus was fused to the 3′ end of the gene for STF2Δ to encode the fusion protein, STF2Δ.HA1-2(PR8). This protein differs from STF2.HA1-2(PR8) only in the deletion of the hinge region of flagellin in STF2Δ.HA1-2(PR8). Both proteins were purified from E. coli and retained TLR5 activity. Both proteins also were recognized by sera from mice which had recovered from a sublethal dose of live PR8 virus (convalescent mice), suggesting that the appropriate HA epitopes are displayed by both fusion proteins. Mice immunized with both proteins produced equivalent titers of HA-specific antibodies. However, the flagellin antibody titers in mice immunized with STF2Δ.HA1-2(PR8) were significantly lower than those in mice immunize with STF2.HA1-2(PR8). These data confirm that deletion of the hinge region of flagellin in fusion proteins did not abrogate TLR5 activity of the fusion protein or immunogenicity of the antigen component of the fusion protein, while it did decrease immunogenicity (i.e., antibody production) to the flagellin component of the fusion protein, which is desirable for fusion proteins of the invention employed in methods to stimulate an immune response, in particular, a protective immune response, in a subject.

### Example 8

### Construction and Expression of Fusion Proteins of a Flagellin without a Hinge Region and Matrix 2 Protein

### Materials and Methods

Cloning of STF2Δ.4×M2e(PR8): The method described in detail in Example 6 was utilized. A synthetic gene encoding 4 tandem copies of the M2e sequence of influenza virus strain A/Puerto Rico/8/34 (PR8) was designed with cloning sites compatible with the STF2blp and STF2Δblp vector cassettes. To avoid inappropriate disulfide bond formation, the cysteine residues in M2e were mutated to serines. The codon-optimized gene was obtained commercially (DNA2.0, Menlo Park, Calif.) and cloned into pET/STF2Δblp to yield the construct pET/STF2Δ.4×M2e(PR8). The construct was verified by DNA sequencing and used to transform the expression host, BLR3(DE3). Transformants were selected on plates containing kanamycin (about 50 μg/ml), tetracycline (about 5 μg/ml) and glucose (about 0.5%). Several colonies were picked for overnight cultures which were then used to inoculate fresh LB cultures supplemented with about 25 μg/ml kanamycin, about 12.5 μg/ml tetracycline and about 0.5% glucose. At an OD₆₀₀=about 0.6 protein expression was induced with 1 mM IPTG for 3 hours at 37° C. The cells were harvested and protein expression was monitored by Coomassie blue staining of the SDS-PAGE gel and by Western blot analysis using the anti-flagellin mab, 6H11, and the anti-M2e mab, 14C2 (Affinity BioReagents, Golden, Colo., Catalog #1030). Positive clones were selected for large scale production.

Purification of STF2Δ.4×M2e(PR8) expressed in E. coli: The purification methods described in Examples 6 and 7 were utilized to purify STF2Δ.4×M2e(PR8) from inclusion bodies (IB) in E. Coli host cells.

Protein QC: Methods for determining protein concentration (BCA), purity and identity (SDS-PAGE and western blot with ferret antisera), TLR5 activity (bioassay), and endotoxin (LAL) are described in Example 6.

Protein antigenicity ELISA: The antigenicity of the purified STF2Δ.HA1-2(PR8) fusion protein was examined by ELISA with the anti-flagellin mAb (6H11) and anti-M2e mAb (14C2). Plates were coated with serial dilutions of STF2.4×M2e(PR8) and STF2Δ.4×M2e(PR8) recombinant proteins. Following incubation with primary antibody or immune sera, reactivity was detected with HRP-conjugated anti-mouse IgG and developed with TMB substrate.

Assessment of immunogenicity and efficacy: Mice were immunized on days 0 and 14, and bled on day 21, as described in Example 6. Methods for measuring serum antibody by ELISA are also described in Example 6, except the ELISA plates were coated with M2e peptide rather than HA subunit protein. To assess efficacy, the mice were challenged on day 28 by intranasal administration of an LD₉₀ of PR8 virus. Survival, weight loss, and clinical scores were monitored for 21 days post challenge.

### Results

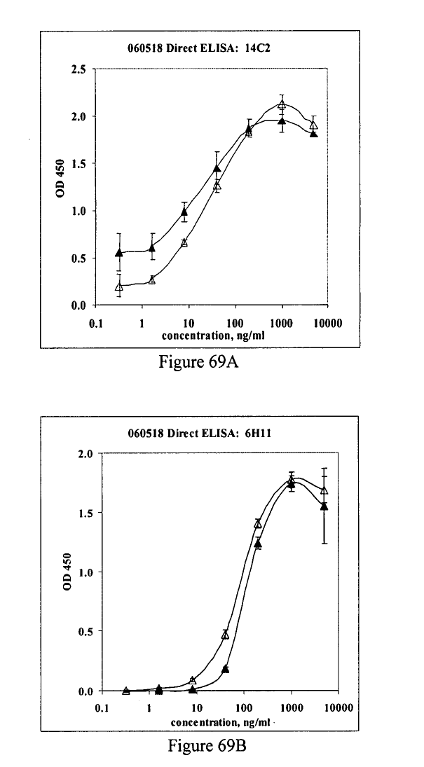

Antigenicity and immunogenicity of STF2.4×M2e and STF2Δ.4×M2e: Proper display of the M2e epitope was examined by coating ELISA plates with each protein and probing with monoclonal antibody to M2e (14C2 antibody) or, as a positive control, a monoclonal antibody to flagellin (6H11 antibody). The results in FIGS. 69A and 69B demonstrate that recognition of STF2Δ.4×M2e by either antibody was indistinguishable from recognition of STF2.4×M2e.

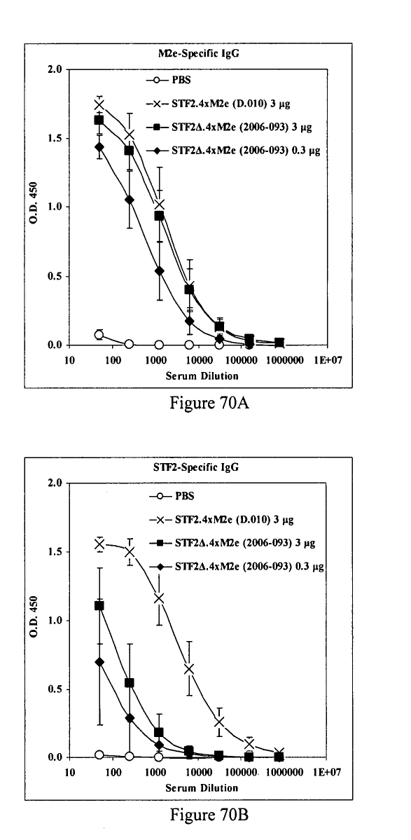

The immunogenicity of STF2.4×M2e (about 3 μg) and STF2Δ.4×M2e (about 3 or about 0.3 μg) was examined in BALB/c mice (10/group) immunized s.c. on day 0 and 14. On day 21 mice were bled and M2e(PR8)-specific and flagellin-specific IgG responses were examined by ELISA. The results in FIG. 70A demonstrate that immunization with about 3 or about 0.3 μg STF2Δ.4×M2e induced M2e(PR8)-specific IgG responses, with the higher dose demonstrating responses comparable to those induced by the same dose of STF2.4×M2e. By contrast, the antibody responses specific for flagellin (FIG. 70B) are lower in the STF2Δ.4×M2e-immunized mice compared to the STF2.4×M2e-immunized mice. Thus, it appears that deletion of the hinge region of flagellin does not negatively impact immogenicity (i.e., antibody response) of the 4×M2e antigen component of the fusion protein but does reduce the immunogenicity of the flagellin component, a desirable characteristic fusion protein of the invention, in particular, fusion proteins employed in methods for stimulating an immune response and providing protective immunity in a subject.

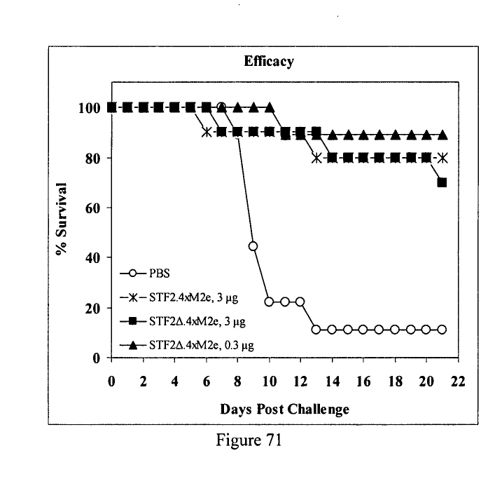

Efficacy of STF2Δ.4×M2e(PR8): To examine protective efficacy of STF2Δ.4×M2e, mice from FIGS. 70A and 70B were challenged on day 28 with an LD₉₀ of PR8. As shown in FIG. 71, mice immunized with PBS alone exhibited about 10% survival while mice immunized with the homologous STF2.4×M2e(PR8) vaccine (lot D.010) demonstrated about 80% survival. Mice immunized with about 3 or about 0.3 μg of STF2Δ.4×M2e(PR8) demonstrated about 90% and about 80% survival, respectively, comparable to the efficacy observed in animals receiving STF2.4×M2e. Thus, the deletion of the flagellin hinge region does not negatively impact immunogenicity of the fused antigen (FIGS. 70A and 70B) or protective efficacy provided by the vaccine (FIG. 71).

### Discussion

A synthethic gene encoding four tandem repeats of the M2e sequence from PR8 virus (with serine substitutions for cysteine) was fused to the 3′ end of the gene for STF2Δ to produce a fusion protein, STF2Δ.4×M2e. This protein differs from STF2.4×M2e only in the deletion of the hinge region of flagellin in STF2Δ.4×M2e, Both proteins were purified from E. coli and retained TLR5 activity and display of a known protective epitope of M2e. Mice immunized with both proteins produced equivalent titers of M2e-specific antibodies and equivalent protection against challenge with a lethal dose of influenza virus. However, similar to the two previous examples, the flagellin antibody titers in mice immunized with STF2Δ.4×M2e were significantly lower than those in mice immunize with STF2.4×M2e. These data show that deletion of the hinge region of flagellin in fusion proteins retains TLR5 activity and immunogenicity (i.e., antibody production) and efficacy of the antigen component of the fusion protein, while it decreases immunogenicity of the flagellin component of the fusion protein.

### Example 9

### Construction and Expression of Fusion Proteins of a Flagellin without a Hinge Region and Matrix 2 Protein

### Materials and Methods

Virus: A stock of influenza A/Puerto Rico/8/34 virus was expanded in embryonated chicken eggs, aliquoted and frozen at −80° C. Individual aliquots were thawed as needed, used once and discarded.

Cloning and expression of recombinant STF2.4×M2e proteins: A construct encoding four tandem copies of M2e representing the consensus sequence of the human influenza A virus H1, H2, and H3 strains (SLLTEVETPIRNEWGSRSNDSSDP; SEQ ID NO: 13) was chemically synthesized (DNA2GO, Menlo Park Calif.) as a DNA concatemer. In this synthetic gene the cysteine residues have been modified to serine, to prevent disulphide bond formation. The plasmid DNA served as a template to generate the 4×M2e fusion gene employing the Seamless Cloning kit by Stratagene (LaJolla, Calif.). The PCR product was ligated to the 3′ end of the Salmonella typhimurium fljB gene (STF2) in a pET24A vector (Novagen, San Diego, Calif.) and the ligation mix was used to transform XL1-Blue MRF′ cells, and positive clones were identified by PCR screening using pET24A specific primers and by restriction mapping analysis.

The construct, pET/STF2.4×M2eHu (also referenced as pET/STF2.4×M2e(PR8)) was confirmed by DNA sequencing. The plasmid DNA was used to transform competent BLR(DE3)pLysS cells and several transformants were picked and grown overnight for induction with 1 mM IPTG. Two hours after induction the bacteria were harvested and the lysate was analyzed by SDS-PAGE. A protein band of about 67 kDa, corresponding to STF2.4×M2eHu protein, was visible by Coommassie Blue staining and by immunoblotting using the anti-M2 monoclonal antibody 14C2. A clone selected in this manner was used for scale up and production. The 4×M2e gene was generated by PCR using the pET/STF2.4×M2e as template and employing NdeF1 (5′GAATTCCATATGAGCTTGCTGACTGAGGTTGAGACCCCGATTCGCA; SEQ ID NO: 243) and BlpR (5′ GACGTGGCTCAGCTTATTAATGGTGATGATGGTGATGTCTAGACGGGTCT GAGCTATCGTTAGAGCG; SEQ ID NO: 244) as forward and reverse primers respectively. The 270 bp fragment was digested with NdeI and BlpI enzymes and inserted into pET24A vector that has been previously digested with the same enzymes. The construct, pET/4×M2e which contains the hexa-His at the C-terminus of the M2e protein was used to transform BLR DE3 cells as described above.

Protein purification: The 4×M2e-his and STF2-his proteins were purified using nickel affinity chromatography as described previously. STF2.4×M2e protein was expressed and purified as follows. Bacterial cultures of BLR(DE3) that carry the plasmid pET/STF2.4×M2e were grown in LB medium containing 15 μg/ml kanamycin and 12.5 μg/ml teteracycline to about OD₆₀₀=0.6 and induced with 1 mM IPTG for about 3 h at about 37° C. Cells were harvested by centrifugation (about 8000×g for about 7 minutes) and resuspended in 2× phosphate buffered saline (2×PBS: 24 mM KH₂PO₄/Na₂HPO₄, 274 mM NaCl, 5.4 mM KCl), 1% glycerol, DNAse, 1 mM PMSF, protease inhibitor cocktail and 1 mg/ml lysozyme.

To disrupt cells, the cell suspension was passed twice through a microfluidizer at about 18,000 psi and soluble protein was separated from insoluble material and cell debris by centrifugation (about 40,000×g for about 1 hour). The soluble fraction was applied to a 30 ml Q Sepharose Fast Flow column (XK16, GE/Amersham) pre-equilibrated with 100 mM Tris-HCl, pH 8.0, 0.2 M NaCl. The flow-through fraction was diluted 1:10 in 100 mM Tris-HCl, pH 8.0, and loaded onto a 45 ml Source Q column (XK16, GE/Amersham). Bound protein was eluted with a linear salt gradient from 0 to 1.0 M NaCl in 100 mM Tris-HCl, pH 8.0. For final polishing and endotoxin removal, peak fractions were pooled and loaded directly onto a Superdex 200 gel filtration column (10/300 GL, GE/Amersham) pre-equlibrated in 100 mM Tris, 150 mM NaCl, 1% glycerol and 1% Na-deoxycholate. Peak fractions from the included volume of the column were pooled, dialyzed against 1×PBS and stored at about −80° C.

Protein QC: Methods for determining protein concentration (BCA), purity and identity (SDS-PAGE and western blot), and endotoxin (LAL) are described in Example 6.

Cells and TLR bioassays: The bioactivity of purified recombinant proteins was tested in cell culture. RAW264.7 cells were obtained from ATCC (Rockville, Md.). This cell line expresses TLR2 and TLR4, but not TLR5. RAW264.7 cells were transfected with a plasmid encoding human TLR5 (InVitrogen, San Diego Calif.) to generate RAW/TLR5 cells. TLR5-specific activity of fusion proteins was evaluated by measuring induction of TNFα production using established methods. In brief, RAW264.7 and RAW/TLR5 cells were cultured in 96-well microtiter plates (Costar) at a seeding density of about 3−5×10⁴ cells in about 100 μl/well in DMEM medium supplemented with 10% FCS and antibiotics. The next day, cells were treated for about 5 hours with serial dilutions of test proteins starting at about 5 μg/ml. For positive controls RAW264.7 cells were treated with the TLR4 agonist LPS (Sigma Aldrich). At the completion of the assay, supernatants were harvested and TNFα expression was evaluated by ELISA (InVitrogen, Carlsbad, Calif.). Absorbance and luminescence were evaluated using a TECAN microplate spectrophotometer running Magellan software (Amersham).

Animal studies: Female C57BL/6 or BALB/c mice of about 6 to about 8 weeks were purchased from the Jackson Laboratory (Bar Harbor, Me.). Animals were housed in the Yale University Animal facility (New Haven, Conn.). All studies were performed in accordance with the Yale University Institutional Animal Care and Use Committee (IACUC). Animals were immunized with purified endotoxin-free recombinant fusion proteins in sterile phosphate buffered saline (PBS), equimolar concentrations of M2e peptide, recombinant 4×M2e alone or formulated with aluminum hydroxide (Alum)(Pierce) or PBS alone. All immunizations were delivered subcutaneously (s.c.) in a volume of about 100 μl. Mice were divided into groups of 5 mice, and immunized subcutaneously on each side of the base of the tail on days 0 and 21 with the indicated concentrations of STF2.4×M2e fusion protein. On days 10 (primary) and 28 (boost), individual mice were bled by retro-orbital puncture. Sera were harvested by clotting and centrifugation of the heparin-free blood samples.

Studies with female and male New Zealand White rabbits were performed at Covance Research Products (Denver, Pa.). Animals (6/group) were immunized on days 0 and 21 with about 5 μg to about 500 μg of STF2.4×M2e. Sera were harvested on indicated days and evaluated for M2e-specific IgG responses by ELISA. For the rabbit ELISA, results are reported as change in OD (ΔOD) (pre-immune subtracted) values.

Peptides: Peptides corresponding to the 24 amino acid ectodomain of influenza A M2 from PR/8, Viet Nam, Hong Kong and Duck isolates were synthesized by Bachem Bioscience Inc., (King of Prussia, Pa.) using solid phase synthesis methodologies and FMOC chemistry. An overlapping peptide array that spanned the 24-amino acid sequence of M2e (PR/8) and consisted of thirteen (13) 12-amino acid residue peptides offset by one amino acid was synthesized by Sigma Genosys (Woodlands, Tex.) using their PEP-screen peptide synthesis platform.

Serum antibody determination: M2e-specific IgG levels were determined by ELISA. ELISA plates (96 well) were coated overnight at 4° C. with about 100 μl/well M2e peptide in PBS (about 5 μg/ml). Plates were blocked with 200 μl/well of Assay Diluent Buffer (ADB; BD Pharmingen) for one hour at room temperature. The plates were washed 3× in PBS-T. Dilutions of the sera in ADB were added (about 100 μl/well) and the plates were incubated overnight at about 4° C. The plates were washed 3× with PBS-T. HRP-labeled goat anti-mouse or goat anti-rabbit IgG antibodies (Jackson Immunochemical) diluted in ADB were added (about 100 μl/well) and the plates were incubated at room temperature for 1 hour. The plates were washed 3× with PBS-T. After adding TMB Ultra substrate (Pierce) and monitoring color development, A₄₅₀ was measured on a microplate spectrophotometer.

MDCK whole cell ELISA: Sera were tested for reactivity with influenza A infected MDCK cells. In brief, MDCK cells (ATCC Catalog #CCL-34, Manassas, Va.) were grown in 96-well culture plates (BD Catalog #353075, Corning, N.Y.) in DMEM complete medium containing 10% FCS at about 37° C. for one to two days or until cells were near confluence. Wells were then incubated with about 1×10⁶ EID of PR8 virus (about 50 μl) in DMEM medium without FCS or with medium alone (for uninfected controls). Following about a 60 minute incubation at about 37° C., about 200 μl of complete medium was added to each well and plates were incubated overnight at about 37° C. The next day plates were washed with PBS and fixed with 10% formalin at room temperature for about 10 minutes. Wells were washed three times with PBS/0.1% BSA and blocked with about 200 μl/well ADB (BD Pharmingen, San Diego, Calif.) for about one hour at room temperature or overnight at about 4° C. Serial dilutions of test sera were added to the wells and incubated for one to two hours at room temperature. Wells were washed and incubated with HRP-conjugated goat anti-mouse IgG (Jackson Immunochemical, West Grove, Pa.) for about 30 minutes at room temperature, followed by TMB Ultra substrate (Pierce catalog #34028, Rockford, Ill.) for about two minutes at room temperature. The reaction was stopped with the addition of about 25 μl of 1 N H₂SO₄ and the OD₄₅₀ was measured on a microplate spectrophotometer. Data reflect the mean ΔOD (infected-uninfected cells) of triplicate wells per sample.

Influenza virus challenge of mice. To assess efficacy, mice were challenged on day 28 by intranasal administration of an LD₉₀ (dose lethal to about 90% of mice; about 8×10³ EID) of influenza A isolate PR8. Animals were monitored daily for 21 days following the challenge for survival, weight loss and clinical presentation. The percent weight loss was calculated for each individual animal per group. Clinical scores were assigned as follows: 4 pts=healthy, 3 pts=reduced grooming, 2 pts=reduced physical activity and 1 pt=moribund. Experimental results for clinical scores and weight loss reflect the results based on surviving animals on the day evaluated.

### Results

Design and expression of an M2e based vaccine: The ectodomain of the influenza A matrix protein (M2e) represents a highly conserved viral determinant that is expressed on the surface of influenza infected cells (Ito, et al. J Virol 1991, 65(10), 5491-5498; Zebedee, et. al., Nucleic Acids Res 1989, 17(7), 2870). The ectodomain of M2 comprises a relatively small polypeptide of 24 amino acids that is poorly immunogenic when delivered alone. A construct containing four tandem repeats of the H1/H2/H3 consensus M2e sequence SLLTEVETPIRNEWGSRSNDSSDP (SEQ ID NO: 13) fused to the C-terminus of Salmonella typhimurium fljB (STF2), designated pET/STF2.4×M2e, was cloned and expressed in E. coli. Recombinant fusion protein expression was induced in shake flask cultures and the protein was purified by a multi-step chromatographic method. Purity of STF2.4×M2e recombinant protein was evaluated by SDS-PAGE and Western blot analysis with the M2e-specific mAb 14C2, which indicated a single polypeptide band with a molecular weight of about 66 kDa and purity greater than about 95%.

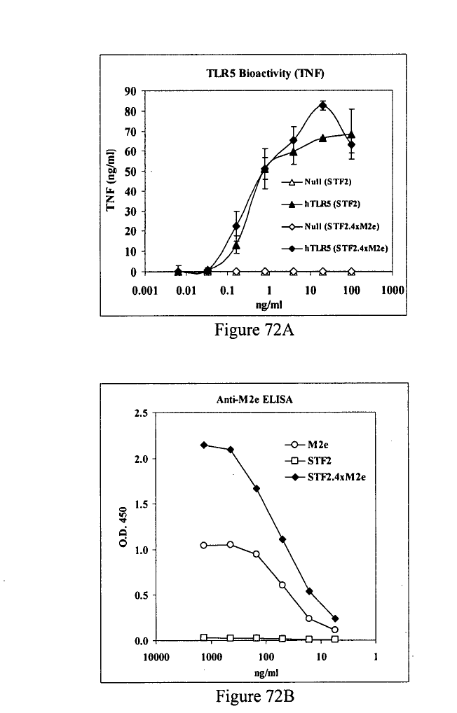

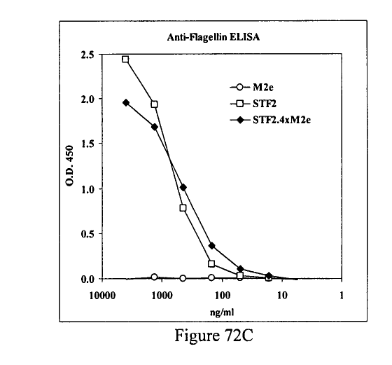

Bioactivity of the recombinant STF2.4×M2e protein: TLR5 biological activity of STF2.4×M2e protein was examined by determining the ability of the protein to induce TNFα expression in cells expressing TLR5. Serial dilutions of the purified protein were added to the RAW264.7 (TLR5-) and RAW/TLR5 (TLR5+) cells and incubated overnight. Culture supernatants were harvested and analyzed for TNFα content by sandwich ELISA. STF2.4×M2e specifically activated RAW/TLR5 cells but not RAW264.7 cells (FIG. 72A). Evaluation of STF2.4×M2e protein in LAL assays demonstrated the final preparation contained endotoxin levels <0.05 EU/μg, in agreement with the inability of the protein to activate TLR4+RAW264.7 cells.

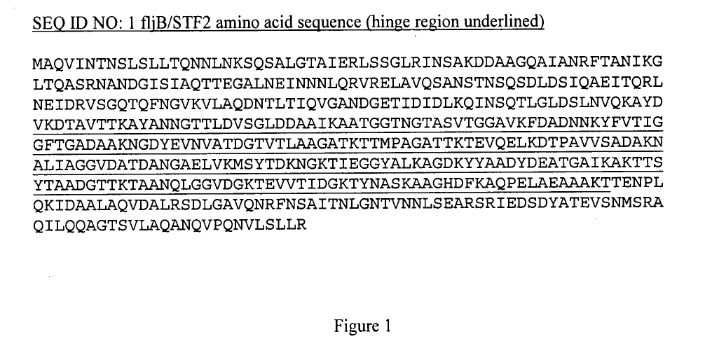

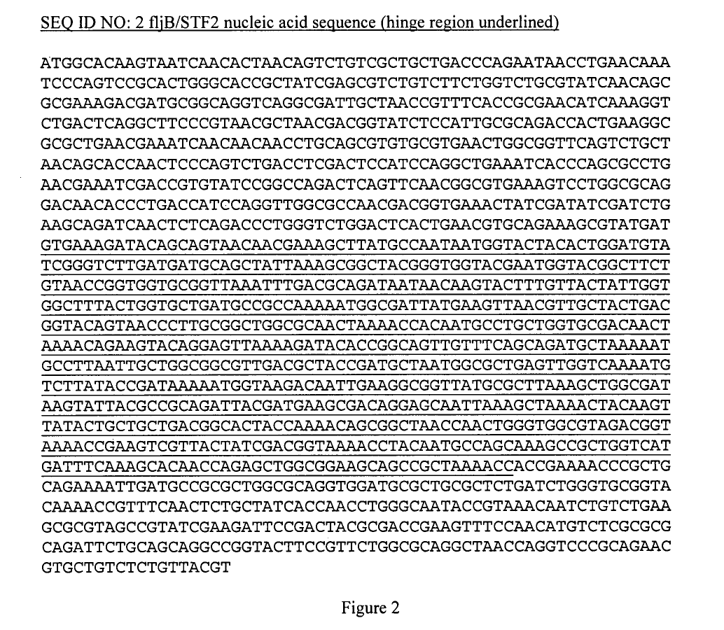

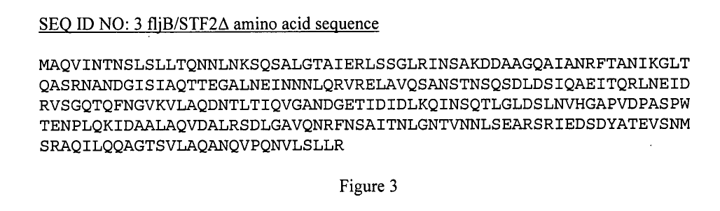

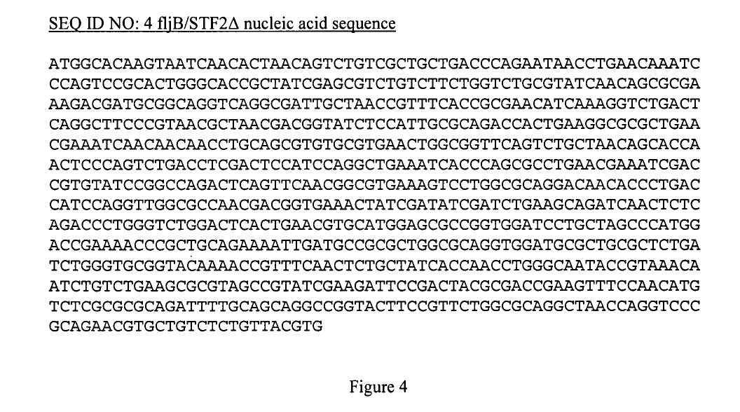

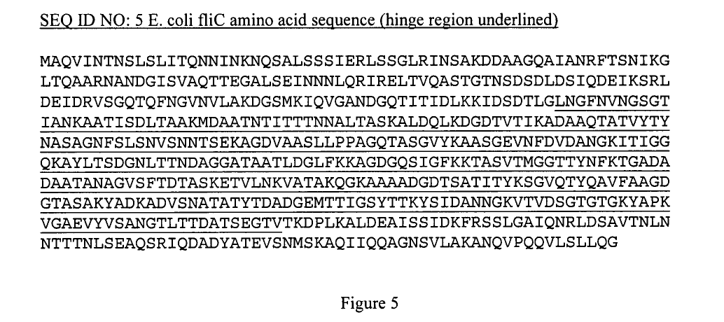

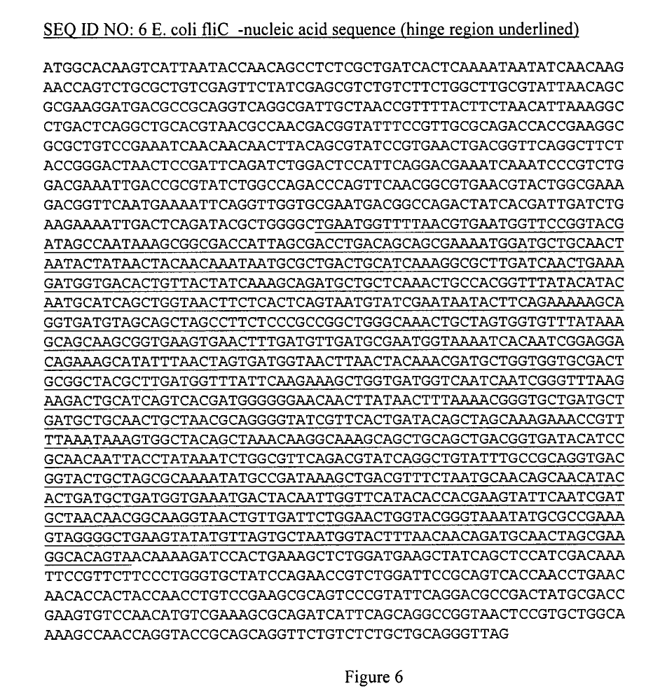

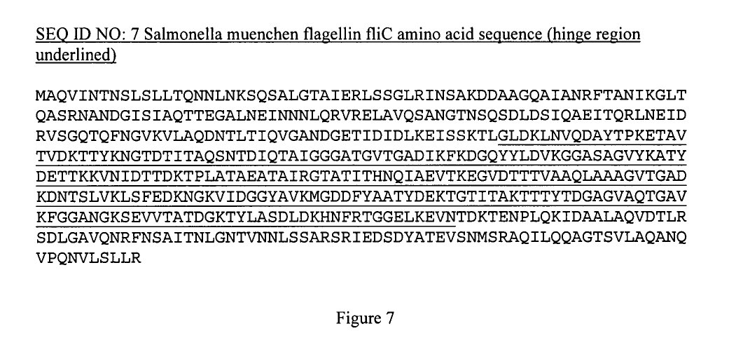

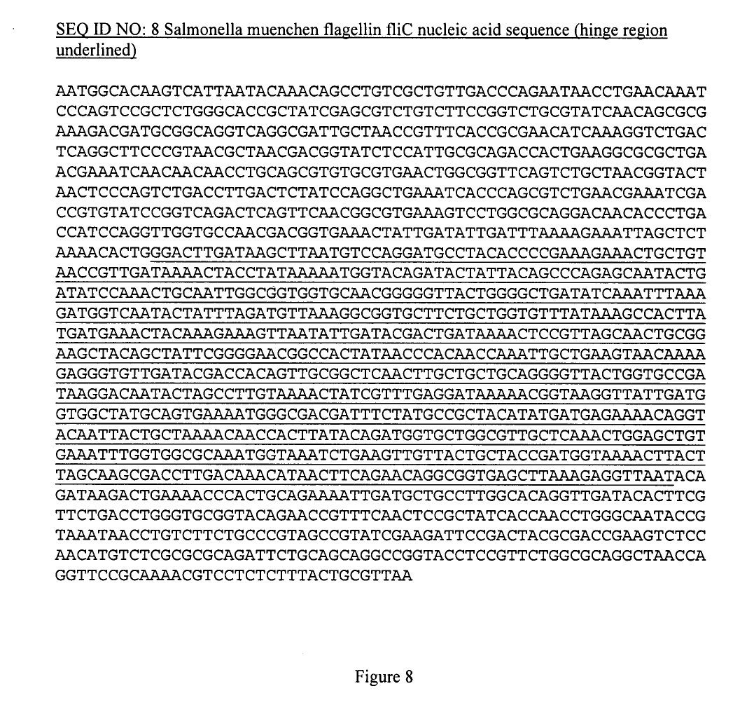

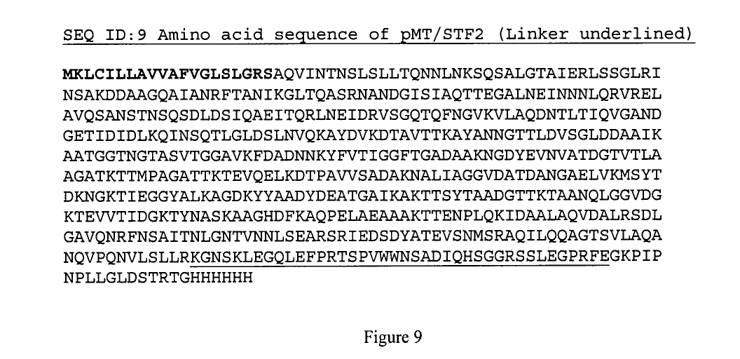

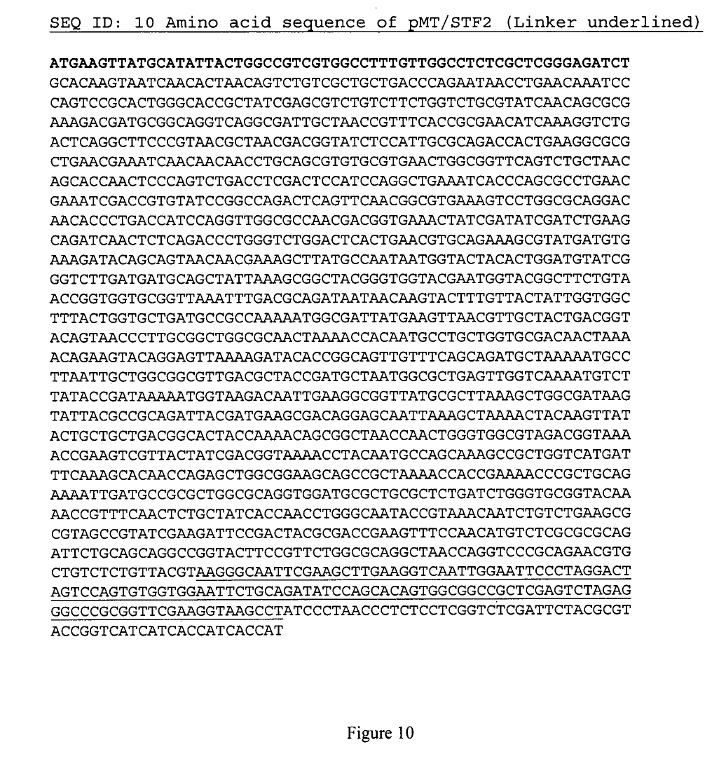

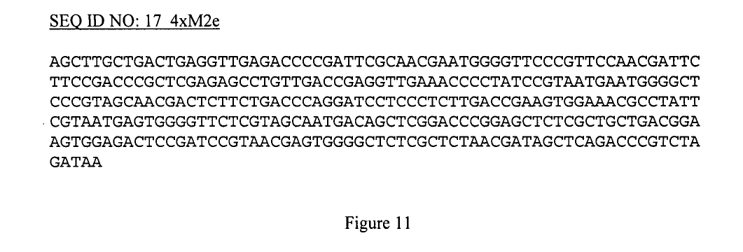

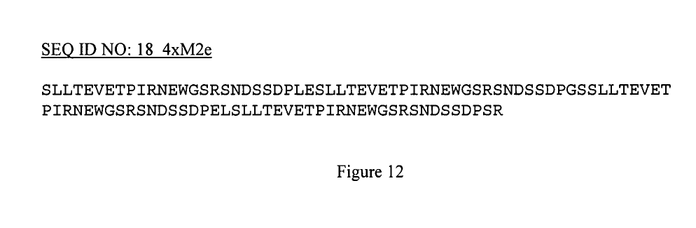

Antigenicity of STF2.4×M2e protein: Appropriate display of the M2e epitope in the STF2.4×M2e protein was examined by ELISA. Plates were coated with serial dilutions of STF2.4×M2e protein, then probed with the M2e-specific and protective mAb 14C2 and the flagellin specific mAb 6H11. The results confirm the presence of M2e (FIG. 1B) and flagellin (FIG. 72C) in the purified protein. Passive transfer studies demonstrate that mAb 14C2 recognizes a protective epitope in M2 (Treanor et al J Virol 1990, 64(3), 1375-1377; Liu et al. Immunol Lett 2004, 93(2-3), 131-136; Zou et al Int Immunopharmacol 2005, 5(4), 631-635.). The data indicate that this defined protective epitope of M2e is properly presented in the fusion protein.

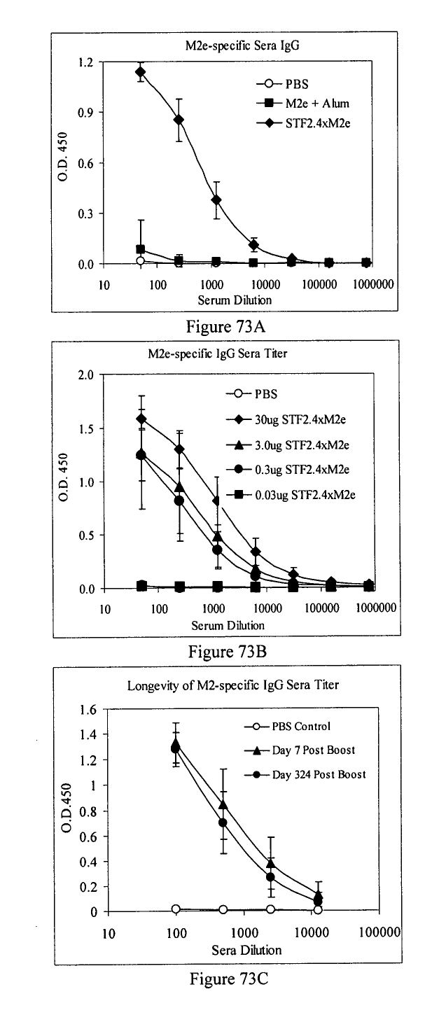

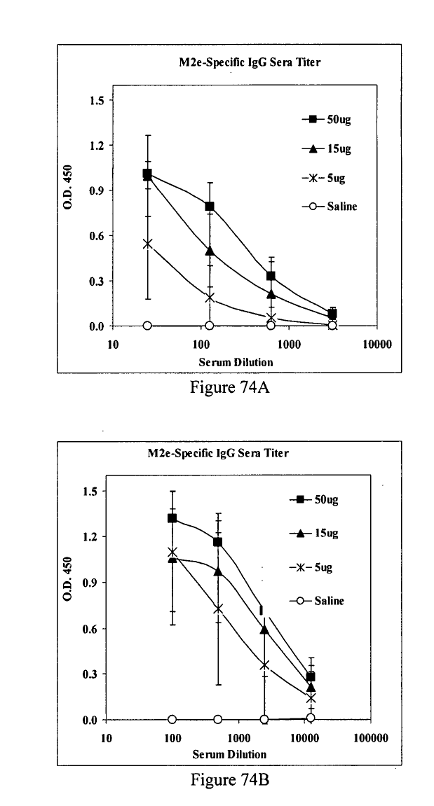

Immunogenicity of STF2.4×M2e fusion protein in mice: The immunogenicity of STF2.4×M2e was examined in BALB/c mice immunized on day 0 and 14 with STF2.4×M2e or an equimolar dose of M2e peptide in alum or PBS alone. On day 21 animals were bled and the sera of individual animals were examined for M2e-specific IgG by ELISA. Mice immunized with STF2.4×M2e exhibited significantly higher levels of M2e-specific IgG than mice immunizated with M2e peptide adsorbed to alum (FIG. 73A). These data demonstrate STF2.4×M2e, administered in saline with no conventional carrier or adjuvant, elicits a more potent antigen-specific IgG response than that observed with M2e formulated with the conventional adjuvant alum. To more fully characterize the potency of the vaccine, BALB/c mice were immunized on day 0 and 14 with 30, 3, 0.3 or 0.03 μg of STF2.4×M2e and examined for antigen-specific antibody responses to M2e on day 21. The results in FIG. 73B demonstrate that immunization with as little as about 0.3 μg of STF2.4×M2e induced levels of M2e-specific responses that were comparable to those in mice receiving about 30 μg of the same recombinant fusion protein.

The longevity of the M2e-specific IgG responses was evaluated in BALB/c mice immunized on days 0 and 21 with about 3 μg of STF2.4×M2e, and bled on days 7 and 324 post-boost. M2e-specific IgG responses were comparable 7 and 324 days post-boost (FIG. 73C). The persistence of the M2e-specific IgG response was consistent with responses we have observed to other recombinant TLR-ligand fusion proteins (Huleatt et al. Vaccine 2007, 25(4), 763-775; McDonald et al. J Infect Dis 2007, 195, 1607-1617).

Immunogenicity of STF2.4×M2e fusion protein in rabbits: The immunogenicity of the STF2.4×M2e proteins was also examined in rabbits. Groups of six New Zealand White rabbits were immunized on days 0 and 21 with about 5 μg to about 500 μg of STF2.4×M2e. Sera were harvested about 14 days post-prime or about 7 days post-boost and evaluated for M2e-specific IgG responses by ELISA. For the rabbit ELISA, results are reported as ΔOD (pre-immune subtracted) values. The results shown in FIGS. 74A and 74B demonstrate the M2e-specific IgG responses following the priming immunization (FIG. 74A) and the boost (FIG. 74B). All animals in all dose groups sero-converted post-prime, with the exception of the about 5 μg dose group in which four of the six animals sero-converted post-prime and all sero-converted post-boost. These data demonstrate that STF2.4×M2e is immunogenic at low doses in mice and rabbits.

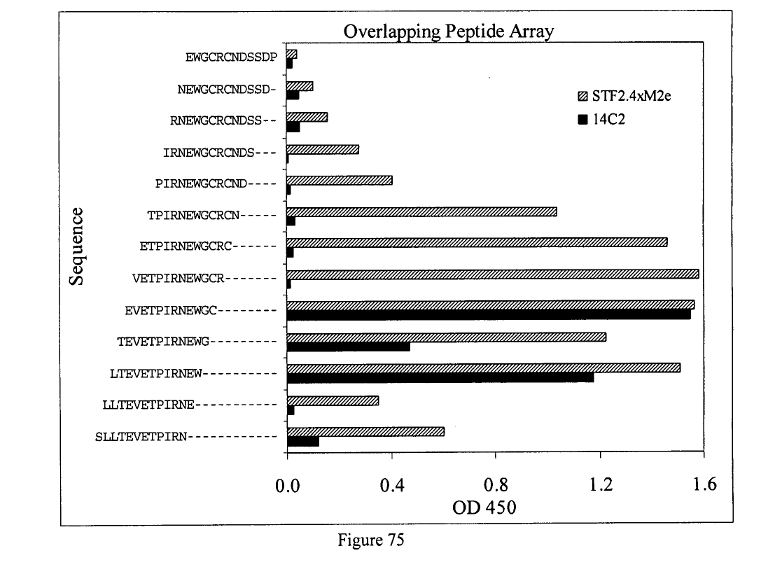

Epitope specificity of STF2.4×M2e-immune animals: The fine epitope specificity of antisera from mice immunized with STF2.4×M2e was examined by ELISA using overlapping subunit peptides based on the immunizing consensus M2e sequence. Overlapping 12-mer peptides (SEQ ID NOS: 245-257) offset by one amino acid were synthesized and probed with the antisera and with the protective monoclonal antibody 14C2. The reactivity of mAb 14C2 confirms the prior identification of peptide EVETPIRN (SEQ ID NO: 20) epitope as the defined target of the M2e-specific and protective antibodies (Liu et al. Immunol Lett 2004, 93(2-3), 131-136; Zou et al. Int Immunopharmacol 2005, 5(4), 631-635). The reactivity of sera from STF2.4×M2e-immune mice (FIG. 75) and rabbits demonstrates that reactivity encompasses not only the 14C2-defined epitope but also flanking sequences not recognized by the monoclonal antibody. The broader epitope recognition in the polyclonal response induced by STF2.4×M2e offers the potential of broader recognition across multiple M2e sequences that differ by one or two amino acids.

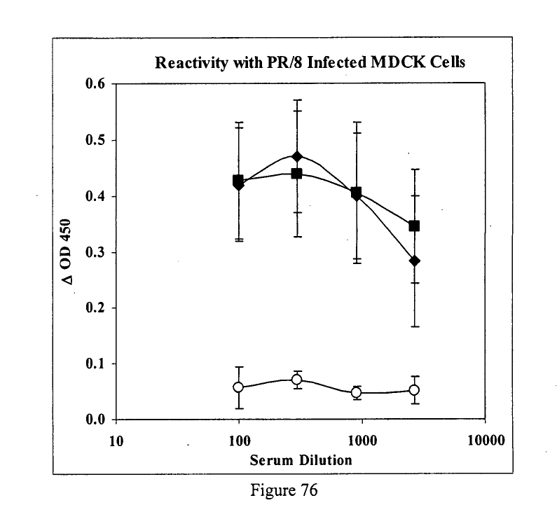

Serum from STF2.4×M2e-immune animals reacts with influenza-infected cells: The data described above demonstrate that the antibody response induced by STF2.4×M2e reacted with synthetic peptides corresponding to the consensus M2e. In order to determine whether the antibody response recognizes native M2, a whole cell ELISA assay was utilized. MDCK cells were grown in tissue culture plates and infected with influenza A PR8 virus, then fixed and incubated with serial dilutions of immune and non-immune sera. The results shown in FIG. 76 demonstrate that sera from mice immunized with either about 30 or about 3.0 μg of STF2.4×M2e bound specifically to PR/8/34-infected MDCK cells, indicating that the anti-M2e antibodies elicited by immunization with STF2.4×M2e recognize native M2 on the surface of influenza-infected cells. Since antibodies specific for M2e are known to not neutralize viral infectivity, but rather exert their protective effect at the level of the infected cell, these data indicate that the antibody response raised against STF2.4×M2e will protect the host against virus challenge.

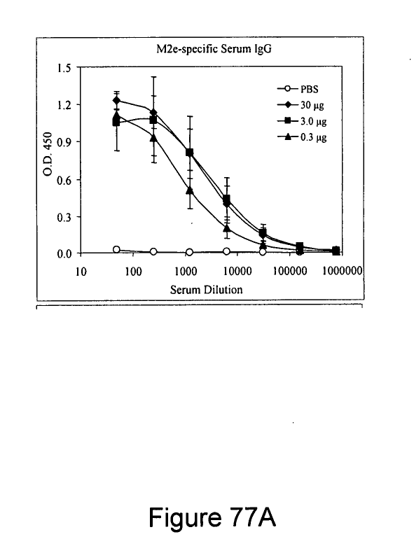

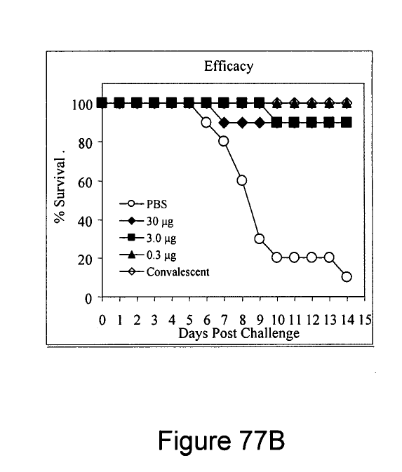

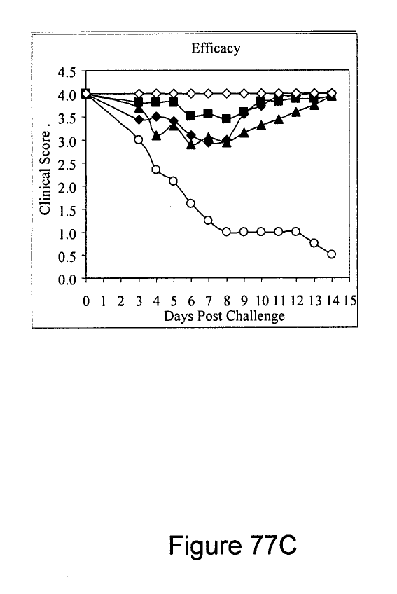

STF2.4×M2e provides protection against lethal influenza virus challenge: Based on these data that immunization with STF2.4×M2e induced a potent, long-lasting antibody response that recognized a known protective epitope of M2e and bound to virus-infected cells, the protective capacity of STF2.4×M2e immunization was evaluated. BALB/c mice (10/group) were immunized twice (on day 0 and 14) by subcutaneous injection of about 30, about 3 or about 0.3 μg of STF2.4×M2e. On day 21 the sera of immunized animals were examined for M2e-specific IgG by ELISA (FIG. 77A). These data show a very potent antibody response in mice immunized with as little as about 0.3 μg, with a maximal response achieved with a dose of about 3 μg. Mice were challenged on day 28 by intranasal administration of 1×LD₉₀ of influenza PR8 virus. Mice were observed for clinical score and survival for two weeks post-challenge. The results demonstrate that mice immunized with as little as about 0.3 μg demonstrated enhanced survival post-challenge with the PR8 virus compared to naïve mice (FIG. 77B). Observations of clinical scores (FIG. 77C) show that immunized mice experienced significantly milder disease symptoms than naïve mice.

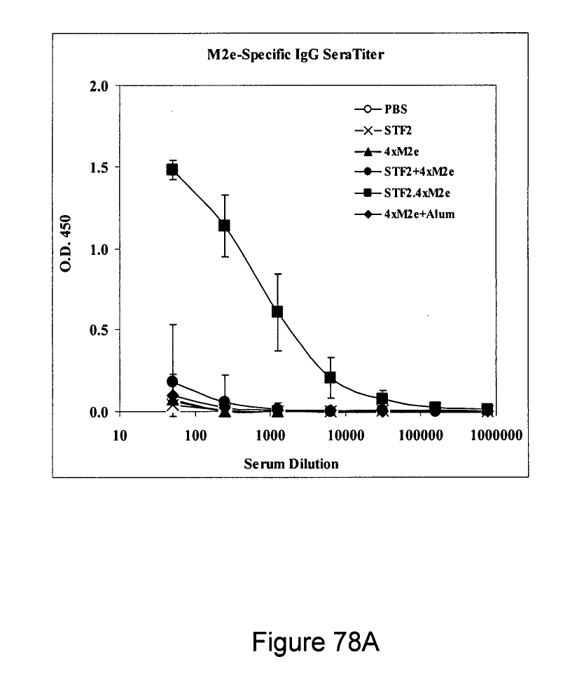

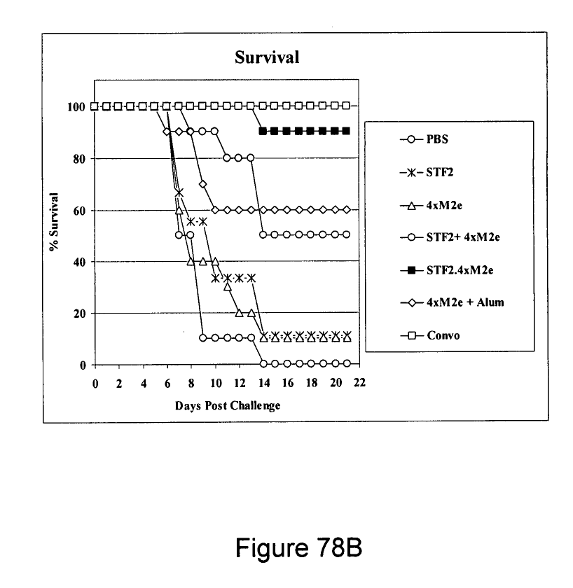

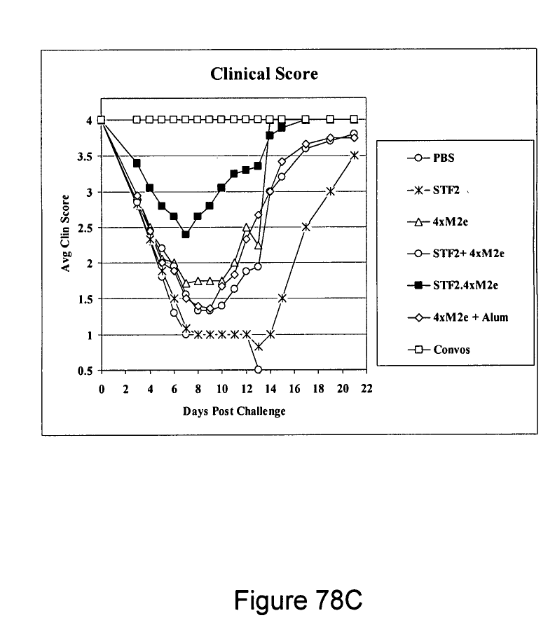

Other studies have examined responses following the co-delivery of antigens with flagellin in cocktail or in separate DNA vectors (McSorley et al. J Immunol 2002, 169(7), 3914-3919; Cuadros et al. Infect Immun 2004, 72(5), 2810-2816; Didierlaurent et al. J Immunol 2004, 172(11), 6922-6930; Applequist et al. J Immunol 2005, 175(6), 3882-3891). In order to assess fused of a flagellin to an M2e antigen increased immunogenicity, a mixing comparison was performed. BALB/c mice were immunized s.c. on days 0 and 14 with STF2.4×M2e, an equimolar dose of 4×M2e alone, an equimolar dose of STF2 alone, an equimolar dose of 4×M2e mixed with STF2, or an equimolar dose of 4×M2e on alum. One week following the boost, sera were harvested and examined for M2e-specific IgG responses by ELISA. The results demonstrate that mice immunized with STF2.4×M2e fusion protein demonstrated significant antibody responses to M2e, even though all three groups received the same dose of M2e either alone or mixed with STF2 (FIG. 78A). Physical fusion of the antigen 4×M2e to the TLR ligand flagellin can increase immunogenicity of the M2e epitope.

The mice were challenged by intranasal administration of about 1×LD₉₀ of PR/8 virus on day 28 and monitored for survival for 21 days. Mice immunized with STF2.4×M2e were protected from lethal virus challenge, while mice immunized with 4×M2e alone were not protected (FIGS. 78B and 78C), similar to results previously obtained in other systems (Huleatt et al. Vaccine 2007, 25(4), 763-775; McDonald et al. J Infect Dis 2007, 195, 1607-1617). Interestingly, mice immunized with STF2+4×M2e demonstrated protection that was intermediate to those receiving the fusion protein or 4×M2e alone. The partial protection provided by the mixture of STF2 and 4×M2e may be a consequence of the multimeric nature of 4×M2e antigen.

### Discussion

A synthetic gene encoding four tandem copies of the influenza A M2e epitope was fused to the 3′ end of the gene encoding full-length flagellin (STF2) to yield the vaccine candidate construct, STF2.4×M2e(PR8). The protein was expressed in E. coli and purified to homogeneity. The immunogenicity of the protein was tested in BALB/c mice. ELISA results demonstrate that mice immunized with STF2.4×M2e fusion protein, but not those immunized with equivalent doses of STF2 mixed with 4×M2e or with 4×M2e alone, developed potent antibody responses which recognized a known protective epitope within M2e. Furthermore, mice immunized with STF2.4×M2e protein survived a subsequent challenge with a dose of influenza virus that was lethal to about 90% of naïve mice, suggesting that the anti-M2e antibody response induced by immunization with the fusion protein correlated with protection from pathogenic challenge. Finally, the antibody response induced by immunization with STF2.4×M2e was long-lived, as it did not decline between 7 and 324 days post-immunization.

The teachings of all of the references cited herein are hereby incorporated by reference in their entirety.

### EQUIVALENTS

While this invention has been particularly shown and described with references to preferred embodiments thereof, it will be understood by those skilled in the art that various changes in form and details may be made therein without departing from the scope of the invention encompassed by the appended claims.

1. A composition comprising at least one Pam3Cys and at least a portion of at least one integral membrane protein of an influenza viral protein.

2. The composition of claim 1, wherein the influenza viral protein is an influenza A viral protein.

3. The composition of claim 1, wherein the influenza protein is an influenza B viral protein.

4. The composition of claim 1, wherein the influenza protein is an influenza C viral protein.

5. The composition of claim 2, wherein the integral membrane protein is at least one member selected from the group consisting of a haemagglutinin membrane protein, a neuraminidase membrane protein and an M2 membrane protein.

6. The composition of claim 5, wherein the integral membrane protein includes an M2 protein and wherein the M2 protein includes at least a portion of SEQ ID NO: 13.

7. The composition of claim 5, wherein the M2 protein includes at least one member selected from the group consisting of SEQ ID NO: 15, SEQ ID NO: 19 and SEQ ID NO: 34.

8. The composition of claim 5, wherein the integral membrane protein includes a haemagglutinin protein that includes at least a portion of at least one member selected from the group consisting of SEQ ID NO: 64 and SEQ ID NO: 67.

9. The composition of claim 8, wherein the haemagglutinin protein includes at least one member selected from the group consisting of SEQ ID NO: 35, SEQ ID NO: 36, SEQ ID NO: 37 and SEQ ID NO: 38.

10. The composition of claim 1, further including at least one Pam2Cys.

11. The composition of claim 10, wherein the Pam3Cys, the Pam2Cys and the integral membrane protein are components of a fusion protein.

12. The composition of claim 1, wherein the Pam3Cys and the integral membrane protein are components of a fusion protein.

13. The composition of claim 12, further including a linker between at least one Pam3Cys and at least one integral membrane protein of the composition.

14. The composition of claim 13, wherein the linker is an amino acid linker.

15. The composition of claim 1, further including a linker between at least two integral membrane proteins of the composition.

16. The composition of claim 15, wherein the linker is an amino acid linker.

17. The composition of claim 1, further including a TLR5 agonist.

18. The composition of claim 17, wherein the TLR5 agonist is a flagellin.

19. The composition of claim 18, wherein the flagellin is at least one member selected from the group consisting of a Fljb/STF2, a E. coli fliC, and a S. muenchen fliC.

20. A fusion protein comprising at least one pathogen-associated molecular pattern and at least one influenza M2 protein, wherein the pathogen-associated molecular pattern is not a Pam2Cys.

21. The fusion protein of claim 20, wherein the M2 protein includes at least a portion of SEQ ID NO: 13.

22. The fusion protein of claim 21, further including a linker between at least one pathogen-associated molecular pattern and at least one M2 protein.

23. The fusion protein of claim 21, further including a linker between at least two M2 proteins.

24. The fusion protein of claim 21, wherein the M2 protein includes SEQ ID NO: 15.

25. The fusion protein of claim 20, wherein the pathogen-associated molecular pattern is a TLR5 agonist.

26. The fusion protein of claim 25, wherein the TLR5 agonist is a flagellin.

27. The fusion protein of claim 26, wherein the flagellin is at least one member selected from the group consisting of a fljB/STF2, a E. coli fliC, and a S. muenchen fliC.

28. The fusion protein of claim 27, wherein the flagellin includes the fljB/STF2, and wherein the fljB/STF2 includes at least a portion of SEQ ID NO: 1.

29. The fusion protein of claim 28, wherein the fljB/STF2 includes SEQ ID NO: 3.

30. The fusion protein of claim 27, wherein the flagellin includes the E. coli fliC, and wherein the E. coli fliC that includes at least a portion of SEQ ID NO: 5.

31. The fusion protein of claim 30, wherein the E. coli fliC includes SEQ ID NO: 66.

32. The fusion protein of claim 27, wherein the flagellin includes the S. muenchen fliC and wherein the S. muenchen fliC includes at least a portion of SEQ ID NO: 7.

33. The fusion protein of claim 32, wherein the S. muenchen fliC includes SEQ ID NO: 98.

34. The fusion protein of claim 20, wherein the pathogen-associated molecular pattern is fused to a carboxy-terminus of the influenza M2 protein.

35. The fusion protein of claim 20, wherein the pathogen-associated molecular pattern is fused to an amino-terminus of the influenza M2 protein.

36. The fusion protein of claim 20, wherein at least one pathogen-associated molecular pattern is between at least two influenza M2 proteins.

37. The fusion protein of claim 20, wherein the pathogen-associated molecular pattern is a TLR2 agonist.

38. The fusion protein of claim 37, wherein the TLR2 agonist is a Pam3Cys.

39. The fusion protein of claim 20, further including at least a portion of a haemagglutinin membrane protein.

40. The fusion protein of claim 20, further including at least a portion of a neuraminidase membrane protein.

41. The fusion protein of claim 20, further including at least one member selected from the group consisting of an influenza B viral protein and an influenza C viral protein.

42. The fusion protein of claim 41, wherein the influenza B viral protein is an integral membrane protein.

43. The fusion protein of claim 41, wherein the influenza C viral protein is an integral membrane protein.

44. A composition comprising a pathogen-associated molecular pattern and an M2 protein, wherein the pathogen-associated molecular pattern is not a Pam2Cys.

45. A composition comprising at least a portion of at least one pathogen-associated molecular pattern and at least a portion of at least one influenza M2 protein, wherein, if the pathogen-associated molecular pattern includes a Pam2Cys, at least a portion of the Pam2Cys is not fused to the influenza M2 protein and at least a portion of the influenza M2 protein is not fused to the Pam2Cys.

46. A fusion protein comprising at least a portion of at least one pathogen-associated molecular pattern and at least a portion of at least one influenza M2 protein, wherein, if the pathogen-associated molecular pattern includes a Pam2Cys, at least a portion of the Pam2Cys is not fused to the influenza M2 protein and at least a portion of the influenza M2 protein is not fused to the Pam2Cys.

47. A method of stimulating an immune response in a subject, comprising the step of administering to the subject a composition that includes at least one Pam3Cys and at least a portion of at least one integral membrane protein of an influenza viral protein.

48. A method of stimulating an immune response in a subject, comprising the step of administering to the subject a composition that includes a fusion protein comprising at least one pathogen-associated molecular pattern and at least one influenza M2 protein, wherein the pathogen-associated molecular pattern is not a Pam2Cys.

49. A method of stimulating an immune response in a subject, comprising the step of administering to the subject a composition that includes at least one pathogen-associated molecular pattern and at least one influenza M2 protein, wherein the pathogen-associated molecular pattern is not a Pam2Cys and the M2 protein is not an M2e protein.

50. A method of stimulating an immune response in a subject, comprising the step of administering to the subject a composition that includes a composition comprising at least a portion of at least one pathogen-associated molecular pattern and at least a portion of at least one influenza M2 protein, wherein, if the pathogen-associated molecular pattern includes a Pam2Cys, at least a portion of the Pam2Cys is not fused to the influenza M2 protein and at least a portion of the influenza M2 protein is not fused to the Pam2Cys.

51. A method of stimulating an immune response in a subject, comprising the step of administering to the subject a composition that includes a fusion protein comprising at least a portion of at least one pathogen-associated molecular pattern and at least a portion of at least one influenza M2 protein, wherein, if the pathogen-associated molecular pattern includes a Pam2Cys, at least a portion of the Pam2Cys is not fused to the influenza M2 protein and at least a portion of the influenza M2 protein is not fused to the Pam2Cys.

52. A method of decreasing an antibody response to at least a portion of a flagellin that is a component of a fusion protein, wherein the fusion protein activates a Toll-like Receptor 5 and includes at least one antigen, comprising the step of deleting at least a portion of a hinge region of the flagellin.

53. The method of claim 52, wherein the hinge region is deleted prior to fusion of the flagellin to the antigen.

54. The method of claim 52, wherein the hinge region is deleted from a fusion protein that includes an influenza antigen.

55. The method of claim 54, wherein the influenza antigen is at least one member selected from the group consisting of an influenza A antigen, an influenza B antigen and an influenza C antigen.

56. The method of claim 54, wherein the influenza antigen is an integral membrane protein antigen.

57. The method of claim 56, wherein the integral membrane protein antigen is at least one member selected from the group consisting of a hemagglutinin integral membrane protein antigen, a matrix 2 protein antigen and a neuraminidase protein antigen.

58. The method of claim 54, wherein the influenza antigen is a nucleoprotein antigen.

59. A method of increasing an in vitro yield of a fusion protein, wherein the fusion protein activates a Toll-like Receptor 5 and includes at least a portion of at least one flagellin and at least a portion of at least one antigen, comprising the step of forming a fusion protein lacking at least a portion of a naturally occurring hinge region.

60. The method of claim 47, wherein the immune response is a protective immune response.

61. The method of claim 48, wherein the immune response is a protective immune response.

62. The method of claim 49, wherein the immune response is a protective immune response.

63. The method of claim 50, wherein the immune response is a protective immune response.

64. The method of claim 51, wherein the immune response is a protective immune response.


SEQUENCE LISTING (257 sequences)

SEQ ID NO: 1 - protein, 506 aa, s. typhimurium
MAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDSLNVQKAYDVKDTAVTTKAYANNGTTLDVSGLDDAAIKAATGGTNGTASVTGGAVKFDADNNKYFVTIGGFTGADAAKNGDYEVNVATDGTVTLAAGATKTTMPAGATTKTEVQELKDTPAVVSADAKNALIAGGVDATDANGAELVKMSYTDKNGKTIEGGYALKAGDKYYAADYDEATGAIKAKTTSYTAADGTTKTAANQLGGVDGKTEVVTIDGKTYNASKAAGHDFKAQPELAEAAAKTTENPLQKIDAALAQVDALRSDLGAVQNRFNSAITNLGNTVNNLSEARSRIEDSDYATEVSNMSRAQILQQAGTSVLAQANQVPQNVLSLLR

SEQ ID NO: 2 - DNA, 1518 bp, s. typhimurium
atggcacaagtaatcaacactaacagtctgtcgctgctgacccagaataacctgaacaaatcccagtccgcactgggcaccgctatcgagcgtctgtcttctggtctgcgtatcaacagcgcgaaagacgatgcggcaggtcaggcgattgctaaccgtttcaccgcgaacatcaaaggtctgactcaggcttcccgtaacgctaacgacggtatctccattgcgcagaccactgaaggcgcgctgaacgaaatcaacaacaacctgcagcgtgtgcgtgaactggcggttcagtctgctaacagcaccaactcccagtctgacctcgactccatccaggctgaaatcacccagcgcctgaacgaaatcgaccgtgtatccggccagactcagttcaacggcgtgaaagtcctggcgcaggacaacaccctgaccatccaggttggcgccaacgacggtgaaactatcgatatcgatctgaagcagatcaactctcagaccctgggtctggactcactgaacgtgcagaaagcgtatgatgtgaaagatacagcagtaacaacgaaagcttatgccaataatggtactacactggatgtatcgggtcttgatgatgcagctattaaagcggctacgggtggtacgaatggtacggcttctgtaaccggtggtgcggttaaatttgacgcagataataacaagtactttgttactattggtggctttactggtgctgatgccgccaaaaatggcgattatgaagttaacgttgctactgacggtacagtaacccttgcggctggcgcaactaaaaccacaatgcctgctggtgcgacaactaaaacagaagtacaggagttaaaagatacaccggcagttgtttcagcagatgctaaaaatgccttaattgctggcggcgttgacgctaccgatgctaatggcgctgagttggtcaaaatgtcttataccgataaaaatggtaagacaattgaaggcggttatgcgcttaaagctggcgataagtattacgccgcagattacgatgaagcgacaggagcaattaaagctaaaactacaagttatactgctgctgacggcactaccaaaacagcggctaaccaactgggtggcgtagacggtaaaaccgaagtcgttactatcgacggtaaaacctacaatgccagcaaagccgctggtcatgatttcaaagcacaaccagagctggcggaagcagccgctaaaaccaccgaaaacccgctgcagaaaattgatgccgcgctggcgcaggtggatgcgctgcgctctgatctgggtgcggtacaaaaccgtttcaactctgctatcaccaacctgggcaataccgtaaacaatctgtctgaagcgcgtagccgtatcgaagattccgactacgcgaccgaagtttccaacatgtctcgcgcgcagattctgcagcaggccggtacttccgttctggcgcaggctaaccaggtcccgcagaacgtgctgtctctgttacgt

SEQ ID NO: 3 - protein, 277 aa, s. typhimurium
MAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDSLNVHGAPVDPASPWTENPLQKIDAALAQVDALRSDLGAVQNRFNSAITNLGNTVNNLSEARSRIEDSDYATEVSNMSRAQILQQAGTSVLAQANQVPQNVLSLLR

SEQ ID NO: 4 - DNA, 832 bp, s. typhimurium
atggcacaagtaatcaacactaacagtctgtcgctgctgacccagaataacctgaacaaatcccagtccgcactgggcaccgctatcgagcgtctgtcttctggtctgcgtatcaacagcgcgaaagacgatgcggcaggtcaggcgattgctaaccgtttcaccgcgaacatcaaaggtctgactcaggcttcccgtaacgctaacgacggtatctccattgcgcagaccactgaaggcgcgctgaacgaaatcaacaacaacctgcagcgtgtgcgtgaactggcggttcagtctgctaacagcaccaactcccagtctgacctcgactccatccaggctgaaatcacccagcgcctgaacgaaatcgaccgtgtatccggccagactcagttcaacggcgtgaaagtcctggcgcaggacaacaccctgaccatccaggttggcgccaacgacggtgaaactatcgatatcgatctgaagcagatcaactctcagaccctgggtctggactcactgaacgtgcatggagcgccggtggatcctgctagcccatggaccgaaaacccgctgcagaaaattgatgccgcgctggcgcaggtggatgcgctgcgctctgatctgggtgcggtacaaaaccgtttcaactctgctatcaccaacctgggcaataccgtaaacaatctgtctgaagcgcgtagccgtatcgaagattccgactacgcgaccgaagtttccaacatgtctcgcgcgcagattttgcagcaggccggtacttccgttctggcgcaggctaaccaggtcccgcagaacgtgctgtctctgttacgtg

SEQ ID NO: 5 - protein, 595 aa, E. coli
MAQVINTNSLSLITQNNINKNQSALSSSIERLSSGLRINSAKDDAAGQAIANRFTSNIKGLTQAARNANDGISVAQTTEGALSEINNNLQRIRELTVQASTGTNSDSDLDSIQDEIKSRLDEIDRVSGQTQFNGVNVLAKDGSMKIQVGANDGQTITIDLKKIDSDTLGLNGFNVNGSGTIANKAATISDLTAAKMDAATNTITTTNNALTASKALDQLKDGDTVTIKADAAQTATVYTYNASAGNFSLSNVSNNTSEKAGDVAASLLPPAGQTASGVYKAASGEVNFDVDANGKITIGGQKAYLTSDGNLTTNDAGGATAATLDGLFKKAGDGQSIGFKKTASVTMGGTTYNFKTGADADAATANAGVSFTDTASKETVLNKVATAKQGKAAAADGDTSATITYKSGVQTYQAVFAAGDGTASAKYADKADVSNATATYTDADGEMTTIGSYTTKYSIDANNGKVTVDSGTGTGKYAPKVGAEVYVSANGTLTTDATSEGTVTKDPLKALDEAISSIDKFRSSLGAIQNRLDSAVTNLNNTTTNLSEAQSRIQDADYATEVSNMSKAQIIQQAGNSVLAKANQVPQQVLSLLQG

SEQ ID NO: 6 - DNA, 1788 bp, E. coli
atggcacaagtcattaataccaacagcctctcgctgatcactcaaaataatatcaacaagaaccagtctgcgctgtcgagttctatcgagcgtctgtcttctggcttgcgtattaacagcgcgaaggatgacgccgcaggtcaggcgattgctaaccgttttacttctaacattaaaggcctgactcaggctgcacgtaacgccaacgacggtatttccgttgcgcagaccaccgaaggcgcgctgtccgaaatcaacaacaacttacagcgtatccgtgaactgacggttcaggcttctaccgggactaactccgattcagatctggactccattcaggacgaaatcaaatcccgtctggacgaaattgaccgcgtatctggccagacccagttcaacggcgtgaacgtactggcgaaagacggttcaatgaaaattcaggttggtgcgaatgacggccagactatcacgattgatctgaagaaaattgactcagatacgctggggctgaatggttttaacgtgaatggttccggtacgatagccaataaagcggcgaccattagcgacctgacagcagcgaaaatggatgctgcaactaatactataactacaacaaataatgcgctgactgcatcaaaggcgcttgatcaactgaaagatggtgacactgttactatcaaagcagatgctgctcaaactgccacggtttatacatacaatgcatcagctggtaacttctcactcagtaatgtatcgaataatacttcagaaaaagcaggtgatgtagcagctagccttctcccgccggctgggcaaactgctagtggtgtttataaagcagcaagcggtgaagtgaactttgatgttgatgcgaatggtaaaatcacaatcggaggacagaaagcatatttaactagtgatggtaacttaactacaaacgatgctggtggtgcgactgcggctacgcttgatggtttattcaagaaagctggtgatggtcaatcaatcgggtttaagaagactgcatcagtcacgatggggggaacaacttataactttaaaacgggtgctgatgctgatgctgcaactgctaacgcaggggtatcgttcactgatacagctagcaaagaaaccgttttaaataaagtggctacagctaaacaaggcaaagcagctgcagctgacggtgatacatccgcaacaattacctataaatctggcgttcagacgtatcaggctgtatttgccgcaggtgacggtactgctagcgcaaaatatgccgataaagctgacgtttctaatgcaacagcaacatacactgatgctgatggtgaaatgactacaattggttcatacaccacgaagtattcaatcgatgctaacaacggcaaggtaactgttgattctggaactggtacgggtaaatatgcgccgaaagtaggggctgaagtatatgttagtgctaatggtactttaacaacagatgcaactagcgaaggcacagtaacaaaagatccactgaaagctctggatgaagctatcagctccatcgacaaattccgttcttccctgggtgctatccagaaccgtctggattccgcagtcaccaacctgaacaacaccactaccaacctgtccgaagcgcagtcccgtattcaggacgccgactatgcgaccgaagtgtccaacatgtcgaaagcgcagatcattcagcaggccggtaactccgtgctggcaaaagccaaccaggtaccgcagcaggttctgtctctgctgcagggttag

SEQ ID NO: 7 - protein, 506 aa, s. muenchen
MAQVINTNSLSLLTQNNLNKSQSALGTAIERLSSGLRINSAKDDAAGQAIANRFTANIKGLTQASRNANDGISIAQTTEGALNEINNNLQRVRELAVQSANGTNSQSDLDSIQAEITQRLNEIDRVSGQTQFNGVKVLAQDNTLTIQVGANDGETIDIDLKEISSKTLGLDKLNVQDAYTPKETAVTVDKTTYKNGTDTITAQSNTDIQTAIGGGATGVTGADIKFKDGQYYLDVKGGASAGVYKATYDETTKKVNIDTTDKTPLATAEATAIRGTATITHNQIAEVTKEGVDTTTVAAQLAAAGVTGADKDNTSLVKLSFEDKNGKVIDGGYAVKMGDDFYAATYDEKTGTITAKTTTYTDGAGVAQTGAVKFGGANGKSEVVTATDGKTYLASDLDKHNFRTGGELKEVNTDKTENPLQKIDAALAQVDTLRSDLGAVQNRFNSAITNLGNTVNNLSSARSRIEDSDYATEVSNMSRAQILQQAGTSVLAQANQVPQNVLSLLR

SEQ ID NO: 8 - DNA, 1522 bp, s. muenchen
aatggcacaagtcattaatacaaacagcctgtcgctgttgacccagaataacctgaacaaatcccagtccgctctgggcaccgctatcgagcgtctgtcttccggtctgcgtatcaacagcgcgaaagacgatgcggcaggtcaggcgattgctaaccgtttcaccgcgaacatcaaaggtctgactcaggcttcccgtaacgctaacgacggtatctccattgcgcagaccactgaaggcgcgctgaacgaaatcaacaacaacctgcagcgtgtgcgtgaactggcggttcagtctgctaacggtactaactcccagtctgaccttgactctatccaggctgaaatcacccagcgtctgaacgaaatcgaccgtgtatccggtcagactcagttcaacggcgtgaaagtcctggcgcaggacaacaccctgaccatccaggttggtgccaacgacggtgaaactattgatattgatttaaaagaaattagctctaaaacactgggacttgataagcttaatgtccaggatgcctacaccccgaaagaaactgctgtaaccgttgataaaactacctataaaaatggtacagatactattacagcccagagcaatactgatatccaaactgcaattggcggtggtgcaacgggggttactggggctgatatcaaatttaaagatggtcaatactatttagatgttaaaggcggtgcttctgctggtgtttataaagccacttatgatgaaactacaaagaaagttaatattgatacgactgataaaactccgttagcaactgcggaagctacagctattcggggaacggccactataacccacaaccaaattgctgaagtaacaaaagagggtgttgatacgaccacagttgcggctcaacttgctgctgcaggggttactggtgccgataaggacaatactagccttgtaaaactatcgtttgaggataaaaacggtaaggttattgatggtggctatgcagtgaaaatgggcgacgatttctatgccgctacatatgatgagaaaacaggtacaattactgctaaaacaaccacttatacagatggtgctggcgttgctcaaactggagctgtgaaatttggtggcgcaaatggtaaatctgaagttgttactgctaccgatggtaaaacttacttagcaagcgaccttgacaaacataacttcagaacaggcggtgagcttaaagaggttaatacagataagactgaaaacccactgcagaaaattgatgctgccttggcacaggttgatacacttcgttctgacctgggtgcggtacagaaccgtttcaactccgctatcaccaacctgggcaataccgtaaataacctgtcttctgcccgtagccgtatcgaagattccgactacgcgaccgaagtctccaacatgtctcgcgcgcagattctgcagcaggccggtacctccgttctggcgcaggctaaccaggttccgcaaaacgtcctctctttactgcgttaa

SEQ ID NO: 9 - protein, 588 aa, pArtificial Sequence
  pMT/STF2
MKLCILLAVVAFVGLSLGRSAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDSLNVQKAYDVKDTAVTTKAYANNGTTLDVSGLDDAAIKAATGGTNGTASVTGGAVKFDADNNKYFVTIGGFTGADAAKNGDYEVNVATDGTVTLAAGATKTTMPAGATTKTEVQELKDTPAVVSADAKNALIAGGVDATDANGAELVKMSYTDKNGKTIEGGYALKAGDKYYAADYDEATGAIKAKTTSYTAADGTTKTAANQLGGVDGKTEVVTIDGKTYNASKAAGHDFKAQPELAEAAAKTTENPLQKIDAALAQVDALRSDLGAVQNRFNSAITNLGNTVNNLSEARSRIEDSDYATEVSNMSRAQILQQAGTSVLAQANQVPQNVLSLLRKGNSKLEGQLEFPRTSPVWWNSADIQHSGGRSSLEGPRFEGKPIPNPLLGLDSTRTGHHHHHH

SEQ ID NO: 10 - DNA, 1764 bp, Artificial Sequence
  pMT/STF2
atgaagttatgcatattactggccgtcgtggcctttgttggcctctcgctcgggagatctgcacaagtaatcaacactaacagtctgtcgctgctgacccagaataacctgaacaaatcccagtccgcactgggcaccgctatcgagcgtctgtcttctggtctgcgtatcaacagcgcgaaagacgatgcggcaggtcaggcgattgctaaccgtttcaccgcgaacatcaaaggtctgactcaggcttcccgtaacgctaacgacggtatctccattgcgcagaccactgaaggcgcgctgaacgaaatcaacaacaacctgcagcgtgtgcgtgaactggcggttcagtctgctaacagcaccaactcccagtctgacctcgactccatccaggctgaaatcacccagcgcctgaacgaaatcgaccgtgtatccggccagactcagttcaacggcgtgaaagtcctggcgcaggacaacaccctgaccatccaggttggcgccaacgacggtgaaactatcgatatcgatctgaagcagatcaactctcagaccctgggtctggactcactgaacgtgcagaaagcgtatgatgtgaaagatacagcagtaacaacgaaagcttatgccaataatggtactacactggatgtatcgggtcttgatgatgcagctattaaagcggctacgggtggtacgaatggtacggcttctgtaaccggtggtgcggttaaatttgacgcagataataacaagtactttgttactattggtggctttactggtgctgatgccgccaaaaatggcgattatgaagttaacgttgctactgacggtacagtaacccttgcggctggcgcaactaaaaccacaatgcctgctggtgcgacaactaaaacagaagtacaggagttaaaagatacaccggcagttgtttcagcagatgctaaaaatgccttaattgctggcggcgttgacgctaccgatgctaatggcgctgagttggtcaaaatgtcttataccgataaaaatggtaagacaattgaaggcggttatgcgcttaaagctggcgataagtattacgccgcagattacgatgaagcgacaggagcaattaaagctaaaactacaagttatactgctgctgacggcactaccaaaacagcggctaaccaactgggtggcgtagacggtaaaaccgaagtcgttactatcgacggtaaaacctacaatgccagcaaagccgctggtcatgatttcaaagcacaaccagagctggcggaagcagccgctaaaaccaccgaaaacccgctgcagaaaattgatgccgcgctggcgcaggtggatgcgctgcgctctgatctgggtgcggtacaaaaccgtttcaactctgctatcaccaacctgggcaataccgtaaacaatctgtctgaagcgcgtagccgtatcgaagattccgactacgcgaccgaagtttccaacatgtctcgcgcgcagattctgcagcaggccggtacttccgttctggcgcaggctaaccaggtcccgcagaacgtgctgtctctgttacgtaagggcaattcgaagcttgaaggtcaattggaattccctaggactagtccagtgtggtggaattctgcagatatccagcacagtggcggccgctcgagtctagagggcccgcggttcgaaggtaagcctatccctaaccctctcctcggtctcgattctacgcgtaccggtcatcatcaccatcaccat

SEQ ID NO: 11 - protein, 97 aa, Artificial Sequence
  M2 Influenza A
MSLLTEVETPIRNEWGCRCNDSSDPLVVAASIIGILHLILWILDRLFFKCIYRLFKHGLKRGPSTEGVPESMREEYRKEQQNAVDADDSHFVSIELE

SEQ ID NO: 12 - DNA, 294 bp, Artificial Sequence
  M2 Influenza A
atgagccttctaaccgaggtcgaaacacctatcagaaacgaatgggggtgcagatgcaacgattcaagtgacccgcttgttgttgccgcgagtatcattgggatcttgcacttgatattgtggattcttgatcgtctttttttcaaatgcatctatcgactcttcaaacacggccttaaaagagggccttctacggaaggagtacctgagtctatgagggaagaatatcgaaaggaacagcagaatgctgtggatgctgacgacagtcattttgtcagcatagagttggagtaa

SEQ ID NO: 13 - protein, 24 aa, Artificial Sequence
  M2
SLLTEVETPIRNEWGCRCNDSSDP

SEQ ID NO: 14 - DNA, 75 bp, Artificial Sequence
  M2
atgagcctgctgaccgaggtcgaaacaccgatccgcaacgaatgggggtgccgctgcaacgattcaagtgacccg

SEQ ID NO: 15 - protein, 11 aa, Artificial Sequence
  M2
EVETPIRNEWG

SEQ ID NO: 16 - DNA, 30 bp, Artificial Sequence
  M2
gaggtcgaaacacctatcagaaacgaatgg

SEQ ID NO: 17 - DNA, 315 bp, Artificial Sequence
  4XM2e
agcttgctgactgaggttgagaccccgattcgcaacgaatggggttcccgttccaacgattcttccgacccgctcgagagcctgttgaccgaggttgaaacccctatccgtaatgaatggggctcccgtagcaacgactcttctgacccaggatcctccctcttgaccgaagtggaaacgcctattcgtaatgagtggggttctcgtagcaatgacagctcggacccggagctctcgctgctgacggaagtggagactccgatccgtaacgagtggggctctcgctctaacgatagctcagacccgtctagataa

SEQ ID NO: 18 - protein, 104 aa, Artificial Sequence
  4XM2e
SLLTEVETPIRNEWGSRSNDSSDPLESLLTEVETPIRNEWGSRSNDSSDPGSSLLTEVETPIRNEWGSRSNDSSDPELSLLTEVETPIRNEWGSRSNDSSDPSR

SEQ ID NO: 19 - protein, 9 aa, Artificial Sequence
  M2
EVETPIRNE

SEQ ID NO: 20 - protein, 8 aa, Artificial Sequence
  M2
EVETPIRN

SEQ ID NO: 21 - protein, 24 aa, Artificial Sequence
  M2
MKATKLVLGAVILGSTLLAGCSSN

SEQ ID NO: 22 - DNA, 75 bp, Artificial Sequence
  M2
atgaaagctactaaactggtactgggcgcggtaatcctgggttctactctgctgctggcaggttgctccagcaac

SEQ ID NO: 23 - DNA, 48 bp, Artificial Sequence
  Linker
aagggcaattcgaagcttgaaggtcaattggaattccctaggactagt

SEQ ID NO: 24 - protein, 16 aa, Artificial Sequence
  Linker
KGNSKLEGQLEFPRTS

SEQ ID NO: 25 - DNA, 36 bp, Artificial Sequence
  Linker
gaattctgcagatatccagcacagtggcggccgctc

SEQ ID NO: 26 - protein, 12 aa, Artificial Sequence
  Linker
EFCRYPAQWRPL

SEQ ID NO: 27 - protein, 12 aa, Artificial Sequence
  Linker
EFSRYPAQWRPL

SEQ ID NO: 28 - DNA, 36 bp, Artificial Sequence
  Linker
gaattctctagatatccagcacagtggcggccgctc

SEQ ID NO: 29 - protein, 45 aa, Artificial Sequence
  Linker
KGNSKLEGQLEFPRTSPVWWNSADIQHSGGRQCDGYLQNSPLRPL

SEQ ID NO: 30 - DNA, 135 bp, Artificial Sequence
  Linker
aagggcaattcgaagcttgaaggtcaattggaattccctaggactagtccagtgtggtggaattctgcagatatccagcacagtggcggccgccagtgtgatggatatctgcagaattcgcccttgcggccgctc

SEQ ID NO: 31 - protein, 611 aa, Artificial Sequence
  STF2-4XM2e
MAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDSLNVQKAYDVKDTAVTTKAYANNGTTLDVSGLDDAAIKAATGGTNGTASVTGGAVKFDADNNKYFVTIGGFTGADAAKNGDYEVNVATDGTVTLAAGATKTTMPAGATTKTEVQELKDTPAVVSADAKNALIAGGVDATDANGAELVKMSYTDKNGKTIEGGYALKAGDKYYAADYDEATGAIKAKTTSYTAADGTTKTAANQLGGVDGKTEVVTIDGKTYNASKAAGHDFKAQPELAEAAAKTTENPLQKIDAALAQVDALRSDLGAVQNRFNSAITNLGNTVNNLSEARSRIEDSDYATEVSNMSRAQILQQAGTSVLAQANQVPQNVLSLLRLSLLTEVETPIRNEWGSRSNDSSDPLESLLTEVETPIRNEWGSRSNDSSDPGSSLLTEVETPIRNEWGSRSNDSSDPELSLLTEVETPIRNEWGSRSNDSSDPSR

SEQ ID NO: 32 - DNA, 1836 bp, Artificial Sequence
  STF2-4XM2e
atggcacaagtaatcaacactaacagtctgtcgctgctgacccagaataacctgaacaaatcccagtccgcactgggcaccgctatcgagcgtctgtcttctggtctgcgtatcaacagcgcgaaagacgatgcggcaggtcaggcgattgctaaccgtttcaccgcgaacatcaaaggtctgactcaggcttcccgtaacgctaacgacggtatctccattgcgcagaccactgaaggcgcgctgaacgaaatcaacaacaacctgcagcgtgtgcgtgaactggcggttcagtctgctaacagcaccaactcccagtctgacctcgactccatccaggctgaaatcacccagcgcctgaacgaaatcgaccgtgtatccggccagactcagttcaacggcgtgaaagtcctggcgcaggacaacaccctgaccatccaggttggcgccaacgacggtgaaactatcgatatcgatctgaagcagatcaactctcagaccctgggtctggactcactgaacgtgcagaaagcgtatgatgtgaaagatacagcagtaacaacgaaagcttatgccaataatggtactacactggatgtatcgggtcttgatgatgcagctattaaagcggctacgggtggtacgaatggtacggcttctgtaaccggtggtgcggttaaatttgacgcagataataacaagtactttgttactattggtggctttactggtgctgatgccgccaaaaatggcgattatgaagttaacgttgctactgacggtacagtaacccttgcggctggcgcaactaaaaccacaatgcctgctggtgcgacaactaaaacagaagtacaggagttaaaagatacaccggcagttgtttcagcagatgctaaaaatgccttaattgctggcggcgttgacgctaccgatgctaatggcgctgagttggtcaaaatgtcttataccgataaaaatggtaagacaattgaaggcggttatgcgcttaaagctggcgataagtattacgccgcagattacgatgaagcgacaggagcaattaaagctaaaaccacaagttatactgctgctgacggcactaccaaaacagcggctaaccaactgggtggcgtagacggtaaaaccgaagtcgttactatcgacggtaaaacctacaatgccagcaaagccgctggtcatgatttcaaagcacaaccagagctggcggaagcagccgctaaaaccaccgaaaacccgctgcagaaaattgatgccgcgctggcgcaggtggatgcgctgcgctctgatctgggtgcggtacaaaaccgtttcaactctgctatcaccaacctgggcaataccgtaaacaatctgtctgaagcgcgtagccgtatcgaagattccgactacgcgaccgaagtttccaacatgtctcgcgcgcagattttgcagcaggccggtacttccgttctggcgcaggctaaccaggtcccgcagaacgtgctgtctctgttacgtctgagcttgctgactgaggttgagaccccgattcgcaacgaatggggttcccgttccaacgattcttccgacccgctcgagagcctgttgaccgaggttgaaacccctatccgtaatgaatggggctcccgtagcaacgactcttctgacccaggatcctccctcttgaccgaagtggaaacgcctattcgtaatgagtggggttctcgtagcaatgacagctcggacccggagctctcgctgctgacggaagtggagactccgatccgtaacgagtggggctctcgctctaacgatagctcagacccgtctagataa

SEQ ID NO: 33 - protein, 19 aa, Artificial Sequence
  HA
PAKLLKERGRRGAIAGFLE

SEQ ID NO: 34 - protein, 10 aa, Artificial Sequence
  HA
EVETPIRNEW

SEQ ID NO: 35 - protein, 26 aa, Artificial Sequence
  HA FluB
SLWSEEPAKLLKERGFFGAIAGFLEE

SEQ ID NO: 36 - protein, 26 aa, Artificial Sequence
  HA FluA H1/H0
SLWSEENIPSIQSRGLFGAIAGFIEE

SEQ ID NO: 37 - protein, 26 aa, Artificial Sequence
  HA Flu A H3/H0
SLWSEENVPEKQTRGIFGAIAGFIEE

SEQ ID NO: 38 - protein, 30 aa, Artificial Sequence
  HA Flu A H5/H0
SLWSEEEWEERERRRKKRGLFGAIAGFIEE

SEQ ID NO: 39 - protein, 24 aa, Artificial Sequence
  M2
SLLTEVETPTRNEWESRSSDSSDP

SEQ ID NO: 40 - protein, 24 aa, Artificial Sequence
  M2
SLLTEVETPTRNEWECRCSDSSDP

SEQ ID NO: 41 - protein, 24 aa, Artificial Sequence
  M2
SLLTEVETLTRNGWGSRSSDSSDP

SEQ ID NO: 42 - protein, 24 aa, Artificial Sequence
  M2
SLLTEVETLTRNGWGCRCSDSSDP

SEQ ID NO: 43 - protein, 24 aa, Artificial Sequence
  M2
SLLTEVETPTRNGWESKSSDSSDP

SEQ ID NO: 44 - protein, 24 aa, Artificial Sequence
  M2
SLLTEVETPTRNGWECKCSDSSDP

SEQ ID NO: 45 - protein, 24 aa, Artificial Sequence
  M2
SLLTEVETLTRNGWESKSRDSSDP

SEQ ID NO: 46 - protein, 24 aa, Artificial Sequence
  M2
SLLTEVETLTRNGWECKCRDSSDP

SEQ ID NO: 47 - protein, 24 aa, Artificial Sequence
  M2
SLLTEVETPIRNEWGSRSNDSSDP

SEQ ID NO: 48 - protein, 24 aa, Artificial Sequence
  M2
SLLTEVETPIRNEWGCRCNGSSDP

SEQ ID NO: 49 - protein, 24 aa, Artificial Sequence
  M2
SLLTEVETPIRNEWGCKCNDSSDP

SEQ ID NO: 50 - protein, 24 aa, Artificial Sequence
  M2
SLLTEVETPIRNEWGCRCNDSSDP

SEQ ID NO: 51 - protein, 24 aa, Artificial Sequence
  M2
SLLTEVETPIRNEWGCRCNDSSDP

SEQ ID NO: 52 - protein, 24 aa, Artificial Sequence
  M2
SLLTEVETPIRNEWECRCNGSSDP

SEQ ID NO: 53 - protein, 24 aa, Artificial Sequence
  M2
SLLTEVETPTRNEWECRCSDSSDP

SEQ ID NO: 54 - protein, 24 aa, Artificial Sequence
  M2
SLLTEVETPIRNEWGSRSNGSSDP

SEQ ID NO: 55 - protein, 24 aa, Artificial Sequence
  M2
SLLTEVETLTRNGWGCRCSDSSDP

SEQ ID NO: 56 - protein, 24 aa, Artificial Sequence
  M2
SLLTEVETPTRNEWECRCSDSSDP

SEQ ID NO: 57 - protein, 24 aa, Artificial Sequence
  M2
SLLTEVETPTRNGWECKCSDSSDP

SEQ ID NO: 58 - protein, 24 aa, Artificial Sequence
  M2
SLLTEVETPTRNGWGCRCSGSSDP

SEQ ID NO: 59 - protein, 24 aa, Artificial Sequence
  M2
SLLTEVETLTRNGWECKCRDSSDP

SEQ ID NO: 60 - protein, 811 aa, Artificial Sequence
  STF2delta.HA
MAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDSLNVHGAPVDPASPWTENPLQKIDAALAQVDALRSDLGAVQNRFNSAITNLGNTVNNLSEARSRIEDSDYATEVSNMSRAQILQQAGTSVLAQANQVPQNVLSLLREFSRYPAQWRPLDQICIGYHANNSTEQVDTIMEKNVTVTHAQDILEKKHNGKLCDLDGVKPLILRDCSVAGWLLGNPMCDEFINVPEWSYIVEKANPVNDLCYPGDFNDYEELKHLLSRINHFEKIQIIPKSSWSSHEASLGVSSACPYQGKSSFFRNVVWLIKKNSTYPTIKRSYNNTNQEDLLVLWGIHHPNDAAEQTKLYQNPTTYISVGTSTLNQRLVPRIATRSKVNGQSGRMEFFWTILKPNDAINFESNGNFIAPEYAYKIVKKGDSTIMKSELEYGNCNTKCQTPMGAINSSMPFHNIHPLTIGECPKYVKSNRLVLATGLRNSPQRERRRKKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESIGIYQILSIYST

SEQ ID NO: 61 - DNA, 2439 bp, Artificial Sequence
  STF2delta.HA
atggcacaagtaatcaacactaacagtctgtcgctgctgacccagaataacctgaacaaatcccagtccgcactgggcaccgctatcgagcgtctgtcttctggtctgcgtatcaacagcgcgaaagacgatgcggcaggtcaggcgattgctaaccgtttcaccgcgaacatcaaaggtctgactcaggcttcccgtaacgctaacgacggtatctccattgcgcagaccactgaaggcgcgctgaacgaaatcaacaacaacctgcagcgtgtgcgtgaactggcggttcagtctgctaacagcaccaactcccagtctgacctcgactccatccaggctgaaatcacccagcgcctgaacgaaatcgaccgtgtatccggccagactcagttcaacggcgtgaaagtcctggcgcaggacaacaccctgaccatccaggttggcgccaacgacggtgaaactatcgatatcgatctgaagcagatcaactctcagaccctgggtctggactcactgaacgtgcatggagcgccggtggatcctgctagcccatggaccgaaaacccgctgcagaaaattgatgccgcgctggcgcaggtggatgcgctgcgctctgatctgggtgcggtacaaaaccgtttcaactctgctatcaccaacctgggcaataccgtaaacaatctgtctgaagcgcgtagccgtatcgaagattccgactacgcgaccgaagtttccaacatgtctcgcgcgcagattttgcagcaggccggtacttccgttctggcgcaggctaaccaggtcccgcagaacgtgctgtctctgttacgtgaattctctagatatccagcacagtggcggccgctcgaccagatctgtatcggttatcatgctaacaattctactgaacaagtagatactatcatggagaagaacgttacagttacacatgcacaagatatcctggaaaagaagcataatggaaaactgtgtgaccttgatggtgttaaaccactaatattgcgtgactgctcagttgctgggtggttgttggggaatccaatgtgcgacgaatttatcaacgttccagaatggagttacattgttgaaaaagctaaccctgttaacgacttgtgttacccaggcgattttaatgactacgaggaacttaagcatttgttgtcaagaattaaccacttcgagaaaattcaaattattccaaagtcatcttggtcctcccatgaagcatccctaggagtctcttccgcttgcccttaccaaggcaagagttccttttttcgtaatgtcgtctggctgatcaaaaagaactccacctatccaactataaagagatcatacaacaacacaaatcaggaggatctgctagttctgtggggcattcaccaccccaatgacgcagctgagcagactaaattgtaccaaaacccaactacctatatatcagttggtacctcaactcttaaccagcgactagtcccccgtattgctactaggtcaaaggttaatggtcaaagtggacgaatggagtttttctggactattttgaagcccaacgatgccatcaacttcgaaagtaatggaaatttcatagcccctgagtacgcttacaaaatcgttaaaaagggtgattccactatcatgaaatctgaactggaatacggaaactgtaacaccaaatgccagacgccaatgggtgccatcaactcttctatgccttttcacaacattcatcctttgactattggtgaatgcccaaagtacgtcaaatctaaccgtttggtgttggctactggtctaaggaactcccctcagcgtgaaagaagaagaaagaagaggggattattcggtgctatcgctggatttattgagggaggatggcagggaatggtcgatggctggtatggttaccatcactcaaatgaacagggaagtggatacgcagctgataaagaatctactcaaaaggctatcgacggtgttacaaacaaggtcaattctattatcgataagatgaatacacagtttgaggctgttggtagagagttcaataatcttgagagaagaatcgaaaacctgaacaagaaaatggaagacggatttttagatgtatggacttacaatgctgagttgttggtcttgatggagaatgaacgaacgttggacttccatgactccaatgtgaagaacctatatgacaaagtgaggctgcaacttagagacaacgccaaggaattgggaaacgggtgcttcgagttttaccacaaatgcgacaacgaatgtatggaatcagtgagaaacggtacctatgattacccccaatattccgaggaggcaagactgaagagagaagagatatctggtgtaaagttggaatccatcggtatttatcagattctatctatatattctacctaatag

SEQ ID NO: 62 - protein, 800 aa, Artificial Sequence
  STF2delta.HA (PR8)
MAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDSLNVHGAPVDPASPWTENPLQKIDAALAQVDALRSDLGAVQNRFNSAITNLGNTVNNLSEARSRIEDSDYATEVSNMSRAQILQQAGTSVLAQANQVPQNVLSLLREFSRYPAQWRPLDTICIGYHANNSTDTVDTVLEKNVTVTHSVNLLEDSHNGKLCRLKGIAPLQLGKCNIAGWLLGNPECDPLLPVRSWSYIVETPNSENGICYPGDFIDYEELREQLSSVSSFERFEIFPKESSWPNHNTNGVTAACSHEGKSSFYRNLLWLTEKEGSYPKLKNSYVNKKGKEVLVLWGIHHPPNSKEQQNLYQNENAYVSVVTSNYNRRFTPEIAERPKVRDQAGRMNYYWTLLKPGDTIIFEANGNLIAPMYAFALSRGFGSGIITSNASMHECNTKCQTPLGAINSSLPYQNIHPVTIGECPKYVRSAKLRMVTGLRNIPSIQSRGLFGAIAGFIEGGWTGMIDGWYGYHHQNEQGSGYAADQKSTQNAINGITNKVNTVIEKMNIQFTAVGKEFNKLEKRMENLNKKVDDGFLDIWTYNAELLVLLENERTLDFHDSNVKNLYEKVKSQLKNNAKEIGNGCFEFYHKCDNECMESVRNGTYDYPKYSEESKLNREKVDGVKLESMGIYQ

SEQ ID NO: 63 - DNA, 2439 bp, Artificial Sequence; identical to SEQ ID NO: 61
  STF2delta.HA (PR8)

SEQ ID NO: 64 - protein, 511 aa, Artificial Sequence
  HA (PR8)
DTICIGYHANNSTDTVDTVLEKNVTVTHSVNLLEDSHNGKLCRLKGIAPLQLGKCNIAGWLLGNPECDPLLPVRSWSYIVETPNSENGICYPGDFIDYEELREQLSSVSSFERFEIFPKESSWPNHNTNGVTAACSHEGKSSFYRNLLWLTEKEGSYPKLKNSYVNKKGKEVLVLWGIHHPPNSKEQQNLYQNENAYVSVVTSNYNRRFTPEIAERPKVRDQAGRMNYYWTLLKPGDTIIFEANGNLIAPMYAFALSRGFGSGIITSNASMHECNTKCQTPLGAINSSLPYQNIHPVTIGECPKYVRSAKLRMVTGLRNIPSIQSRGLFGAIAGFIEGGWTGMIDGWYGYHHQNEQGSGYAADQKSTQNAINGITNKVNTVIEKMNIQFTAVGKEFNKLEKRMENLNKKVDDGFLDIWTYNAELLVLLENERTLDFHDSNVKNLYEKVKSQLKNNAKEIGNGCFEFYHKCDNECMESVRNGTYDYPKYSEESKLNREKVDGVKLESMGIYQ

SEQ ID NO: 65 - DNA, 1572 bp, Artificial Sequence
  HA (PR8)
gaccagatctgtatcggttatcatgctaacaattctactgaacaagtagatactatcatggagaagaacgttacagttacacatgcacaagatatcctggaaaagaagcataatggaaaactgtgtgaccttgatggtgttaaaccactaatattgcgtgactgctcagttgctgggtggttgttggggaatccaatgtgcgacgaatttatcaacgttccagaatggagttacattgttgaaaaagctaaccctgttaacgacttgtgttacccaggcgattttaatgactacgaggaacttaagcatttgttgtcaagaattaaccacttcgagaaaattcaaattattccaaagtcatcttggtcctcccatgaagcatccctaggagtctcttccgcttgcccttaccaaggcaagagttccttttttcgtaatgtcgtctggctgatcaaaaagaactccacctatccaactataaagagatcatacaacaacacaaatcaggaggatctgctagttctgtggggcattcaccaccccaatgacgcagctgagcagactaaattgtaccaaaacccaactacctatatatcagttggtacctcaactcttaaccagcgactagtcccccgtattgctactaggtcaaaggttaatggtcaaagtggacgaatggagtttttctggactattttgaagcccaacgatgccatcaacttcgaaagtaatggaaatttcatagcccctgagtacgcttacaaaatcgttaaaaagggtgattccactatcatgaaatctgaactggaatacggaaactgtaacaccaaatgccagacgccaatgggtgccatcaactcttctatgccttttcacaacattcatcctttgactattggtgaatgcccaaagtacgtcaaatctaaccgtttggtgttggctactggtctaaggaactcccctcagcgtgaaagaagaagaaagaagaggggattattcggtgctatcgctggatttattgagggaggatggcagggaatggtcgatggctggtatggttaccatcactcaaatgaacagggaagtggatacgcagctgataaagaatctactcaaaaggctatcgacggtgttacaaacaaggtcaattctattatcgataagatgaatacacagtttgaggctgttggtagagagttcaataatcttgagagaagaatcgaaaacctgaacaagaaaatggaagacggatttttagatgtatggacttacaatgctgagttgttggtcttgatggagaatgaacgaacgttggacttccatgactccaatgtgaagaacctatatgacaaagtgaggctgcaacttagagacaacgccaaggaattgggaaacgggtgcttcgagttttaccacaaatgcgacaacgaatgtatggaatcagtgagaaacggtacctatgattacccccaatattccgaggaggcaagactgaagagagaagagatatctggtgtaaagttggaatccatcggtatttatcagattctatctatatattctacctaatag

SEQ ID NO: 66 - protein, 261 aa, E. coli
MAQVINTNSLSLITQNNINKNQSALSSSIERLSSGLRINSAKDDAAGQAIANRFTSNIKGLTQAARNANDGISVAQTTEGALSEINNNLQRIRELTVQASTGTNSDSDLDSIQDEIKSRLDEIDRVSGQTQFNGVNVLAKDGSMKIQVGANDGQTITIDLKKIDSDTLGTKDPLKALDEAISSIDKFRSSLGAIQNRLDSAVTNLNNTTTNLSEAQSRIQDADYATEVSNMSKAQIIQQAGNSVLAKANQVPQQVLSLLQG

SEQ ID NO: 67 - protein, 522 aa, Artificial Sequence
  H5N1 HA
DQICIGYHANNSTEQVDTIMEKNVTVTHAQDILEKKHNGKLCDLDGVKPLILRDCSVAGWLLGNPMCDEFINVPEWSYIVEKANPVNDLCYPGDFNDYEELKHLLSRINHFEKIQIIPKSSWSSHEASLGVSSACPYQGKSSFFRNVVWLIKKNSTYPTIKRSYNNTNQEDLLVLWGIHHPNDAAEQTKLYQNPTTYISVGTSTLNQRLVPRIATRSKVNGQSGRMEFFWTILKPNDAINFESNGNFIAPEYAYKIVKKGDSTIMKSELEYGNCNTKCQTPMGAINSSMPFHNIHPLTIGECPKYVKSNRLVLATGLRNSPQRERRRKKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESIGIYQILSIYST

SEQ ID NO: 68 - DNA, 1572 bp, Artificial Sequence; identical to SEQ ID NO: 65
  H5N1 HA

SEQ ID NO: 69 - DNA, 43 bp, Artificial Sequence
  Primer
cgctcttcamtgagcttgctgactgaggttgagaccccgattc

SEQ ID NO: 70 - DNA, 50 bp, Artificial Sequence
  Primer
cgctcttcacgcttattatctagacgggtctgagctatcgttagagcgag

SEQ ID NO: 71 - DNA, 41 bp, Artificial Sequence
  Primer
gctcttcagcggctgagcaataactagcataaccccttggg

SEQ ID NO: 72 - DNA, 39 bp, Artificial Sequence
  Primer
cgctcttcacagacgtaacagagacagcacgttctgcgg

SEQ ID NO: 73 - protein, 24 aa, Artificial Sequence
  M2
SLLTEVETPIRNEWGSKSNDSSDP

SEQ ID NO: 74 - protein, 24 aa, Artificial Sequence
  M2
SLLTEVETPIRNEWGSRSNDSSDP

SEQ ID NO: 75 - protein, 24 aa, Artificial Sequence
  M2
SLLTEVETPIRNEWESRSNGSSDP

SEQ ID NO: 76 - protein, 24 aa, Artificial Sequence
  M2
SLLTEVETPTRNEWESRSSDSSDP

SEQ ID NO: 77 - protein, 24 aa, Artificial Sequence
  M2
SLLTEVETLTRNGWGSRSSDSSDP

SEQ ID NO: 78 - protein, 24 aa, Artificial Sequence
  M2
SLLTEVETPTRNEWESRSSDSSDP

SEQ ID NO: 79 - protein, 24 aa, Artificial Sequence
  M2
SLLTEVETPTRNGWESKSSDSSDP

SEQ ID NO: 80 - protein, 24 aa, Artificial Sequence
  M2
SLLTEVETPTRNGWGSRSSGSSDP

SEQ ID NO: 81 - protein, 24 aa, Artificial Sequence
  M2
SLLTEVETLTRNGWESKSRDSSDP

SEQ ID NO: 82 - protein, 641 aa, Artificial Sequence
  pMT/STF2.4XM2e (H1)
MKLCILLAVVAFVGLSLGRSAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDSLNVQKAYDVKDTAVTTKAYANNGTTLDVSGLDDAAIKAATGGTNGTASVTGGAVKFDADNNKYFVTIGGFTGADAAKNGDYEVNVATDGTVTLAAGATKTTMPAGATTKTEVQELKDTPAVVSADAKNALIAGGVDATDANGAELVKMSYTDKNGKTIEGGYALKAGDKYYAADYDEATGAIKAKTTSYTAADGTTKTAANQLGGVDGKTEVVTIDGKTYNASKAAGHDFKAQPELAEAAAKTTENPLQKIDAALAQVDALRSDLGAVQNRFNSAITNLGNTVNNLSEARSRIEDSDYATEVSNMSRAQILQQAGTSVLAQANQVPQNVLSLLRKGNSKLEGQLEFSLLTEVETPIRNEWGSRSNDSSDPLESLLTEVETPIRNEWGSRSNDSSDPGSSLLTEVETPIRNEWGSRSNDSSDPELSLLTEVETPIRNEWGSRSNDSSDPSR

SEQ ID NO: 83 - DNA, 1926 bp, Artificial Sequence
  pMT/STF2.4XM2e (H1)
atgaagttatgcatattactggccgtcgtggcctttgttggcctctcgctcgggagatctgcacaagtaatcaacactaacagtctgtcgctgctgacccagaataacctgaacaaatcccagtccgcactgggcaccgctatcgagcgtctgtcttctggtctgcgtatcaacagcgcgaaagacgatgcggcaggtcaggcgattgctaaccgtttcaccgcgaacatcaaaggtctgactcaggcttcccgtaacgctaacgacggtatctccattgcgcagaccactgaaggcgcgctgaacgaaatcaacaacaacctgcagcgtgtgcgtgaactggcggttcagtctgctaacagcaccaactcccagtctgacctcgactccatccaggctgaaatcacccagcgcctgaacgaaatcgaccgtgtatccggccagactcagttcaacggcgtgaaagtcctggcgcaggacaacaccctgaccatccaggttggcgccaacgacggtgaaactatcgatatcgatctgaagcagatcaactctcagaccctgggtctggactcactgaacgtgcagaaagcgtatgatgtgaaagatacagcagtaacaacgaaagcttatgccaataatggtactacactggatgtatcgggtcttgatgatgcagctattaaagcggctacgggtggtacgaatggtacggcttctgtaaccggtggtgcggttaaatttgacgcagataataacaagtactttgttactattggtggctttactggtgctgatgccgccaaaaatggcgattatgaagttaacgttgctactgacggtacagtaacccttgcggctggcgcaactaaaaccacaatgcctgctggtgcgacaactaaaacagaagtacaggagttaaaagatacaccggcagttgtttcagcagatgctaaaaatgccttaattgctggcggcgttgacgctaccgatgctaatggcgctgagttggtcaaaatgtcttataccgataaaaatggtaagacaattgaaggcggttatgcgcttaaagctggcgataagtattacgccgcagattacgatgaagcgacaggagcaattaaagctaaaactacaagttatactgctgctgacggcactaccaaaacagcggctaaccaactgggtggcgtagacggtaaaaccgaagtcgttactatcgacggtaaaacctacaatgccagcaaagccgctggtcatgatttcaaagcacaaccagagctggcggaagcagccgctaaaaccaccgaaaacccgctgcagaaaattgatgccgcgctggcgcaggtggatgcgctgcgctctgatctgggtgcggtacaaaaccgtttcaactctgctatcaccaacctgggcaataccgtaaacaatctgtctgaagcgcgtagccgtatcgaagattccgactacgcgaccgaagtttccaacatgtctcgcgcgcagattctgcagcaggccggtacttccgttctggcgcaggctaaccaggtcccgcagaacgtgctgtctctgttacgtaagggcaattcgaagcttgaaggtcaattggaattcagcttgctgactgaggttgagaccccgattcgcaacgaatggggttcccgttccaacgattcttccgacccgctcgagagcctgttgaccgaggttgaaacccctatccgtaatgaatggggctcccgtagcaacgactcttctgacccaggatcctccctcttgaccgaagtggaaacgcctattcgtaatgagtggggttctcgtagcaatgacagctcggacccggagctctcgctgctgacggaagtggagactccgatccgtaacgagtggggctctcgctctaacgatagctcagacccgtctagataa

SEQ ID NO: 84 - protein, 641 aa, Artificial Sequence
  pMT/STF2.4XM2e (H5)
MKLCILLAVVAFVGLSLGRSAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDSLNVQKAYDVKDTAVTTKAYANNGTTLDVSGLDDAAIKAATGGTNGTASVTGGAVKFDADNNKYFVTIGGFTGADAAKNGDYEVNVATDGTVTLAAGATKTTMPAGATTKTEVQELKDTPAVVSADAKNALIAGGVDATDANGAELVKMSYTDKNGKTIEGGYALKAGDKYYAADYDEATGAIKAKTTSYTAADGTTKTAANQLGGVDGKTEVVTIDGKTYNASKAAGHDFKAQPELAEAAAKTTENPLQKIDAALAQVDALRSDLGAVQNRFNSAITNLGNTVNNLSEARSRIEDSDYATEVSNMSRAQILQQAGTSVLAQANQVPQNVLSLLRKGNSKLEGQLEFSLLTEVETPTRNEWESRSSDSSDPLESLLTEVETPTRNEWESRSSDSSDPESSLLTEVETPTRNEWESRSSDSSDPGSSLLTEVETPTRNEWESRSSDSSDPSR

SEQ ID NO: 85 - DNA, 1927 bp, Artificial Sequence
  pMT/STF2.4XM2e (H5)
atgaagttatgcatattactggccgtcgtggcctttgttggcctctcgctcgggagatctgcacaagtaatcaacactaacagtctgtcgctgctgacccagaataacctgaacaaatcccagtccgcactgggcaccgctatcgagcgtctgtcttctggtctgcgtatcaacagcgcgaaagacgatgcggcaggtcaggcgattgctaaccgtttcaccgcgaacatcaaaggtctgactcaggcttcccgtaacgctaacgacggtatctccattgcgcagaccactgaaggcgcgctgaacgaaatcaacaacaacctgcagcgtgtgcgtgaactggcggttcagtctgctaacagcaccaactcccagtctgacctcgactccatccaggctgaaatcacccagcgcctgaacgaaatcgaccgtgtatccggccagactcagttcaacggcgtgaaagtcctggcgcaggacaacaccctgaccatccaggttggcgccaacgacggtgaaactatcgatatcgatctgaagcagatcaactctcagaccctgggtctggactcactgaacgtgcagaaagcgtatgatgtgaaagatacagcagtaacaacgaaagcttatgccaataatggtactacactggatgtatcgggtcttgatgatgcagctattaaagcggctacgggtggtacgaatggtacggcttctgtaaccggtggtgcggttaaatttgacgcagataataacaagtactttgttactattggtggctttactggtgctgatgccgccaaaaatggcgattatgaagttaacgttgctactgacggtacagtaacccttgcggctggcgcaactaaaaccacaatgcctgctggtgcgacaactaaaacagaagtacaggagttaaaagatacaccggcagttgtttcagcagatgctaaaaatgccttaattgctggcggcgttgacgctaccgatgctaatggcgctgagttggtcaaaatgtcttataccgataaaaatggtaagacaattgaaggcggttatgcgcttaaagctggcgataagtattacgccgcagattacgatgaagcgacaggagcaattaaagctaaaactacaagttatactgctgctgacggcactaccaaaacagcggctaaccaactgggtggcgtagacggtaaaaccgaagtcgttactatcgacggtaaaacctacaatgccagcaaagccgctggtcatgatttcaaagcacaaccagagctggcggaagcagccgctaaaaccaccgaaaacccgctgcagaaaattgatgccgcgctggcgcaggtggatgcgctgcgctctgatctgggtgcggtacaaaaccgtttcaactctgctatcaccaacctgggcaataccgtaaacaatctgtctgaagcgcgtagccgtatcgaagattccgactacgcgaccgaagtttccaacatgtctcgcgcgcagattctgcagcaggccggtacttccgttctggcgcaggctaaccaggtcccgcagaacgtgctgtctctgttacgtaagggcaattcgaagcttgaaggtcaattggaattctctctgctgactgaagtagaaactccaacgcgtaatgaatgggaatcccgttctagcgactcctctgatcctctcgagtccctgctgacggaggttgaaaccccgacccgcaacgagtgggaaagccgttcctccgattcctctgatccggagagcagcctgctgaccgaggtagaaaccccgacccgtaatgagtgggaatctcgctcctctgattcttctgacccgggatcctctctgctgaccgaagtggagactccgactcgcaacgaatgggagagccgttcttctgactcctctgacccgtctagataat

SEQ ID NO: 86 - protein, 749 aa, Artificial Sequence
  pMT/STF2.4XM2e (H1 H5)
MKLCILLAVVAFVGLSLGRSAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDSLNVQKAYDVKDTAVTTKAYANNGTTLDVSGLDDAAIKAATGGTNGTASVTGGAVKFDADNNKYFVTIGGFTGADAAKNGDYEVNVATDGTVTLAAGATKTTMPAGATTKTEVQELKDTPAVVSADAKNALIAGGVDATDANGAELVKMSYTDKNGKTIEGGYALKAGDKYYAADYDEATGAIKAKTTSYTAADGTTKTAANQLGGVDGKTEVVTIDGKTYNASKAAGHDFKAQPELAEAAAKTTENPLQKIDAALAQVDALRSDLGAVQNRFNSAITNLGNTVNNLSEARSRIEDSDYATEVSNMSRAQILQQAGTSVLAQANQVPQNVLSLLRKGNSKLEGQLEFSLLTEVETPIRNEWGSRSNDSSDPLESLLTEVETPIRNEWGSRSNDSSDPGSSLLTEVETPIRNEWGSRSNDSSDPELSLLTEVETPIRNEWGSRSNDSSDPSRSREFSLLTEVETPTRNEWESRSSDSSDPLESLLTEVETPTRNEWESRSSDSSDPESSLLTEVETPTRNEWESRSSDSSDPGSSLLTEVETPTRNEWESRSSDSSDPSR

SEQ ID NO: 87 - DNA, 2250 bp, Artificial Sequence
  pMT/STF2.4XM2e (H1 H5)
atgaagttatgcatattactggccgtcgtggcctttgttggcctctcgctcgggagatctgcacaagtaatcaacactaacagtctgtcgctgctgacccagaataacctgaacaaatcccagtccgcactgggcaccgctatcgagcgtctgtcttctggtctgcgtatcaacagcgcgaaagacgatgcggcaggtcaggcgattgctaaccgtttcaccgcgaacatcaaaggtctgactcaggcttcccgtaacgctaacgacggtatctccattgcgcagaccactgaaggcgcgctgaacgaaatcaacaacaacctgcagcgtgtgcgtgaactggcggttcagtctgctaacagcaccaactcccagtctgacctcgactccatccaggctgaaatcacccagcgcctgaacgaaatcgaccgtgtatccggccagactcagttcaacggcgtgaaagtcctggcgcaggacaacaccctgaccatccaggttggcgccaacgacggtgaaactatcgatatcgatctgaagcagatcaactctcagaccctgggtctggactcactgaacgtgcagaaagcgtatgatgtgaaagatacagcagtaacaacgaaagcttatgccaataatggtactacactggatgtatcgggtcttgatgatgcagctattaaagcggctacgggtggtacgaatggtacggcttctgtaaccggtggtgcggttaaatttgacgcagataataacaagtactttgttactattggtggctttactggtgctgatgccgccaaaaatggcgattatgaagttaacgttgctactgacggtacagtaacccttgcggctggcgcaactaaaaccacaatgcctgctggtgcgacaactaaaacagaagtacaggagttaaaagatacaccggcagttgtttcagcagatgctaaaaatgccttaattgctggcggcgttgacgctaccgatgctaatggcgctgagttggtcaaaatgtcttataccgataaaaatggtaagacaattgaaggcggttatgcgcttaaagctggcgataagtattacgccgcagattacgatgaagcgacaggagcaattaaagctaaaactacaagttatactgctgctgacggcactaccaaaacagcggctaaccaactgggtggcgtagacggtaaaaccgaagtcgttactatcgacggtaaaacctacaatgccagcaaagccgctggtcatgatttcaaagcacaaccagagctggcggaagcagccgctaaaaccaccgaaaacccgctgcagaaaattgatgccgcgctggcgcaggtggatgcgctgcgctctgatctgggtgcggtacaaaaccgtttcaactctgctatcaccaacctgggcaataccgtaaacaatctgtctgaagcgcgtagccgtatcgaagattccgactacgcgaccgaagtttccaacatgtctcgcgcgcagattctgcagcaggccggtacttccgttctggcgcaggctaaccaggtcccgcagaacgtgctgtctctgttacgtaagggcaattcgaagcttgaaggtcaattggaattcagcttgctgactgaggttgagaccccgattcgcaacgaatggggttcccgttccaacgattcttccgacccgctcgagagcctgttgaccgaggttgaaacccctatccgtaatgaatggggctcccgtagcaacgactcttctgacccaggatcctccctcttgaccgaagtggaaacgcctattcgtaatgagtggggttctcgtagcaatgacagctcggacccggagctctcgctgctgacggaagtggagactccgatccgtaacgagtggggctctcgctctaacgatagctcagacccgtctagatctagagaattctctctgctgactgaagtagaaactccaacgcgtaatgaatgggaatcccgttctagcgactcctctgatcctctcgagtccctgctgacggaggttgaaaccccgacccgcaacgagtgggaaagccgttcctccgattcctctgatccggagagcagcctgctgaccgaggtagaaaccccgacccgtaatgagtgggaatctcgctcctctgattcttctgacccgggatcctctctgctgaccgaagtggagactccgactcgcaacgaatgggagagccgttcttctgactcctctgacccgtctagataa

SEQ ID NO: 88 - protein, 318 aa, Artificial Sequence
  pMT/STF2delta
MKLCILLAVVAFVGLSLGRSAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDSLNVHGAPVDPASPWTENPLQKIDAALAQVDALRSDLGAVQNRFNSAITNLGNTVNNLSEARSRIEDSDYATEVSNMSRAQILQQAGTSVLAQANQVPQNVLSLLREFSRYPAQWRPLTRTGHHHHHH

SEQ ID NO: 89 - DNA, 957 bp, Artificial Sequence
  pMT/STF2delta
atgaagttatgcatattactggccgtcgtggcctttgttggcctctcgctcgggagatctgcacaagtaatcaacactaacagtctgtcgctgctgacccagaataacctgaacaaatcccagtccgcactgggcaccgctatcgagcgtctgtcttctggtctgcgtatcaacagcgcgaaagacgatgcggcaggtcaggcgattgctaaccgtttcaccgcgaacatcaaaggtctgactcaggcttcccgtaacgctaacgacggtatctccattgcgcagaccactgaaggcgcgctgaacgaaatcaacaacaacctgcagcgtgtgcgtgaactggcggttcagtctgctaacagcaccaactcccagtctgacctcgactccatccaggctgaaatcacccagcgcctgaacgaaatcgaccgtgtatccggccagactcagttcaacggcgtgaaagtcctggcgcaggacaacaccctgaccatccaggttggcgccaacgacggtgaaactatcgatatcgatctgaagcagatcaactctcagaccctgggtctggactcactgaacgtgcatggagcgccggtggatcctgctagcccatggaccgaaaacccgctgcagaaaattgatgccgcgctggcgcaggtggatgcgctgcgctctgatctgggtgcggtacaaaaccgtttcaactctgctatcaccaacctgggcaataccgtaaacaatctgtctgaagcgcgtagccgtatcgaagattccgactacgcgaccgaagtttccaacatgtctcgcgcgcagattttgcagcaggccggtacttccgttctggcgcaggctaaccaggtcccgcagaacgtgctgtctctgttacgtgaattctctagatatccagcacagtggcggccgctcacgcgtaccggtcatcatcaccatcaccattga

SEQ ID NO: 90 - protein, 402 aa, Artificial Sequence
  pMT/STF2delta.4XM2e (H1)
MKLCILLAVVAFVGLSLGRSAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDSLNVHGAPVDPASPWTENPLQKIDAALAQVDALRSDLGAVQNRFNSAITNLGNTVNNLSEARSRIEDSDYATEVSNMSRAQILQQAGTSVLAQANQVPQNVLSLLREFSLLTEVETPIRNEWGSRSNDSSDPLESLLTEVETPIRNEWGSRSNDSSDPGSSLLTEVETPIRNEWGSRSNDSSDPELSLLTEVETPIRNEWGSRSNDSSDPSR

SEQ ID NO: 91 - DNA, 1203 bp, Artificial Sequence
  pMT/STF2delta.4XM2e (H1)
atgtgcatattactggccgtcgtggcctttgttggcctctcgctcgggagatctgcacaagtaatcaacactaacagtctgtcgctgctgacccagaataacctgaacaaatcccagtccgcactgggcaccgctatcgagcgtctgtcttctggtctgcgtatcaacagcgcgaaagacgatgcggcaggtcaggcgattgctaaccgtttcaccgcgaacatcaaaggtctgactcaggcttcccgtaacgctaacgacggtatctccattgcgcagaccactgaaggcgcgctgaacgaaatcaacaacaacctgcagcgtgtgcgtgaactggcggttcagtctgctaacagcaccaactcccagtctgacctcgactccatccaggctgaaatcacccagcgcctgaacgaaatcgaccgtgtatccggccagactcagttcaacggcgtgaaagtcctggcgcaggacaacaccctgaccatccaggttggcgccaacgacggtgaaactatcgatatcgatctgaagcagatcaactctcagaccctgggtctggactcactgaacgtgcatggagcgccggtggatcctgctagcccatggaccgaaaacccgctgcagaaaattgatgccgcgctggcgcaggtggatgcgctgcgctctgatctgggtgcggtacaaaaccgtttcaactctgctatcaccaacctgggcaataccgtaaacaatctgtctgaagcgcgtagccgtatcgaagattccgactacgcgaccgaagtttccaacatgtctcgcgcgcagattttgcagcaggccggtacttccgttctggcgcaggctaaccaggtcccgcagaacgtgctgtctctgttacgtgaattcagcttgctgactgaggttgagaccccgattcgcaacgaatggggttcccgttccaacgattcttccgacccgctcgagagcctgttgaccgaggttgaaacccctatccgtaatgaatggggctcccgtagcaacgactcttctgacccaggatcctccctcttgaccgaagtggaaacgcctattcgtaatgagtggggttctcgtagcaatgacagctcggacccggagctctcgctgctgacggaagtggagactccgatccgtaacgagtggggctctcgctctaacgatagctcagacccgtctagataa

SEQ ID NO: 92 - protein, 402 aa, Artificial Sequence
  pMT/STF2delta.4XM2e (H5)
MKLCILLAVVAFVGLSLGRSAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDSLNVHGAPVDPASPWTENPLQKIDAALAQVDALRSDLGAVQNRFNSAITNLGNTVNNLSEARSRIEDSDYATEVSNMSRAQILQQAGTSVLAQANQVPQNVLSLLREFSLLTEVETPTRNEWESRSSDSSDPLESLLTEVETPTRNEWESRSSDSSDPESSLLTEVETPTRNEWESRSSDSSDPGSSLLTEVETPTRNEWESRSSDSSDPSR

SEQ ID NO: 93 - DNA, 1206 bp, Artificial Sequence
  pMT/STF2delta.4XM2e (H5)
atgtgcatattactggccgtcgtggcctttgttggcctctcgctcgggagatctgcacaagtaatcaacactaacagtctgtcgctgctgacccagaataacctgaacaaatcccagtccgcactgggcaccgctatcgagcgtctgtcttctggtctgcgtatcaacagcgcgaaagacgatgcggcaggtcaggcgattgctaaccgtttcaccgcgaacatcaaaggtctgactcaggcttcccgtaacgctaacgacggtatctccattgcgcagaccactgaaggcgcgctgaacgaaatcaacaacaacctgcagcgtgtgcgtgaactggcggttcagtctgctaacagcaccaactcccagtctgacctcgactccatccaggctgaaatcacccagcgcctgaacgaaatcgaccgtgtatccggccagactcagttcaacggcgtgaaagtcctggcgcaggacaacaccctgaccatccaggttggcgccaacgacggtgaaactatcgatatcgatctgaagcagatcaactctcagaccctgggtctggactcactgaacgtgcatggagcgccggtggatcctgctagcccatggaccgaaaacccgctgcagaaaattgatgccgcgctggcgcaggtggatgcgctgcgctctgatctgggtgcggtacaaaaccgtttcaactctgctatcaccaacctgggcaataccgtaaacaatctgtctgaagcgcgtagccgtatcgaagattccgactacgcgaccgaagtttccaacatgtctcgcgcgcagattttgcagcaggccggtacttccgttctggcgcaggctaaccaggtcccgcagaacgtgctgtctctgttacgtgaattctctctgctgactgaagtagaaactccaacgcgtaatgaatgggaatcccgttctagcgactcctctgatcctctcgagtccctgctgacggaggttgaaaccccgacccgcaacgagtgggaaagccgttcctccgattcctctgatccggagagcagcctgctgaccgaggtagaaaccccgacccgtaatgagtgggaatctcgctcctctgattcttctgacccgggatcctctctgctgaccgaagtggagactccgactcgcaacgaatgggagagccgttcttctgactcctctgacccgtctagataataa

SEQ ID NO: 94 - protein, 508 aa, Artificial Sequence
  pMT/STF2delta.4XM2e (H1H5)
MKLCILLAVVAFVGLSLGRSAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDSLNVHGAPVDPASPWTENPLQKIDAALAQVDALRSDLGAVQNRFNSAITNLGNTVNNLSEARSRIEDSDYATEVSNMSRAQILQQAGTSVLAQANQVPQNVLSLLREFSLLTEVETPIRNEWGSRSNDSSDPLESLLTEVETPIRNEWGSRSNDSSDPGSSLLTEVETPIRNEWGSRSNDSSDPELSLLTEVETPIRNEWGSRSNDSSDPSRQFSLLTEVETPTRNEWESRSSDSSDPLESLLTEVETPTRNEWESRSSDSSDPESSLLTEVETPTRNEWESRSSDSSDPGSSLLTEVETPTRNEWESRSSDSSDPSR

SEQ ID NO: 95 - DNA, 1527 bp, Artificial Sequence
  pMT/STF2delta.4XM2e (H1H5)
atgtgcatattactggccgtcgtggcctttgttggcctctcgctcgggagatctgcacaagtaatcaacactaacagtctgtcgctgctgacccagaataacctgaacaaatcccagtccgcactgggcaccgctatcgagcgtctgtcttctggtctgcgtatcaacagcgcgaaagacgatgcggcaggtcaggcgattgctaaccgtttcaccgcgaacatcaaaggtctgactcaggcttcccgtaacgctaacgacggtatctccattgcgcagaccactgaaggcgcgctgaacgaaatcaacaacaacctgcagcgtgtgcgtgaactggcggttcagtctgctaacagcaccaactcccagtctgacctcgactccatccaggctgaaatcacccagcgcctgaacgaaatcgaccgtgtatccggccagactcagttcaacggcgtgaaagtcctggcgcaggacaacaccctgaccatccaggttggcgccaacgacggtgaaactatcgatatcgatctgaagcagatcaactctcagaccctgggtctggactcactgaacgtgcatggagcgccggtggatcctgctagcccatggaccgaaaacccgctgcagaaaattgatgccgcgctggcgcaggtggatgcgctgcgctctgatctgggtgcggtacaaaaccgtttcaactctgctatcaccaacctgggcaataccgtaaacaatctgtctgaagcgcgtagccgtatcgaagattccgactacgcgaccgaagtttccaacatgtctcgcgcgcagattttgcagcaggccggtacttccgttctggcgcaggctaaccaggtcccgcagaacgtgctgtctctgttacgtgaattcagcttgctgactgaggttgagaccccgattcgcaacgaatggggttcccgttccaacgattcttccgacccgctcgagagcctgttgaccgaggttgaaacccctatccgtaatgaatggggctcccgtagcaacgactcttctgacccaggatcctccctcttgaccgaagtggaaacgcctattcgtaatgagtggggttctcgtagcaatgacagctcggacccggagctctcgctgctgacggaagtggagactccgatccgtaacgagtggggctctcgctctaacgatagctcagacccgtctagatctagagaattctctctgctgactgaagtagaaactccaacgcgtaatgaatgggaatcccgttctagcgactcctctgatcctctcgagtccctgctgacggaggttgaaaccccgacccgcaacgagtgggaaagccgttcctccgattcctctgatccggagagcagcctgctgaccgaggtagaaaccccgacccgtaatgagtgggaatctcgctcctctgattcttctgacccgggatcctctctgctgaccgaagtggagactccgactcgcaacgaatgggagagccgttcttctgactcctctgacccgtctagataa

SEQ ID NO: 96 - DNA, 33 bp, Artificial Sequence
  M2
gaggttgagaccccgattcgcaacgaatggggt

SEQ ID NO: 97 - protein, 10 aa, Artificial Sequence
  Linker
GAPVDPASPW

SEQ ID NO: 98 - protein, 277 aa, Artificial Sequence
  S. muenchen
MAQVINTNSLSLLTQNNLNKSQSALGTAIERLSSGLRINSAKDDAAGQAIANRFTANIKGLTQASRNANDGISIAQTTEGALNEINNNLQRVRELAVQSANGTNSQSDLDSIQAEITQRLNEIDRVSGQTQFNGVKVLAQDNTLTIQVGANDGETIDIDLKEISSKTLDKHNFRTGGELKEVNTDKTENPLQKIDAALAQVDTLRSDLGAVQNRFNSAITNLGNTVNNLSSARSRIEDSDYATEVSNMSRAQILQQAGTSVLAQANQVPQNVLSLLR

SEQ ID NO: 99 - DNA, 789 bp, Artificial Sequence
  S. muenchen
atggcacaagtcattaatacaaacagcctgtcgctgttgacccagaataacctgaacaaatcccagtccgctctgggcaccgctatcgagcgtctgtcttccggtctgcgtatcaacagcgcgaaagacgatgcggcaggtcaggcgattgctaaccgtttcaccgcgaacatcaaaggtctgactcaggcttcccgtaacgctaacgacggtatctccattgcgcagaccactgaaggcgcgctgaacgaaatcaacaacaacctgcagcgtgtgcgtgaactggcggttcagtctgctaacggtactaactcccagtctgaccttgactctatccaggctgaaatcacccagcgtctgaacgaaatcgaccgtgtatccggtcagactcagttcaacggcgtgaaagtcctggcgcaggacaacaccctgaccatccaggttggtgccaacgacggtgaaactattgatattgatttaaaagaaattagctctaaaacactgacagataagactgaaaacccactgcagaaaattgatgctgccttggcacaggttgatacacttcgttctgacctgggtgcggtacagaaccgtttcaactccgctatcaccaacctgggcaataccgtaaataacctgtcttctgcccgtagccgtatcgaagattccgactacgcgaccgaagtctccaacatgtctcgcgcgcagattctgcagcaggccggtacctccgttctggcgcaggctaaccaggttccgcaaaacgtcctctctttactgcgttaa

SEQ ID NO: 100 - protein, 102 aa, Artificial Sequence
  H1 and H5
SLLTEVETPTRNEWESRSSDSSDPLESLLTEVETPTRNEWESRSSDSSDPESSLLTEVETPTRNEWESRSSDSSDPGSSLLTEVETPTRNEWESRSSDSSDP

SEQ ID NO: 101 - DNA, 306 bp, Artificial Sequence
  H1 and H5
tctctgctgactgaagtagaaactccaacgcgtaatgaatgggaatcccgttctagcgactcctctgatcctctcgagtccctgctgacggaggttgaaaccccgacccgcaacgagtgggaaagccgttcctccgattcctctgatccggagagcagcctgctgaccgaggtagaaaccccgacccgtaatgagtgggaatctcgctcctctgattcttctgacccgggatcctctctgctgaccgaagtggagactccgactcgcaacgaatgggagagccgttcttctgactcctctgacccg

SEQ ID NO: 102 - protein, 20 aa, Artificial Sequence
  HA
PAKLLKERGFFGAIAGFLEE

SEQ ID NO: 103 - protein, 20 aa, Artificial Sequence
  HA
NIPSIQSRGLFGAIAGFIEE

SEQ ID NO: 104 - protein, 20 aa, Artificial Sequence
  HA
NVPEKQTRGIFGAIAGFIEE

SEQ ID NO: 105 - protein, 20 aa, Artificial Sequence
  HA
RERRRKKRGLFGAIAGFIEE

SEQ ID NO: 106 - DNA, 72 bp, Artificial Sequence
  M2
agcttgctgactgaggttgagaccccgattcgcaacgaatggggttcccgttccaacgattcttccgacccg

SEQ ID NO: 107 - protein, 478 aa, Artificial Sequence
  PR8 HA
MKAKLLVLLCTFTATYADTICIGYHANNSTDTVDTVLEKNVTVTHSVNLLEDSHNGKLCLLKGIAPLQLGNCSVAGWILGNPECELLISKESWSYIVETPNPENGTCYPGYFADYEELREQLSSVSSFERFEIFPKESSWPNHTVTGVSASCSHNGKSSFYRNLLWLTGKNGPNLSKSYVNNKEKEVLVLWGVHHPPNIGNQRALYHTENAYVSVVSSHYSRRFTPEIAKRPKVRDQEGRINYYWTLLEPGDTIIFEANGNLIAPWYAFALSRGFGSGIITSNAPMDECDAKCQTPQGAINSSLPFQNVHPVTIGECPKYVRSAKLRMVTGLRNIPSIQSRGLFGAIAGFIEGGWTGMVDGWYGYHHQNEQGSGYAADQKSTQNAINGITNKVNSVIEKMNTQFTAVGKEFNKLERRMENLNKKVDDGFLDIWTYNAELLVLLENERTLDFHDSNVKNLYEKVKSQLKNNAKEIGNGC

SEQ ID NO: 108 - protein, 573 aa, Artificial Sequence
  New Caledonia HA
QKQGIIKTTKMKVKLLVLLCTFTATYADTICIGYHANNSTDTVDTVLEKNVTVTHSVNLLEDSHNGKLCLLKGIAPLQLGNCSVAGWILGNPECELLISRESWSYIVEKPNPENGTCYPGHFADYEELREQLSSVSSFERFEIFPKESSWPNHTTTGVSASCSHNGESSFYKNLLWLTGKNGPNLSKSYANNKEKEVLVLWGVHHPPNIGDQRALYHKENAYVSVVSSHYSRKFTPEIAKRPKVRDQEGRINYYWTLLEPGDTIIFEANGNLIAPRYAFALSRGFGSGIINSNAPMDECDAKCQTPQGAINSSLPFQNVHPVTIGECPKYVRSAKLRMVTGLRNIPSIQSRGLFGAIAGFIEGGWTGMVDGWYGYHHQNEQGSGYAADQKSTQNAINGITNKVNSVIEKMNTQFTAVGKEFNKLERRMENLNKKVDDGFIDIWTYNAELLVLLENERTLDFHDSNVKNLYEKVKSQLKNNAKEIGNGCFEFYHKCNDECMESVKNGTYDYPKYSEESKLNREKIDGVKLESMGVYQILAIYSTVASSLVLLVSLGAISFWMCSNGSLQCRICI

SEQ ID NO: 109 - protein, 563 aa, Artificial Sequence
  Solomon Islands HA
MKANLLVLLSAAADADTICIGYHANNSTDTVDTVLEKNVTVTHSVNLLEDSHNGKLCRLKGIAPLQLGKCNIAGWLLGNPECDPLLPVRSWSYIVETPNSENGICYPGDFIDYEELREQLSSVSSFERFEIFPKESSWPNHNTNGVTAACSHEGKSSFYRNLLWLTEKEGSYPKLKNSYVNKKGKEVLVLWGIHHPPNSKEQQNIYQNENAYVSVVTSNYNRRFTPEIAERPKVRDQAGRMNYYWTLLKPGDTIIFEANGNLIAPMYAFALSRGFGSGIITSNASMHECNTKCQTPLGAINSSLPYQNIHPVTIGECPKYVRSAKLRMVTGLRNTPSIQSRGLFGAIAGFIEGGWTGMIDGWYGYHHQNEQGSGYAADQKSTQNAINGITNKVNTVIEKMNIQFTAVGKEFNKLEKRMENLNKKVDDGFLDIWTYNAELLVLLENERTLDFHDSNVKNLYEKVKSQLKNNAKEIGNGCFEFYHKCDNECMESVRNGTYDYPKYSEESKLNREKVDGVKLESMGIYQILAIYSTVASSLVLLVSLGAISFWMCSNGSLQCRICI

SEQ ID NO: 110 - protein, 575 aa, Artificial Sequence
  Conserved Sequence
QKQGIIKTTKMKAKLLVLLCTFTATYADTICIGYHANNSTDTVDTVLEKNVTVTHSVNLLEDSHNGKLCLLKGIAPLQLGNCSVAGWILGNPECELLISXESWSYIVETPNPENGTCYPGXFADYEELREQLSSVSSFERFEIFPKESSWPNHTTTGVSASCSHNGKSSFYRNLLWLTGKNGLYPNLSKSYVNNKEKEVLVLWGVHHPPNIGXQRALYHXENAYVSVVSSHYSRRFTPEIAKRPKVRDQEGRINYYWTLLEPGDTIIFEANGNLIAPXYAFALSRGFGSGIITSNAPMDECDAKCQTPQGAINSSLPFQNVHPVTIGECPKYVRSAKLRMVTGLRNIPSIQSRGLFGAIAGFIEGGWTGMVDGWYGYHHQNEQGSGYAADQKSTQNAINGITNKVNSVIEKMNTQFTAVGKEFNKLERRMENLNKKVDDGFLDIWTYNAELLVLLENERTLDFHDSNVKNLYEKVKSQLKNNAKEIGNGCFEFYHKCXXECMESVXNGTYDYPKYSEESKLNREKXDGVKLESMGXYQILAIYSTVASSLVLLVSLGAISFWMCSNGSLQCRICI

SEQ ID NO: 111 - protein, 729 aa, Artificial Sequence
  STF2.HA1-2 SI
MAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDSLNVQKAYDVKDTAVTTKAYANNGTTLDVSGLDDAAIKAATGGTNGTASVTGGAVKFDADNNKYFVTIGGFTGADAAKNGDYEVNVATDGTVTLAAGATKTTMPAGATTKTEVQELKDTPAVVSADAKNALIAGGVDATDANGAELVKMSYTDKNGKTIEGGYALKAGDKYYAADYDEATGAIKAKTTSYTAADGTTKTAANQLGGVDGKTEVVTIDGKTYNASKAAGHDFKAQPELAEAAAKTTENPLQKIDAALAQVDALRSDLGAVQNRFNSAITNLGNTVNNLSEARSRIEDSDYATEVSNMSRAQILQQAGTSVLAQANQVPQNVLSLLAKGIAPLQLGNCSVAGWILGNPECELLISRESWSYIVEKPNPENGTCYPGHFADYEELREQLSSVSSFERFEIFPKESSWPNHTTTGVSASCSHNGESSFYKNLLWLTGKNGLYPNLSKSYANNKEKEVLVLWGVHHPPNIGDQRALYHKENAYVSVVSSHYSRKFTPEIAKRPKVRDQEGRINYYWTLLEPGDTIIFEANGNLIAPRYAFALSRGFGSGIINS

SEQ ID NO: 112 - protein, 500 aa, Artificial Sequence
  STF2delta.HA1-2 SI
MAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDSLNVHGAPVDPASPWTENPLQKIDAALAQVDALRSDLGAVQNRFNSAITNLGNTVNNLSEARSRIEDSDYATEVSNMSRAQILQQAGTSVLAQANQVPQNVLSLLAKGIAPLQLGNCSVAGWILGNPECELLISRESWSYIVEKPNPENGTCYPGHFADYEELREQLSSVSSFERFEIFPKESSWPNHTTTGVSASCSHNGESSFYKNLLWLTGKNGLYPNLSKSYANNKEKEVLVLWGVHHPPNIGDQRALYHKENAYVSVVSSHYSRKFTPEIAKRPKVRDQEGRINYYWTLLEPGDTIIFEANGNLIAPRYAFALSRGFGSGIINS

SEQ ID NO: 113 - protein, 301 aa, Artificial Sequence
  Mel-HA1-1.his SI
RSMKFLVNVALVFMVVYISYIYASHNGKLCLLKGIAPLQLGNCSVAGWILGNPECELLISRESWSYIVEKPNPENGTCYPGHFADYEELREQLSSVSSFERFEIFPKESSWPNHTTTGVSASCSHNGESSFYKNLLWLTGKNGLYPNLSKSYANNKEKEVLVLWGVHHPPNIGDQRALYHKENAYVSVVSSHYSRKFTPEIAKRPKVRDQEGRINYYWTLLEPGDTIIFEANGNLIAPRYAFALSRGFGSGIINSNAPMDECDAKCQTPQGAINSSLPFQNVHPVTIGECPKYVRHHHHHH

SEQ ID NO: 114 - protein, 272 aa, PRArtificial Sequence
  PR/8 HA1-1
SHNGKLCRLKGIAPLQLGKCNIAGWLLGNPECDPLLPVRSWSYIVETPNSENGICYPGDFIDYEELREQLSSVSSFERFEIFPKESSWPNHNTNGVTAACSHEGKSSFYRNLLWLTEKEGSYPKLKNSYVNKKGKEVLVLWGIHHPPNSKEQQNLYQNENAYVSVVTSNYNRRFTPEIAERPKVRDQAGRMNYYWTLLKPGDTIIFEANGNLIAPMYAFALSRGFGSGIITSNASMHECNTKCQTPLGAINSSLPYQNIHPVTIGECPKYVR

SEQ ID NO: 115 - protein, 274 aa, Artificial Sequence
  VN HA1-1
EKKHNGKLCDLDGVKPLILRDCSVAGWLLGNPMCDEFINVPEWSYIVEKANPVNDLCYPGDFNDYEELKHLLSRINHFEKIQIIPKSSWSSHEASLGVSSACPYQGKSSFFRNVVWLIKKNSTYPTIKRSYNNTNQEDLLVLWGIHHPNDAAEQTKLYQNPTTYISVGTSTLNQRLVPRIATRSKVNGQSGRMEFFWTILKPNDAINFESNGNFIAPEYAYKIVKKGDSTIMKSELEYGNCNTKCQTPMGAINSSMPFHNIHPLTIGECPKYVK

SEQ ID NO: 116 - protein, 274 aa, Artificial Sequence
  IND HA1-1
EKTHNGKLCDLDGVKPLILRDCSVAGWLLGNPMCDEFINVPEWSYIVEKANPTNDLCYPGSFNDYEELKHLLSRINHFEKIQIIPKSSWSDHEASSGVSSACPYLGSPSFFRNVVWLIKKNSTYPTIKKSYNNTNQEDLLVLWGIHHPNDAAEQTRLYQNPTTYISIGTSTLNQRLVPKIATRSKVNGQSGRMEFFWTILKPNDAINFESNGNFIAPEYAYKIVKKGDSAIMKSELEYGNCNTKCQTPMGAINSSMPFHNIHPLTIGECPKYVK

SEQ ID NO: 117 - protein, 272 aa, Artificial Sequence
  NC HA1-1
SHNGKLCLLKGIAPLQLGNCSVAGWILGNPECELLISKESWSYIVETPNPENGTCYPGYFADYEELREQLSSVSSFERFEIFPKESSWPNHTVTGVSASCSHNGKSSFYRNLLWLTGKNGLYPNLSKSYVNNKEKEVLVLWGVHHPPNIGNQRALYHTENAYVSVVSSHYSRRFTPEIAKRPKVRDQEGRINYYWTLLEPGDTIIFEANGNLIAPWYAFALSRGFGSGIITSNAPMDECDAKCQTPQGAINSSLPFQNVHPVTIGECPKYVR

SEQ ID NO: 118 - protein, 267 aa, Artificial Sequence
  WIS HA1-1
QSSSTGGICDSPHQILDGENCTLIDALLGDPQCDGFQNKKWDLFVERSKAYSNCYPYDVPDYASLRSLVASSGTLEFNDESFNWTGVTQNGTSSACKRRSNNSFFSRLNWLTHLKFKYPALNVTMPNNEKFDKLYIWGVHHPGTDNDQIFLHAQASGRITVSTKRSQQTVIPNIGSRPRIRNIPSRISIYWTIVKPGDILLINSTGNLIAPRGYFKIRSGKSSIMRSDAPIGKCNSECITPNGSIPNDKPFQNVNRITYGACPRYVK

SEQ ID NO: 119 - protein, 293 aa, Artificial Sequence
  B/Lee/40 HA1-1
TTTPTKSHFANLKGTQTRGKLCPNCFNCTDLDVALGRPKCMGNTPSAKVSILHEVKPATSGCFPIMHDRTKIRQLPNLLRGYENIRLSTSNVINTETAPGGPYKVGTSGSCPNVANGNGFFNTMAWVIPKDNNKTAINPVTVEVPYICSEGEDQITVWGFHSDDKTQMERLYGDSNPQKFTSSANGVTTHYVSQIGGFPNQTEDEGLKQSGRIVVDYMVQKPGKTGTIVYQRGILLPQKVWCASGRSKVIKGSLPLIGEADCLHEKYGGLNKSKPYYTGEHAKAIGNCPIWVK

SEQ ID NO: 120 - protein, 267 aa, Artificial Sequence
  A/Aichi/2/68 HA1-1
QSSSTGKICNNPHRILDGIDCTLIDALLGDPHCDVFQNETWDLFVERSKAFSNCYPYDVPDYASLRSLVASSGTLEFITEGFTWTGVTQNGGSNACKRGPGSGFFSRLNWLTKSGSTYPVLNVTMPNNDNFDKLYIWGIHHPSTNQEQTSLYVQASGRVTVSTRRSQQTIIPNIGSRPWVRGLSSRISIYWTIVKPGDVLVINSNGNLIAPRGYFKMRTGKSSIMRSDAPIDTCISECITPNGSIPNDKPFQNVNKITYGACPKYVK

SEQ ID NO: 121 - protein, 294 aa, Artificial Sequence
  B/Malaysia/2506/2004 HA1-1
TTTPTKSHFANLKGTETRGKLCPKCLNCTDLDVALGRPKCTGNIPSARVSILHEVRPVTSGCFPIMHDRTKIRQLPNLLRGYEHIRLSTHNVINAENAPGGSYKIGTSGSCPNVTNGNGFFATMAWAVPKNDNNKTATNSLTIEVPYICTEGEDQITVWGFHSDNEAQMAKLYGDSKPQKFTSSANGVTTHYVSQIGGFPNQTEDGGLPQSGRIVVDYMVQKSGKTGTITYQRGILLPQKVWCASGRSKVIKGSLPLIGEADCLHEKYGGLNKSKPYYTGEHAKAIGNCPIWVK

SEQ ID NO: 122 - protein, 294 aa, Artificial Sequence
  B/Ohio/1/2005 HA1-1
TTTPTKSHFANLKGTKTRGKLCPKCLNCTDLDVALGRPKCTGNIPSAEVSILHEVRPVTSGCFPIMHDRTKIRQLPNLLRGYEHIRLSTHNVINAEKAPGGPYKIGTSGSCPNVTNGNGFFATMAWAVPKNDNNKTATNSLTIEVPYICTEGEDQITIWGFHSDSETQMAKLYGDSKPQKFTSSANGVTTHYVSQIGGFPNQTEDGGLPQSGRIVVDYMVQKSGKTGTITYQRGILLPQKVWCASGRSKVIKGSLPLIGEADCLHEKYGGLNKSKPYYTGEHAKAIGNCPIWVK

SEQ ID NO: 123 - protein, 223 aa, Artificial Sequence
  PR/8 HA1-2
KGIAPLQLGKCNIAGWLLGNPECDPLLPVRSWSYIVETPNSENGICYPGDFIDYEELREQLSSVSSFERFEIFPKESSWPNHNTNGVTAACSHEGKSSFYRNLLWLTEKEGSYPKLKNSYVNKKGKEVLVLWGIHHPPNSKEQQNLYQNENAYVSVVTSNYNRRFTPEIAERPKVRDQAGRMNYYWTLLKPGDTIIFEANGNLIAPMYAFALSRGFGSGIITS

SEQ ID NO: 124 - protein, 223 aa, Artificial Sequence
  VN HA1-2
GVKPLILRDCSVAGWLLGNPMCDEFINVPEWSYIVEKANPVNDLCYPGDFNDYEELKHLLSRINHFEKIQIIPKSSWSSHEASLGVSSACPYQGKSSFFRNVVWLIKKNSTYPTIKRSYNNTNQEDLLVLWGIHHPNDAAEQTKLYQNPTTYISVGTSTLNQRLVPRIATRSKVNGQSGRMEFFWTILKPNDAINFESNGNFIAPEYAYKIVKKGDSTIMKSE

SEQ ID NO: 125 - protein, 223 aa, Artificial Sequence
  IND HA1-2
GVKPLILRDCSVAGWLLGNPMCDEFINVPEWSYIVEKANPTNDLCYPGSFNDYEELKHLLSRINHFEKIQIIPKSSWSDHEASSGVSSACPYLGSPSFFRNVVWLIKKNSTYPTIKKSYNNTNQEDLLVLWGIHHPNDAAEQTRLYQNPTTYISIGTSTLNQRLVPKIATRSKVNGQSGRMEFFWTILKPNDAINFESNGNFIAPEYAYKIVKKGDSAIMKSE

SEQ ID NO: 126 - protein, 223 aa, Artificial Sequence
  NC HA1-2
KGIAPLQLGNCSVAGWILGNPECELLISKESWSYIVETPNPENGTCYPGYFADYEELREQLSSVSSFERFEIFPKESSWPNHTVTGVSASCSHNGKSSFYRNLLWLTGKNGLYPNLSKSYVNNKEKEVLVLWGVHHPPNIGNQRALYHTENAYVSVVSSHYSRRFTPEIAKRPKVRDQEGRINYYWTLLEPGDTIIFEANGNLIAPWYAFALSRGFGSGIITS

SEQ ID NO: 127 - protein, 218 aa, Artificial Sequence
  WIS HA1-2
SPHQILDGENCTLIDALLGDPQCDGFQNKKWDLFVERSKAYSNCYPYDVPDYASLRSLVASSGTLEFNDESFNWTGVTQNGTSSACKRRSNNSFFSRLNWLTHLKFKYPALNVTMPNNEKFDKLYIWGVHHPGTDNDQIFLHAQASGRITVSTKRSQQTVIPNIGSRPRIRNIPSRISIYWTIVKPGDILLINSTGNLIAPRGYFKIRSGKSSIMRSD

SEQ ID NO: 128 - protein, 223 aa, Artificial Sequence
  PR/8 HA1-2mut
KGAAPLQLGKCNIAGWLLGNPECDPLLPVRSWSDIAETPNSENGICYPGDFIDYEELREQLSSVSSFERFEIFPKESSWPNHNTNGVTAACSHEGKSSFYRNLLWLTEKEGSYPKLKNSYVNKKGKEVLVLWGIHHPPNSKEQQNLYQNENAYVSVVTSNYNRRFTPEIAERPKVRDQAGRMNYYWTLLKPGDTIIFEANGNLIAPMYAFALSRGFGSGIITS

SEQ ID NO: 129 - protein, 223 aa, Artificial Sequence
  VN HA1-2mut
GAKPLSLRDCSVAGWLLGNPMCDEFINVPEWSDIAEKANPVNDLCYPGDFNDYEELKHLLSRINHFEKIQIIPKSSWSSHEASLGVSSACPYQGKSSFFRNVVWLIKKNSTYPTIKRSYNNTNQEDLLVLWGIHHPNDAAEQTKLYQNPTTYISVGTSTLNQRLVPRIATRSKVNGQSGRMEFFWTILKPNDAINFESNGNFIAPEYAYKIVKKGDSTIMKSE

SEQ ID NO: 130 - protein, 223 aa, Artificial Sequence
  IND HA1-2mut
GAKPLSLRDCSVAGWLLGNPMCDEFINVPEWSDIAEKANPTNDLCYPGSFNDYEELKHLLSRINHFEKIQIIPKSSWSDHEASSGVSSACPYLGSPSFFRNVVWLIKKNSTYPTIKKSYNNTNQEDLLVLWGIHHPNDAAEQTRLYQNPTTYISIGTSTLNQRLVPKIATRSKVNGQSGRMEFFWTILKPNDAINFESNGNFIAPEYAYKIVKKGDSAIMKSE

SEQ ID NO: 131 - protein, 223 aa, Artificial Sequence
  NC HA1-2mut
KGAAPLQLGNCSVAGWILGNPECELLISKESWSDIAETPNPENGTCYPGYFADYEELREQLSSVSSFERFEIFPKESSWPNHTVTGVSASCSHNGKSSFYRNLLWLTGKNGLYPNLSKSYVNNKEKEVLVLWGVHHPPNIGNQRALYHTENAYVSVVSSHYSRRFTPEIAKRPKVRDQEGRINYYWTLLEPGDTIIFEANGNLIAPWYAFALSRGFGSGIITS

SEQ ID NO: 132 - protein, 218 aa, Artificial Sequence
  WIS HA1-2mut
SPHQALDGENCTLIDALLGDPQCDGFQNKKWDDFAERSKAYSNCYPYDVPDYASLRSLVASSGTLEFNDESFNWTGVTQNGTSSACKRRSNNSFFSRLNWLTHLKFKYPALNVTMPNNEKFDKLYIWGVHHPGTDNDQIFLHAQASGRITVSTKRSQQTVIPNIGSRPRIRNIPSRISIYWTIVKPGDILLINSTGNLIAPRGYFKIRSGKSSIMRSD

SEQ ID NO: 133 - protein, 176 aa, Artificial Sequence
  PR/8 HA1-3
NSENGICYPGDFIDYEELREQLSSVSSFERFEIFPKESSWPNHNTNGVTAACSHEGKSSFYRNLLWLTEKEGSYPKLKNSYVNKKGKEVLVLWGIHHPPNSKEQQNLYQNENAYVSVVTSNYNRRFTPEIAERPKVRDQAGRMNYYWTLLKPGDTIIFEANGNLIAPMYAFALSRG

SEQ ID NO: 134 - protein, 174 aa, Artificial Sequence
  VN HA1-3
NDLCYPGDFNDYEELKHLLSRINHFEKIQIIPKSSWSSHEASLGVSSACPYQGKSSFFRNVVWLIKKNSTYPTIKRSYNNTNQEDLLVLWGIHHPNDAAEQTKLYQNPTTYISVGTSTLNQRLVPRIATRSKVNGQSGRMEFFWTILKPNDAINFESNGNFIAPEYAYKIVKKG

SEQ ID NO: 135 - protein, 174 aa, Artificial Sequence
  IND HA1-3
NDLCYPGSFNDYEELKHLLSRINHFEKIQIIPKSSWSDHEASSGVSSACPYLGSPSFFRNVVWLIKKNSTYPTIKKSYNNTNQEDLLVLWGIHHPNDAAEQTRLYQNPTTYISIGTSTLNQRLVPKIATRSKVNGQSGRMEFFWTILKPNDAINFESNGNFIAPEYAYKIVKKG

SEQ ID NO: 136 - protein, 176 aa, Artificial Sequence
  NC HA1-3
NPENGTCYPGYFADYEELREQLSSVSSFERFEIFPKESSWPNHTVTGVSASCSHNGKSSFYRNLLWLTGKNGLYPNLSKSYVNNKEKEVLVLWGVHHPPNIGNQRALYHTENAYVSVVSSHYSRRFTPEIAKRPKVRDQEGRINYYWTLLEPGDTIIFEANGNLIAPWYAFALSRG

SEQ ID NO: 137 - protein, 169 aa, Artificial Sequence
  WIS HA1-3
SNCYPYDVPDYASLRSLVASSGTLEFNDESFNWTGVTQNGTSSACKRRSNNSFFSRLNWLTHLKFKYPALNVTMPNNEKFDKLYIWGVHHPGTDNDQIFLHAQASGRITVSTKRSQQTVIPNIGSRPRIRNIPSRISIYWTIVKPGDILLINSTGNLIAPRGYFKIRSG

SEQ ID NO: 138 - protein, 176 aa, Artificial Sequence
  PR/8 HA1-3mut
NSENEICYPGDFIDKEELREQLSSVSSFERFEIFPKESSWPNHNTNGVTAACSHEGKSSFYRNLLWLTEKEGSYPKLKNSYVNKKGKEVLVLWGIHHPPNSKEQQNLYQNENAYVSVVTSNYNRRFTPEIAERPKVRDQAGRMNYYWTLLKPGDTIIFEANGNLIAPMYAAALSRG

SEQ ID NO: 139 - protein, 174 aa, Artificial Sequence
  VN HA1-3mut
NDLCYPGDFNDKEELKHLLSRINHFEKIQIIPKSSWSSHEASLGVSSACPYQGKSSFFRNVVWLIKKNSTYPTIKRSYNNTNQEDLLVLWGIHHPNDAAEQTKLYQNPTTYISVGTSTLNQRLVPRIATRSKVNGQSGRMEFFWTILKPNDAINFESNGNFIAPEYAYKIAKKG

SEQ ID NO: 140 - protein, 174 aa, Artificial Sequence
  IND HA1-3mut
NDLCYPGSFNDKEELKHLLSRINHFEKIQIIPKSSWSDHEASSGVSSACPYLGSPSFFRNVVWLIKKNSTYPTIKKSYNNTNQEDLLVLWGIHHPNDAAEQTRLYQNPTTYISIGTSTLNQRLVPKIATRSKVNGQSGRMEFFWTILKPNDAINFESNGNFIAPEYAYKIAKKG

SEQ ID NO: 141 - protein, 8 aa, Artificial Sequence
  TLR4 peptide
GGKSGRTG

SEQ ID NO: 142 - protein, 9 aa, Artificial Sequence
  TLR4 peptide
KGYDWLVVG

SEQ ID NO: 143 - protein, 10 aa, Artificial Sequence
  TLR4 peptide
EDMVYRIGVP

SEQ ID NO: 144 - protein, 6 aa, Artificial Sequence
  TLR4 peptide
VKLSGS

SEQ ID NO: 145 - protein, 8 aa, Artificial Sequence
  TLR4 peptide
GMLSLALF

SEQ ID NO: 146 - protein, 7 aa, Artificial Sequence
  TLR4 peptide
CVVGSVR

SEQ ID NO: 147 - protein, 8 aa, Artificial Sequence
  TLR4 peptide
IVRGCLGW

SEQ ID NO: 148 - protein, 8 aa, Artificial Sequence
  TLR4 peptide
AAEERTLG

SEQ ID NO: 149 - protein, 9 aa, Artificial Sequence
  TLR4 peptide
WARVVGWLR

SEQ ID NO: 150 - protein, 9 aa, Artificial Sequence
  TLR4 peptide
SEGYRLFGG

SEQ ID NO: 151 - protein, 10 aa, Artificial Sequence
  TLR4 peptide
LVGGVVRRGS

SEQ ID NO: 152 - protein, 10 aa, Artificial Sequence
  TLR4 peptide
GRVNDLWLAA

SEQ ID NO: 153 - protein, 10 aa, Artificial Sequence
  TLR4 peptide
SGWMLWREGS

SEQ ID NO: 154 - protein, 10 aa, Artificial Sequence
  TLR4 peptide
ERMEDRGGDL

SEQ ID NO: 155 - protein, 9 aa, Artificial Sequence
  TLR4 peptide
KLCCFTECM

SEQ ID NO: 156 - protein, 10 aa, Artificial Sequence
  TLR4 peptide
AVGSMERGRG

SEQ ID NO: 157 - protein, 9 aa, Artificial Sequence
  TLR4 peptide
RDWVGGDLV

SEQ ID NO: 158 - protein, 10 aa, Artificial Sequence
  TLR4 peptide
FFEVAKISQQ

SEQ ID NO: 159 - protein, 5 aa, Artificial Sequence
  TLR4 peptide
WWYWC

SEQ ID NO: 160 - protein, 7 aa, Artificial Sequence
  TLR4 peptide
MHLCSHA

SEQ ID NO: 161 - protein, 7 aa, Artificial Sequence
  TLR4 peptide
WLFRRIG

SEQ ID NO: 162 - protein, 7 aa, Artificial Sequence
  TLR4 peptide
YWFWRIG

SEQ ID NO: 163 - protein, 7 aa, Artificial Sequence
  TLR4 peptide
MHLYCIA

SEQ ID NO: 164 - protein, 8 aa, Artificial Sequence
  TLR4 peptide
WPLFPWIV

SEQ ID NO: 165 - protein, 7 aa, Artificial Sequence
  TLR4 peptide
DMRSHAR

SEQ ID NO: 166 - protein, 7 aa, Artificial Sequence
  TLR4 peptide
MHLCTHA

SEQ ID NO: 167 - protein, 6 aa, Artificial Sequence
  TLR4 peptide
NLFPFY

SEQ ID NO: 168 - protein, 7 aa, Artificial Sequence
  TLR4 peptide
MHLCTRA

SEQ ID NO: 169 - protein, 7 aa, Artificial Sequence
  TLR4 peptide
RHLWYHA

SEQ ID NO: 170 - protein, 7 aa, Artificial Sequence
  TLR4 peptide
WPFSAYW

SEQ ID NO: 171 - protein, 6 aa, Artificial Sequence
  TLR4 peptide
WYLRGS

SEQ ID NO: 172 - protein, 7 aa, Artificial Sequence
  TLR4 peptide
GKGTDLG

SEQ ID NO: 173 - protein, 6 aa, Artificial Sequence
  TLR4 peptide
IFVRMR

SEQ ID NO: 174 - protein, 7 aa, Artificial Sequence
  TLR4 peptide
WLFRPVF

SEQ ID NO: 175 - protein, 7 aa, Artificial Sequence
  TLR4 peptide
FLGWLMG

SEQ ID NO: 176 - protein, 7 aa, Artificial Sequence
  TLR4 peptide
MHLWHHA

SEQ ID NO: 177 - protein, 7 aa, Artificial Sequence
  TLR4 peptide
WWFPWKA

SEQ ID NO: 178 - protein, 7 aa, Artificial Sequence
  TLR4 peptide
WYLPWLG

SEQ ID NO: 179 - protein, 7 aa, Artificial Sequence
  TLR4 peptide
WPFPRTF

SEQ ID NO: 180 - protein, 7 aa, Artificial Sequence
  TLR4 peptide
WPFPAYW

SEQ ID NO: 181 - protein, 7 aa, Artificial Sequence
  TLR4 peptide
FLGLRWL

SEQ ID NO: 182 - protein, 10 aa, Artificial Sequence
  TLR4 peptide
SRTDVGVLEV

SEQ ID NO: 183 - protein, 10 aa, Artificial Sequence
  TLR4 peptide
REKVSRGDKG

SEQ ID NO: 184 - protein, 10 aa, Artificial Sequence
  TLR4 peptide
DWDAVESEYM

SEQ ID NO: 185 - protein, 10 aa, Artificial Sequence
  TLR4 peptide
VSSAQEVRVP

SEQ ID NO: 186 - protein, 10 aa, Artificial Sequence
  TLR4 peptide
LTYGGLEALG

SEQ ID NO: 187 - protein, 10 aa, Artificial Sequence
  TLR4 peptide
VEEYSSSGVS

SEQ ID NO: 188 - protein, 10 aa, Artificial Sequence
  TLR4 peptide
VCEVSDSVMA

SEQ ID NO: 189 - protein, 5 aa, Artificial Sequence
  TLR2 peptide
NPPTT

SEQ ID NO: 190 - protein, 5 aa, Artificial Sequence
  TLR2 peptide
MRRIL

SEQ ID NO: 191 - protein, 4 aa, Artificial Sequence
  TLR2 peptide
MISS

SEQ ID NO: 192 - protein, 5 aa, Artificial Sequence
  TLR2 peptide
RGGSK

SEQ ID NO: 193 - protein, 4 aa, Artificial Sequence
  TLR2 peptide
RGGF

SEQ ID NO: 194 - protein, 5 aa, Artificial Sequence
  TLR2 peptide
NRTVF

SEQ ID NO: 195 - protein, 5 aa, Artificial Sequence
  TLR2 peptide
NRFGL

SEQ ID NO: 196 - protein, 5 aa, Artificial Sequence
  TLR2 peptide
SRHGR

SEQ ID NO: 197 - protein, 5 aa, Artificial Sequence
  TLR2 peptide
IMRHP

SEQ ID NO: 198 - protein, 5 aa, Artificial Sequence
  TLR2 peptide
EVCAP

SEQ ID NO: 199 - protein, 5 aa, Artificial Sequence
  TLR2 peptide
ACGVY

SEQ ID NO: 200 - protein, 5 aa, Artificial Sequence
  TLR2 peptide
CGPKL

SEQ ID NO: 201 - protein, 5 aa, Artificial Sequence
  TLR2 peptide
AGCFS

SEQ ID NO: 202 - protein, 5 aa, Artificial Sequence
  TLR2 peptide
SGGLF

SEQ ID NO: 203 - protein, 5 aa, Artificial Sequence
  TLR2 peptide
AVRLS

SEQ ID NO: 204 - protein, 5 aa, Artificial Sequence
  TLR2 peptide
GGKLS

SEQ ID NO: 205 - protein, 5 aa, Artificial Sequence
  TLR2 peptide
VSEGV

SEQ ID NO: 206 - protein, 5 aa, Artificial Sequence
  TLR2 peptide
KCQSF

SEQ ID NO: 207 - protein, 5 aa, Artificial Sequence
  TLR2 peptide
FCGLG

SEQ ID NO: 208 - protein, 5 aa, Artificial Sequence
  TLR2 peptide
PESGV

SEQ ID NO: 209 - protein, 5 aa, Artificial Sequence
  TLR2 peptide
DPDSG

SEQ ID NO: 210 - protein, 5 aa, Artificial Sequence
  TLR2 peptide
IGRFR

SEQ ID NO: 211 - protein, 5 aa, Artificial Sequence
  TLR2 peptide
MGTLP

SEQ ID NO: 212 - protein, 5 aa, Artificial Sequence
  TLR2 peptide
ADTHQ

SEQ ID NO: 213 - protein, 5 aa, Artificial Sequence
  TLR2 peptide
HLLPG

SEQ ID NO: 214 - protein, 5 aa, Artificial Sequence
  TLR2 peptide
GPLLH

SEQ ID NO: 215 - protein, 5 aa, Artificial Sequence
  TLR2 peptide
NYRRW

SEQ ID NO: 216 - protein, 5 aa, Artificial Sequence
  TLR2 peptide
LRQGR

SEQ ID NO: 217 - protein, 5 aa, Artificial Sequence
  TLR2 peptide
IMWFP

SEQ ID NO: 218 - protein, 5 aa, Artificial Sequence
  TLR2 peptide
RVVAP

SEQ ID NO: 219 - protein, 5 aa, Artificial Sequence
  TLR2 peptide
IHVVP

SEQ ID NO: 220 - protein, 5 aa, Artificial Sequence
  TLR2 peptide
MFGVP

SEQ ID NO: 221 - protein, 5 aa, Artificial Sequence
  TLR2 peptide
CVWLQ

SEQ ID NO: 222 - protein, 5 aa, Artificial Sequence
  TLR2 peptide
IYKLA

SEQ ID NO: 223 - protein, 4 aa, Artificial Sequence
  TLR2 peptide
KGWF

SEQ ID NO: 224 - protein, 5 aa, Artificial Sequence
  TLR2 peptide
KYMPH

SEQ ID NO: 225 - protein, 5 aa, Artificial Sequence
  TLR2 peptide
VGKND

SEQ ID NO: 226 - protein, 5 aa, Artificial Sequence
  TLR2 peptide
THKPK

SEQ ID NO: 227 - protein, 5 aa, Artificial Sequence
  TLR2 peptide
SHIAL

SEQ ID NO: 228 - protein, 5 aa, Artificial Sequence
  TLR2 peptide
AWAGT

SEQ ID NO: 229 - protein, 20 aa, Artificial Sequence
  TLR2 peptide
KGGVGPVRRSSRLRRTTQPG

SEQ ID NO: 230 - protein, 23 aa, Artificial Sequence
  TLR2 peptide
GRRGLCRGCRTRGRIKQLQSAHK

SEQ ID NO: 231 - protein, 22 aa, Artificial Sequence
  TLR2 peptide
RWGYHLRDRKYKGVRSHKGVPR

SEQ ID NO: 232 - protein, 24 aa, Artificial Sequence
  honey bee mellitin
MKFLVNVALVFMVVYISYIYADPS

SEQ ID NO: 233 - protein, 535 aa, Artificial Sequence
  Cross-reactive mutant (CRM) of diptheria toxin including, CRM197
GADDVVDSSKSFVMENFSSYHGTKPGYVDSIQKGIQKPKSGTQGNYDDDWKGFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLHDGYAVSWNTVEDSIIRTGFQGESGHDIKITAENTPLPIAGVLLPTIPGKLDVNKSKTHISVNGRKIRMRCRAIDGDVTFCRPKSPVYVGNGVHANLHVAFHRSSSEKIHSNEISSDSIGVLGYQKTVDHTKVNSKLSLFFEIKS

SEQ ID NO: 234 - protein, 164 aa, Artificial Sequence
  Coat protein of Tobacco mosaic virus (TMV)
MMAYSIPTPSQLVYFTENYADYIPFVNRLINARSNSFQTQSGRDELREILIKSQVSVVSPISRFPAEPAYYIYLRDPSISTVYTALLQSTDTRNRVIEVENSTNVTTAEQLNAVRRTDDASTAIHNNLEQLLSLLTNGTGVFNRTSFESASGLTWLVTTTPRTA

SEQ ID NO: 235 - protein, 221 aa, Artificial Sequence
  Coat protein of alfalfa mosaic virus (AMV)
MSSSQKKAGGKAGKPTKRSQNYAALRKAQLPKPPALKVPVAKPTNTILPQTGCVWQSLGTPLSLSSSNGLGARFLYSFLKDFAAPRILEEDLIFRMVFSITPSHAGSFCLTDDVTTEDGRAVAHGNPMQEFPHGAFHANEKFGFELVFTAPTHAGMQNQNFKHSYAVALCLDFDALPEGSRNPSYRFNEVWVERKAFPRAGPLRSLITVGLFDDADDLDRQ

SEQ ID NO: 236 - protein, 237 aa, Artificial Sequence
  Coat protein of Potato virus X
MTTPANTTQATGSTTSTTTKTAGATPATTSGLFTIPDGEFFSTARAIVASNAVATNEDLSKIEAIWKDMKVPTDTMAQAAWDLVRHCADVGSSAQTEMIDTGPYSNGISRARLAAAIKEVCTLRQFCMKYAPVVWNWMLTNNSPPANWQAQGFKPEHKFAAFDFFNGVTNPAAIMPKEGLIRPPSEAEMNAAQTAAFVKITKARAQSNDFASLDAAVTRGRITGTTTAEAVVTLPPP

SEQ ID NO: 237 - protein, 395 aa, Artificial Sequence
  class I outer membrane protein of Neisseria meningitidis
MRKKLTALVLSALPLAAVADVSLYGEIKAGVEGRNYQLQLTEAQAANGGASGQVKVTKVTKAKSRIRTKISDFGSFIGFKGSEDLGEGLKAVWQLEQDVSVAGGGATQWGNRESFIGLAGEFGTLRAGRVANQFDDASQAIDPWDSNNDVASQLGIFKRHDDMPVSVRYDSPEFSGFSGSVQFVPAQNSKSAYKPAYWTTVNTGSATTTTFVPAVVGKPGSDVYYAGLNYKNGGFAGNYAFKYARHANVGRDAFELFLLGSGSDQAKGTDPLKNHQVHRLTGGYEEGGLNLALAAQLDLSENGDKTKNSTTEIAATASYRFGNAVPRISYAHGFDFIERGKKGENTSYDQIIAGVDYDFSKRTSAIVSGAWLKRNTGIGNYTQINAASVGLRHKF

SEQ ID NO: 238 - protein, 347 aa, Artificial Sequence
  Major fimbrial subunit protein type I (Fimbrillin)
MVLKTSNSNRAFGVGDDESKVAKLTVMVYNGEQQEAIKSAENATKVEDIKCSAGQRTLVVMANTGAMELVGKTLAEVKALTTELTAENQEAAGLIMTAEPKTIVLKAGKNYIGYSGTGEGNHIENDPLKIKRVHARMAFTEIKVQMSAAYDNIYTFVPEKIYGLIAKKQSNLFGATLVNADANYLTGSLTTFNGAYTPANYANVPWLSRNYVAPAADAPQGFYVLENDYSANGGTIHPTILCVYGKLQKNGADLAGADLAAAQAANWVDAEGKTYYPVLVNFNSNNYTYDSNYTPKNKIERNHKYDIKLTITGPGTNNPENPITESAHLNVQCTVAEWVLVGQNATW

SEQ ID NO: 239 - protein, 428 aa, Artificial Sequence
  Mycoplasma fermentans macrophage activating lipopeptide (MALP-2)
MKKSKKILLGLSPIAAVLPAVAVSCGNNDESNISFKEKDISKYTTTNANGKQVVKNAELLKLKPVLITDEGKIDDKSFNQSAFEALKAINKQTGIEINSVEPSSNFESAYNSALSAGHKIWVLNGFKHQQSIKQYIDAHREELERNQIKIIGIDFDIETEYKWFYSLQFNIKESAFTTGYAIASWLSEQDESKRVVASFGVGAFPGVTTFNEGFAKGILYYNQKHKSSKIYHTSPVKLDSGFTAGEKMNTVINNVLSSTPADVKYNPHVILSVAGPATFETVRLANKGQYVIGVDSDQGMIQDKDRILTSVLKHIKQAVYETLLDLILEKEEGYKPYVVKDKKADKKWSHFGTQKEKWIGVAENHFSNTEEQAKINNKIKEAIKMFKELPEDFVKYINSDKALKDGNKIDNVSERLEAIISAINKAAK

SEQ ID NO: 240 - protein, 143 aa, Artificial Sequence
  p19 protein of Mycobacterium tuberculosis
ATTLPVQRHPRSLFPEFSELFAAFPSFAGLRPTFDTRLMRLEDEMKEGRYEVRAELPGVDPDKDVDIMVRDGQLTIKAERTEQKDFDGRSEFAYGSFVRTVSLPVGADEDDIKATYDKGILTVSVAVSEGKPTEKHIQIRSTN

SEQ ID NO: 241 - protein, 393 aa, pseudomonas aerugienosa
ALTVNTNIASLNTQRNLNNSSASLNTSLQRLSTGSRINSAKDDAAGLQIANRLTSQVNGLNVATKNANDGISLAQTAEGALQQSTNILQRMRDLSLQSANGSNSDSERTALNGEVKQLQKELDRISNTTTFGGRKLLDGSFGVASFQVGSAANEIISVGIDEMSAESLNGTYFKADGGGAVTAATASGTVDIAIGITGGSAVNVKVDMKGNETAEQAAAKIAAAVNDANVGIGAFSDGDTISYVSKAGKDGSGAITSAVSGVVIADTGSTGVGTAAGVTPSATAFAKTNDTVAKIDISTAKGAQSAVLVIDEAIKQIDAQRADLGAVQNRFDNTINNLKNIGENVSAARGRIEDTDFAAETANLTKNQVLQQAGTAILAQANQLPQSVLSLLR

SEQ ID NO: 242 - protein, 287 aa, Listeria monocytogenes
MKVNTNIISLKTQEYLRKNNEGMTQAQERLASGKRINSSLDDAAGLAVVTRMNVKSTGLDAASKNSSMGIDLLQTADSALSSMSSILQRMRQLAVQSSNGSFSDEDRKQYTAEFGSLIKELDHVADTTNYNNIKLLDQTATNAATQVSIQASDKANDLINIDLFNAKGLSAGTITLGSGSTVAGYSALSVADADSSQEATEAIDELINNISNGRALLGAGMSRLSYNVSNVNNQSIATKASASSIEDADMAAEMSEMTKYKILTQTSISMLSQANQTPQMLTQLINS

SEQ ID NO: 243 - DNA, 46 bp, Artificial Sequence
  primers
gaattccatatgagcttgctgactgaggttgagaccccgattcgca

SEQ ID NO: 244 - DNA, 67 bp, Artificial Sequence
  primers
gacgtggctcagcttattaatggtgatgatggtgatgtctagacgggtctgagctatcgttagagcg

SEQ ID NO: 245 - protein, 12 aa, Artificial Sequence
  M2e epitopes
EWGCRCNDSSDP

SEQ ID NO: 246 - protein, 12 aa, Artificial Sequence
  M2e epitopes
NEWGCRCNDSSD

SEQ ID NO: 247 - protein, 12 aa, Artificial Sequence
  M2e epitopes
RNEWGCRCNDSS

SEQ ID NO: 248 - protein, 12 aa, Artificial Sequence
  M2e epitopes
IRNEWGCRCNDS

SEQ ID NO: 249 - protein, 12 aa, Artificial Sequence
  M2e epitopes
PIRNEWGCRCND

SEQ ID NO: 250 - protein, 12 aa, Artificial Sequence
  M2e epitopes
TPIRNEWGCRCN

SEQ ID NO: 251 - protein, 12 aa, Artificial Sequence
  M2e epitopes
ETPIRNEWGCRC

SEQ ID NO: 252 - protein, 12 aa, Artificial Sequence
  M2e epitopes
VETPIRNEWGCR

SEQ ID NO: 253 - protein, 12 aa, Artificial Sequence
  M2e epitopes
EVETPIRNEWGC

SEQ ID NO: 254 - protein, 12 aa, Artificial Sequence
  M2e epitopes
TEVETPIRNEWG

SEQ ID NO: 255 - protein, 12 aa, Artificial Sequence
  M2e epitopes
LTEVETPIRNEW

SEQ ID NO: 256 - protein, 12 aa, Artificial Sequence
  M2e epitopes
LLTEVETPIRNE

SEQ ID NO: 257 - protein, 12 aa, Artificial Sequence
  M2e epitopes
SLLTEVETPIRN